(19) United States
(12) Patent Application Publication
(10) Pub. No.: US 2020/0102584 A1
(43) Pub. Date: Apr. 2, 2020

(54) FDCA-decarboxylating monooxygenase-deficient host cells for producing FDCA

(71) Applicant: Purac Biochem B.V., Gorinchem (NL)

(72) Inventors: Johannes Adrianus Maria de Bont, Wageningen (NL); Harald Johan Ruijssenaars, Gorinchem (NL); Jan Werij, Gorinchem (NL)

(73) Assignee: Purac Biochem B.V., Gorinchem (NL)

(21) Appl. No.: 16/495,845

(22) PCT Filed: Mar. 21, 2018
(86) PCT No.: PCT/EP2018/057140

§ 371 Date: Sep. 20, 2019

(30) Foreign Application Priority Data
Mar. 21, 2017 (EP) 17162104.8

(51) Int. Cl. C12P 17/04; C07K 14/385; C12N 9/02

(52) U.S. Cl. CPC C12P 17/04; C12N 9/0073; C07K 14/385

(57) ABSTRACT

The invention relates to fungal cells for the production of FDCA. The fungal cell has genetic modification that reduces specific 2,5-furandicarboxylic acid (FDCA) decarboxylating monooxygenase activity in the cell, as compared to a corresponding parent cell lacking the genetic modification. The fungal cell can further be genetically modified to increase the cell's ability to oxidize furanic aldehydes to the corresponding furanic carboxylic acids. The invention also relates to a process for the production of 2,5-furan-dicarboxylic acid (FDCA) wherein the cells of the invention are used for oxidation of a furanic precursors of FDCA to FDCA.

### FIELD OF THE INVENTION

The invention relates to the fields of molecular genetics, metabolic engineering, biotransformation and fermentation technology. In particular, the invention relates to fungi that are genetically modified to produce 2,5-furandicarboxylic acid from hydroxymethylfurfural. The invention further relates to the use of such fungi in processes for the biotransformation of hydroxymethylfurfural into 2,5-furandicarboxylic acid.

### BACKGROUND OF THE INVENTION

2,5-furandicarboxylic acid (FDCA) is a monomeric compound which can be applied in the production of polyesters which have a tremendous economic impact. A very important compound in the field is polyethyleneterephthalate (PET) which is produced from terephthalic acid (PTA) and ethylene glycol. FDCA may substitute for PTA in the polyester PET in which case polyethylenefurandicarboxylate (PEF) results. PEF has a good potential in replacing PET in the large polyester market. Not only because it has superior properties when compared to PET, but also because it can be derived from renewable feedstocks. FDCA can be produced from sugars either chemically (De Jong et al., 2012. In: Biobased Monomers, Polymers, and Materials; Smith, P., et al.; ACS Symposium Series; American Chemical Society: Washington, D.C.) or in a combined chemical-biological route (Wiercks et al., 2011. Appl Microbiol Biotechnol 92:1095-1105). In the latter case, a monomeric sugar such as glucose or fructose is chemically transformed into 5-(hydroxymethyl)-2-furaldehyde (HMF) which subsequently can be oxidized by enzymes into FDCA.

A biological route for producing FDCA from HMF has been developed based on the isolation of the HMF-degrading strain of Cupriavidus basilensis HMF14 (Wierckx et al., 2010. Microbial Technology 3:336-343). A cluster of genes encoding enzymes involved in the HMF degradation route in C. basilensis HMF14 was identified and relevant genes heterologously expressed in a Pseudomonas putida S12 strain (Koopman et al., 2010. PNAS 107:4919-4924) which thereby acquired the ability to metabolize HMF. The heterologous expression of only the hmfH gene—encoding a HMF oxidoreductase that acts as an oxidase mainly at HMF-acid (HMFCA), but it also may oxidize HMF or FFCA—enables P. putida S12 to produce FDCA from HMF (Koopman et al., 2010. Bioresource Technology 101:6291-6296; and WO 2011/026913). In further optimization work (Wierckx et al., 2011, supra; and WO 2012/064195), two additional genes were expressed in P. putida S12 that encode for an HMFCA transporter and for an aldehyde dehydrogenase with unknown specificity, respectively.

U.S. Pat. No. 7,067,303 disclose that the fungus Coniochaeta ligniaria (teleomorph), or its Lecythophora anamorph state, are capable of significantly depleting the toxic levels of furans, particularly furfural and HMF, in agricultural biomass hydrolysate. The use of C. ligniaria as a biological agent in detoxifying sugar-containing hydrolysates was further demonstrated in a number of subsequent papers (López et al., 2004. Appl. Microbiol Biotechnol 64:125-131; Nichols et al., 2005. Appl Biochem Biotechnol. Spring; 121-124:379-90; Nichols et al., 2008. Enzyme and Microbial Technology 42:624-630; Nichols et al., 2010. Bioresource Technol 19:7545-50; Nichols et al., 2014. Biomass and Bioenergy 67:79-88). Apart from detoxification of HMF to less toxic compounds, the organism was also able to metabolize HMF for growth.

Zhang et al. (2010, Biotechnology for Biofuels 3:26) described the isolation of two HMF-metabolizing fungi that detoxified their corn stover hydrolysate, which were identified as Amorphotheca resinae and Eupenicillium baarnense, respectively. In a subsequent paper (Ran et al., 2014, Biotechnology for Biofuels 7:51) growth of the A. resinae strain, designated as ZN1, was reported to be supported by many compounds including HMF. HMF was degraded and HMF alcohol and HMFCA accumulated over time but no accumulation of FDCA was reported.

Govinda Rajulu et al. (2014, Mycological Progress 13:1049-1056) similarly isolated a number of fungi with the ability to utilize furfural and/or HMF as sole carbon source but again, no accumulation of FDCA was reported.

Thus, several fungi have been described that either grow at the expense of HMF or detoxify HMF-containing feedstocks. As with yeasts, the organisms were studied from the perspective of reducing HMF into HMF-alcohol for the purpose of detoxifying feedstocks. Production of FDCA by yeast or filamentous fungi, however, has not been described. Yet, fungal production of FDCA from HMF would offer several intrinsic advantages over the bacterial processes in the art. E.g., many fungi tolerate low pH values down to pH=3 or lower for growth, whereas most bacteria prefer neutral pH environments. In the specific situation of large-scale production of FDCA it would be of great advantage if whole-cell production methodologies at low pH-values would be available because of advantages in downstream processing (DSP) and for combating infections.

It is therefore an object of the present invention to provide for fungal cells and their use in processes for the production of FDCA from HMF.

### SUMMARY OF THE INVENTION

In a first aspect the present invention relates to a fungal cell comprising a genetic modification that reduces specific 2,5-furandicarboxylic acid (FDCA) decarboxylating monooxygenase activity in the cell, as compared to a corresponding parent cell lacking the genetic modification, wherein the genetic modification eliminates the expression of an endogenous gene encoding an FDCA decarboxylating monooxygenase by deletion of at least a part of at least one of the promoter and the coding sequence of the gene. Preferably, in the cell, the endogenous gene in the corresponding parent cell encodes a FDCA decarboxylating monooxygenase comprising an amino acid sequence with at least 45% sequence identity to at least one of SEQ ID NO: 4. It is further preferred that the genetic modification comprises or consists of the deletion of at least the complete coding sequence of an endogenous gene encoding an FDCA decarboxylating monooxygenase from the gene in the cell's genome. In cells comprising more than one copy of the endogenous gene, preferably, the expression of all copies of the endogenous gene encoding an FDCA decarboxylating monooxygenase is eliminated.

The fungal cell according to the invention preferably is a cell that has the natural ability to oxidise HMF to FDCA.

A preferred fungal cell according to the invention preferably comprises a further genetic modification that is at least one of: a) a genetic modification that confers to the cell the ability to oxidize 5-hydroxymethyl-2-furancarboxylic acid (HMFCA) to 5-formyl-2-furoic acid (FFCA) or that increases in the cell the specific activity of a enzyme that oxidizes HMFCA to FFCA as compared to a corresponding wild type cell lacking the genetic modification; and, b) a genetic modification that confers to the cell the ability to oxidize furanic aldehydes to the corresponding furanic carboxylic acids or a genetic modification that increases in the cell the specific activity of a enzyme that oxidizes furanic aldehydes to the corresponding furanic carboxylic acids, as compared to a corresponding wild type cell lacking the genetic modification. Preferably, in the cell, the genetic modification in a) is a modification that increases expression of a nucleotide sequence encoding a polypeptide with HMFCA dehydrogenase activity, which polypeptide comprises an amino acid sequence that has at least 45% sequence identity with the amino acid sequence of at least one of SEQ ID NO.'s: 1 and 2; and/or, the genetic modification in b) is a modification that increases expression of a nucleotide sequence encoding a polypeptide having furanic aldehyde dehydrogenase activity, which aldehyde dehydrogenase has at least one of the abilities of i) oxidizing HMF to HMFCA, ii) oxidizing DFF to FFCA, and, iii) oxidizing FFCA into FDCA, which polypeptide comprises an amino acid sequence that has at least 45% sequence identity with the amino acid sequence of SEQ ID NO.: 3.

A fungal cell according to the invention preferably further comprises a genetic modification selected from: a) a genetic modification that reduces or eliminates the expression of a gene encoding a short chain dehydrogenase that reduces HMF and/or FFCA to the corresponding alcohol, wherein preferably the gene is at least one of a gene encoding polypeptide comprising an amino acid sequence with at least 45% sequence identity to at least one of SEQ ID NO: 5 and 25; b) a genetic modification that increases expression of a nucleotide sequence encoding a polypeptide that transports at least one furanic compound, which polypeptide preferably comprises an amino acid sequence that has at least 45% sequence identity with the amino acid sequence of at least one of SEQ ID NO.'s: 6-10; and, c) a genetic modification that alters the expression of a gene encoding a transcriptional activator of genes involved in furan catabolism, wherein preferably the gene is a gene encoding a polypeptide comprising an amino acid sequence with at least 45% sequence identity to SEQ ID NO: 11.

In a separate aspect the invention pertains to a fungal cell that has the ability to oxidise HMF to FDCA and which cell comprises a genetic modification that increases expression of a nucleotide sequence encoding a polypeptide that transports at least one furanic compound, which polypeptide preferably comprises an amino acid sequence that has at least 45% sequence identity with the amino acid sequence of at least one of SEQ ID NO.'s: 9 and 10. Preferably a cell according to this aspect comprises a further genetic modification selected from: a) a genetic modification that eliminates or reduces specific FDCA decarboxylating monooxygenase activity in the cell, as compared to a corresponding parent cell lacking the genetic modification; b) a genetic modification that confers to the cell the ability to oxidize HMFCA to FFCA or that increases in the cell the specific activity of a enzyme that oxidizes HMFCA to FFCA as compared to a corresponding wild type cell lacking the genetic modification; c) a genetic modification that confers to the cell the ability to oxidize furanic aldehydes to the corresponding furanic carboxylic acids or a genetic modification that increases in the cell the specific activity of a enzyme that oxidizes furanic aldehydes to the corresponding furanic carboxylic acids, as compared to a corresponding wild type cell lacking the genetic modification; d) a genetic modification that reduces or eliminates the expression of a gene encoding a short chain dehydrogenase that reduces HMF and/or FFCA to the corresponding alcohol, wherein preferably the gene is at least one of a gene encoding polypeptide comprising an amino acid sequence with at least 45% sequence identity to at least one of SEQ ID NO: 5 and 25; e) a genetic modification that increases expression of a nucleotide sequence encoding a polypeptide that transports at least one furanic compound, which polypeptide preferably comprises an amino acid sequence that has at least 45% sequence identity with the amino acid sequence of at least one of SEQ ID NO.'s: 6-8; and, f) a genetic modification that alters the expression of a gene encoding a transcriptional activator of genes involved in furan catabolism, wherein preferably the gene is a gene encoding a polypeptide comprising an amino acid sequence with at least 45% sequence identity to SEQ ID NO: 11.

A cell according to the above aspects of the invention preferably is a filamentous fungal cell selected from a genus from the group consisting of: Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, Trichoderma, and Ustilago, more preferably the cell is a filamentous fungal cell selected from a species from the group consisting of: Aspergillus niger, Aspergillus awamori, Aspergillus foetidus, Aspergillus sojae, Aspergillus fumigatus, Talaromyces emersonii, Aspergillus oryzae, Myceliophthora thermophila, Trichoderma reesei, Penicillium chrysogenum, Penicillium simplicissimum and Penicillium brasilianum; or, the cell is a yeast cell selected from a genus from the group consisting of: Saccharomyces, Kluyveromyces, Candida, Pichia, Schizosaccharomyces, Hansenula, Kloeckera, Schwanniomyces, Yarrowia, Cryptococcus, Debaromyces, Saccharomycecopsis, Saccharomycodes, Wickerhamia, Debayomyces, Hanseniaspora, Ogataea, Kuraishia, Komagataella, Metschnikowia, Williopsis, Nakazawaea, Torulaspora, Bullera, Rhodotorula, and Sporobolomyces, more preferably the cell is a yeast cell selected from a species from the group consisting of Kluyveromyces lactis, S. cerevisiae, Hansenula polymorpha, Yarrowia lipolytica, Candida tropicalis and Pichia pastoris.

In a further aspect the invention relates to a process for oxidizing HMFCA to FFCA, the process comprising the step of incubating a fungal cell according to the invention, in the presence of HMFCA, under conditions conducive to the oxidation of HMFCA by the cell, wherein the cell expresses enzymes that have the ability to oxidize HMFCA to FFCA.

In another aspect, the invention relates to a process for producing FDCA, the process comprising the step of incubating a fungal cell according to the invention, in a medium comprising one or more furanic precursors of FDCA, preferably under conditions conducive to the oxidation of furanic precursors of FDCA by the cell to FDCA, and, optionally recovery of the FDCA, wherein preferably, at least one furanic precursor of FDCA is selected from the group consisting of HMF, 2,5-dihydroxymethyl furan (DHF), HMFCA, FFCA and 2,5-diformyl furan (DFF), of which HMF is most preferred, wherein the furanic precursors of FDCA are obtained from one or more hexose sugars, preferably one or more hexose sugars obtained from lignocellulosic biomass, preferably by acid-catalyzed dehydration, and, wherein preferably the FDCA is recovered from the medium by a process comprising acid precipitation followed by cooling crystallization and/or solvent extraction. Preferably, in the process, the medium has a pH in the range of 2.0-3.0, wherein preferably the FDCA precipitates from the acidic medium in which it is produced and is recovered from the medium by a process comprising acid precipitation followed by cooling crystallization.

In yet a further aspect the invention pertians to a process for producing a polymer from at least two FDCA monomers, the process comprising the steps of: a) preparing an FDCA monomer in a process according to the invention; and, b) producing a polymer from the FDCA monomer obtained in a), wherein preferably the polymer is produced by mixing the FDCA monomer and a diol monomer and bringing the mixture in a condition under which the FDCA monomer and the diol monomer polymerise.

In a final aspect, the invention relates to the use of a fungal cell according to the invention, for the biotransformation of one or more of furanic precursors to FDCA or a fungal cell expressing one or more bacterial enzymes with the ability to convert a furanic precursors of FDCA into FDCA, wherein preferably, at least one furanic precursor of FDCA is selected from the group consisting of HMF, DHF, HMFCA, FFCA and DFF, of which HMF is most preferred.

### DESCRIPTION OF THE INVENTION

### Definitions

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWlN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall lengths, local alignments, such as those using the Smith Waterman algorithm, are preferred.

Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), |
| | Asn (N), and Gln (Q) |
| Aliphatic Uncharged Residues | Gly (G), Ala (A), Val (V), |
| | Leu (L), and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes.

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative Physical and Functional Classifications of Amino Acid Residues.

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, |
| | M, R, T, V, W, and Y |
| Negatively charged residues | D and E |
| Polar residues | C, D, E, H, K, |
| | N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, |
| | N, Q, R, S, P and T |
| Flexible residues | Q, T, K, S, G, P, D, E, and R |

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6× sodium chloride/sodium citrate (SSC) at about 45° C., followed by one or more washes in 1×SSC, 0.1% SDS at about 50° C., preferably at about 55° C., preferably at about 60° C. and even more preferably at about 65° C.

Highly stringent conditions include, for example, hybridization at about 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3^{rd} edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. The terms “expression vector” or “expression construct” refer to nucleotide sequences that are capable of effecting expression of a gene in host cells or host organisms compatible with such sequences. These expression vectors typically include at least suitable transcription regulatory sequences and optionally, 3′ transcription termination signals. Additional factors necessary or helpful in effecting expression may also be present, such as expression enhancer elements. The expression vector will be introduced into a suitable host cell and be able to effect expression of the coding sequence in an in vitro cell culture of the host cell. The expression vector will be suitable for replication in the host cell or organism of the invention.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer.

The term “selectable marker” is a term familiar to one of ordinary skill in the art and is used herein to describe any genetic entity which, when expressed, can be used to select for a cell or cells containing the selectable marker. The term “reporter” may be used interchangeably with marker, although it is mainly used to refer to visible markers, such as green fluorescent protein (GFP). Selectable markers may be dominant or recessive or bidirectional.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide. The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which it is introduced, but have been obtained from another cell or synthetically or recombinantly produced. Generally, though not necessarily, such nucleic acids encode proteins, i.e. exogenous proteins, that are not normally produced by the cell in which the DNA is transcribed or expressed. Similarly exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

The “specific activity” of an enzyme is herein understood to mean the amount of activity of a particular enzyme per amount of total host cell protein, usually expressed in units of enzyme activity per mg total host cell protein. In the context of the present invention, the specific activity of a particular enzyme may be increased or decreased as compared to the specific activity of that enzyme in an (otherwise identical) wild type host cell.

“Furanic compounds” are herein understood to be 2,5-furan-dicarboxylic acid (FDCA) as well as any compound having a furan group which may be oxidized to FDCA, the latter being referred to herein as a “precursor of FDCA” or a “furanic precursor of FDCA”. Precursors of FDCA at least include: 5-hydroxymethylfurfural (HMF), 2,5-dihydroxymethyl furan (DHF) or 2,5-bis(hydroxymethyl)furan (BHF) referred to as HMF-OH, 5-hydroxymethyl-2-furancarboxylic acid or 5-hydroxymethyl-2-furoic acid (HMFCA), 5-formyl-2-furoic acid (FFCA), and 2,5-diformyl furan (DFF). It is further understood that in the “furanic compounds”, the furan ring or any or its substitutable sidegroup may be substituted, e.g. with OH, C1-C10 alkyl, alkyl, allyl, aryl or RO-ether moiety, including cyclic groups, in the furan ring on any available position.

“Aerobic conditions” “Oxic conditions” or an aerobic or oxic fermentation process is herein defined as conditions or a fermentation process run in the presence of oxygen and in which oxygen is consumed, preferably at a rate of at least 0.5, 1, 2, 5, 10, 20 or 50 mmol/L/h, and wherein organic molecules serve as electron donor and oxygen serves as electron acceptor.

“Anaerobic or anoxic conditions” or an “anaerobic or anoxic fermentation process” is herein defined as conditions or a fermentation process run substantially in the absence of oxygen and wherein organic molecules serve as both electron donor and electron acceptors. Under anoxic conditions substantially no oxygen is consumed, preferably less than 5, 2, 1, or 0.5 mmol/L/h, more preferably 0 mmol/L/h is consumed (i.e. oxygen consumption is not detectable), or substantially no dissolved oxygen can be detected in the fermentation medium, preferably the dissolved oxygen concentration in the medium is less than 2, 1, 0.5, 0.2, 0.1% of air saturation, i.e. below the detection limit of commercial oxygen probes.

Any reference to nucleotide or amino acid sequences accessible in public sequence databases herein refers to the version of the sequence entry as available on the filing date of this document.

### Description of the Embodiments

### The Parent Host Cell

The present invention concerns the genetic modification of a host cell so as to enable the host cell to produce 2,5-furandicarboxylic acid (FDCA) from suitable furanic precursors. To this end a number of genetic modifications can be introduced in a parent host cell in accordance with the invention. These modifications include the introduction of expression of a number of heterologous genes, as well as, the modification of the expression of a number of endogenous genes already present in the parent host cell, by reducing or eliminating of some endogenous genes and/or by increasing the expression, i.e. overexpressing, other endogenous genes. These genetic modification are further set out below herein. A parent host cell is thus understood to be a host cell prior to that any of the genetic modifications in accordance with the invention have been introduced in the host cell.

A parent host cell of the invention preferably is a host cell that naturally has the ability to metabolise furanic compounds. More preferably the parent host cell at least has the natural ability to degrade FDCA. The parent host cell of the invention can further have the natural ability to oxidise HMF to FDCA. Whether or not a given host cell strain naturally has the ability to oxidise HMF to FDCA and/or to degrade FDCA can be tested by determining the strain's ability to grow at the expense of one or more of HMF, HMF-alcohol, HMFCA and FDCA, preferably as sole carbon source, as e.g. described in the Examples herein. Preferably, the parent host cell comprises an endogenous gene encoding an enzyme that catalyses the degradation of FDCA. Preferably the endogenous gene is a functional gene expressing an enzyme that catalyses the degradation of FDCA. Preferably, the enzyme that catalyses the degradation of FDCA is an FDCA-decarboxylating monooxygenase as further defined herein.

A parent host cell of the invention can be any suitable host cell including e.g. eukaryotic cells such as a mammalian, insect, plant, fungal, or algal cell. Preferably, however, the host cell is a microbial cell. The microbial host cell can be a prokaryotic cell, preferably a bacterial cell, including e.g. both Gram-negative and Gram-positive microorganisms.

More preferably, however, a parent host cell of the invention is a eukaryotic microbial host cell, such as e.g. a fungal host cell. A most preferred parent host cell to be modified in accordance with the invention is a yeast or filamentous fungal host cell.

“Fungi” are herein defined as eukaryotic microorganisms and include all species of the subdivision Eumycotina (Alexopoulos, C. J., 1962, In: Introductory Mycology, John Wiley & Sons, Inc., New York). The terms “fungus” and “fungal” thus include or refers to both filamentous fungi and yeast.

“Filamentous fungi” are herein defined as eukaryotic microorganisms that include all filamentous forms of the subdivision Eumycotina and Oomycota (as defined in “Dictionary of The Fungi”, 10th edition, 2008, CABI, UK, www.cabi.org). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. Filamentous fungal strains include, but are not limited to, strains of Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, Trichoderma, and Ustilago.

Preferred filamentous fungal species as parent host cells for the invention belong to a species of an Aspergillus, Myceliophthora, Penicillium, Talaromyces or Trichoderma genus, and more preferably a species selected from Aspergillus niger, Aspergillus awamori, Aspergillus foetidus, Aspergillus sojae, Aspergillus fumigatus, Talaromyces emersonii, Aspergillus oryzae, Myceliophthora thermophila, Trichoderma reesei, Penicillium chrysogenum, Penicillium simplicissimum and Penicillium brasilianum. Suitable strains of these filamentous fungal species are available from depository institutions known per se to the skilled person.

“Yeasts” are herein defined as eukaryotic microorganisms and include all species of the subdivision Eumycotina (Yeasts: characteristics and identification, J. A. Barnett, R. W. Payne, D. Yarrow, 2000, 3rd ed., Cambridge University Press, Cambridge UK; and, The yeasts, a taxonomic study, CP. Kurtzman and J. W. Fell (eds) 1998, 4th ed., Elsevier Science Publ. B. V., Amsterdam, The Netherlands) that predominantly grow in unicellular form. Yeasts may either grow by budding of a unicellular thallus or may grow by fission of the organism. Preferred yeasts cells for use in the present invention belong to the genera Saccharomyces, Kluyveromyces, Candida, Pichia, Schizosaccharomyces, Hansenula, Kloeckera, Schwanniomyces, Yarrowia, Cryptococcus, Debaromyces, Saccharomycecopsis, Saccharomycodes, Wickerhamia, Debayomyces, Hanseniaspora, Ogataea, Kuraishia, Komagataella, Metschnikowia, Nakazawaea, Torulaspora, Bullera, Rhodotorula, and Sporobolomyces. A parental yeast host cell can be a cell that is naturally capable of anaerobic fermentation, more preferably alcoholic fermentation and most preferably anaerobic alcoholic fermentation. More preferably yeasts from species such as Kluyveromyces lactis, S. cerevisiae, Hansenula polymorpha (new name: Ogataea henricii), Yarrowia lipolytica, Candida tropicalis and Pichia pastoris (new name: Komagataella pastoris).

Particularly when compared to bacteria, fungi, have many attractive features for industrial fermentation processes, including e.g. their high tolerance to acids, ethanol and other harmful compounds, their high osmo-tolerance, their high fermentative capacity and for some yeasts their capability of anaerobic growth.

The host cell further preferably has a high tolerance to low pH, i.e. capable of growth at a pH equal to or lower than 5.0, 4.0, 3.5, 3.2, 3.0, 2.9, 2.8, 2.7, 2.6, 2.5, 2.4, 2.3 or 2.28 and towards organic acids like lactic acid, acetic acid or formic acid and furanic acids and a high tolerance to elevated temperatures. Any of these characteristics or activities of the host cell may be naturally present in the host cell or may be introduced or modified by genetic modification, preferably by self cloning or by the methods of the invention described below.

A suitable cell is a cultured cell, a cell that may be cultured in fermentation process e.g. in submerged or solid state fermentation.

For specific uses of a compound produced in a fungal host cell according to the invention, the selection of the host cell may be made according to such use. Where e.g. the compound produced in a host cell according to the invention is to be used in food applications, a host cell may be selected from a food-grade organism such as e.g. a Saccharomyces species, e.g. S. cerevisiae, a food-grade Penicillium species or Yarrowia lipolitica. Specific uses include, but are not limited to, food, (animal) feed, pharmaceutical, agricultural such as crop-protection, and/or personal care applications.

### A Genetically Modified Cell

In a first aspect, the invention pertains to a cell, preferably a fungal cell comprising a genetic modification. The genetic modification of the cell preferably is at least a genetic modification that reduces or eliminates the specific activity of an enzyme that catalyses the degradation of 2,5-furandicarboxylic acid (FDCA), as compared to a corresponding wild type cell lacking the genetic modification.

A cell of the invention further preferably comprises one of: a) a genetic modification that confers to the cell the ability to oxidize 5-hydroxymethyl-2-furancarboxylic acid (HMFCA) to 5-formyl-2-furoic acid (FFCA) or that increases in the cell the specific activity of an enzyme that oxidizes HMFCA to FFCA as compared to a corresponding wild type cell lacking the genetic modification; and/or, b) a genetic modification that confers to the cell the ability to oxidize furanic aldehydes to the corresponding furanic carboxylic acids or that increases in the cell the specific activity of an enzyme that oxidizes furanic aldehydes to the corresponding furanic carboxylic acids, as compared to a corresponding wild type cell lacking the genetic modification.

Preferred cells having these genetic modifications are further specified herein below.

### Reducing or Eliminating Specific 2,5-Furandicarboxylic Acid (FDCA) Decarboxylating Monooxygenase Activity

A cell of the invention preferably is a cell that lacks the ability to degrade FDCA. A cell of the invention will usually be a genetically modified cell of fungal species that naturally has the ability to degrade FDCA, which cell has been genetically modified to reduce or eliminate its natural ability to degrade FDCA. Whether or not a given fungal strain naturally has the ability to degrade FDCA can be tested by determining the strains ability to grow at the expense of one or more of HMF, HMF-alcohol, HMFCA and FDCA as sole carbon source, as e.g. described in the Examples herein. An example of a fungal species that naturally has the ability to degrade FDCA is Penicillium brasilianum as shown in the Examples herein, or Aspergillus niger (Rumbold et al., 2010, Bioengineered Bugs 1:5, 359-366). In contrast, yeasts such as Saccharomyces and Yarrowia species, are examples of fungal species that naturally lack the ability to degrade FDCA.

Thus, in one embodiment of the invention, the cell is genetically modified to reduce or eliminate the cell's natural ability to degrade FDCA. A gene to be modified for reducing or eliminating the cell's ability to degrade FDCA preferably is a gene encoding an FDCA decarboxylating monooxygenase.

A gene encoding an FDCA decarboxylating monooxygenase to be modified for reducing or eliminating the specific FDCA decarboxylating monooxygenase activity in the cell of the invention, preferably is a gene encoding an amino acid sequence with at least 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 96, 97, 98, 99 or 100% sequence identity to at least one of SEQ ID NO.: 4 (hmfK1). A suitable hmfK1 orthologue be modified for reducing or eliminating the specific FDCA decarboxylating monooxygenase activity in the cell is e.g. the Aspergillus niger hmfK1 orthologue with acc. no. XP 001397353.2 (SEQ ID NO: 26). In the cells of the invention, the specific FDCA decarboxylating monooxygenase activity is preferably reduced by at least a factor 1.05, 1.1, 1.2, 1.5, 2.0, 5.0, 10, 20, 50 or 100 as compared to cells of a strain which is genetically identical except for the genetic modification causing the reduction in activity.

Whether or not an orthologue of the Penicillium brasilianum hmfK1 encodes a polypeptide that has FDCA decarboxylating monooxygenase activity can be assayed by expression of the polypeptide in a suitable host cell that is incapable of degrading FDCA and detecting whether or not expression of the polypeptide confers to the cell the ability to degrade FDCA. FDCA decarboxylating monooxygenase activity can e.g. be assayed using an expression construct wherein a nucleotide sequence encoding the polypeptide to be assayed for FDCA decarboxylating monooxygenase is expressed in a P. putida host cell and testing transformant for their ability to degrade FDCA, as is e.g. described in the Examples of PCT/EP2016/072406. Alternatively, the hmfK1 orthologue can be assayed for its ability to complement a Penicillium brasilianum hmfK1⁻ disruption mutant as described in the Examples herein by testing for their ability to restore the mutant ability to grow on FDCA as sole carbon source.

The nucleotide sequences of the invention, encoding an FDCA decarboxylating monooxygenase, the specific activities of which are preferably reduced or eliminated in a cell of the invention, are obtainable from and may be identified in genomic and/or cDNA of a fungus, yeast or bacterium, e.g. one that belongs to the same phylum, class or genus as the source organisms described above, using methods for isolation of nucleotide sequences that are well known in the art per se (see e.g. Sambrook and Russell (2001) “Molecular Cloning: A Laboratory Manual (3^{rd} edition), Cold Spring Harbor Laboratory, Cold Spring Harbor Laboratory Press, New York). The nucleotide sequences of the invention are e.g. obtainable in a process wherein a) degenerate PCR primers (designed on the basis of conserved amino acid sequences) are used on genomic and/or cDNA of a suitable organism to generate a PCR fragment comprising part of the nucleotide sequences encoding enzymes the specific activities of which are preferably reduced or eliminated in a cell of the invention; b) the PCR fragment obtained in a) is used as probe to screen a cDNA and/or genomic library of the organism; and c) producing a cDNA or genomic DNA comprising the nucleotide sequence encoding the enzyme the specific activities of which is preferably reduced or eliminated in a cell of the invention. Such conserved sequences can be identified in the sequences alignments presented in Table 4, wherein invariable positions are indicated with a “*” and strongly conserved positions are indicated with a “:”. Also suitable host cells of the invention can be derived from Table 4 wherein the host preferably is a non-pathogenic fungus or yeast that belongs to the same phylum, class, order, family or genus as the source organism of an orthologue identified in Table 4. Table 4 presents the amino acid sequence alignments of Penicillium brasilianum hmfK1 with its 10 closest orthologues as available in public databases. Table 4A provides the percentages amino acid identities among the P. brasilianum sequence and its orthologues, as well as the accession numbers of the orthologues.

In a preferred embodiment therefore, the invention pertains to a fungal cell comprising a genetic modification that reduces specific FDCA decarboxylating monooxygenase activity in the cell, as compared to a corresponding parent cell lacking the genetic modification, wherein the genetic modification eliminates the expression of an endogenous gene encoding an FDCA decarboxylating monooxygenase by deletion of at least a part of at least one of the promoter and the coding sequence of the gene from the genome of the cell. Preferably at least a part of the coding sequence of the FDCA decarboxylating monooxygenase gene is deleted from the cell's genome, e.g. at least 10, 20, 30, 40, 50, 60, 70, 80, 90 or 100% of the coding sequence is deleted. A deletion of at least a part of the coding sequence preferably is a deletion that causes a frameshift and/or a deletion that removes the translation initiation codon. Most preferably however at least the complete coding sequence of an endogenous gene encoding an FDCA decarboxylating monooxygenase is deleted.

It is further preferred that in the genetically modified fungal cell of the invention, the expression of all copies of the endogenous gene encoding an FDCA decarboxylating monooxygenase is eliminated. Thus, if the corresponding parental cell lacking the genetic modification comprises more than one endogenous copies of the gene encoding an FDCA decarboxylating monooxygenase, e.g. as a result of di- aneu- or polyploidy and/or gene amplification, preferably the expression of each and every copy of the gene encoding an FDCA decarboxylating monooxygenase is eliminated.

Methods and means for effecting deletion of sequences from fungal genomes are described herein below.

A genetically modified fungal cell of the invention can further comprises a genetic modification that reduces or eliminates the specific activity of another hydroxylase that was found to be induced by HMF, i.e. the P. brasilianum hmfK3 encoded hydroxylase or an orthologue thereof. Although under the conditions used in the Examples herein disruption of the hmfK3-encoded hydroxylase in P. brasilianum did not affect the ability of the fungus to degrade FDCA, this may be different in other fungi and/or under different condition. Under such circumstances it can be useful that a fungal cell of the invention comprises a genetic modification that reduces or eliminates the specific activity of and hydroxylase comprising an amino acid sequence with at least 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 96, 97, 98, 99 or 100% sequence identity to at least one of SEQ ID NO.: 23 (hmfK3). Preferably, in the cells of the invention, the specific hmfK3-encoded hydroxylase activity is preferably reduced by at least a factor 1.05, 1.1, 1.2, 1.5, 2.0, 5.0, 10, 20, 50 or 100 as compared to cells of a strain which is genetically identical except for the genetic modification causing the reduction in activity.

However, the Examples herein show that disruption of the hmfK1-encoded FDCA decarboxylating monooxygenase is sufficient for eliminating the cell's ability to degrade FDCA. Therefore, a genetically modified fungal cell comprising an intact and/or functional endogenous hmfK3 gene or orthologue thereof is expressly included in the invention.

### Introducing or Increasing HMFCA Dehydrogenase Activity

A cell of the invention preferably is a cell that has the ability of oxidizing 5-hydroxymethyl-2-furancarboxylic acid (HMFCA) to 5-formylfuroic acid (FFCA). The cell's ability of oxidizing HMFCA to FFCA can be an endogenous activity of the cell, such as in e.g. Penicillium brasilianum, or it can be an exogenous activity to be conferred to the cell, such as e.g. for Aspergillus niger. Preferably, the ability of oxidizing HMFCA to FFCA is conferred to the cell or increased in the cell by a genetic modification of the cell, e.g. a transformation of the cell with a nucleic acid construct comprising a nucleotide sequence encoding a dehydrogenase or an oxidase that has the ability to oxidize HMFCA to FFCA. The dehydrogenase preferably is an alcohol dehydrogenase (i.e. having EC 1.1 activity). Thus, the cell is preferably a cell comprising an expression construct for expression of a nucleotide sequence encoding a dehydrogenase or an oxidase that has the ability to oxidize HMFCA to FFCA. In a preferred cell of the invention, the expression construct is expressible in the cell and expression of the dehydrogenase or oxidase preferably confers to in the cell the ability to oxidize HMFCA to FFCA or increases in the cell the specific activity of an enzyme that oxidizes HMFCA to FFCA, as compared to a corresponding cell lacking the expression construct, e.g. a wild type cell. The specific activity of the enzyme that oxidizes HMFCA to FFCA is preferably increased in the cell by at least a factor 1.05, 1.1, 1.2, 1.5, 2.0, 5.0, 10, 20, 50 or 100 as compared to a corresponding cell lacking the expression construct.

The enzyme that has the ability to oxidize HMFCA to FFCA preferably is an alcohol dehydrogenase. A preferred enzyme that has the ability to oxidize HMFCA to FFCA is an alcohol dehydrogenase that has HMFCA dehydrogenase activity. Whether or not a polypeptide has HMFCA dehydrogenase activity can be assayed by expression of the polypeptide in a suitable host cell that is incapable of oxidizing HMFCA to FFCA and detecting whether or not expression of the polypeptide confers to the cell the ability to oxidize HMFCA to FFCA. HMFCA dehydrogenase activity can e.g. be assayed using an expression construct wherein a nucleotide sequence encoding the polypeptide to be assayed for HMFCA dehydrogenase activity replaces the C. basilensis hmfH gene in pBT′hmfH-adh (described in WO2012/064195), after which the plasmid comprising coding sequence of the polypeptide to be assayed for HMFCA dehydrogenase activity is introduced into P. putida KT2440Δgcd containing pJNNhmfT1(t) (also described in WO2012/064195). The P. putida transformants expressing the polypeptide to be assayed for HMFCA dehydrogenase activity are incubated with HMF and samples are drawn at regular intervals for analysis of FDCA. An increase of production of FDCA, as compared to corresponding P. putida transformants lacking the polypeptide to be assayed for HMFCA dehydrogenase activity (and the C. basilensis hmfH gene) is taken as an indication that the polypeptide has HMFCA dehydrogenase activity. Alternatively, a nucleotide sequence encoding the polypeptide to be assayed for HMFCA dehydrogenase activity can be expressed in a fungal host cell, preferably a S. cerevisiae host cell, as e.g. described in the Examples of PCT/EP2016/072406 and detecting whether expression of the polypeptide confers to a fungal host cell the ability to produce both FFCA and/or FDCA from HMF.

The HMFCA dehydrogenase expressed in the cell of the invention preferably is a dehydrogenase that is dependent on a cofactor selected from an adenine dinucleotide, such as NADH or NADPH, a flavin adenine dinucleotide (FAD), a flavin mononucleotide (FMN), and pyrroloquinoline quinolone (PQQ). The HMFCA dehydrogenase expressed in the cell of the invention preferably binds a divalent cation, more preferably the HMFCA dehydrogenase is Zn-binding dehydrogenase.

The HMFCA dehydrogenase expressed in the cell of the invention further preferably is an alcohol dehydrogenase that (also) has the ability of oxidizing other furanic alcohols, preferably furanic alcohols with an hydroxy group in the 2-position, to the corresponding aldehydes. Thus, HMFCA dehydrogenase preferably has the ability of oxidizing 5-hydroxymethylfurfural (HMF) to 2,5-diformyl furan (DFF).

In one embodiment the nucleotide sequence encoding the dehydrogenase with the ability to oxidize HMFCA to FFCA is selected from the group consisting of:
(a) a nucleotide sequence encoding a polypeptide with HMFCA dehydrogenase activity, which polypeptide comprises an amino acid sequence that has at least 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 96, 97, 98, 99 or 100% sequence identity with the amino acid sequence of at least one of SEQ ID NO.'s: 1 and 2 (hmfL1 and hmfL2, respectively), more preferably SEQ ID NO.: 1;
(b) a nucleotide sequence the complementary strand of which hybridises to a nucleotide sequence of (a); and,
(c) a nucleotide sequence the sequence of which differs from the sequence of a nucleotide sequence of (b) due to the degeneracy of the genetic code.

A preferred nucleotide sequence of the invention thus encodes a HMFCA dehydrogenase with an amino acid sequence that is identical to that of a HMFCA dehydrogenase that is obtainable from (or naturally occurs in) a fungus of a genus selected from the group consisting of Aspergillus, Byssochiamys, Coccidioides, Chaetomium, Eutypa, Endocarpon, Fusarium, Microsporum, Neosartorya, Penicillium, Sporothrix and Trichophyton, more preferably, a fungus of a species selected from the group consisting of Coccidioides immitis, Coccidioides posadasii, Endocarpon pusillum, Microsporum gypseum, Penicillium brasilianum and Sporothrix schenckii, most preferably a fungus, which is the strain P. brasilianum C1 or MG11.

In one embodiment the nucleotide sequence encodes a polypeptide with HMFCA dehydrogenase activity as it occurs in nature, e.g. as it can isolated from a wild type source organism. Alternatively, the nucleotide sequence can encode engineered forms of any of the HMFCA dehydrogenase defined above and that comprise one or more amino acid substitutions, insertions and/or deletions as compared to the corresponding naturally occurring HMFCA dehydrogenase but that are within the ranges of identity or similarity as defined herein. Therefore, in one embodiment the nucleotide sequence of the invention encodes a HMFCA dehydrogenase the amino acid sequence of which at least comprises in each of the invariable positions (that are indicated in Tables 1 and 2 with a “*”), the amino acid present in a invariable position. Preferably, the amino acid sequence also comprises in the strongly conserved positions (that are indicated in Tables 1 and 2 with a “:”) one of the amino acids present in a strongly conserved position. More preferably, the amino acid sequence further also comprises in the less strongly conserved positions (that are indicated in Tables 1 and 2 with a “.”) one of the amino acids present in a less strongly conserved position. Amino acid substitutions outside of these invariable and conserved positions are less unlikely to affect HMFCA dehydrogenase activity. Tables 1 and 2 present the amino acid sequence alignments of each of Penicillium brasilianum hmfL1 and hmfL2, respectively with their 10 closest orthologues as available in public databases. Tables 1A and 2A provide the percentages amino acid identities among the P. brasilianum sequences and their orthologues, as well as the accession numbers of the orthologues.

The nucleotide sequences of the invention, encoding polypeptides with HMFCA dehydrogenase activity, are obtainable from genomic and/or cDNA of a fungus, yeast or bacterium, e.g. one that belongs to the same phylum, class or genus as the source organisms described above, using methods for isolation of nucleotide sequences that are well known in the art per se (see e.g. Sambrook and Russell (2001) “Molecular Cloning: A Laboratory Manual (3^{rd} edition), Cold Spring Harbor Laboratory, Cold Spring Harbor Laboratory Press, New York). The nucleotide sequences of the invention are e.g. obtainable in a process wherein a) degenerate PCR primers (designed on the basis of conserved amino acid sequences) are used on genomic and/or cDNA of a suitable organism to generate a PCR fragment comprising part of the nucleotide sequences encoding the polypeptides with HMFCA dehydrogenase activity; b) the PCR fragment obtained in a) is used as probe to screen a cDNA and/or genomic library of the organism; and c) producing a cDNA or genomic DNA comprising the nucleotide sequence encoding a polypeptide with HMFCA dehydrogenase activity.

To increase the likelihood that a HMFCA dehydrogenase of the invention is expressed at sufficient levels and in active form in the cells of the invention, the nucleotide sequence encoding these enzymes, as well as other enzymes of the invention (see below), are preferably adapted to optimise their codon usage to that of the host cell in question. The adaptiveness of a nucleotide sequence encoding a polypeptide to the codon usage of a host cell may be expressed as codon adaptation index (CAI). The codon adaptation index is herein defined as a measurement of the relative adaptiveness of the codon usage of a gene towards the codon usage of highly expressed genes in a particular host cell or organism. The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Jansen et al., 2003, Nucleic Acids Res. 31(8):2242-51). An adapted nucleotide sequence preferably has a CAI of at least 0.2, 0.3, 0.4, 0.5, 0.6, 0.7, 0.8 or 0.9. Suitable codon optimised sequences are e.g. listed in SEQ ID NO's: 27-29, which have been codon optimised for expression in yeast cells, preferably S. cerevisiae cells.

The fungal host cell to be transformed with a nucleic acid construct for expression of the nucleotide sequence encoding a HMFCA dehydrogenase of the invention can in principle be any fungal host cell in which the HMFCA dehydrogenase invention can suitably be expressed, preferably in functional, i.e. active form. The fungal host cell of the invention, preferably is a host cell capable of active or passive transport of furanic compounds into as well as out of the cell. A preferred host cell of the invention lacks or has no detectable activities that degrade (e.g. decarboxylate) carboxylated furanic compounds, such as in particular HMFCA, FFCA and FDCA. Such a host cell preferably naturally lacks the ability to degrade carboxylated furanic compounds. Alternatively, a fungal host cell can be genetically modified to reduce or eliminate the specific activities of one or more enzymes that catalyses the degradation of carboxylated furanic compounds, as described herein below.

The expression construct for expression of a nucleotide sequence encoding a HMFCA dehydrogenase of the invention, preferably is an expression construct that is heterologous or exogenous to the host cell transformed with the construct. A construct is herein understood to be heterologous or exogenous to the host cell comprising the construct when the construct comprises at least one sequence or sequence element that does not naturally occur in the host cell and/or when construct comprises at least two sequence elements in a combination and/or order that does not naturally occur in the host cell, even if the elements themselves do naturally occur in the host cell.

Vectors and expression constructs for expression of a nucleotide sequence encoding a HMFCA dehydrogenase of the invention in appropriate host cells are described in more detail herein below.

### Introducing or Increasing Furanic Aldehyde Dehydrogenase Activity

A cell expressing an HMFCA dehydrogenase of the invention, as described above, further preferably has aldehyde dehydrogenase activity (i.e. having EC 1.2 activity). Preferably, the aldehyde dehydrogenase is capable of converting furanic aldehydes. More preferably the aldehyde dehydrogenase activity is capable of oxidizing furanic aldehydes to the corresponding furanic carboxylic acids. More specifically, the aldehyde dehydrogenase activity is preferably capable of at least one of i) oxidizing HMF to HMFCA, ii) oxidizing 2,5-diformyl furan (DFF) to 5-formyl-2-furoic acid (FFCA), and iii) FFCA into FDCA. Such furanic aldehyde dehydrogenase activity can be an endogenous activity of the cell or it can be an exogenous activity conferred to the cell. Preferably, the furanic aldehyde dehydrogenase activity is conferred to or increased in the cell by transformation of the cell with an expression construct, e.g. a second expression construct if the cell already comprises a first expression construct for expression of the HMFCA dehydrogenase.

In a preferred cell of the invention, the expression construct for expression of the furanic aldehyde dehydrogenase is expressible in the cell and expression of the furanic aldehyde dehydrogenase preferably confers to the ability to oxidize at least one of i) oxidizing HMF to HMFCA, ii) oxidizing DFF to FFCA, and iii) oxidizing FFCA into FDCA, or increases in the cell the specific activity of a furanic aldehyde dehydrogenase with at least one of these abilities, as compared to a corresponding cell lacking the expression construct, e.g. a wild type cell. The specific activity of the furanic aldehyde dehydrogenase is preferably increased in the cell by at least a factor 1.05, 1.1, 1.2, 1.5, 2.0, 5.0, 10, 20, 50 or 100 as compared to a corresponding cell lacking the expression construct.

The ability of a polypeptide to oxidize at least one of i) HMF to HMFCA, ii) oxidizing DFF to FFCA, and iii) FFCA to FDCA, may be assayed by co-expression of a nucleotide sequence encoding the polypeptide in a P. putida host cell, preferably an P. putida KT2440 host cell, together with the HmfH and HmfT1 genes from C. basilensis HMF 14, incubating the P. putida cells in 10 mM HMF and detecting an increase in the accumulation FDCA as compared to corresponding P. putida cells that do not express the polypeptide, e.g. as described in Example IV of WO2012/064195. The ability of a polypeptide to oxidize HMF to HMFCA may also be assayed as described by Koopman et al 2010, PNAS supra). Strains expressing the hmfT1 gene from C. basilensis HMF14 are herein understood to express a gene product having the amino acid sequence of SEQ ID NO: 55. Alternatively, a nucleotide sequence encoding the polypeptide to be assayed for its ability to oxidize at least one of i) HMF to HMFCA, ii) oxidizing DFF to FFCA, and iii) FFCA to FDCA can be co-expressed in a fungal host cell, preferably a S. cerevisiae host cell, with an HMFCA dehydrogenase as e.g. described in the Examples of PCT/EP2016/072406 and detecting whether expression of the polypeptide causes an increase in the accumulation FDCA as compared to corresponding fungal host cells that do not express the polypeptide.

The furanic aldehyde dehydrogenase expressed in the cell of the invention preferably is a dehydrogenase that is dependent on a cofactor selected from an adenine dinucleotide, such as NADH or NADPH, a flavin adenine dinucleotide (FAD), a flavin mononucleotide (FMN), and pyrroloquinoline quinolone (PQQ).

In one embodiment, the nucleotide sequence encoding the furanic aldehyde dehydrogenase is selected from the group consisting of:
a) a nucleotide sequence encoding a polypeptide having at least one of the abilities of i) oxidizing HMF to HMFCA, ii) oxidizing DFF to FFCA, and, iii) oxidizing FFCA into FDCA, which polypeptide comprising an amino acid sequence that has at least 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 96, 97, 98, 99 or 100% sequence identity with the amino acid sequence of at least one of SEQ ID NO's: 3 (the hmfN1-encoded aldehyde dehydrogenase);
b) a nucleotide sequence the complementary strand of which hybridises to a nucleotide sequence of (a); and,
c) a nucleotide sequence the sequence of which differs from the sequence of a nucleotide sequence of (b) due to the degeneracy of the genetic code.

A preferred nucleotide sequence of the invention thus encodes a furanic aldehyde dehydrogenase with an amino acid sequence that is identical to that of a furanic aldehyde dehydrogenase that is obtainable from (or naturally occurs in) a fungus of a genus selected from the group consisting of Aspergillus, Eutypa, Neosartorya, Penicillium, Podospora, Scedosporium and Sporothrix, more preferably, a fungus of a species selected from the group consisting of Eutypa lata, Penicillium brasilianum, Podospora anserina, Scedosporium apiospermum and Sporothrix schenckii, most preferably a fungus, which is the strain P. brasilianum C1 or MG11.

In one embodiment the nucleotide sequence encodes a polypeptide with furanic aldehyde dehydrogenase activity as it occurs in nature, e.g. as it can isolated from a wild type source organism. Alternatively, the nucleotide sequence can encode engineered forms of any of the furanic aldehyde dehydrogenase defined above and that comprise one or more amino acid substitutions, insertions and/or deletions as compared to the corresponding naturally occurring furanic aldehyde dehydrogenase but that are within the ranges of identity or similarity as defined herein. Therefore, in one embodiment the nucleotide sequence of the invention encodes a furanic aldehyde dehydrogenase, the amino acid sequence of which at least comprises in each of the invariable positions (that are indicated in Table 3 with a “*”), the amino acid present in a invariable position. Preferably, the amino acid sequence also comprises in the strongly conserved positions (that are indicated in Table 3 with a “:”) one of the amino acids present in a strongly conserved position. More preferably, the amino acid sequence further also comprises in the less strongly conserved positions (that are indicated in Table 3 with a “.”) one of the amino acids present in a less strongly conserved position. Amino acid substitutions outside of these invariable and conserved positions are less unlikely to affect furanic aldehyde dehydrogenase activity. Table 3 present the amino acid sequence alignments of Penicillium brasilianum hmfN1 with its 10 closest orthologues as available in public databases. Tables 3A provide the percentages amino acid identities among the P. brasilianum sequence and its orthologues, as well as the accession numbers of the orthologues.

The nucleotide sequences of the invention, encoding polypeptides with furanic aldehyde dehydrogenase activity, are obtainable from genomic and/or cDNA of a fungus, yeast or bacterium, e.g. one that belongs to the same phylum, class or genus as the source organisms described above, using methods for isolation of nucleotide sequences that are well known in the art per se (see e.g. Sambrook and Russell (2001) “Molecular Cloning: A Laboratory Manual (3^{rd} edition), Cold Spring Harbor Laboratory, Cold Spring Harbor Laboratory Press, New York). The nucleotide sequences of the invention are e.g. obtainable in a process wherein a) degenerate PCR primers (designed on the basis of conserved amino acid sequences) are used on genomic and/or cDNA of a suitable organism to generate a PCR fragment comprising part of the nucleotide sequences encoding the polypeptides with furanic aldehyde dehydrogenase activity; b) the PCR fragment obtained in a) is used as probe to screen a cDNA and/or genomic library of the organism; and c) producing a cDNA or genomic DNA comprising the nucleotide sequence encoding a polypeptide with furanic aldehyde dehydrogenase activity.

The fungal host cell to be transformed with a nucleic acid construct for expression of the nucleotide sequence encoding a furanic aldehyde dehydrogenase of the invention preferably is a fungal host cell as described above for transformation with a nucleic acid construct for expression of the nucleotide sequence encoding the HMFCA dehydrogenase, and wherein also the furanic aldehyde dehydrogenase can suitably be expressed, preferably in functional, i.e. active form. Preferably, the fungal host cell to be transformed with a nucleic acid construct for expression of the nucleotide sequence encoding a furanic aldehyde dehydrogenase also expresses nucleotide sequence encoding the HMFCA dehydrogenase, more preferably the cell comprises an expression construct for the HMFCA dehydrogenase that confers to or increases in the cell the ability to oxidize HMFCA to FFCA. As described above, such a fungal host cell, preferably is capable of active or passive transport of furanic compounds into as well as out of the cell and preferably lacks or has no detectable activities that degrade (e.g. decarboxylate) carboxylated furanic compounds.

The expression construct for expression of a nucleotide sequence encoding a furanic aldehyde dehydrogenase of the invention, preferably is an expression construct that is heterologous or exogenous to the host cell transformed with the construct. A construct is herein understood to be heterologous or exogenous to the host cell comprising the construct when the construct comprises at least one sequence or sequence element that does not naturally occur in the host cell and/or when construct comprises at least two sequence elements in a combination and/or order that does not naturally occur in the host cell, even if the elements themselves do naturally occur in the host cell.

Vectors and expression constructs for expression of a nucleotide sequence encoding a furanic aldehyde dehydrogenase of the invention in appropriate host cells are described in more detail herein below.

### Reduction or Elimination of Alternative Routes for Metabolism of Furanic Compounds

Alternative endogenous routes for metabolism of HMF and other furanic precursors of FDCA may also be present in a cell of the invention. Such alternative routes compete with the production of FDCA from HMF and other furanic precursors of FDCA. Preferably therefore the specific activity of enzymes in such alternative routes is also reduced or eliminated in a cell of the invention. One such endogenous alternative route is e.g. the reduction of HMF and/or FFCA to the corresponding alcohol by an dehydrogenase, such as e.g. a short chain dehydrogenase. A gene encoding such a short chain dehydrogenase to be modified for reducing or eliminating the specific activity of an alternative route for metabolising HMF and other furanic precursors of FDCA, preferably is a gene encoding an amino acid sequence with at least 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 96, 97, 98, 99 or 100% sequence identity to SEQ ID NO: 5 (hmfM). In the cells of the invention, the specific short chain dehydrogenase activity is preferably reduced by at least a factor 1.05, 1.1, 1.2, 1.5, 2.0, 5.0, 10, 20, 50 or 100 as compared to cells of a strain which is genetically identical except for the genetic modification causing the reduction in activity.

Nucleotide sequences encoding short chain dehydrogenase the specific activities of which are preferably reduced or eliminated in a cell of the invention, are obtainable from and may be identified in genomic and/or cDNA of a fungus, yeast or bacterium, e.g. one that belongs to the same phylum, class or genus as the source organisms described above, using methods for isolation of nucleotide sequences that are well known in the art per se (see e.g. Sambrook and Russell (2001) “Molecular Cloning: A Laboratory Manual (3^{rd} edition), Cold Spring Harbor Laboratory, Cold Spring Harbor Laboratory Press, New York). The nucleotide sequences of the invention are e.g. obtainable in a process wherein a) degenerate PCR primers (designed on the basis of conserved amino acid sequences) are used on genomic and/or cDNA of a suitable organism to generate a PCR fragment comprising part of the nucleotide sequences encoding enzymes the specific activities of which are preferably reduced or eliminated in a cell of the invention; b) the PCR fragment obtained in a) is used as probe to screen a cDNA and/or genomic library of the organism; and c) producing a cDNA or genomic DNA comprising the nucleotide sequence encoding the enzyme the specific activities of which is preferably reduced or eliminated in a cell of the invention. Such conserved sequences can be identified in the sequences alignments presented in Table 5, wherein invariable positions are indicated with a “*” and strongly conserved positions are indicated with a “:”. Also suitable host cells of the invention can be derived from Table 5 wherein the host preferably is a non-pathogenic fungus or yeast that belongs to the same phylum, class, order, family or genus as the source organism of an orthologue identified in Table 5. Table 5 presents the amino acid sequence alignments of each of Penicillium brasilianum hmfM with its 10 closest orthologues as available in public databases. Table 5A provides the percentages amino acid identities among the P. brasilianum hmfM sequence and its orthologues, as well as the accession numbers of the orthologues.

Another endogenous dehydrogenase known to reduce HMF to HMF-alcohol is the NADPH-dependent alcohol dehydrogenase encoded by the S. cerevisiae ADH6 gene as described by Petersson et al. (2006, Yeast, 23:455-464). Therefore, a gene to be modified for reducing or eliminating the specific activity of alternative route for metabolising HMF, preferably is the S. cerevisiae ADH6 gene or an orthologue thereof in another fungal host species. Orthologues of the S. cerevisiae ADH6 gene in filamentous fungi such as Aspergillus and Penicillium with amino acid sequence identities in the range of 50-60% can be identified in public sequence databases. Preferably therefore, the gene to be modified for reducing or eliminating the specific activity of an NADPH-dependent HMF-reducing dehydrogenase is a gene encoding an amino acid sequence with at least 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 96, 97, 98, 99 or 100% sequence identity to SEQ ID NO: 25 (S. cerevisiae ADH6). In the cells of the invention, the activity specific of the NADPH-dependent HMF-reducing dehydrogenase is preferably reduced by at least a factor 1.05, 1.1, 1.2, 1.5, 2.0, 5.0, 10, 20, 50 or 100 as compared to cells of a strain which is genetically identical except for the genetic modification causing the reduction in activity.

### Cells Expressing a Transporter of Furanic Compounds

A cell of the invention, as described above, further preferably expresses one or more nucleotide sequences encoding a polypeptide having furanic compound transport capabilities. Such polypeptides having furanic compound transport capabilities can be an endogenous activity of the cell or it can be an exogenous activity conferred to the cell. Preferably, the activity of a polypeptides having furanic compound transport capabilities is conferred to or increased in the cell by transformation of the cell with an expression construct, e.g. a third expression construct if the cell already comprises a first expression construct for expression of the HMFCA dehydrogenase and a second expression construct for expression of the furanic aldehyde dehydrogenase or oxidase.

Preferably the cell is transformed with an expression construct for expression of a nucleotide sequence encoding a polypeptide having furanic compound transport capabilities. The polypeptide having furanic compound transport capabilities preferably is a polypeptide having HMFCA transport capabilities, which at least includes the capability to transport HMFCA into the cell. Preferably the cell comprises an expression construct for expression of a nucleotide sequence encoding a polypeptide having the ability to transport at least HMFCA into the cell, the polypeptide comprising an amino acid sequence with at 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 96, 97, 98, 99 or 100% sequence identity to at least one of SEQ ID NO: 6-10 (respectively, hmfT3, hmfT4, hmfT5, hmfT6, and hmfT7), wherein, the expression construct is expressible in the cell and expression of the polypeptide confers to or increases in the cell the ability to transport at least HMFCA into the cell, as compared to a corresponding wild type cell lacking the expression construct.

The ability of a polypeptide to transport furanic compounds, in particular HMFCA, into the cell may be assayed by co-expression of a nucleotide sequence encoding the transporter polypeptide in a yeast host cell, preferably a S. cerevisiae CEN.PK host cell, together with the hmfH gene from C. basilensis HMF 14 and a gene encoding a furanic aldehyde dehydrogenase associated with the HMF-degradation operon from C. basilensis HMF 14 (having the amino acid sequence of SEQ ID NO: 19 of WO2012/064195), incubating the transformed S. cerevisiae cells in 4 mM HMF and detecting an increase in the accumulation FDCA as compared to corresponding (i.e. otherwise identical) S. cerevisiae cells that do not express the transporter polypeptide, as e.g. described in the Examples of PCT/EP2016/072406.

A preferred nucleotide sequence of the invention thus encodes a furanic compound transporter polypeptide with an amino acid sequence that is identical to that of a furanic compound transporter polypeptide that is obtainable from (or naturally occurs in) a fungus of a genus selected from the group consisting of Aspergillus, Fusarium, Nectria, Penicillium, Sporothrix and Togninia, more preferably, a fungus of a species selected from the group consisting of Aspergillus terreus, Penicillium brasilianum, Penicillium digitatum, Penicillium rubens, Sporothrix schenckii and Togninia minima, most preferably a fungus, which is the strain P. brasilianum C1 or MG11.

In one embodiment the nucleotide sequence encodes a furanic compound transporter polypeptide as it occurs in nature, e.g. as it can isolated from a wild type source organism. Alternatively, the nucleotide sequence can encode engineered forms of any of the furanic compound transporter polypeptides defined above and that comprise one or more amino acid substitutions, insertions and/or deletions as compared to the corresponding naturally occurring furanic compound transporter polypeptide but that are within the ranges of identity or similarity as defined herein. Therefore, in one embodiment the nucleotide sequence of the invention encodes a furanic compound transporter polypeptide, the amino acid sequence of which at least comprises in each of the invariable positions (that are indicated in Tables 6-10 with a “*”), the amino acid present in a invariable position. Preferably, the amino acid sequence also comprises in the strongly conserved positions (that are indicated in Tables 6-10 with a “:”) one of the amino acids present in a strongly conserved position. More preferably, the amino acid sequence further also comprises in the less strongly conserved positions (that are indicated in Tables 6-10 with a “.”) one of the amino acids present in a less strongly conserved position. Amino acid substitutions outside of these invariable and conserved positions are less unlikely to affect furanic compound transporter polypeptide activity. Tables 6-10 present the amino acid sequence alignments of each of Penicillium brasilianum hmfT3, hmfT4, hmfT5, hmfT6 and hmfT7, respectively, with their 10 closest orthologues as available in public databases. Tables 6A-10A provide the percentages amino acid identities among the P. brasilianum sequences and their orthologues, as well as the accession numbers of the orthologues.

The nucleotide sequences of the invention, encoding polypeptides with furanic compound transporter activity, are obtainable from genomic and/or cDNA of a fungus, yeast or bacterium, e.g. one that belongs to the same phylum, class or genus as the source organisms described above, using methods for isolation of nucleotide sequences that are well known in the art per se (see e.g. Sambrook and Russell (2001) “Molecular Cloning: A Laboratory Manual (3^{rd} edition), Cold Spring Harbor Laboratory, Cold Spring Harbor Laboratory Press, New York). The nucleotide sequences of the invention are e.g. obtainable in a process wherein a) degenerate PCR primers (designed on the basis of conserved amino acid sequences) are used on genomic and/or cDNA of a suitable organism to generate a PCR fragment comprising part of the nucleotide sequences encoding the polypeptides with the activity of a furanic compound transporter; b) the PCR fragment obtained in a) is used as probe to screen a cDNA and/or genomic library of the organism; and c) producing a cDNA or genomic DNA comprising the nucleotide sequence encoding a furanic compound transporter polypeptide.

The fungal host cell to be transformed with a nucleic acid construct for expression of the nucleotide sequence encoding a furanic compound transporter polypeptide preferably is a fungal host cell of the invention as described above.

The expression construct for expression of a nucleotide sequence encoding a furanic compound transporter polypeptide, preferably is an expression construct that is heterologous or exogenous to the host cell transformed with the construct. A construct is herein understood to be heterologous or exogenous to the host cell comprising the construct when the construct comprises at least one sequence or sequence element that does not naturally occur in the host cell and/or when construct comprises at least two sequence elements in a combination and/or order that does not naturally occur in the host cell, even if the elements themselves do naturally occur in the host cell.

In an separate aspect, the invention relates to a fungal cell having the ability to oxidise HMF to FDCA and comprising a genetic modification that increases expression of a nucleotide sequence encoding a polypeptide that transports at least one furanic compound, which polypeptide preferably comprises an amino acid sequence that has at least 45% sequence identity with the amino acid sequence of at least one of SEQ ID NO.'s: 9 and 10. Preferably, the fungal cell further comprises one or more genetic modifications selected from: a) a genetic modification that eliminates or reduces specific 2,5-furandicarboxylic acid (FDCA) decarboxylating monooxygenase activity in the cell, as compared to a corresponding parent cell lacking the genetic modification, as described herein above; b) a genetic modification that confers to the cell the ability to oxidize 5-hydroxymethyl-2-furancarboxylic acid (HMFCA) to 5-formyl-2-furoic acid (FFCA) or that increases in the cell the specific activity of a enzyme that oxidizes HMFCA to FFCA as compared to a corresponding wild type cell lacking the genetic modification, as described herein above; c) a genetic modification that confers to the cell the ability to oxidize furanic aldehydes to the corresponding furanic carboxylic acids or a genetic modification that increases in the cell the specific activity of a enzyme that oxidizes furanic aldehydes to the corresponding furanic carboxylic acids, as compared to a corresponding wild type cell lacking the genetic modification, as described herein above; d) a genetic modification that reduces or eliminates the expression of a gene encoding a short chain dehydrogenase that reduces HMF and/or FFCA to the corresponding alcohol, wherein preferably the gene is at least one of a gene encoding polypeptide comprising an amino acid sequence with at least 45% sequence identity to at least one of SEQ ID NO: 5 and 25, as described herein above; e) a genetic modification that increases expression of a nucleotide sequence encoding a polypeptide that transports at least one furanic compound, which polypeptide preferably comprises an amino acid sequence that has at least 45% sequence identity with the amino acid sequence of at least one of SEQ ID NO.'s: 6-8, as described herein above; and, f) a genetic modification that alters the expression of a gene encoding a transcriptional activator of genes involved in furan catabolism, wherein preferably the gene is a gene encoding a polypeptide comprising an amino acid sequence with at least 45% sequence identity to SEQ ID NO: 11, as described herein below.

Vectors and expression constructs for expression of a nucleotide sequence encoding a furanic compound transporter polypeptide of the invention in appropriate host cells are described in more detail herein below.

Cell with Altered Regulation of Expression of a Transcriptional Activator

In one embodiment of a cell of the invention, the regulation of expression of a transcriptional activator of genes involved in furan catabolism is altered. The expression of the transcriptional activator can be reduced or eliminated to prevent degradation of FDCA in cells containing endogenous genes for FDCA degradation, and preferably containing genes coding for enzymes for converting HMF to FDCA that expressed independent from the transcriptional activator. Alternatively, the expression of the transcriptional activator can be increased and/or be made constitutive in cells genetically modified to prevent FDCA degradation, so as to increase expression of endogenous genes for converting HMF, and/or other furanic precursors, to FDCA.

Preferably, in a cell of the invention, the transcriptional activator of which the regulation of expression is altered, is encoded by a nucleotide sequence encoding a polypeptide comprising an amino acid sequence with at least 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 96, 97, 98, 99 or 100% sequence identity to SEQ ID NO: 11 (hmfR), wherein, the polypeptide has the ability to activate transcription of at least one gene involved in furan catabolism.

A preferred nucleotide sequence of the invention thus encodes a transcriptional activator with an amino acid sequence that is identical to that of a transcriptional activator that is obtainable from (or naturally occurs in) a fungus of a genus selected from the group consisting of Fusarium, Penicillium, Scedosporium, Sporothrix and Stachybotrys more preferably, a fungus of a species selected from the group consisting of Fusarium oxysporum, Penicillium brasilianum, Scedosporium apiospermum, Sporothrix schenckii and Stachybotrys chlorohalonata, most preferably a fungus, which is the strain P. brasilianum C1 or MG11.

In one embodiment the nucleotide sequence encodes a transcriptional activator as it occurs in nature, e.g. as it can isolated from a wild type source organism. Alternatively, the nucleotide sequence can encode engineered forms of any of the transcriptional activator polypeptides defined above and that comprise one or more amino acid substitutions, insertions and/or deletions as compared to the corresponding naturally occurring transcriptional activator polypeptide but that are within the ranges of identity or similarity as defined herein. Therefore, in one embodiment the nucleotide sequence of the invention encodes a transcriptional activator polypeptide, the amino acid sequence of which at least comprises in each of the invariable positions (that are indicated in Table 11 with a “*”), the amino acid present in a invariable position. Preferably, the amino acid sequence also comprises in the strongly conserved positions (that are indicated in Table 11 with a “:”) one of the amino acids present in a strongly conserved position. More preferably, the amino acid sequence further also comprises in the less strongly conserved positions (that are indicated in Table 11 with a “.”) one of the amino acids present in a less strongly conserved position. Amino acid substitutions outside of these invariable and conserved positions are less unlikely to affect transcriptional activator activity. Table 11 presents the amino acid sequence alignment of Penicillium brasilianum hmfR, with its 10 closest orthologues as available in public databases. Table 11A provides the percentages amino acid identities among the P. brasilianum sequence and its orthologues, as well as the accession numbers of the orthologues.

The nucleotide sequences of the invention, encoding polypeptides with transcriptional activator activity, are obtainable from genomic and/or cDNA of a fungus, yeast or bacterium, e.g. one that belongs to the same phylum, class or genus as the source organisms described above, using methods for isolation of nucleotide sequences that are well known in the art per se (see e.g. Sambrook and Russell (2001) “Molecular Cloning: A Laboratory Manual (3^{rd} edition), Cold Spring Harbor Laboratory, Cold Spring Harbor Laboratory Press, New York). The nucleotide sequences of the invention are e.g. obtainable in a process wherein a) degenerate PCR primers (designed on the basis of conserved amino acid sequences) are used on genomic and/or cDNA of a suitable organism to generate a PCR fragment comprising part of the nucleotide sequences encoding the polypeptides with the activity of a transcriptional activator; b) the PCR fragment obtained in a) is used as probe to screen a cDNA and/or genomic library of the organism; and c) producing a cDNA or genomic DNA comprising the nucleotide sequence encoding a furanic transcriptional activator.

The fungal host cell to be transformed with a nucleic acid construct for expression of the nucleotide sequence encoding a furanic transcriptional activator polypeptide preferably is a fungal host cell of the invention as described above.

The expression construct for expression of a nucleotide sequence encoding a furanic transcriptional activator polypeptide, preferably is an expression construct that is heterologous or exogenous to the host cell transformed with the construct. A construct is herein understood to be heterologous or exogenous to the host cell comprising the construct when the construct comprises at least one sequence or sequence element that does not naturally occur in the host cell and/or when construct comprises at least two sequence elements in a combination and/or order that does not naturally occur in the host cell, even if the elements themselves do naturally occur in the host cell.

Vectors and expression constructs for expression of a nucleotide sequence encoding a furanic transcriptional activator polypeptide of the invention in appropriate host cells are described in more detail herein below.

### Vectors, Genetic Constructs and Methods for Genetic Modifications of Cells of the Invention

For the genetic modification of the parent host cells of the invention, i.e. for the construction of the modified host cells of the invention, standard genetic and molecular biology techniques are used that are generally known in the art and have e.g. been described by Sambrook and Russell (2001, “Molecular cloning: a laboratory manual” (3rd edition), Cold Spring Harbor Laboratory, Cold Spring Harbor Laboratory Press) and Ausubel et al. (1987, eds., “Current protocols in molecular biology”, Green Publishing and Wiley Interscience, New York).

More specifically, means and methods for genetic modification of yeasts are standard and known to those in the art, including e.g. promoters for (over-)expression of genes, episomal and/or integrating expression constructs and vectors, selectable markers, methods and genetic constructs for disrupting and/or deleting endogenous yeast genes or parts thereof and methods for transforming yeast. Such means and methods are e.g. described in: Sherman et al, Methods Yeast Genetics, Cold Spring Harbor Laboratory, N Y (1978); Guthrie et al. (Eds.) Guide To Yeast Genetics and Molecular Biology Vol. 194, Academic Press, San Diego (1991); Sudbery, P. E. (2001) Genetic Engineering of Yeast, in Biotechnology Set, Second Edition (eds H.-J. Rehm and G. Reed), Wiley-VCH Verlag GmbH, Weinheim, Germany. doi: 10.1002/9783527620999.ch13a; and, Gaillardin, C. and Heslot, H. (1988), Genetic engineering in Yarrowia lipolytica. J. Basic Microbiol., 28: 161-174. doi: 10.1002/jobm.3620280303; all of which are incorporated herein by reference.

Similarly, means and methods for genetic modification of filamentous fungi are standard and known to those in the art, including e.g. promoters for (over-)expression of genes, episomal and/or integrating expression constructs and vectors, selectable markers, and methods and genetic constructs for disrupting and/or deleting endogenous fungal genes or parts thereof and methods for transforming filamentous fungi. Such means and methods are e.g. described in Moore, M. M. (2007, “Genetic engineering of fungal cells”, In Biotechnology Vol. III. (Ed. H. W. Doelle and E. J. Dasilva), EOLSS, Ontario, Canada. pp. 36-63; Lubertozzi, D., & Keasling, J. D. (2009), “Developing Aspergillus as a host for heterologous expression”, Biotechnology advances, 27(1), 53-75; Meyer, V. (2008) “Genetic engineering of filamentous fungi—progress, obstacles and future trends”, Biotechnology Advances, (26), 177-85; Kuck and Hoff (2010) “New tools for the genetic manipulation of filamentous fungi. Applied microbiology and biotechnology”, 86(1), 51-62; and, WO2014/142647, all of which are incorporated herein by reference.

Thus in another aspect, the invention pertains to nucleic acid constructs, such as vectors, including cloning and expression vectors, comprising a polynucleotide of the invention, e.g. a nucleotide sequence encoding a HMFCA dehydrogenase or a furanic aldehyde dehydrogenase of the invention or a functional equivalent thereof and methods of transforming or transfecting a suitable host cell with such vectors. As used herein, the terms “vector” and “construct” are used interchangeably and refers to a constructed nucleic acid molecule comprising a polynucleotide of the invention.

A vector according to the invention may be an autonomously replicating vector, i.e. a vector which exists as an extra-chromosomal entity, the replication of which is independent of chromosomal replication, e.g. a plasmid. Alternatively, the vector may be one which, when introduced into a host cell, is integrated into the host cell genome and replicated together with the chromosome (s) into which it has been integrated. For convenience the vector can be a shuttle vector, also comprising a origin of replication and selectable marker for use in a bacterium such as E. coli, for ease of manipulation and production.

In one embodiment, the nucleic acid constructs is an expression construct or expression vector, comprising a nucleotide sequence encoding a polypeptide of the invention to be (over-) expressed and wherein the nucleotide sequence encoding the polypeptide is operably linked to regulatory sequences that are capable of effecting and controlling (the rate of) expression of the coding nucleotide sequence in the host cells in question. Such regulatory sequences typically at least include a promoter that functions to control the transcription of the coding sequence, which is usually located upstream of, and preferably operably linked the coding sequence. In addition to the promoter, the upstream transcription regulatory sequences may comprises further elements such as enhancers, upstream activating sequences, transcription factor binding sites, repressor and activator protein binding sites and the like. The promoter sequence will usually include the transcription initiation site(s). Suitable promoters and transcription regulatory sequences for expression of coding sequences in yeast or filamentous fungi are described in the above-cited references. Downstream of the promoter and transcription initiation site(s), the expression construct will comprise the translation initiation sequences, such as the eukaryotic Kozak consensus sequence, surrounding the translation initiation codon, i.e. the first codon of the coding sequence. The coding sequence is terminated with a translation stop codon. Downstream of the coding sequence, the expression construct may comprise a 3′-untranslated region containing one or more transcription termination sites, e.g. a terminator, which preferably also includes a polyadenylation site. The origin of the terminator is less critical. The terminator can, for example, be native to the DNA sequence encoding the polypeptide. However, preferably a yeast terminator is used in yeast host cells and a filamentous fungal terminator is used in filamentous fungal host cells. More preferably, the terminator is endogenous to the host cell (in which the nucleotide sequence encoding the polypeptide is to be expressed). A functional expression unit comprising a coding sequence operably linked to the appropriate upstream- and downstream regulatory sequences may be referred to as an expression cassette. An expression vector or expression construct of the invention may comprise more than one expression cassette, optionally for the expression of more than one different coding sequences.

In addition to at least one expression cassette, an expression vector or expression construct of the invention preferably also comprises a selectable marker for selection of host cells transformed with the vector or construct. In a preferred embodiment, the selectable marker in the expression vector or expression construct in a configuration that allows excision of the marker from the expression construct/vector, once in the host cell after initial selection of the transformants, e.g. using homologous recombination as described in EP 0 635 574, or using the Cre-lox system as described by Güldener et al. (1996, Nucleic Acids Res. 24:2519-2524).

The invention further relates to method for the preparation of a polypeptide of the invention, e.g. a polypeptide having HMFCA dehydrogenase activity, a polypeptide having furanic aldehyde dehydrogenase activity and including polypeptides the expression of which is to be reduced/eliminated in the cell of the invention. The method comprises cultivating a cell according to the invention under conditions conducive to expression of the polypeptide and, optionally, recovering the expressed polypeptide. The invention also relates to a polypeptide obtainable by such a method.

Thus in another aspect, the invention pertains to means and methods for modifying endogenous target genes in the cells of the invention so as to reduce or eliminate the expression and/or activity of the encoded target proteins. Modifications that may be used to reduce or eliminate expression of a target protein are disruptions that include, but are not limited to, deletion of the entire gene or a portion of the gene encoding the target protein, inserting a DNA fragment into the target gene (in either the promoter or coding region) so that the protein is not expressed or expressed at lower levels, introducing a mutation into the target coding region which adds a stop codon or frame shift such that a functional protein is not expressed, and introducing one or more mutations into a target coding region to alter amino acids so that a non-functional target protein, or a target protein with reduced enzymatic activity is expressed. In addition, expression of the target gene may be blocked by expression of an antisense RNA or an interfering RNA, and constructs may be introduced that result in co-suppression. Moreover, a target coding sequence may be synthesized whose expression will be low because rare codons are substituted for plentiful ones, when this suboptimal coding sequence is substituted for the corresponding endogenous target coding sequence. Preferably such a suboptimal coding sequence will have a codon adaptation index (see above) of less than 0.5, 0.4, 0.3 0.2, or 0.1. Such a suboptimal coding sequence will produce the same polypeptide but at a lower rate due to inefficient translation. In addition, the synthesis or stability of the transcript may be reduced by mutation. Similarly the efficiency by which a protein is translated from mRNA may be modulated by mutation, e.g. by using suboptimal translation initiation codons. All of these methods may be readily practiced by one skilled in the art making use of the sequences encoding target proteins.

In particular, genomic DNA sequences surrounding a target coding sequence are useful for modification methods using homologous recombination. For example, in this method sequences flanking the target gene are placed on either site of a selectable marker gene to mediate homologous recombination whereby the marker gene replaces the target gene. Also partial target gene sequences and target gene flanking sequences bounding a selectable marker gene may be used to mediate homologous recombination whereby the marker gene replaces a portion of the target gene. In addition, the selectable marker in the inactivation construct can be configured in such a way so as to allow excision of the marker from the inactivation construct expression construct/vector, once integrated in the host cell's genome, e.g. using homologous recombination as described in EP 0 635 574, or using the Cre-lox system as described by Güldener et al. (1996, Nucleic Acids Res. 24:2519-2524).

Deletions of target genes may also be effected using mitotic recombination as described in Wach et al. (1994, Yeast 10:1793-1808). This method involves preparing a DNA fragment that contains a selectable marker between genomic regions that may be as short as 20 bp, and which bound, i.e. flank the target DNA sequence. This DNA fragment can be prepared by PCR amplification of the selectable marker gene using as primers oligonucleotides that hybridize to the ends of the marker gene and that include the genomic regions that can recombine with the fungal genome. The linear DNA fragment can be efficiently transformed into yeast or filamentous fungi and recombined into the genome resulting in gene replacement including with deletion of the target DNA sequence (as described in Methods in Enzymology, 1991, v 194, pp 281-301). Moreover, promoter replacement methods may be used to exchange the endogenous transcriptional control elements allowing another means to modulate expression such as described in Mnaimneh et al. (2004, Cell 118(1):31-44) and in the Examples herein.

In addition, the activity of target proteins or genes in any cell may be disrupted using random mutagenesis, which is followed by screening to identify strains with reduced activity of the target proteins. Using this type of method, the DNA sequence coding for the target protein, or any other region of the genome affecting expression of the target protein, need not even be known. Methods for creating genetic mutations are common and well known in the art and may be applied to the exercise of creating mutants. Commonly used random genetic modification methods (reviewed in Methods in Yeast Genetics, 2005, Cold Spring Harbor Laboratory Press, Cold Spring Harbor, N.Y.) include spontaneous mutagenesis, mutagenesis caused by mutator genes, chemical mutagenesis, irradiation with UV or X-rays, or transposon mutagenesis.

Chemical mutagenesis of fungi commonly involves treatment of cells with one of the following DNA mutagens: ethyl methanesulfonate (EMS), nitrous acid, diethyl sulfate, or N-methyl-N′-nitro-N-nitroso-guanidine (MNNG). These methods of mutagenesis have been reviewed in Spencer et al (Mutagenesis in Yeast, 1996, Yeast Protocols: Methods in Cell and Molecular Biology. Humana Press, Totowa, N.J.). Chemical mutagenesis with EMS may be performed as described in Methods in Yeast Genetics, 2005, Cold Spring Harbor Laboratory Press, Cold Spring Harbor, N.Y. Irradiation with ultraviolet (UV) light or X-rays can also be used to produce random mutagenesis in yeast cells. The primary effect of mutagenesis by UV irradiation is the formation of pyrimidine dimers which disrupt the fidelity of DNA replication. Protocols for UV-mutagenesis of yeast can be found in Spencer et al (Mutagenesis in Yeast, 1996, Yeast Protocols: Methods in Cell and Molecular Biology. Humana Press, Totowa, N.J.). Introduction of a mutator phenotype can also be used to generate random chromosomal mutations in yeast. Common mutator phenotypes can be obtained through disruption of one or more of the following genes: PMS1, MAG1, RAD18 or RAD51 or their orthologues in fungi other than S. cerevisae. Restoration of the non-mutator phenotype can be easily obtained by insertion of the wild type allele. Collections of modified cells produced from any of these or other known random mutagenesis processes may be screened for reduced activity of the target protein (US20090305363).

### Processes for the Oxidation of Furanic Compounds

In a further aspect, the invention pertains to processes for oxidizing furanic compounds. In particular the invention pertain to process wherein furanic precursors of FDCA are oxidized. A process of the invention may comprise a single oxidation reaction step resulting in a product (e.g. the oxidation of HMFCA to FFCA). Alternatively a process of the invention may comprise more than one oxidation reaction step, each step resulting in an intermediate, where the last intermediate is the final product. Examples of such a series of steps, wherein HMF is oxidized in sequential oxidation steps to FDCA include e.g.: 1) HMF is first oxidized to HMFCA, which in a second step is oxidized to FFCA, which is then finally oxidized to FDCA, or alternatively, as described by Dijkman et al. (2014, Angew. Chem. 53 (2014) 6515-8) 2) HMF is first oxidized to DFF, which in a second step is oxidized to FFCA, which is then finally oxidized to FDCA. Thus, in a preferred process of the invention one or more furanic precursors of FDCA are oxidized in a series of steps to ultimately FDCA.

In one embodiment, the invention relates to processes comprising at least the oxidation of HMFCA to FFCA. Preferably, the process is a process for oxidizing HMFCA to FFCA, wherein the process comprises the step of incubating a cell in the presence of HMFCA. The cell preferably is a cell expressing enzymes that have the ability to oxidize HMFCA to FFCA. The cell can be cell that is genetically modified to have the ability to oxidize HMFCA to FFCA. In a preferred embodiment, the cell is a genetically modified fungal cell as herein defined above. Preferably the cell is incubated in the presence of HMFCA under conditions conducive to the oxidation of HMFCA by the cell, as e.g. specified below.

In another embodiment, the invention relates to processes for producing FDCA. A process for producing FDCA preferably comprises the step of incubating a cell in a medium comprising one or more furanic precursors of FDCA. The cell preferably is a cell expressing one or more enzymes that have the ability to convert a furanic precursor of FDCA into FDCA. The enzymes with the ability to convert a furanic precursors of FDCA into FDCA can be an enzyme having alcohol and/or aldehyde dehydrogenase activities as described above, including the exemplified fungal enzymes. Thus, in a preferred embodiment, the cell is a genetically modified fungal cell, as herein defined above.

Preferably the cell is incubated in the presence of one or more furanic precursors of FDCA under conditions conducive to the oxidation furanic precursors of FDCA by the cell to FDCA, as e.g. specified below.

Preferably in the process, at least one furanic precursor of FDCA is selected from the group consisting of HMF, DHF, HMFCA, FFCA and DFF, of which HMF is most preferred. The furanic precursors of FDCA are preferably obtained from one or more hexose sugars, preferably by acid-catalysed dehydration, e.g. by heating in presence of acid, in a conventional manner. The technology to generate HMF from fructose is well established and robust (see e.g. van Putten et al., 2013, Chem. Rev. 113, 1499-1597). Also glucose-rich feedstock can be utilized, but the thermochemical formation of HMF proceeds more efficiently from fructose. Therefore, an additional enzymatic step can be included to convert glucose to fructose, using glucose isomerase. The latter process is well-established in food industry e.g. for producing high fructose corn syrup (HFCS) from hydrolysed starch. Glucose can also be chemically isomerised to fructose using combinations of catalysts and solvents as e.g. described in van Putten et al. (2013, supra).

The hexose sugars will usually be obtained from biomass. The term “biomass” is understood to mean the biodegradable fraction of products, waste and residues from biological origin from agriculture (including vegetal, such as crop residues, and animal substances), forestry (such as wood resources) and related industries including fisheries and aquaculture, as well as biodegradable fraction of industrial and municipal waste, such as municipal solid waste or wastepaper. In a preferred embodiment, the biomass is plant biomass, more preferably a (fermentable) hexose/glucose/sugar-rich biomass, such as e.g. sugarcane, a starch-containing biomass, for example, wheat grain, or corn straw, or even cereal grains, such as corn, wheat, barley or mixtures thereof. Preferred are agricultural crops naturally rich in fructans (e.g., topinambur or chicory roots).

The hexose sugars can be obtained by hydrolysis of such biomass Methods for hydrolysis of biomass are known in the art per se and include the use of e.g. vapour and/or carbohydrases such as glucoamylases.

Another preferred type of biomass for use in the process of the invention is a so-called “second generation” lignocellulosic feedstock, which are preferred if large volumes of FDCA are to be produced in a more sustainable way. Lignocellulosic feedstocks can be obtained from dedicated energy crops, e.g. grown on marginal lands, thus not competing directly with food crops. Or lignocellulosic feedstocks can be obtained as by-products, e.g. municipal solid wastes, wastepaper, wood residues (including sawmill and paper mill discards) and crop residues can be considered. Examples of crop residues include bagasse from sugar cane and also several corn and wheat wastes. In the case of corn by-products, three wastes are fiber, cobs and stover. Furthermore, forestry biomass may be used as feedstock. In order to convert second generation feedstocks into fermentation products of the invention, the cellulose and hemicellulose need to be released as monosaccharides. Hereto, either thermochemical approaches (usually referred to as pretreatment), enzymatic approaches or a combination of the two methodologies are applied. A pretreatment can serve to either completely liberate the sugars, or to make the polymeric compounds more accessible to subsequent enzymatic attack. Different types of pretreatment include liquid hot water, steam explosion, acid pretreatment, alkali pretreatment, and ionic liquid pretreatments. The relative amounts of the various compounds will depend both on the feedstock used and the pretreatment employed. For release of monosaccharide sugars from such lignocellulosic feedstock, appropriate carbohydrases are employed, including e.g. arabinases, xylanases, glucanases, amylases, cellulases, glucanases and the like.

The oxidation process of the invention is preferably conducted at temperature most optimal to the cell and/or the oxidoreductase enzymes contained is the cell. Thus, in case of thermophilic cells and/or thermophilic enzymes the temperature preferably is in the range between 40 and 65° C., e.g. at least 40, 42, or 45° C. and/or not higher than e.g. 65, 60, 55 or 50° C. However, in the case of a mesophilic cell and/or enzymes from mesophiles, the oxidation reactions are preferably conducted at a relatively mild temperature, e.g. in the range of 10-45° C., more preferably 20-40° C., e.g. at least 10, 15, 18, 20, 22 or 25° C. and/or not higher than e.g. 45, 42, 40, 38, 35, 32 or 30° C.

The oxidation process of the invention is preferably conducted at acidic pH. Downstream processing (DSP), i.e. recovery and purification, is of general concern in any biotechnological process but in particular in productions of monomeric compounds for polymer productions because the purity of the monomer is essential in controlled polymer formation. FDCA has a very limited solubility at pH-values below 3 (with a pKa of around 2.28). When the process is carried out at acidic pH, the FDCA produced will precipitate from the medium in which it is produced, preferably already during its production, thereby greatly facilitating its recovery. Preferably therefore, in the process of the invention, the cell, preferably a fungal cell is incubated in the presence of one or more furanic at a pH equal to or lower than 5.0, 4.0, 3.0, 2.9, 2.8, 2.7, 2.6, 2.5 or 2.4, and preferably at a pH that is equal to or higher than 2.0, 2.1, 2.2 or 2.25, 2.27 or 2.28. Preferably, in the process of the invention a cell, preferably a fungal host cell is selected that has a high tolerance to a pH in this range. An additional advantage of carrying out the process at acidic pH is that microbial contaminations of the process will be less of a problem since almost all bacteria are adversely affected at low pH. Yeasts and fungi are less of a problem compared to bacteria as source of infections and will be relatively easy to deal with.

The reaction time may be 6-150 hrs, more preferably 6-18 hrs. Preferably oxygen is supplied to the cells in the reaction medium from an oxygen source, such as molecular oxygen, e.g. as pure oxygen or in air, or water, or a different source of oxygen depending on the requirements of the furanic oxidizing enzyme. Air may be used conveniently as a source of molecular oxygen.

The reactor may be any suitable (aerated) bioreactor. It may be operated in batch, continuous or preferably in fed-batch.

The process of the invention further preferably comprises the step of recovery of the oxidation product(s) produced in the process, such as FDCA, or HMFCA. Preferably, the oxidation product is recovered from the medium in which the cell carrying out the oxidation steps is incubated. Oxidation products such as FDCA, HMFCA, etc. may be recovered from the reaction mixture or medium by e.g. (acid) precipitation, subsequent cooling crystallisation, and separation of the crystallized oxidation product, e.g., crystallized FDCA. However, other recovery methods are suitable, such as e.g. acid precipitation and solvent extraction, as known in the art.

The processes of the invention for oxidizing furanic compounds may advantageously be applied for the elimination of furanic compounds from feedstocks wherein furanic compounds are considered to be detrimental, such as feedstocks for fermentations for the production of biofuels and biochemicals. More preferably, the processes for oxidizing furanic compounds are applied in the bioproduction of FDCA as a monomeric precursor for the production of polyesters (plastics), wherein FDCA may substitute for PTA in the polyester PET in which case biobased polyethylenefurandicarboxylate (PEF) results. FDCA may also be used as a substrate for a large variety of valuable compounds, including e.g. as substrate for the production of succinic acid, 2,5-bis(aminomethyl)-tetrahydrofuran, 2,5-dihydroxmethyl-tetrahydrofuran, 2,5-dihydroxymethylfuran and 2,5-furandicarbaldehyde. FDCA may be used in the production of coatings, e.g. in alkyd resin and thermoplastic coatings. It may also be used as a xylene equivalent in biofuels and as solvent. FDCA may be esterified, and the esters may be used as plasticizers. FDCA may converted to its diol, that may be used in PET-like polyesters and polyurethanes. Further FDCA may be converted into its diamine, the diamine may be used as chain extender and the diamine may be converted into di-isocyanate, which can be used in the production of polyurethanes.

Thus, in a further aspect the invention relates to a process for producing a polymer from one or more, or at least two FDCA monomers, the process comprising the steps of: a) preparing an FDCA monomer in an oxidation process of the invention as described above; and, b) producing a polymer from the FDCA monomer(s) obtained in a). In a preferred process, the polymer is produced by mixing FDCA monomers with one or more types of diol monomers and bringing the mixture in a condition under which the FDCA monomers and the diol monomers polymerise. The polymerization can thus be an esterification or a trans-esterification, both being also referred to as (poly)condensation reactions. The diol monomer can be an aromatic, aliphatic or cycloaliphatic diol, e.g. an alkylene glycol. Examples of suitable diol and polyol monomers therefore include ethylene glycol, diethylene glycol, 1,2-propanediol, 1,3-propanediol, 1,4-butanediol, 1,5-pentanediol, 1,6-hexanediol, 1,4-cyclohexanedimethanol, 1,1,3,3-tetramethylcyclobutanediol, 1,4-benzenedimethanol, 2,2-dimethyl-1,3-propanediol, poly(ethylene glycol), poly(tetrahydofuran), 2,5-di(hydroxymethyl)tetrahydrofuran, isosorbide, glycerol, 25 pentaerythritol, sorbitol, mannitol, erythritol, threitol. Ethylene glycol, 1,3-propanediol, 1,4-butanediol, 1,4-cyclohexanedimethanol, 1,6-hexanediol, 2,2-dimethyl-1,3-propanediol, poly(ethylene glycol), poly(tetrahydofuran), glycerol, and pentaerythritol, are preferred diols. Ethylene glycol is paricularly preferred. Ethylene Glycol (MonoEthylene Glycol—MEG) is preferably biosourced. For example biosourced MEG can be obtained from ethanol which can also be prepared by fermentation from sugars, (e.g. glucose, fructose, xylose) that can be obtained from crop or agricultural by-products, forestry byproducts or solid municipal waste by hydrolysis of starch, cellulose, or hemicellulose. Alternatively, biosourced MEG can be obtained from glycerol, that itself can be obtained as waste from biodiesel. Preferably the polymer is polyethylenefurandicarboxylate (PEF).

The polymer can also comprise other contain a (minor) amount of other reactive monomers having a structure different from the FDCA and/or alkylene glycol monomer such as other diacid monomers, e.g dicarboxylic acid or polycarboxylic acid, for instance therephthalic acid, isophtahalic acid, cyclohexane dicarboxylic acid, maleic acid, succinic acid, 1,3,5-benzenetricarboxylic acid. Lactones can also be used in combination with the 2,5-furandicarboxylate ester: Pivalolactone, eppilon-caprolactone and lactides (L,L; D,D; D,L). Even if it is not the most preferred embodiment of the invention, the polymer can be non linear, branched, thanks to the use of polyfunctional monomers (more than 2 acid or hydroxyl functions per molecule), either acid and/or hydroxylic monomers, e.g. polyfunctional aromatic, aliphatic or cycloaliphatic polyols, or polyacids. In one embodiment, the alkylene glycol in the polymer is partly or fully replaced by an alkylene diamine.

In yet another aspect, the invention pertains to the use of a cell, preferably a cell of the invention, for the biotransformation of one or more of furanic precursors to FDCA to FDCA, wherein the cell is a cell expressing an HMFCA dehydrogenase as herein defined above, or a cell expressing polypeptide having furanic compound transport capabilities and further comprising a HMFCA dehydrogenase activities as herein defined above. Preferably, at least one furanic precursor of FDCA that is biotransformed to FDCA is selected from the group consisting of HMF, DHF, HMFCA, FFCA and DFF, of which HMF is most preferred.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

The following examples are offered for illustrative purposes only, and are not intended to limit the scope of the present invention in any way.

TABLE 1
Amino acid sequence alignment of Penicillium brasilianum hmfL1
and 10 closest orthologues.

| | |
|---|---|
| P_brasilianum_hmfL1 | MGSLSLPETSLAAIQDK--ETKAISVAKRPTPVPVGTQVLVKLHYSGVCA |
| Spo_sche_ERT02385 | ---MAVPTTSTAAIRDD--QGK-ISVQQRPTPVPERTQILVKVHYSGVCA |
| Asp_kawa_GAA84694 | -MSTDNPATRKVAVCID--TQH-IKVEERPLPIPNDSEVVVLIEASGICA |
| Bys_spec_GAD98038 | -MGSTIPATRKVAVTTS--PPT-VSITSLPIPQPAGTEVLLQIEATGICA |
| Asp_nige_XP_001397354 | -MTTNVPATRKVAVCID--TQH-IELEERPMPTPSGSEVVVKIQATGICA |
| Eut_lata_XP_007796771 | -MSCSPPTQSRVVVAKG--THD-LVVQERQTPEPTGKQILLRIEATGVCA |
| Asp_nige_EHA21652 | ------------------------------MPTPSGSEVVVKIQATGICA |
| Fus_gram_EYB30957 | -MSVQIPSQQRAAVRQGSGPDARAPIKTVPVPSPGQGQILVKVNWTGLCG |
| Fus_gram_XP_011318199 | -MSVQIPSQQRAAVRQGSGPDARAPIKTVPVPSPGPGQILVKVNWTGLCG |
| Rhi_phas_WP_016737077 | -MKIMTSKMMKAAVVRE--FGKPLAIECVPVPVPGPGEILVKVAACGVCH |
| Dye_jian_WP_038619920 | ----MAPRTMKAAVAHR--FGEPLRIEEVPVPAPGRGEVLVKIVSSGVCH |
| | * * :::: : *:* |
| P_brasilianum_hmfL1 | TD---LHLARGSVPYLQPKVS--VGGHEGTGVIASLGPDVDAAEWHVGDR |
| Spo_sche_ERT02385 | TD---VHIARGLIPYLRPKVA--VGGHEGTGVIAALGPDVDASQWAIGDR |
| Asp_kawa_GAA84694 | TD---LHLVRRSIPYLQREVD--VCGHEGVGRIVALGPDVDTSEWRLGDR |
| Bys_spec_GAD98038 | TD---LHIVQRSLSYFQPKVD--IHGHEGIGRIVALGPDVDASKWKIGDK |
| Asp_nige_XP_001397354 | TD---LHLVRRTIPYLQRKVD--VCGHEGVGHIVAVGPDVDTSKWHMGDR |
| Eut_lata_XP_007796771 | TD---LHLIRRSIPYLQPKVD--ICGHEGIGRIVRLGPEADQKRWSVGDR |
| Asp_nige_EHA21652 | TD---LHLVRRTIPYLQRKVD--VCGHEGVGHIVAVGPDVDTSKWHMGDR |
| Fus_gram_EYB30957 | SDKSLLHDEWSDFGICMKDVTNGIAGHEGAGSVVAVGQGMEQR-WKIGDR |
| Fus_gram_XP_011318199 | SDKSLLHDEWSDFGICMKDVTNGIAGHEGAGSVVAVGQGMEQR-WKIGDR |
| Rhi_phas_WP_016737077 | TD---LHAAEGDWPVMPVPPF--IPGHEAAGIVAALGPDVTEF--KEGDA |
| Dye_jian_WP_038619920 | TD---VHAVDGDWPVKPQPPF--IPGHEGVGVVVALGEGVDNL--KVGDA |
| | :* :* : ***. * :. :* ** |
| P_brasilianum_hmfL1 | VAVRWVHIVCGKCEVCTTGF-ENLCQSRKLAGKDVEGTFAEYAIADSSYM |
| Spo_sche_ERT02385 | VAVRWVHIVCGTCESCTTGH-ENLCAGRKLAGKDVDGTFAEYAIADSAYA |
| Asp_kawa_GAA84694 | VAHRWIFDVCRNCEMCQEGN-EQLCDSRKLSGKDVEGCWGEYTIVNSKYL |
| Bys_spec_GAD98038 | VAHRWIYRWCKECEPCRAGL-EQFCDKRQLSGLQVEGCWAEYTVADTEYM |
| Asp_nige_XP_001397354 | VAHRWVFDVCLNCDMCQGGN-EQLCDSRKLSGKDVEGCWGEYTIVNSMYL |
| Eut_lata_XP_007796771 | VAHRWIYRWCGECESCEDGN-EQLCDRRELSGKDIDGCWAEYTLVDSDYL |
| Asp_nige_EHA21652 | VAHRWVFDVCLNCDMCQGGN-EQLCDSRKLSGKDVEGCWGEYTIVNSMYL |
| Fus_gram_EYB30957 | AGVKWIASVCGECDFCMVGSDEVHCPEQTNSGFSVPGTFQEYVVADGKYS |
| Fus_gram_XP_011318199 | AGVKWIASVCGECDFCMVGSDEVHCPEQTNSGFSVPGTFQEYVVADGKYS |
| Rhi_phas_WP_016737077 | VGVAWLHDACLRCEYCETGW-ETLCAHQHNTGYSCNGGFAEYVIASAAFA |
| Dye_jian_WP_038619920 | VGIAWLHDACGHCEYCITGW-ETLCEAQHDSGYSVNGSFAEYAIGNAAYV |
| | .. *: * *: * * * * : :* . * : **.: . : |
| P_brasilianum_hmfL1 | VRLPAGVSDADAAPILCAGVTVYKALKIASLRAGSWVAVAGAGGGLGHLA |
| Spo_sche_ERT02385 | VRLPENVGDAEAAPILCAGVTVYKALKIARLRKGSWVAVAGAGGGLGHLA |
| Asp_kawa_GAA84694 | MRISEDISATEAAPTLCAGTTAYRAIRTTGLTSGQWIAIIGAGGGLGHLA |
| Bys_spec_GAD98038 | LRIPEGLDSAEAAPILCAGTTVYRALRTSELSPGQWVAIVGAGGGLGHLA |
| Asp_nige_XP_001397354 | MRIPEDISAAEAAPTLCAGTTAYRAIRTAGLTSGQWIAIVGAGGGLGHLA |
| Eut_lata_XP_007796771 | LRIPEEIDPVAAAPILCAG---------------HWVAIVGAGGGLGHLA |
| Asp_nige_EHA21652 | MRIPEDISAAEAAPTLCAGTTAYRAIRTAGLTSGQWIAIVGAGGGLGHLA |
| Fus_gram_EYB30957 | SKLPDGVTDEEAGPIMCGGVTAYTACKRSGVTPGQWLVIPGAGGGLGHFA |
| Fus_gram_XP_011318199 | SKLPDGVTDEEAGPIMCGGVTAYTACKRSGVTPGQWLVIPGAGGGLGHFA |
| Rhi_phas_WP_016737077 | ARLPAGVDFAEIAPILCAGVTTYKGLKETEARPGEWVAISGVGG-LGHVA |
| Dye_jian_WP_038619920 | ARLPKDVDYAAMAPILCAGVTTYKGIRETEARPGEWIAISGIGG-LGHLA |
| | ::. : .* :*.* *:.: * ** ***.* |
| P_brasilianum_hmfL1 | IQYARAMGLKVVALDAR--KRDLCLSLGAESYIDVLET---DDCVAQVIK |
| Spo_sche_ERT02385 | VQYAKALGLKVVALDAN--KKDLCLSLGADAYVDVLAPGHDDGCVGAVVA |
| Asp_kawa_GAA84694 | IQYAKASGLRVLGIDTGPSKRELSCKLGVTSYIDFMDT---PDLTADVIR |
| Bys_spec_GAD98038 | IQYAKVQGLKVLAIDGGKEKEKLCTDLGADVYIDFTST---KDITATVID |
| Asp_nige_XP_001397354 | VQYAKANDLQVLGIDTGPSKWELCSRLGVTSYIDFMET---RDLTADVTR |
| Eut_lata_XP_007796771 | IQYAKVKGLKVLAIDAGGEKGAMCTKLGADAFVDFTQT---KDITSDVVK |
| Asp_nige_EHA21652 | VQYAKANDLQVLGIDTGPSKWELCSRLGVTSYIDFMET---RDLTADVTR |
| Fus_gram_EYB30957 | IQYAKAMGMRVIAIDGGDEKRDLCLKLGAEVFIDFKTT---KDIATQVLK |
| Fus_gram_XP_011318199 | IQYAKAMGMRVIAIDGGDEKRDLCLKLGAEVFIDFKTT---KDIATQVLK |
| Rhi_phas_WP_016737077 | IQYAKAMGLKVVALDVAAAKLDLARQVGADLALNARSE----DTVEKVLK |
| Dye_jian_WP_038619920 | IQYATAMGLNVVAVDVAEEKLALARKLGASAAVDARSP----NAVEEVLD |
| | :*** . .:.*:.:* * :. :*. :: . . * |
| P_brasilianum_hmfL1 | VTDG-GAHGALICASSGQAYDDAVKFLRWTGTLVCIGLP----------- |
| Spo_sche_ERT02385 | ATDGVGAHGALICASSGVAYADAVKYLRKSGVLVCIGLP----------- |
| Asp_kawa_GAA84694 | VTDG-GPHGVIVVSSSSMAYEQALQYVRKMGIIVCIGIT----------- |
| Bys_spec_GAD98038 | ITSG-GAHGILVTSSSPRAYEQAITYVRKMGIIVCIGAT----------- |
| Asp_nige_XP_001397354 | VSGG-GPHGVIVVSSSTRAYEQALTYVRKMGIIVCIGISKLRWYLRATPQ |
| Eut_lata_XP_007796771 | ITNG-GAHAILVTSSSVRAYEQAITYVRKRGIIICIGIT----------- |
| Asp_nige_EHA21652 | VSGG-GPHGVIVVSSSTRAYEQALTYVRKMGIIVCIGIK----------- |
| Fus_gram_EYB30957 | VTTH-GAHGVIVTAATRAAYESAPNYLRPNGTVVAVGLP----------- |
| Fus_gram_XP_011318199 | VTTH-GAHGVIVTAATRAAYESAPNYLRPNGTVVAVGLP----------- |
| Rhi_phas_WP_016737077 | ATNG-GAHGVVVTAVSPSAFSQALGMVRRKGTVSLVGLP----------- |
| Dye_jian_WP_038619920 | ATGG-GAHGVLVTAVSPKAFSQALNFTRRRGTMSLVGLP----------- |
| | : *.*. :: : : *: .* * * : :* |
| P_brasilianum_hmfL1 | ------PKPTLLSLGPADFVARG-IKVMGTSTGDRQDTVEALAFVAKGQV |
| Spo_sche_ERT02385 | ------LRPTPIPVLPEDFVARG-LRLEGTSTGDRTDTAEALEFVARGQV |
| Asp_kawa_GAA84694 | ------PNKMHFPIGPEYFVARG-VRLTGSSTGTMEDTREALQYVRDGRV |
| Bys_spec_GAD98038 | ------STKMTFPIGPEYFVGKG-VRLTGTSTGTLRDTEEALELVRQGKV |
| Asp_nige_XP_001397354 | ANIPQAPNKMHFPIGPEYFVARG-VRLTGSSTGTMEDTCQALQYVRDGRV |
| Eut_lata_XP_007796771 | ------PQKMSFPIGPEYFVARG-VRLTGTSTGTIEDTKEALEYVKTGQV |
| Asp_nige_EHA21652 | -----------------YFVARG-VRLTGSSTGTMEDTCQALQYVRDGRV |
| Fus_gram_EYB30957 | ------QDPTVLAGAPPMLVALRRLKIVGSVTGSMKDVEEALEFTARGLV |
| Fus_gram_XP_011318199 | ------QDPTVLAGAPPMLVALRRLKIVGSVTGSMKDVEEALEFTARGLV |
| Rhi_phas_WP_016737077 | --------PGNFPTPIFDVVLKR-ITIRGSIVGTRRDLDEALAFAAEGRV |
| Dye_jian_WP_038619920 | --------PGDFATPIFDVVLKR-LTIRGSIVGTRKDLAEAVAFAAEGKV |
| | .* : : *: .* * :*: . * * |
| P_brasilianum_hmfL1 | KPQLTERRLEDVEEILKEIENGTMQGKAVIRIA-------------- |
| Spo_sche_ERT02385 | KPQIVERQLGEIEAILEEIEKGTVHGKSVIKIA-------------- |
| Asp_kawa_GAA84694 | KPMIVEVRLEDIGACLQALEKGEGDGRFVVKF--------------- |
| Bys_spec_GAD98038 | KPIIVEKKLEDIPECLDLLAKGDAVGKFVVKL--------------- |
| Asp_nige_XP_001397354 | KPIIVEARLEEIEACLQALEKGEADGRFVVSFS-------------- |
| Eut_lata_XP_007796771 | KPITIEKRLEDIAECLSILEKGDAVGRYVVRL--------------- |
| Asp_nige_EHA21652 | KPIIVEARLEEIEACLQALEKGEADGRFVVSFS-------------- |
| Fus_gram_EYB30957 | HPILSKGKLEDLDDWVHKLATGQVAGRCVLKVAA------------- |
| Fus_gram_XP_011318199 | HPILSKGKLEDLDDWVHKLATGQVAGRCVLKVAA------------- |
| Rhi_phas_WP_016737077 | RAEIAKAPLDDINDIFASLKAGTIEGRMVLDIAGEAGVSAAAEQSAA |
| Dye_jian_WP_038619920 | VPTIERRKLEDVNDVLQGLREGHIQGRVVLDIGTPU---SAGE---- |
| | . . * :: . : * *: *: . |

TABLE 2
Amino acid sequence alignment of Penicillium brasilianum hmfL2
and 10 closest orthologues.

| | |
|---|---|
| P_brasilianum_hmfL2 | MS--LPSHYKRAAFKEAGGPLTIEEVDLTMPDAGEVLVKVEACGVCFSDT |
| Coc_immi_XP_001244132.2 | MA--LPQTFKQAVFKGAGKPLVIEEVSLALPGPGEVLVKVEACGVCFSDT |
| Coc_posa_XP_003068662 | MA--LPQTFKQAVFKGAGKPLVIEEVSLALPGPGEVLVKVEACGVCFSDT |
| Coc_posa_EFW20539 | MA--LPQTFKQAVFKGAGKPLVIEEVSLALPGPGEVLVKVEACGVCFSDT |
| Tri_rubr_XP_003235253 | MD--IPKTFKQAIFKEKGAPLVLEEVPMTPPGNGEVLVKVQACGVCHSDV |
| Tri_equi_EGE05431 | MD--IPKTFKQAIFKEKGAPLVLEEVPMTPPGNGEVLVKVQACGVCHSDV |
| Cha_glob_XP_001220755 | MT--LPKTFKQAAFHSQGAALTIEDAELRLPGPGEVLVKVEACGVCFSDM |
| Tri_tons_EGD92820 | MD--IPKTFKQAIFKEKGAPLVLEEVPMTPPGNGEVLVKVQACGVCHSDV |
| Mic_gyps_XP_003173798 | MD--IPKTFKQAIFKEVGAPLVLEEVPMTPPGKGEVLVKVQTCGVCYSDT |
| End_pusi_XP_007800835 | MAPELPKTFKRAVFKEQGAPLTIEEVELRMPERGEVLVKVEACGVCHSDS |
| Art_otae_XP_002844685 | MD--APKTFKQAIFKEAGAPLVLEEVPLTPPEKGEVLVKVQACGVCRSDF |
| | * *. :*:* *: * .*.:*:. : * *******::**** ** |
| P_brasilianum_hmfL2 | VPQAHGLGGKFPIVPGHEIIGHVVATGDGVSDWEVGDRIGEGWHGGHDGT |
| Coc_immi_XP_001244132.2 | YAQKNMLGGGFPIVPGHEIIGRVAAVGDGVSGWGLGDRIGGGWHGAHDGT |
| Coc_posa_XP_003068662 | YAQKNMLGGGFPIVPGHEIIGRVAAVGDGVSGWGLGDRIGGGWHGAHDGT |
| Coc_posa_EFW20539 | YAQKNMLGGGFPIVPGHEIIGRVAAVGDGVSGWGLGDRIGGGWHGAHDGT |
| Tri_rubr_XP_003235253 | FVQNDGLGGGLPRVPGHEIIGHVAATGEGVTQWKVGDRIGGAWHGGHDGT |
| Tri_equi_EGE05431 | FVQNDGLGAGLPRVPGHEIIGHVAAIGEGVTQWKVGDRIGGAWHGGHDGT |
| Cha_glob_XP_001220755 | FAQQNIMGGGFPIVPGHEIIGRVAAVGDGVTAWKVGERVGAGWHGGHDGT |
| Tri_tons_EGD92820 | FVQNDGLGAGLPRVPGHEIIGHVAAIGEGVTQWKVGDRIGGAWHGGHDGT |
| Mic_gyps_XP_003173798 | AVQKNALGGGLPRVPGHEIIGHVAAVGEGVTQWKVGDRIGGAWHGGHDG- |
| End_pusi_XP_007800835 | MAQMNIFGGGFPLVPGHEIIGHVAAVADGETAWKVGDRIGGPWHGGHDGT |
| Art_otae_XP_002844685 | YVQHNAVGS-LPRVPGHEIIGHVAAVGEGVTQWKVGDRIGGAWHGGHDGT |
| | * . .*. :* ********:*.* .:* : * :*:*:* ***.*** |
| P_brasilianum_hmfL2 | CPSCRQGHFQMCDNQSINGVTKNGGYAQYCILRSEAAVRIPTHVSAAEYA |
| Coc_immi_XP_001244132.2 | CKSCKKGLFQMCSNKLINGETRSGGYAEYCTLRAEAAVRVPDHIDAAKYA |
| Coc_posa_XP_003068662 | CKSCKKGLFQMCSNKLINGETRSGGYAEYCTLRAEAAVRVPDHVDAAKYA |
| Coc_posa_EFW20539 | CKSCKKGLFQMCSNKLINGETRSGGYAEYCTLRAEAAVRVPDHVDAAKYA |
| Tri_rubr_XP_003235253 | CRQCKKGYYQMCDNELVNGVNKGGGYAEYCLLRAEAGVRVPADVDAAVYA |
| Tri_equi_EGE05431 | CRQCKKGYYQMCDNELINGVNKGGGYAEYCLLRAEAGVRVPEDVDAAVYA |
| Cha_glob_XP_001220755 | CFACKKGLYQMCDNQVVNGETKAGGYAEYVLLRSEATVRVPEHVSAAKYA |
| Tri_tons_EGD92820 | CRQCKKGYYQMCDNELINGVNKGGGYAEYCLLRAEAGVRVPEDVDAAVYA |
| Mic_gyps_XP_003173798 | -------YYQMCDNALVNGVNKGGGYAEYCLLRSEAGVRIPPDVDAAKFA |
| End_pusi_XP_007800835 | CKACKTGFFQMCDNEKINGITRNGGYAQYCTLRSEAGVSIPSHLDAAEYA |
| Art_otae_XP_002844685 | CKPCKKGYFQMCDNALVNGVNKGGGYAEYCKLRAEAGVRIPADIDAAKYA |
| | :***.* :** .: ****:* **:** * :* .:.** :* |
| P_brasilianum_hmfL2 | PILCAGVTVFNSMRQIGVKPGSTVAIQGLGGLGHLAIQYANRFGFRVVAI |
| Coc_immi_XP_001244132.2 | PILCAGVTVFNSMRHMNVPPGETVAIQGLGGLGHLAIQCANRFGYRVVAI |
| Coc_posa_XP_003068662 | PILCAGVTVFNSMRHMNVPPGETVAIQGLGGLGHLAIQCANRFGYRVVAI |
| Coc_posa_EFW20539 | PILCAGVTVFNSMRHMNVPPGETVAIQGLGGLGHLAIQCANRFGYRVVAI |
| Tri_rubr_XP_003235253 | PILCAGVTVFNSMRNMKLGPGSTVAIQGLGGLGHLAIQYANKFGYRVVAL |
| Tri_equi_EGE05431 | PILCAGVTVFNSMRNMKLMPGSTVAIQGLGGLGHLAIQYANKFGYRVVAL |
| Cha_glob_XP_001220755 | PILCAGMTVFNSLRHMDVQPGETVAVQGLGGLGHLAIQAAQRMGYRVVAI |
| Tri_tons_EGD92820 | PILCAGVTVFNSMRNMKLMPGSTVAIQGLGGLGHLAIQYANKFGYRVVAL |
| Mic_gyps_XP_003173798 | PILCAGVTVFNSMRNMNLIPGSTVAIQGLGGLGHLAIQYANRFGYRVVAL |
| End_pusi_XP_007800835 | PILCAGVTVFNSMRRMQISPGSLVAVQGLGGLGHLALQFANKFGFRVAAL |
| Art_otae_XP_002844685 | PILCAGVTVFNSMRHMNMMPGSTVAVQGLGGLGHLAIQYANKFGYRVVAL |
| | ******:*****:*.: : **. **:**********:* *:::*:**.*: |
| P_brasilianum_hmfL2 | SRDDQKERFVRDLGAHEYINTSEEDVGSALQKLGGASLIVATAPNARAIS |
| Coc_immi_XP_001244132.2 | SRDSKKEKFARALGAHEYIDTSKEDVSKALRRLGKASMIVLTAPNADVVN |
| Coc_posa_XP_003068662 | SRDSKKEKFARALGAHEYIDTSKEDVSKALRRLGKASMIVLTAPNADVVN |
| Coc_posa_EFW20539 | SRDSKKEKFARALGAHEYIDTSKEDVSKALRRLGKASMIVLTAPNADVVN |
| Tri_rubr_XP_003235253 | SRGSDKEKFAKELGAHIYIDGGKGDIGEQLQAIGGADMIVSTAPSRSAVE |
| Tri_equi_EGE05431 | SRGSDKEKFAKELGAHIYIDGGKGDIGEQLQAIGGADMIVSTAPSRSAVE |
| Cha_glob_XP_001220755 | SRGADKEAFARQLGAHEYIDSSKGDVGEALRRLGGARLAMTTAPTAEVMG |
| Tri_tons_EGD92820 | SRGSDKEKFAKELGAHIYIDGGKGDIGEQLQAIGGADMIVSTAPSRSAVE |
| Mic_gyps_XP_003173798 | SRGSDKEKFARDLGAHIYIDGSKGDVGEQLQKLGGVDMIVSTAPSKNAVE |
| End_pusi_XP_007800835 | SRNADKEKFARDLGAHEYIDGSKGDQGEALQKLGGASLIVVTAPDAKVIS |
| Art_otae_XP_002844685 | SRGSEKEKFARDLGAHEYLDASKGDIGEQLQNLGGASMIVSTAPSKDAVE |
| | **. .** *.: **** *:: .: * .. *: :* . : : *** .: |
| P_brasilianum_hmfL2 | PLLKGLRPLGKLLILAVPGEIPLDTRLM----------VARGLSVHGWPS |
| Coc_immi_XP_001244132.2 | PLLNGLEARGKLLMLSGPGEVTINSSLM----------VVSGLSIHAWPS |
| Coc_posa_XP_003068662 | PLLNGLEARGKLLMLSGPGEVPINSSLM----------VVSGLSIHAWPS |
| Coc_posa_EFW20539 | PLLNGLEARGKLLMLSGPGEVPINSSLM----------VVSGLSIHAWPS |
| Tri_rubr_XP_003235253 | PLLKGLGMLGKLLVLSIPGDITVNTGLM----------LRRGLTVQCWPS |
| Tri_equi_EGE05431 | PLLKGLGMLGKLLILSIPGDITINTGLM----------VRRGLTVQCWPS |
| Cha_glob_XP_001220755 | TLLKGLGPMGKLLILSVPGDVPVNTGVM----------LKYALSVQSWPC |
| Tri_tons_EGD92820 | PLLKGLGMLGKLLIPSIPGDITINTGLM----------VRRGLTVQCWPS |
| Mic_gyps_XP_003173798 | PLLKGLGMLGKLLVLSVPGDITINTGLM----------VRRGLSVQCWPS |
| End_pusi_XP_007800835 | PLMKGLGIMGKLLILAAAGEVPVDTGAM----------IHYGLSVHSWPS |
| Art_otae_XP_002844685 | PLLKGLGMLGKLLILSVPGDITINTGLMNKAVDLLASQVRQGLSVQCWPS |
| | .*::** ****: : .*::.::: * : .*::: **. |
| P_brasilianum_hmfL2 | GHALDSEETIRFTELEDIKCMIQTYSLDRANEAFDAMISGSVRFRAVITM |
| Coc_immi_XP_001244132.2 | GHATDSEEAIAFTELQNINCMVETFPLARANDAFEAMLKGTVRFRAVITM |
| Coc_posa_XP_003068662 | GHATDSEEAIAFTELQNINCMVETFPLARANDAFGKNSHKN--------- |
| Coc_posa_EFW20539 | GHATDSEEAIAFTELQNINCMVETFPLARANDAFGNVERDGSV------- |
| Tri_rubr_XP_003235253 | GHATDSEDAIEFTKLENINCMVEKFPLAKVQEAYDAMVKGTVRFRAVITM |
| Tri_equi_EGE05431 | GHATDSEDAIEFTKLENINCMVEKFPLAKVQEAYDAMVKGTVRFRAVITM |
| Cha_glob_XP_001220755 | GHATDSEDAIQFMDLQKVDCIVQTFPLAKANEAFNAMMDGSVRFRTVIVM |
| Tri_tons_EGD92820 | GHATDSEDAIEFTKLENINCMVEKFPLAKVQEAYDAMVKGTVRFRAVITM |
| Mic_gyps_XP_003173798 | GHATDSEDAIEFAKLEGINCMVETFPLAKVNEAYDAMVKGTVRFRAVITM |
| End_pusi_XP_007800835 | GHSLDSEEAIAFTELENIKCMVEKFQLEKCNDAMDAMMKGTVKVEEAAEL |
| Art_otae_XP_002844685 | GHATDSEEAIEFTKLENINCMVETFPLEKVNDAYDAMVKGSEPIMGTPUS |
| | **: ***::* * .*: :.*:::.: * : ::* |
| P_brasilianum_hmfL2 | E------------------------------------------------- |
| Coc_immi_XP_001244132.2 | E------------------------------------------------- |
| Coc_posa_XP_003068662 | -------------------------------------------------- |
| Coc_posa_EFW20539 | -------------------------------------------------- |
| Tri_rubr_XP_003235253 | E------------------------------------------------- |
| Tri_equi_EGE05431 | E------------------------------------------------- |
| Cha_glob_XP_001220755 | E------------------------------------------------- |
| Tri_tons_EGD92820 | E------------------------------------------------- |
| Mic_gyps_XP_003173798 | E------------------------------------------------- |
| End_pusi_XP_007800835 | CRRIGEWFAELEVPGRSSAGWLEDIQPDSWVGHVFCIWKREPGVVVGIEL |
| Art_otae_XP_002844685 | AGE----------------------------------------------- |
| P_brasilianum_hmfL2 | -------------------------------------------------- |
| Coc_immi_XP_001244132.2 | -------------------------------------------------- |
| Coc_posa_XP_003068662 | -------------------------------------------------- |
| Coc_posa_EFW20539 | -------------------------------------------------- |
| Tri_rubr_XP_003235253 | -------------------------------------------------- |
| Tri_equi_EGE05431 | -------------------------------------------------- |
| Cha_glob_XP_001220755 | -------------------------------------------------- |
| Tri_tons_EGD92820 | -------------------------------------------------- |
| Mic_gyps_XP_003173798 | -------------------------------------------------- |
| End_pusi_XP_007800835 | GPVVTDEGCSGPICGVEDPRLNLVIVELLGVVALSGSNVQDCSSSLGKLE |
| Art_otae_XP_002844685 | -------------------------------------------------- |
| P_brasilianum_hmfL2 | -------------------------------------------------- |
| Coc_immi_XP_001244132.2 | -------------------------------------------------- |
| Coc_posa_XP_003068662 | -------------------------------------------------- |
| Coc_posa_EFW20539 | -------------------------------------------------- |
| Tri_rubr_XP_003235253 | -------------------------------------------------- |
| Tri_equi_EGE05431 | -------------------------------------------------- |
| Cha_glob_XP_001220755 | -------------------------------------------------- |
| Tri_tons_EGD92820 | -------------------------------------------------- |
| Mic_gyps_XP_003173798 | -------------------------------------------------- |
| End_pusi_XP_007800835 | ATGSLKEILAPGPMGPKSSHSKFQAVASMMFTVAGMPEESQALLKKVFDV |
| Art_otae_XP_002844685 | -------------------------------------------------- |
| P_brasilianum_hmfL2 | -------------------------------- |
| Coc_immi_XP_001244132.2 | -------------------------------- |
| Coc_posa_XP_003068662 | -------------------------------- |
| Coc_posa_EFW20539 | -------------------------------- |
| Tri_rubr_XP_003235253 | -------------------------------- |
| Tri_equi_EGE05431 | -------------------------------- |
| Cha_glob_XP_001220755 | -------------------------------- |
| Tri_tons_EGD92820 | -------------------------------- |
| Mic_gyps_XP_003173798 | -------------------------------- |
| End_pusi_XP_007800835 | FDRTFVMIPLLLSGLRSQSRPSEDQYNDTNGC |
| Art_otae_XP_002844685 | -------------------------------- |

TABLE 3
Amino acid sequence alignment of Penicillium brasilianum hmfN1
and 10 closest orthologues.

| | |
|---|---|
| P_brasilianum_hmfN1 | ------MTQTNVHVNKSDTSLAAPQQLFISGKYQNSQRNGTFPVKNPMTG |
| Spo_sche_ERT02387 | ---------------MSYPPVSEPLQLYISGQHVASESSTTFPVMNPMTG |
| Sce_apio_KEZ45623 | -------MATNGGVGPKATTLSQVQELFIGGKHKPSSDNVEFQVINPMTG |
| Pod_anse_XP_001908521 | MAPHSPTTSNNGGVSERTSTLSQPQFLFINGKYILSSDNETFPVRNPITG |
| Eut_lata_XP_007794079 | ------------MANNGVSSLSEPQQLVIDGSYTTSSDGTTFQVVNPMKG |
| Sta_char_KEY72856 | ----------MARPRTNNDTLSSPQHLFINGAYRPSSDNSTFHVTNPMTG |
| Gae_gram_XP_009217152 | MVAHP--VAEKG-----PSALSQAQELVINGEAQPSSDGTTFTVRNPMTG |
| Sta_char_KFA73399 | ----------MARPRTNNDTLSSPQHLFINGAYRPSSDNSTFHVTNPMTG |
| Sta_char_KFA53356 | ----------MARPRTNNDTLSSPQHLFINGAYRPSSDNSTFHVTNPMTG |
| Cyp_euro_XP_008712551 | -------MHEKNGTTERRSTLTDEQLLYVNGEYVRPEDDAKFEVLNPATG |
| Sta_chlo_KFA62282 | ----------MAHLTASNDTLSSPQHLFINGAYRPSSNNSTFHVTNPMTG |
| | .:: * :.* .. . * * ** .* |
| P_brasilianum_hmfN1 | ETIYECVSASLDDYAAAIEEADAAQPSWARLGPSARRLILLKAADIMETY |
| Spo_sche_ERT02387 | EAIYQCASASPADYTTAIDAAYTAYQSWSRLGPSARRSVLLKAADIIESY |
| Sce_apio_KEZ45623 | ANIYSCASATVDDVSEAIESAHTAFKSWSRMGPSARRSIFLKAADILEGY |
| Pod_anse_XP_001908521 | SVLYNCASASKVDYETAIENAHSAYQTWSQTGPSARRRIFLKAADIMESY |
| Eut_lata_XP_007794079 | EKIYDCASATVQDYQKAIESASEAFKTWSRTSPSARRLVFLKAADIIEGY |
| Sta_char_KEY72856 | EPIYPCAAATAQDYLDAVAAAHAAYPRWSGTSPSARRLVLLRAADVLEGY |
| Gae_gram_XP_009217152 | QAIYECANATVDDYSRAIDTAHEAFKSWSATGPSARRLIFLKAAEIIESY |
| Sta_char_KFA73399 | EPIYPCAAATAQGYLDAVAAAHAAYPRWSGTSPSARRLVLLRAADVLEGY |
| Sta_char_KFA53356 | EPIYPCAAATAQDYLDAVAAAHAAYPRWSGISPSARRLVLLRAADVLEGY |
| Cyp_euro_XP_008712551 | GKIYDCSSAGVREYELAIKAADAAFTSWSQTAPSARRLIFLRAADTLERY |
| Sta_chlo_KFA62282 | EPIYSCAAATSQDYLDAVAAAHAAYPSWSRTSPSARRLILLRAADVLEGY |
| | :* * * *: * * *: .***** ::*:**: :* * |
| P_brasilianum_hmfN1 | IETDAPAILSAEVSATRGWVRANILSTAGVFRETAALATHIKGEIVPADR |
| Spo_sche_ERT02387 | LDQDAVAILSAEVSATRSWVKANMLSAAGVFRENAALATHIKGEIVPADR |
| Sce_apio_KEZ45623 | IHGDAPEILASEVSATATWVKVNIFSTANVLREAAGLVTHIKGEIVPADR |
| Pod_anse_XP_001908521 | ITGDAPEFMSQEVSATMHWVKINVFATAGLFRETASLATQIRGEIVPADR |
| Eut_lata_XP_007794079 | AKQDAPAILSAEVSATKSWVQVNIGATAGILRESAGLVTHIKGEIVPADR |
| Sta_char_KEY72856 | LESDAPEILASEVSATRSWVALNIRATAGILRETAGLATHIKGEIVPADR |
| Gae_gram_XP_009217152 | LGGDAPEVLSSEVSATAAWVRINMHATAGLFRETASLATHIRGEVVPADR |
| Sta_char_KFA73399 | LESDAPEILASEVSATRSWVALNIRATAGILRETAGLATHIKGEIVPADR |
| Sta_char_KFA53356 | LESDAPEILASEVSATRSWVALNIRATAGILRETAGLATHIKGEIVPADR |
| Cyp_euro_XP_008712551 | LHDDAPEILSAEVSAVSSWIRVNIMATANILRETAGQATQMRGEIVPADR |
| Sta_chlo_KFA62282 | LESDAPDILASEVSATRSWVALNIRATVGILRETAGLATHIKGEVVPADR |
| | ** .:: ****. *: *: ::..::** *. .*:::**:***** |
| P_brasilianum_hmfN1 | PGTTILVSREPVGVVLAISPWNMPATLTARAICCPLICGNSVVLRPSEFS |
| Spo_sche_ERT02387 | PGTTILVNREAVGVVLAISPWNMPVTLTARAVCCPLICGNAVLLKPSEYS |
| Sce_apio_KEZ45623 | PGTTVLITREPLGVMYAISPWNAPVNLTARAIACPLICGNTVVLKPSEYS |
| Pod_anse_XP_001908521 | PGTTIWVERQPVGVVFAISPWNAPINLTARAIAVPLLCGNTVVLKPSEFS |
| Eut_lata_XP_007794079 | PGTTILVERQPVGVVFAISPWNAPVNLTARAIATPL-------------- |
| Sta_char_KEY72856 | PGTTIMVERCPVGVVFAISPWNAPVNLTARAIATPLICGNTVVLKPSEYS |
| Gae_gram_XP_009217152 | PGTTILVERQAVGVVLAISPWNAPVNLTARSVACPLMCGNTVVVKPSEHS |
| Sta_char_KFA73399 | PGTTIMVERCPVGVVFAISPWNAPVNLTARAIAAPLICGNTVVLKPSEYS |
| Sta_char_KFA53356 | PGTTIMVERCPVGVVFAISPWNAPVNLTARAIATPLICGNTVVLKPSEYS |
| Cyp_euro_XP_008712551 | PGTMIMIMREAIGVVFAISPWNAPVNLTARAIASPLICGNTVVLKPSEFS |
| Sta_chlo_KFA62282 | PGTTIMVERCPVGVVFAISPWNAPVNLTARAIATPLICGNTSAPLPP--- |
| | *** : : * .:**: ****** * .****::. ** |
| P_brasilianum_hmfN1 | PKSQHLVVRALTEAGLPAGCLQFLPTSTADTPRAIEFAIRHPKVSRANFT |
| Spo_sche_ERT02387 | PKAQFLVVRALVEAGLPPGVLQFLPTSAADAPRATAFAIAHPKVSRTNFT |
| Sce_apio_KEZ45623 | PKSQHLVIKALTEAGLPAGCINFVPCSPDRAAANTEFAVKHPTVRHINFT |
| Pod_anse_XP_001908521 | PKSQDLAIRALTAAGLPPGCVNVLPTSAERTPEVTELAVKHPKVLRVNFT |
| Eut_lata_XP_007794079 | ----HLVVRALAEAGLPPGCLNFVPTSPERAPEVTEYAVKHPLVRRVNFT |
| Sta_char_KEY72856 | PKSQHLVIRALTAAGLPPGALNFLPTSPALAAAVTEQTVKHRHVLRVNFT |
| Gae_gram_XP_009217152 | PKSQALVVRALLEAGLPPGAIAFLPTSPGRAAEVTEYAVKHARVLRVNFT |
| Sta_char_KFA73399 | PKSQHLVIRALTAAGLPPGALNFLPTSPALAAAVTEQTVKHRHVLRVNFT |
| Sta_char_KFA53356 | PKSQDLVIRALTAAGLPPGALNFLPTSPALAAAVTEQTVKHRHVLRVNFT |
| Cyp_euro_XP_008712551 | PKSQHLVVRAFQEAGLPSGCLNFLPTKASDAAKVTEYATKHSKVRRLNYT |
| Sta_chlo_KFA62282 | -------------PACPPGALNFLPTSPALAAAVTEQTVKHRHVLRVNFT |
| | .. *.* : .:* .. :. : * * : *:* |
| P_brasilianum_hmfN1 | GSDRVGRIIAGLSASCLKPCVLELGGKAPVVVLEDADVEAAVEAVVYGAM |
| Spo_sche_ERT02387 | GGHRVGGIIASLSAKHIKKCLLELGGKAAVLVLHDADLDAAADAVAFGAM |
| Sce_apio_KEZ45623 | GSERVGKIIAGWAASCVKKCVFELGGKAPVIVREDADLDDAVESIIFGGL |
| Pod_anse_XP_001908521 | GSDRVGRIIAGWAATCLKQCVLELGGKAPVIVFEDANIDDAVEAVVFGAL |
| Eut_lata_XP_007794079 | GSDRVGKIIAGWAATCLKQCVLELGGKAPVLVLDDANIEDAVEAVAFGAF |
| Sta_char_KEY72856 | GSDRVGRIIAGWAAEVLKQCVLELGGKAPVLVLEDADVRGAVEAVVFGAL |
| Gae_gram_XP_009217152 | GSDRVGRIIAGHAAACLKQCVFELGGKAPVIVRADANLDDAVEAVVFGAL |
| Sta_char_KFA73399 | GSDRVGRIIAGWAAEVLKQCVLELGGKAPVLVLEDADVRGAVEAVVFGAL |
| Sta_char_KFA53356 | GSDRVGRIIAGWAAEVLKQCVLELGGKAPVLVLEDADVRGAVEAVVFGAL |
| Cyp_euro_XP_008712551 | GSDRVGKIIAGWAASCLKQCVLELGGKAPVIVLEDANIEDAVEAVVFGGF |
| Sta_chlo_KFA62282 | GSDRVGRIIAGWAAQVLKQCVLELGGKAPVLVLEDADVRDAVEAVVFGAL |
| | *..*** ***. :* :* *::******.*:* **:: *.::: :*.: |
| P_brasilianum_hmfN1 | SNSGQICMSTERAIVHRSLAADFKALLVKRAESLRVGNHLEDPDVQLSGL |
| Spo_sche_ERT02387 | SNSGQICMSTERVLVHASVAAAFKQKLVQRVEALRVGNHLDDPTVQLSGL |
| Sce_apio_KEZ45623 | ANNGQVCMSTERVIVHKSISGDFKSRLLARAGALKCGNHHVEKDVSISGL |
| Pod_anse_XP_001908521 | AFSGQVCMSTERVILHKSISREFKEKLLKKVESIKTGNHLEDPAVSISGL |
| Eut_lata_XP_007794079 | ANAGQICMSTERVLVHTSIAAKFKELLIQKSRELKTGNHEDDPEVSISGL |
| Sta_char_KEY72856 | ANAGQICMSTERVVVHDSVAKEFTEALVEKVGDVSVGNHMETPDVAISGL |
| Gae_gram_XP_009217152 | AYSGQVCMSTERAIVHRSVAAEFRTKVLARIAALRCGNHLDDAAVSVSGL |
| Sta_char_KFA73399 | ANTGQICMSTERVVVHDSVAKEFTEALVEKVGDVSVGNHMETPDVAISGL |
| Sta_char_KFA53356 | ANAGQICMSTERVVVHDSVAKEFTEALVEKVGDVSVGNHMETPDVAISGL |
| Cyp_euro_XP_008712551 | CNSGQICMSTERVIVEKAIEQKFTATLLEKVKTINWG---DQEGVSMAGL |
| Sta_chlo_KFA62282 | ANAGQICMSTERVVVHDSVAKEFTEALVKKVGDVSVGNHMETPDVAMSGL |
| | . **:******.::. :: * :: : : * * ::** |
| P_brasilianum_hmfN1 | FTAASAERVLGLIKGAVNAGATLLAGDLALHGPCQTIMAPHILTGVTRDM |
| Spo_sche_ERT02387 | FCAASAKRILGLLQAAVDAGATALTGDLQVHGPNGTILAPHVLEGVSADM |
| Sce_apio_KEZ45623 | FTPASASRVLGLVKSAVDTGATLLMGDMKLDGPNKTIMRPHILEGVTREM |
| Pod_anse_XP_001908521 | FTSAHAKRVMSLVKSAVDGGAKLLAGDLQVTGPRGTIIRPHILEHVSTNM |
| Eut_lata_XP_007794079 | YTPASATRILALMKDAVSSGAKLLCGDMSLAGPNKTIIAPHVFEGVTPEM |
| Sta_char_KEY72856 | YTPSSCTRILGLVREAMSQGATLLTGRLTPSGPNNTILAPMVLSHVTPAM |
| Gae_gram_XP_009217152 | FTPAHALRVLELVQDALAGGAELLAGDLATSGPCGTIVRPHVLSGVGPSA |
| Sta_char_KFA73399 | YTPSSCTRILGLVREAMSQGATLLTGRLTPSGPNNTILAPMVLSHVTPAM |
| Sta_char_KFA53356 | YTPSSCTRILGLVREAMSQGATLLTGRLTPSGPNNTILAPMVLSHVTPAM |
| Cyp_euro_XP_008712551 | YTPQSAERFLAMIEQAIADGAELLAGDRSASGPNRTLVQPHVLGKVTRTM |
| Sta_chlo_KFA62282 | YTPSSCTRILGLVREAMSKGAILLTGCLTPSGPNNTILAPIVLSHVTPAM |
| | : . . *.: ::. *: ** * * ** *:: * :: * |
| P_brasilianum_hmfN1 | DLFHRETFGPVLFVSEFDTDDEAIAQANDTEFSLCASVFSRDVLRAMDTA |
| Spo_sche_ERT02387 | DLYQQETFGPVVIVNTFADEADAVTQANQTDFTLCGSIFSRDVLRAADLA |
| Sce_apio_KEZ45623 | DLYHQESFGPVMILLEFETDEEGVDLANDSDFSLCASVFSRDVMRAMELA |
| Pod_anse_XP_001908521 | DIAHVETFGPVMLLSEFETDDEAVASANDSDFSLCGSVFSKDTMRALDIS |
| Eut_lata_XP_007794079 | DIFHKESFGPLICLTEFNTDEDAIRLANESDFSLCASVFSRDILRALDVG |
| Sta_char_KEY72856 | AIFHQETFGPIICLTTCSSDAEAVSLANDSDFSLAASVFSRDVMRALDVA |
| Gae_gram_XP_009217152 | RMRREEVFGPVLMLAEFDTDDEAVAAANDSDYSLCASVFSRDVMTAMDLA |
| Sta_char_KFA73399 | AIFHQETFGPIICLTTCSSDAEAVSLANDSDFSLAASVFSRDVMRALDVA |
| Sta_char_KFA53356 | AIFHQETFGPIICLTTCSSDAEAVSLANDSDFSLAASVFSRDVMRALDVA |
| Cyp_euro_XP_008712551 | DVFREESFGPVLCLTVVDSQAEAIEVANDSEFSLSAAVFSQDIMKALWLA |
| Sta_chlo_KFA62282 | AIFHQETFGPIICLTTCSSDAEAVSLANDNDFSLAASVFSRDVMRALDVA |
| | : : * ***:: : : :.: **:.:::*..::**:* : * . |
| P_brasilianum_hmfN1 | KRIRTGSCHVNGPTVYIEAPLPNGGVGGGSGYGRFGGVAGIEEFTERQIV |
| Spo_sche_ERT02387 | KQVRVGSCHINGPTVYVEAPLPNGGIGGASGYGRFGGMAGVEEFTERQIV |
| Sce_apio_KEZ45623 | KQVRAGSCHINGPTIYIEPTLPNGGVGGSSGYGRFGGVAGVEEFTERKIV |
| Pod_anse_XP_001908521 | KRLRLGACHINGPSLYVESTLPQGGTGGGSGYGRFGGMAGVEAFTEKKII |
| Eut_lata_XP_007794079 | RQVRAGSCHINGPTVYIEATLPNGGTGGSSGYGRFGGIAGVEEFTERQIL |
| Sta_char_KEY72856 | RQVRAGSCHINGPTVYIEPTLPNGGTGGSSGYGRFGGVAGIEEFTERKII |
| Gae_gram_XP_009217152 | RRVRAGTCHVNGPTIYVESTLPNGGTGGGSGYGRFGGMSGVEAFTEKKVI |
| Sta_char_KFA73399 | RQVRAGSCHINGPTVYIESTLPNGGTGGSSGYGRFGGVAGIEEFTERKII |
| Sta_char_KFA53356 | RQVRAGSCHINGPTVYIEPTLPNGGTGGSSGYGRFGGVAGIEEFTERKII |
| Cyp_euro_XP_008712551 | KQVRAGSCHINGPTVYIEATLPNGGTGGRSGYGRLGGSAGIEEYTERKII |
| Sta_chlo_KFA62282 | REVRAGSCHINGPTVYIEPALPNGGTGGSSGYGRFGGVAGIEEFTERKII |
| | :.:* *:**:***::*:*..**:** ** *****:** :*:* :**:::: |
| P_brasilianum_hmfN1 | SLAKPGIKYAF----- |
| Spo_sche_ERT02387 | SLTRPGLKYAF----- |
| Sce_apio_KEZ45623 | SLAQPGMKYSF----- |
| Pod_anse_XP_001908521 | TVVKPGLKLPL----- |
| Eut_lata_XP_007794079 | SLGKSGMRYRF----- |
| Sta_char_KEY72856 | TLARPGAKYPM----- |
| Gae_gram_XP_009217152 | TLARPGMRFAF----- |
| Sta_char_KFA73399 | TLARPGAKYPM----- |
| Sta_char_KFA53356 | TLARPGAKYPM----- |
| Cyp_euro_XP_008712551 | SLAQSGLKCVF----- |
| Sta_chlo_KFA62282 | TLARPDAKHPMUSAGE |
| | :: :.. : : |

TABLE 4
Amino acid sequence alignment of Penicillium brasilianum hmfK1
and 10 closest orthologues.

| | |
|---|---|
| P_brasilianum_hmfK1 | MPHASRSLNVLIVGAGLGGLAAGLALQTDGHKVTIIDAAPEFAEAGAGIR |
| Sce_apio_KEZ45619 | MPHASRSLNIVIVGAGLGGLAAGLALQTDGHKVTILDSAPEFGEVGAGIR |
| Tog_mini_XP_007916105 | MPQAARSLNVLVVGAGLGGLATGLALQTDGHTVTIIDAAPEFAEAGAGIR |
| Sta_char_KEY72859 | MPAAARSLNIVIVGAGLGGLAASLALQTDGHKVTILDSALEFAEAGAGIR |
| Sta_char_KFA53358 | MPAAARSLNIVIVGAGLGGLAASLALQTDGHKVTILDSALEFAEAGAGIR |
| Spo_sche_ERT02390 | MPQAARSLNVVVVGAGLGGLAAGLALQTDGHKVTILDAAPEFAEAGAGIR |
| Eut_lata_XP_007794919 | -------------------------------------MRLTLFKAGAGIR |
| Sta_chlo_KFA62283 | MPAAARSLNIVIVGAGLGGLAASLALQTDGHKT------------GAGIR |
| Gro_clav_EFX06428 | MPVPSRSLDILVVGAGLGGLAAGLALQTDGHKVTILDAVTEFAEVGAGIR |
| Cyp_euro_XP_008712555 | MPQAQHPRKILIVGAGLGGLAAGLALQTDGHNVTIIDSAPEFAEAGAGIR |
| Bys_spec_GAD98036 | MSKSVIPKEILIVGAGLGGLFASLALRQDGHSVTIIDAVPEFAEAGAGIR |
| | ***** |
| P_brasilianum_hmfK1 | IPPNSSRLLMRWGVDLERMKKSTSQRYHFIRWKDGSTIFDLPFNNIVETH |
| Sce_apio_KEZ45619 | VPPNSSRLLARWGVDLEGMKKSISKRYHFIRWQDGNTIVKLPFDKIVETH |
| Tog_mini_XP_007916105 | VPPNSSRLLLRWGVDLEKMKKSVSKRYHFIRWEDGATICKLPFDNIVETH |
| Sta_char_KEY72859 | VPPNSSRLLIRWGVDMEGMKKSTSNKYHFIRWKDGDTIVKVPFENVVETH |
| Sta_char_KFA53358 | VPPNSSRLLIRWGVDMEGMKKSTSNKYHFIRWKDGDTIVKVPFENVVETH |
| Spo_sche_ERT02390 | IPPNSSRLLMRWGVDLQRMKKSTSNRYHFIRWKDGTTIFDLPFDNNVATH |
| Eut_lata_XP_007794919 | VPPNSSRLLLRWGVDLENMKKSVSKRYHFVRWEDGSTIVKLPFENIVETH |
| Sta_chlo_KFA62283 | LPPNSSRLLIRWGVDMEGMKKSTSNKYHFIRWKDGDTIVKVPFDNVVETH |
| Gro_clav_EFX06428 | IPPNSSRLLIRWGVDLDRIKKSTASRYHFIRWKDGATIFNLPFVDSVQDH |
| Cyp_euro_XP_008712555 | VPPNSSRLLLRWGVDLEKMKKSVSQCYHFLRWKDGSTIVKLPFNDIVKNH |
| Bys_spec_GAD98036 | IPPNSSRLLMRWGVDLDKMKKSVSRSYHFVRWKDGTTITKLPFENIIEVH |
| | :******** *****:: :*** : ***:**:** ** .:** . : * |
| P_brasilianum_hmfK1 | GAPYWLVHRADLHAALLDATLKAGVKVLNNKLVTSYDFEAPSATTQDGET |
| Sce_apio_KEZ45619 | GAPYYLVHRADLHKALLDAAERAAVKVLTNKRITSFDFDAPSATTDDGEV |
| Tog_mini_XP_007916105 | GAPYYLVHRADLHAGLLEAARKAGVDIHTHKRVIEYNFEAPYAKTQEGEI |
| Sta_char_KEY72859 | GAPYYLVHRADLHAGLVEAAVRAGVAIRNNKRVTGYDLEAPAAVTHDGEV |
| Sta_char_KFA53358 | GAPYYLVHRADLHAGLVEAAVRAGVAIRNNKRVTGYDLEAPAAVTHDGEV |
| Spo_sche_ERT02390 | GSPYWLVHRADLHAALLDAAHKAGVQILTNKRVTAYDMDAPSATTADGAV |
| Eut_lata_XP_007794919 | GAPYYLVHRADLHAALLQTAEKAGVKVYNHKRVIAYDFDAPSATTQDGET |
| Sta_chlo_KFA62283 | GAPYYLVHRADLHSGLVEAALRAGVAIHNNKRVTGYDFDAPAAVTHDGEV |
| Gro_clav_EFX06428 | GAPYWLVHRADLHAALLDAARRAGATIVTSSRVVVYDMDAPSVTTADGTA |
| Cyp_euro_XP_008712555 | GAPYYLVHRADLHAGLLEAATRAGVQILNDKRVVEYNFEGPFVVTADGET |
| Bys_spec_GAD98036 | GAPYFLVHRADLHAALLDAAKKAGVEIYANQKVEKYDFSVPCAVTSEGKT |
| | *:**:******** .*:::: :*.. : . : :::. * . * :* |
| P_brasilianum_hmfK1 | FKADLIVGADGIKSICRPLLTGQPDVPRDTGDVAYRILIPGEKLLADPDL |
| Sce_apio_KEZ45619 | FKADLVVAADGIKSICRPLLTGKPDVPRDTGDVAYRILIPGEKLLADPEL |
| Tog_mini_XP_007916105 | FKADLIIGADGIKSIARPLLTGQPDIPRDTGDVAYRILIPGEKLLADPEL |
| Sta_char_KEY72859 | WRADLVLGADGIKSLARPLLTGQPDVPRDTGDVAYRILIPGERLLADPEL |
| Sta_char_KFA53358 | WRADLVLGADGIKSLARPLLTGQPDVPRDTGDVAYRILIPGERLLADPEL |
| Spo_sche_ERT02390 | YTGDLVVAADGIKSLCRPLLTGQADKPRDTGDVAYRILIPAEKLLADPEL |
| Eut_lata_XP_007794919 | FKADLVIGADGIKSIARPLLTGQPDIPRDTGDVAYRILIPGEKVLADPEL |
| Sta_chlo_KFA62283 | WRADLVLGADGIKSLARPLLTGQPDAPRDTGDVAYRILIPGERLLADPEL |
| Gro_clav_EFX06428 | YTADLVIGADGIKSTCRPLLTGRPDVPRDTGDVAYRILIPAEKLLADPDL |
| Cyp_euro_XP_008712555 | WRADLVIGADGIKSLARPALTGQEDVPRDTGDVAYRILIPGKDLLADPEL |
| Bys_spec_GAD98036 | WTADLVVCSDGIKSIARPLLTGQPDVPRDTGDVAYRILIPGKELLADSDL |
| | : .**:: :***** .** ***: * **************.: :***.:* |
| P_brasilianum_hmfK1 | AHLIRDPCTTSWCGPDAHLVGYPIRNGEMYNIVMCATSYNETTDEVWVVK |
| Sce_apio_KEZ45619 | ADLITEPCTTSWCGPDAHLVGYPIRNGEMYNIVVCATSYNETTDEVWVVK |
| Tog_mini_XP_007916105 | ANLITDPCTTSWCGPDAHLVGYPIRNGEMYNIVVCATSYNETTDEVWVIK |
| Sta_char_KEY72859 | APLITDPCTTSWCGPEAHLVGYPVRGGALYNVVVCATSHNETSDEAWVIR |
| Sta_char_KFA53358 | APLITDPCTTSWCGPEAHLVGYPVRGGALYNVVVCATSHNETSDEAWVIR |
| Spo_sche_ERT02390 | APLIQEPCTTSWCGPDAHLVGYPIRNEDTYNIVMCVTSYNETTDEAWVVR |
| Eut_lata_XP_007794919 | SDLITDPCTTSWCGPDAHLVGYPIRNGELYNIVVCATSYNETTDEVWVIK |
| Sta_chlo_KFA62283 | APLITDPCTTSWCGPEAHLVGYPIRGGAMYNIVVCAASHNETSDEAWVIR |
| Gro_clav_EFX06428 | APLITQPCSTSWCGPDAHLVGYPIRAGELYNVVVCATSRNETTSNTWVVR |
| Cyp_euro_XP_008712555 | ADLITDPCTTSWCGPDAHLVGYPIRNGELYNIVVCATSYNETSDEAWVVQ |
| Bys_spec_GAD98036 | KDLITEPATTSWCGPGAHLVGYPIRDGELYNIVVCATSNGETTDEVWVVK |
| | ** :*.:****** *******:* **:*:*.:* .**:.:.**:: |
| P_brasilianum_hmfK1 | GDNSELCKRFASWEPQVRKLCALTGDFMKWRLCDLPNLARWTHPSGKAVL |
| Sce_apio_KEZ45619 | GDNSELCKRFSKWEPRVQKLCALTGDFLKWRLCDLPDLTRWVHPAGKVVL |
| Tog_mini_XP_007916105 | GDNRELCERFGKWEKRVQKLCALTGDFMKWRLCDLPNLTRWAHPSGKAVL |
| Sta_char_KEY72859 | GDNRELCARFAAWEPRVRKLCALTGDFMKWRLCDLPILPRWVHPAGKVAL |
| Sta_char_KFA53358 | GDNRELCARFAAWEPRVRKLCALTGDFMKWRLCDLPILPRWVHPAGKVAL |
| Spo_sche_ERT02390 | GDNSELCQRFAHWETKVQKLCALTGDFMKWRLCDLPNLSRWVHPAGKVVL |
| Eut_lata_XP_007794919 | GDNRELCTRFGGWESRVRKLCALTGDFMKWRLCDLPNISRWAHPSGKVVL |
| Sta_chlo_KFA62283 | GDNRELCTRFAAWEPRVRKLCALTGDFMKWRLCDLPILPRWVHPAGKAAL |
| Gro_clav_EFX06428 | GDNSELRLRFASWTTQVRKLCALTGDFLKWRLCDLPNLTRWVHPSGKVVL |
| Cyp_euro_XP_008712555 | GSPLDLLERFKTWEPRVQKLCKLTPQFMKWRLCDLPILSRWVHPSGKAAL |
| Bys_spec_GAD98036 | GSNEELCERFASWEPRIQKLCKLTRDFMKWRLCDLPILSTWVHPSGKACL |
| | *. :* ** * :::*** ** :*:******** :. *.**:**. * |
| P_brasilianum_hmfK1 | LGDSCHPMLPYLAQGAAQAVEDAAVLRQVLAQDM---------------- |
| Sce_apio_KEZ45619 | LGDSCHPMLPYLAQGAAQAFEDAATLRQVLAQGE---------------- |
| Tog_mini_XP_007916105 | LGDSCHPMLPYLAQGAAQAFEDAAVIRQCLAQDT---------------- |
| Sta_char_KEY72859 | LGDACHPMLPYLAQGAAQSFEDAATLRQCLALDLP--------------- |
| Sta_char_KFA53358 | LGDACHPMLPYLAQGAAQSFEDAATLRQCLALDLP--------------- |
| Spo_sche_ERT02390 | LGDSCHPMLPYLAQGAAQAFEDAAVLRQVLALVG-------GVDGG---- |
| Eut_lata_XP_007794919 | IGDSCHPMLPYLAQGAAQSFEDAAALRQVLAQDV---------------- |
| Sta_chlo_KFA62283 | LGDACHPMLPYLAQGAAQSFEDAATLRQCLALDLP--------------- |
| Gro_clav_EFX06428 | LGDSCHPMLPYLAQGAAQAFEDASVLRQVLRVALSSADLSMGSDGATSSL |
| Cyp_euro_XP_008712555 | LGDSCHPMLPYLAQGAAQAVEDAAALRQCLAGASTAG------------- |
| Bys_spec_GAD98036 | LGDSCHPMLPYLAQGAAQAAEDAAVLRRCLAKFS---------------- |
| | :**:**************: ***:.:*: * |
| P_brasilianum_hmfK1 | ---DMAAALKQYEQIRMPRASLVQAKTREHQYILHVDDGHEQQDRDKKLA |
| Sce_apio_KEZ45619 | ---DLSAALKKYEQIRMPRASLVQAKTREHQYILHIDDGEEQAIRDEKMK |
| Tog_mini_XP_007916105 | ---DLPTGLKNYESIRMPRASLVQAKTREHQYILHIDDGEEQKARDERMR |
| Sta_char_KEY72859 | ----LADALARYEAVRQPRASLVQTKTREHQYILHIADGDEQRLRDDLMK |
| Sta_char_KFA53358 | ----LADALARYEAVRQPRASLVQTKTREHQYILHIADGDEQRLRDDLMK |
| Spo_sche_ERT02390 | --VDLKTALQRYEAIRMPRATLVQAKTREHQHILHVDDGQEQATRDQELA |
| Eut_lata_XP_007794919 | ---DLPTALKRYEQIRMPRASLVQAKTREHQYILHIPDGEEQKARDRQLQ |
| Sta_chlo_KFA62283 | ----LADALARYESVRQPRASLVQSKTREHQYILHIADGDEQRLRDDMMK |
| Gro_clav_EFX06428 | PPPDLHAALLRYERIRMPRASLVQSTTREHQHLLHIDDGLEQEERDHRLS |
| Cyp_euro_XP_008712555 | -ADGLKQALLKYESIRLPRASLVQQKTREHQYILHVDDGETQKQRDVTMK |
| Bys_spec_GAD98036 | ---DLHEALKDYEKIRLPRASTIQGKTREHQYILHIDDGEEQLERDQRMR |
| | : .* ** :* ***: :* .*****::**: ** * ** : |
| P_brasilianum_hmfK1 | LDAAENPVFWGYDDRRKWLFSHDAEVIQKEGANWRDGPN----------- |
| Sce_apio_KEZ45619 | LNAAENPVFWGYDDRRQWLFSHDAENLAKEGANWKDGLN----------- |
| Tog_mini_XP_007916105 | VNAAENPVFWGYDDRRKWLFSHDAEILNKDGANWREASQ----------- |
| Sta_char_KEY72859 | HNGEGNPVFWGHDDRRKWLFSHDAEVLTKEGANWMEAPN----------- |
| Sta_char_KFA53358 | HNGEGNPVFWGHDDRRKWLFSHDAEVLTKEGANWIEAPN----------- |
| Spo_sche_ERT02390 | LDAAENPVFWGHTDRRNWLFGHDAEIITTPGDNWREGQ------------ |
| Eut_lata_XP_007794919 | LNATENPIFWGYDERRKWLFSHDAEVLNTEGANWQKTTP----------- |
| Sta_chlo_KFA62283 | QNGEGNPVFWGHDDRRKWLFSHDAEVLTKEGANWMEAPN----------- |
| Gro_clav_EFX06428 | RDHPDSPVFWGYVERKNWLFGHDADVIIKEGDNWREGAGLHVVQASHVVD |
| Cyp_euro_XP_008712555 | VNGQENPVFWGDDKRRMWLFSHDAENVDSEGANWKSGTG----------- |
| Bys_spec_GAD98036 | QNSETNPIFWGYDKRRKWLFSHDADLLERNEVVWSQPAA----------- |
| | : .*:*** .*: ***.***: : * . |
| P_brasilianum_hmfK1 | ------------MNGVHVA--------- |
| Sce_apio_KEZ45619 | ------------GSAIRSH--------- |
| Tog_mini_XP_007916105 | ------------STGVAAH--------- |
| Sta_char_KEY72859 | ------------ATALKAH--------- |
| Sta_char_KFA53358 | ------------ATALKAH--------- |
| Spo_sche_ERT02390 | ------------TSGVAAH--------- |
| Eut_lata_XP_007794919 | ------------DSGVSAH--------- |
| Sta_chlo_KFA62283 | ------------ATALKAH--------- |
| Gro_clav_EFX06428 | GVQGAGTNGVGGINGVAVH--------- |
| Cyp_euro_XP_008712555 | ------------APLVGAPVATSMLAAH |
| Bys_spec_GAD98036 | ------------ASLUSAGE-------- |

TABLE 5
Amino acid sequence alignment of Penicillium brasilianum hmfM
and 10 closest orthologues.

| | |
|---|---|
| P_brasilianum_hmfM | MSLSGKVVLITGSSKGIGKAAALRVASEGANVVINYLRDPVAANNLVDQI |
| Asp_nidu_XP_664054 | MSLAGKVALITGASKGIGRATAQRLASEGASLVINYNTDAASAQALVDEI |
| Eut_lata_XP_007797627 | MSLQGKVILITGGSKGIGRAIALRVAKSGASVVVNYSSDSNAANEVVSQI |
| Thi_terr_XP_003656972 | MSLSGKVALITGGSKGIGRAVAQRLAADGASVVINFKSDSKAADELVAEI |
| Tri_atro_EHK50353 | MQLPDKVILITGASSGIGKACAQRLYQEGARIVVNYRNDASAANALVDSF |
| Asp_terr_XP_001212987 | MSLAGKVVLITGASKGIGKATAQHLAANGASIVINYLSDAASANALVDEI |
| Tri_rees_XP_006962638 | MSLQDKVILITGASSGIGKATAQRLYKEGARIVVNYHSDDSAANALVESF |
| Fus_oxys_EMT67544 | MSLNGKVVLVTGGSKGIGKAVAERVVADGASVVINYSSDSKPAEDLVIKI |
| Fus_oxys_EGU79882 | MSLNGKVVLVTGGSKGIGKAVAERVVADGASVVINYSSDSKPAEDLVIKI |
| Fus_oxys_EXL52390 | MSLNGKVVLVTGGSKGIGKAVAERVVADGASVVINYSSDSKPAEDLVTKI |
| Fus_oxys_ENH63602 | MSLNGKVVLVTGGSKGIGKAVAERVVADGASVVINYSSDSKPAEDLVIKI |
| | *.* .** *:**.*.***:* * :: .** :*:*: * .*: :* .: |
| P_brasilianum_hmfM | GADRALAVQADASKLADLDRLVNAAVAQFGKIDVLIPNAGILPLRDLEHT |
| Asp_nidu_XP_664054 | GQDRALAVQADASKLADIDRLVDAAVAKFGKIDILIPNAGILPMRDLEHT |
| Eut_lata_XP_007797627 | GSDRALAVKADASTVTGVSSLVDATVKQFGKVDVVIPNAGMMPMQDLEHT |
| Thi_terr_XP_003656972 | GADRALAVQADVSKLDDIEKLVNAAVARFGKIDIVMPNAGVMAMVPLANL |
| Tri_atro_EHK50353 | GADRAIAVQADASNINDIERLVQATVDKFGRIDTIVANAGLMLMRDVEDT |
| Asp_terr_XP_001212987 | GEDRALAVQADASKLDDIRRLVEAAVTKFGHIDVVIPNAGVLLMRDLATT |
| Tri_rees_XP_006962638 | GPDRAIAVRADAANISDIDRLVRTTVDKFGRIDVVVANAGLMLMRDVEDT |
| Fus_oxys_EMT67544 | GSDRALAFKADVSKIAEIEKLVQATVEKFGKIDCVMANAACAPMNDLEST |
| Fus_oxys_EGU79882 | GSDRALAFKADVSNIAEIEKLVQATVEKFGKIDCVMANAACAPMNDLEST |
| Fus_oxys_EXL52390 | GSDRALAFKADVSNIAEIEKLVQATVEKFGKIDCVMANAACAPMNDLEST |
| Fus_oxys_ENH63602 | GSDRALAFKADVSNIAEIEKLVQATVEKFGKIDCVMANAACAPMNDLEST |
| | * ***:*.:**.:.: : ** ::* :**::* ::.**. : : |
| P_brasilianum_hmfM | SEEDFDRTYNLMVKGPYFLAQ--KAVKHMPPGGRIIFVSTSTARFASVAP |
| Asp_nidu_XP_664054 | TEEDFDFTYNLMVKGPYFLAQAQKAAKHIPAGGRIILVSTGVTVLSNIAP |
| Eut_lata_XP_007797627 | TEATFDKIYAINVKGPYFLAQ--KAVPHMPSGGRIIFVSTGIAHNSAVPP |
| Thi_terr_XP_003656972 | TEAEFDRHFNLNVKGALFLVQ--KAVAHVPAGGRIIFVSTGLARQSAVAP |
| Tri_atro_EHK50353 | TEDDFAKSFDLNVKGPYFLAQ--KAVPHMPPGSHVIFISTGVCHHSSVSP |
| Asp_terr_XP_001212987 | TEADFDTAFNLNVKGPYFLVQ--EATRHMPAGGRVIFVSTGVTVHSSISP |
| Tri_rees_XP_006962638 | TEDDFGQMFDINVKGPYFLAQ--KAVPHMPPGSRIIFISTGVCHYSSVPA |
| Fus_oxys_EMT67544 | TEEGFDKAFNLNVKGPYFLVQ--KAVKHMPRDGRVILVSSGVLHQSQVAP |
| Fus_oxys_EGU79882 | TEEGFDKAFNLNVKGPYFLVQ--KAVKHMPRDGRVILVSSGVLHQSQVAP |
| Fus_oxys_EXL52390 | TEEGFDKAFNLNVKGPYFLVQ--KAVKHMPRDGRVILVSSGVLHQSQVAP |
| Fus_oxys_ENH63602 | TEEGFDKAFNLNVKGPYFLVQ--KAVKHMPRDGRVILVSSGVLHQSQVAP |
| | * ***:*.:**.:.: : ** ::* :**::* ::.**. : : |
| P_brasilianum_hmfM | AYLLYTSSKGAIEQMTRIMAKDLARKGILVNAVAPGPTSTELFLEGKPEQ |
| Asp_nidu_XP_664054 | AYLLYASAKAAVEQMARVMAKDLARNGILVNCVAPGPTTTGLFLNGKSDQ |
| Eut_lata_XP_007797627 | PYLLYASTKGAVEQMTRVMAKDLGKKGITVNCVAPGPTATELFFEGKSEA |
| Thi_terr_XP_003656972 | GYLVYAATKGAIEQLVRVLSKDLGAKGITVNAVAPGPTGTELFYQGKSEQ |
| Tri_atro_EHK50353 | KYLLYAATKGAIEQMTRVMAKGLAAKGIIVNAVAPGPTATELFYKGKPEG |
| Asp_terr_XP_001212987 | TYLLYASTKGAIEQMTRITAKELAKKGIFVNAIAPGPTTTELFLRGKSEE |
| Tri_rees_XP_006962638 | KYLLYAATKGAIEQMTRVMAKGLAAKGIIVNAVAPGPTATELFFKGKPES |
| Fus_oxys_EMT67544 | RYLLYASSKGSIEQMTRILAKDLGPKGITVNAIAPGPTATEMFFQGKSQE |
| Fus_oxys_EGU79882 | RYLLYASSKGSIEQMTRILAKDLGPKGITVNAIAPGPTATEMFFQGKSQE |
| Fus_oxys_EXL52390 | RYLLYASSKGSIEQMTRILAKDLGPKGITVNAIAPGPTATEMFFQGKSQE |
| Fus_oxys_ENH63602 | RYLLYASSKGSIEQMTRILAKDLGPKGITVNAIAPGPTATEMFFQGKSQE |
| | **:*:::*.::**:.*: :* *. :** **.:***** * :* .**.: |
| P_brasilianum_hmfM | MIKAISGFSPFNRIGEPEEIAAVMAFLSG-------------------KD |
| Asp_nidu_XP_664054 | MLKMVAGFSPFNRIGEPEEIANAVYFLCS-------------------KD |
| Eut_lata_XP_007797627 | MVKGIASQSPFNRLGDPAEIAELAAFVAG-------------------PE |
| Thi_terr_XP_003656972 | LLQTIRGWSPFNRIGEPAEIAGVVAFLAG-------------------ED |
| Tri_atro_EHK50353 | LVNTIKAWSPFNRLGEPEDIANTVKFLAS-------------------GD |
| Asp_terr_XP_001212987 | TLRAVAGFSPFNRIGEPGEMASVINFLCGPEFGDCPESRSTPETMTETKT |
| Tri_rees_XP_006962638 | VVNAIKGWSPFNRLGQPEEVANTIKFLAS-------------------DE |
| Fus_oxys_EMT67544 | LIDTIAGFSPLGRLGKPEEIAGLAAFLAG-------------------PT |
| Fus_oxys_EGU79882 | LIDTIAGFSPLGRLGKPEEIAGLAAFLAG-------------------PT |
| Fus_oxys_EXL52390 | LIDTIAGFSPLGRLGKPEEIAGLAAFLAG-------------------PT |
| Fus_oxys_ENH63602 | LIDTIAGFSPLGRLGKPEEIAGLAAFLAG-------------------PT |
| | : : . **:.*:*.* ::* *:.. |
| P_brasilianum_hmfM | SSWISG-QVVAVNGAMA--------------------------------- |
| Asp_nidu_XP_664054 | SSWVSG-QTLRVNGGMA--------------------------------- |
| Eut_lata_XP_007797627 | SRWVSG-QVIGANGAAFV-------------------------------- |
| Thi_terr_XP_003656972 | SRWVSG-QVIGANGAMMV-------------------------------- |
| Tri_atro_EHK50353 | SSWVVG-QTVLVNGGIMV-------------------------------- |
| Asp_terr_XP_001212987 | TERVEKPQKGKVAGNTDAKPRAKSLKLTLPLPTDLSADRQPATTKNRNHF |
| Tri_rees_XP_006962638 | SSWVVG-QTVLVNGGIMV-------------------------------- |
| Fus_oxys_EMT67544 | SSWVSG-QVIGANGGSFV-------------------------------- |
| Fus_oxys_EGU79882 | SSWVSG-QVIGVNGGSFV-------------------------------- |
| Fus_oxys_EXL52390 | SSWVSG-QVIGANGGSFV-------------------------------- |
| Fus_oxys_ENH63602 | SSWVSG-QVIGANGGSFVUSAGE--------------------------- |
| | : : * . * |
| P_brasilianum_hmfM | -------------------------------------------------- |
| Asp_nidu_XP_664054 | -------------------------------------------------- |
| Eut_lata_XP_007797627 | -------------------------------------------------- |
| Thi_terr_XP_003656972 | -------------------------------------------------- |
| Tri_atro_EHK50353 | -------------------------------------------------- |
| Asp_terr_XP_001212987 | VKTLTGKTITLDVESSDTIDNVKAKIQDKEGIPPDQQRLIFAGKQLEDGR |
| Tri_rees_XP_006962638 | -------------------------------------------------- |
| Fus_oxys_EMT67544 | -------------------------------------------------- |
| Fus_oxys_EGU79882 | -------------------------------------------------- |
| Fus_oxys_EXL52390 | -------------------------------------------------- |
| Fus_oxys_ENH63602 | -------------------------------------------------- |
| P_brasilianum_hmfM | -------------------------------------------------- |
| Asp_nidu_XP_664054 | -------------------------------------------------- |
| Eut_lata_XP_007797627 | -------------------------------------------------- |
| Thi_terr_XP_003656972 | -------------------------------------------------- |
| Tri_atro_EHK50353 | -------------------------------------------------- |
| Asp_terr_XP_001212987 | TLSDYNIQKESTLHLVLRLRGGIIEPSLKALASKYNCEKSICRKCYARLP |
| Tri_rees_XP_006962638 | -------------------------------------------------- |
| Fus_oxys_EMT67544 | -------------------------------------------------- |
| Fus_oxys_EGU79882 | -------------------------------------------------- |
| Fus_oxys_EXL52390 | -------------------------------------------------- |
| Fus_oxys_ENH63602 | -------------------------------------------------- |
| P_brasilianum_hmfM | ------------------------ |
| Asp_nidu_XP_664054 | ------------------------ |
| Eut_lata_XP_007797627 | ------------------------ |
| Thi_terr_XP_003656972 | ------------------------ |
| Tri_atro_EHK50353 | ------------------------ |
| Asp_terr_XP_001212987 | PRATNCRKKKCGHTNQLRPKKKLK |
| Tri_rees_XP_006962638 | ------------------------ |
| Fus_oxys_EMT67544 | ------------------------ |
| Fus_oxys_EGU79882 | ------------------------ |
| Fus_oxys_EXL52390 | ------------------------ |
| Fus_oxys_ENH63602 | ------------------------ |

TABLE 6
Amino acid sequence alignment of Penicillium brasilianum hmfT3
and 10 closest orthologues.

| | |
|---|---|
| P_brasilianum_hmfT3 | MASLIREAPFGQIVRYLTNNKYFQYPEEKPDFKLPDTWLQLLN------- |
| Pen_rube_XP_002560799 | MASIIRDAPFGQLVRLLTNNKYFQYPEEKPDFKLPDTWLQLLN------- |
| Pen_oxal_EPS29964 | MASVIRDAPFGQLVRYLTNNKYFQYPEERPDFELPEAWRELISGADSIKP |
| Asp_terr_XP_001212020 | MQAVLRESAFGQLVRLVTKNKYFQYPEEKADFKLPDQWIKVMD------- |
| Fus_oxys_ENH73763 | MSDLIRDAPLGQLIRFVTRNKYLQYPEEKPDFKLPESWVAVINNPDAIIE |
| Fus_oxys_EGU73369 | MSDLIRDAPLGQLIRFVTRNKYLQYPEEKPDFKLPESWVAVINNPDAIIE |
| Fus_oxys_EXL94287 | MSDLIRDAPLGQLIRFVTRNKYLQYPEEKPDFKLPESWVAVINNPDAIIE |
| Nec_haem_XP_003040064 | MADIIRDAPLGQVIRFVTRNKYLKYPEEKEDFKLPDPWITLVNNPDAIVE |
| Fus_pseu_XP_009258565 | MSDIIRDAPLGQLIRFVTRNRYFQYPEEKPDFKLPDAWDTVINNPNVIID |
| Fus_gram_XP_011323833 | MSDIIRDAPLGQLIRFVTRNKYLQYPEEKPDFKLPDAWDTVINNPNVIVD |
| Fus_fuji_CCT64241 | MSDLIRDAPLGQLIRFVTRNKYLQYPEEKPDFKLPESWVAVINNPDAIIE |
| | * ::*::.:**::* :*.*:*::****: **:**: * ::. |
| P_brasilianum_hmfT3 | -------------ESDAATIADPEKTEPEPEGQGYDAT------------ |
| Pen_rube_XP_002560799 | -------------SNGDE---DDEKKAIQQDSNRSPED------------ |
| Pen_oxal_EPS29964 | VRDLEKAP---VAGTPASLTDEDASVRGQSPDAESETTT----------- |
| Asp_terr_XP_001212020 | -------------GLDAAASSEHAQTDAQTP-TRQPDS------------ |
| Fus_oxys_ENH73763 | ESSPHDNT--VLT------------------------------------- |
| Fus_oxys_EGU73369 | ESSPHDNT--VLTGTALASSASSTVAAEEDPKLKAENENEKNEKSEKNNE |
| Fus_oxys_EXL94287 | ESSPHDNT--VLTGTALASSASSTVAAEEDPKLKAENE--KNEKSEKTNE |
| Nec_haem_XP_003040064 | DAPIENLT------------------------------------------ |
| Fus_pseu_XP_009258565 | ESPANNNN-ALLTGTALASSASSTVAATEDPKIKSETD----------KE |
| Fus_gram_XP_011323833 | ESPANNNNNALLTGTALASSASSTVAATEDPKIKSETD----------KE |
| Fus_fuji_CCT64241 | ESSPNDNT--VLTGTALASSASSTVAAEEDPKLKGDNE--KNDKSEKNDE |
| P_brasilianum_hmfT3 | -----------SEAISRASTQNSLPFTEARLEADEQHEIEKIKSIPIQPK |
| Pen_rube_XP_002560799 | -----------SEPLSRASTQASIEFTEARLEADEQHEIEKIKSIPIAPK |
| Pen_oxal_EPS29964 | --------ATATEAIARVNTKETLAYTQSRLEADEEHEIQKLQSIPIQPK |
| Asp_terr_XP_001212020 | -----------DESLSQVTTNYSLSFTEARLEADQQHEIEKVKSIPIAPK |
| Fus_oxys_ENH73763 | ------------------------AYTVDRLEADEEHDVEKVKSIPVVPK |
| Fus_oxys_EGU73369 | NDDIERADPQPMRLHRSRSPQETQAYTVDRLEADEEHDVEKVKSIPVVPK |
| Fus_oxys_EXL94287 | NDDIERADPQPMRLHRSRSPQETQAYTVDRLEADEEHDVEKVKSIPVVPK |
| Nec_haem_XP_003040064 | ---------------------DTQAYTADRMRVDEEHEIEKVQSIPIVPK |
| Fus_pseu_XP_009258565 | TEDVERADSVPVRLHRSRSPQETQAYTIDRLEADEEHDVEKVKSIPVVPK |
| Fus_gram_XP_011323833 | TEDVERADSVPVRLYRSRSPQETQAYTIDRLEADEEHDVEKVKSIPVVPK |
| Fus_fuji_CCT64241 | NDDIERADPQPMRLHRSRSPQETQAYTVDRLEADEEHDVEKVKSIPVVPK |
| | :* *:..*::*:::*::***: ** |
| P_brasilianum_hmfT3 | KTKDGAILVDWYYTDDAENPHNWSNRKRALLTTLICLYTFVVYTTSAIYT |
| Pen_rube_XP_002560799 | KTKDGSILVDWYYTDDLENPHNWSNGKRAFITILICLYTFVVYTTSAIYT |
| Pen_oxal_EPS29964 | KTKDGTILVDWYYTDDQENPHNWSNRKRALLTTIICLYTFVVYTTSAIYT |
| Asp_terr_XP_001212020 | KTKDGAILVDWYYTDDAENPHNWSNLKRALVATIICLYTFVVYTTSAIYT |
| Fus_oxys_ENH73763 | RTKDGSILVDWYFSDDNENPHNWTNNRRLGVSLIICLYTFVVYTSSAIYT |
| Fus_oxys_EGU73369 | RTKDGSILVDWYFSDDNENPHNWTNNRRLGVSLIICLYTFVVYTSSAIYT |
| Fus_oxys_EXL94287 | RTKDGSILVDWYFSDDNENPHNWTNNRRLGVSLIICLYTFVVYTSSAIYT |
| Nec_haem_XP_003040064 | KTKDGAILVDWYYSDDADNPHNWSNNKRLGISLIICLYTFVVYTSSAIYT |
| Fus_pseu_XP_009258565 | RTKDGHILVDWYYSDDNENPHNWTNNRRLGVALIICLYTFVVYTSSAIYT |
| Fus_gram_XP_011323833 | RTKDGHILVDWYYSDDKENPHNWTNNRRLGVALIICLYTFVVYTSSAIYT |
| Fus_fuji_CCT64241 | RTKDGSILVDWYFSDDNENPHNWTNNRRLGVSLIICLYTFVVYTSSAIYT |
| | :**** ******::** :*****:* :* :: :**********:***** |
| P_brasilianum_hmfT3 | SSVPGIMKEFGVSDLVATLGLSLYVLGYGTGPLIFSPLSEIPVIGRNPVY |
| Pen_rube_XP_002560799 | SSTQGVMKEFGVSTLVATLGLSLYVLGYGTGPLVFSPLSEIPVIGRNPVY |
| Pen_oxal_EPS29964 | ASVPGVMEDFGVSNLLATLGLSLYVLGYGMGPLVFSPLSEIPLIGRNPVY |
| Asp_terr_XP_001212020 | SSVGGIIAQFGVSELLATLGLSLYVLGYGIGPLLFSPMSEIPIIGRNPVY |
| Fus_oxys_ENH73763 | SSTEGVMRAFGVSQLKATLGLSLYVLGYGIGPLIFSPLSEIPRIGRNPVY |
| Fus_oxys_EGU73369 | SSTEGVMRAFGVSQLKATLGLSLYVLGYGIGPLIFSPLSEIPRIGRNPVY |
| Fus_oxys_EXL94287 | SSTEGVMHAFGVSQLKATLGLSLYVLGYGIGPLIFSPLSEIPRIGRNPVY |
| Nec_haem_XP_003040064 | SSTEGVMKAFGVSQLKATLGLALYVLGYGIGPLLFSPLSEIPRIGRNPVY |
| Fus_pseu_XP_009258565 | SSTEGVMRAFGVSQLKATLGLSLYVLGYGTGPLIFSPLSEIPRIGRNPVY |
| Fus_gram_XP_011323833 | SSTEGVMRAFGVSQLKATLGLSLYVLGYGTGPLIFSPLSEIPRIGRNPVY |
| Fus_fuji_CCT64241 | SSTEGVMRAFGVSQLKASLGLALYVLGYGIGPLIFSPLSEIPRIGRNPVY |
| | :*. *:: **** * *:***:******* ***:***:**** ******* |
| P_brasilianum_hmfT3 | IVTMFLFVILSIPTAFVGNFAGLMVLRFLQGFFGSPCLASGGASIGDMYS |
| Pen_rube_XP_002560799 | IITMFLFVIISIPTAFVGNFAGLMVLRFLQGFFGSPCLASGGASIGDMYS |
| Pen_oxal_EPS29964 | IVTMFLFVILSIPTALVHNFAGLIVLRFLQGFFGSPCLASGGASIGDMYS |
| Asp_terr_XP_001212020 | IVTMFLFVIISIPTAFAGNFPGLMVLRFLQGFFGSPCLASGGASIGDMYS |
| Fus_oxys_ENH73763 | IVTMFLFVIISIPTALVNNYPGLMVLRFLQGFFGSPCLASGGASLGDIYS |
| Fus_oxys_EGU73369 | IVTMFLFVIISIPTALVNNYPGLMVLRFLQGFFGSPCLASGGASLGDIYS |
| Fus_oxys_EXL94287 | IVTMFLFVIISIPTALVNNYPGLMVLRFLQGFFGSPCLASGGASLGDIYS |
| Nec_haem_XP_003040064 | IVTMFLFVIISIPTAFVGNYPGLMVLRFLQGFFGSPCLASGGASLGDIYS |
| Fus_pseu_XP_009258565 | IVTMFLFVIISIPTALVKNYPGLMVLRFLQGFFGSPCLASGGASLGDIYS |
| Fus_gram_XP_011323833 | IVTMFLFVIISIPTALVKNYPGLMVLRFLQGFFGSPCLASGGASLGDIYS |
| Fus_fuji_CCT64241 | IVTMFLFVIISIPTALVDNYPGLMVLRFLQGFFGSPCLASGGASLGDIYS |
| | *:*******:*****:. *:.**:********************:**:** |
| P_brasilianum_hmfT3 | LMSLPYAMMSWVSAAYCGPALGPLISGFAVPAETWRWSLFESIWMSAPVL |
| Pen_rube_XP_002560799 | LMNLPFAMMAWVAAAYCGPALGPLLSGFAVPVKGWRWSLFESIWASAPVF |
| Pen_oxal_EPS29964 | LLSLPYAMMTWVSAAYCGPALGPLLSGFAVAAKNWRWSLYESIWMSAPVF |
| Asp_terr_XP_001212020 | LMSLPYAMMAWVAAAYCGPALGPLLSGFAVPAKSWRWSLFESIWASAPVF |
| Fus_oxys_ENH73763 | LMALPYAMMAWVSAAYCGPALGPLISGFAVPAKNWRWSLYESIWASAPIF |
| Fus_oxys_EGU73369 | LMALPYAMMAWVSAAYCGPALGPLISGFAVPAKNWRWSLYESIWASAPIF |
| Fus_oxys_EXL94287 | LMALPYAMMAWVSAAYCGPALGPLISGFAVPAKNWRWSLYESIWASAPIF |
| Nec_haem_XP_003040064 | LMALPYAMMAWVSAAYCGPALGPLLSGFAVPAKSWRWSLYESIWASAPIF |
| Fus_pseu_XP_009258565 | FMALPYAMMAWVAAAYCGPALGPLLSGFAVPAKGWRWSLYESIWASAPIF |
| Fus_gram_XP_011323833 | FMALPYAMMAWVAAAYCGPALGPLLSGFAVPAKGWRWSLYESIWASAPIF |
| Fus_fuji_CCT64241 | LMALPYAMMAWVSAAYCGPALGPLISGFAVPAKNWRWSLYESIWASAPIF |
| | :: **:***:**:***********:*****..: *****:**** ***:: |
| P_brasilianum_hmfT3 | ILMFFFLPETSSATILLRRAARLRKIHNNARFMAQSEIDQRNMKVSAVAV |
| Pen_rube_XP_002560799 | ILMFMFLPETSSATILLRRAARLRKIHNTNRFMSQSELDQRNMRVSDIAV |
| Pen_oxal_EPS29964 | ILMLVFLPETSSATILLRRAARLRKIYNTDLFMSQSEIDQRNMKVSDIAV |
| Asp_terr_XP_001212020 | LLMFFFLPETSTSTILLRRASRLRRIFKDDRFMSQSEIDQRNMRISDVTV |
| Fus_oxys_ENH73763 | ILMFLLLPETSGANILLRRAERLRKLTGNQRFMSQSEIDQRHMKVSAIAV |
| Fus_oxys_EGU73369 | ILMFLLLPETSGANILLRRAERLRKLTGNQRFMSQSEIDQRHMKVSAIAV |
| Fus_oxys_EXL94287 | ILMFLLLPETSGANILLRRAERLRKLTGNQRFMSQSEIDQRHMKVSAIAV |
| Nec_haem_XP_003040064 | ILMFLLLPETSGANILLRRAKRLRKLTGNDRFMSQSEIDQRNMKVSSIAL |
| Fus_pseu_XP_009258565 | ILMFLLLPETSGANILLRRAERLRKLTGNERFMSQSEIDQRHMKVSAIAL |
| Fus_gram_XP_011323833 | ILMFLLLPETSGANILLRRAERLRKLTGNERFMSQSEIDQRHMKVSAIAL |
| Fus_fuji_CCT64241 | ILMFLLLPETSGANILLRRAERLRKLTGNQRFMSQSEIDQRHMKVSAIAV |
| | :**:.:***** :.****** ***:: **:***:***:*::* ::: |
| P_brasilianum_hmfT3 | DALIKPLEITIKDPAVLFVQVYTAIIYGIYYSFFEVFPLVYPVDYGMNLG |
| Pen_rube_XP_002560799 | DALIKPMEITIKDPAVLFVQIYTAIIYGIYYSFFEVFPLVYPVDYNMNLG |
| Pen_oxal_EPS29964 | DALLKPLQITIMDPAVLFVQVYTAITYGIYYSFFEVFPLVYPVYYHMNMG |
| Asp_terr_XP_001212020 | DALIKPLEITIKDPAVLFVQIYTAIIYGIYYSFFEVFPLVYPVDYNMNLG |
| Fus_oxys_ENH73763 | DALIKPMEITIKDPAVLFVQVYTAIIYGIYYSFFEVFPRVYPVYYNMNLG |
| Fus_oxys_EGU73369 | DALIKPMEITIKDPAVLFVQVYTAIIYGIYYSFFEVFPRVYPVYYNMNLG |
| Fus_oxys_EXL94287 | DALIKPMEITIKDPAVLFVQVYTAIIYGIYYSFFEVFPRVYPVYYNMNLG |
| Nec_haem_XP_003040064 | DALIKPMEITIKDPAVLFVQVYTAIIYGIYYSFFEVFPRVYPVYYGMNLG |
| Fus_pseu_XP_009258565 | DALIKPMEITIKDPAVLFVQIYTAIIYGIYYSFFEVFPRVYPVYYNMNLG |
| Fus_gram_XP_011323833 | DALIKPMEITIKDPAVLFVQIYTAIIYGIYYSFFEVFPRVYPVYYNMNLG |
| Fus_fuji_CCT64241 | DALIKPMEITIKDPAVLFVQVYTAIIY-----VFEVFPRVYPVYYNMNLG |
| | ***:**::*** ********:**** * .***** **** * **:* |
| P_brasilianum_hmfT3 | QVGLVFLCILVSCIIGIAIYWSYLYFWMNPRIERFGFPAQESRLIPALPA |
| Pen_rube_XP_002560799 | QIGLVFLCVLVSCIIGIAVYASYIHFWMNRRIRRFGFPVNEKLLIPALPA |
| Pen_oxal_EPS29964 | QIGLVFLCILVSCLIGIAAYSAYLYYWMNPRIHRFGFPVQEARLIPALPA |
| Asp_terr_XP_001212020 | QIGLVFLCILVSCILGIAIYFSYLYFWMNPRIARFGFPEQETRLVPALPA |
| Fus_oxys_ENH73763 | QIGLVFLCVLVSCMIGVGLYVSYLYFYMDPRIAKRGWPIQESRLVPALPA |
| Fus_oxys_EGU73369 | QIGLVFLCVLVSCMIGVGLYVSYLYFYMDPRIAKRGWPIQESRLVPALPA |
| Fus_oxys_EXL94287 | QIGLVFLCVLVSCMIGVGLYVSYLYFYMDPRIAKRGWPIQESRLVPALPA |
| Nec_haem_XP_003040064 | EIGLVFLCVLVSCIIGVAIYVAYLYYYMDPRIAKRGWPVQEARLAPALLA |
| Fus_pseu_XP_009258565 | EIGLVFLCVLVSCMIGVGVYLSYLYFYMDPRIAKRGWPIQESRLVPALPA |
| Fus_gram_XP_011323833 | EIGLVFLCVLVSCMIGVGVYLSYLYFYMDPRIAKRGWPVQESRLVPALPA |
| Fus_fuji_CCT64241 | QIGLVFLCVLVSCMIGVGLYLSYLYFYMDPRIAKRGWPIQESRLVPALPA |
| | ::******:****::*:. * :*::::*: ** : *:* :* * *** * |
| P_brasilianum_hmfT3 | SIGPTIGLFLFAWTARASIHWIAPTIGITIYGATVFIVMQCLFVYIPLSY |
| Pen_rube_XP_002560799 | SFGPLIGLFLFAWTARASIHWIAPTIGITIYGATVFIVMQCIFMYIPLTY |
| Pen_oxal_EPS29964 | ALGPTIGLFIFAWTARASIHWIVPTIGITIYGATVFVVMQCLFVYIPLSY |
| Asp_terr_XP_001212020 | SFGPTIGLFLFAWTARASIHWIAPTIGITIYGATVFVVMQCIFVYIPLSY |
| Fus_oxys_ENH73763 | ALGPTIGLFLFAWTARASIHWIVPTIGITIYGATVFIVMQCIFVYIPLSY |
| Fus_oxys_EGU73369 | ALGPTIGLFLFAWTARASIHWIVPTIGITIYGATVFIVMQCIFVYIPLSY |
| Fus_oxys_EXL94287 | ALGPTIGLFLFAWTARASIHWIVPTIGITIYGATVFIVMQCIFVYIPLSY |
| Nec_haem_XP_003040064 | SIGPTIGLFLFAWTARKSIHWIAPTIGITIYGATVFIVMQCIFVYIPLSY |
| Fus_pseu_XP_009258565 | SIGPTIGLFLFAWTARASIHWIVPTIGITIYGATVFVVMQCIFVYIPLSY |
| Fus_gram_XP_011323833 | SIGPTIGLFLFAWTARASIHWIVPTIGITIYGATVFVVMQCIFVYIPLSY |
| Fus_fuji_CCT64241 | ALGPTIGLFLFAWTARSSIHWIVPTIGITIYGATVFIVMQCIFVYIPLSY |
| | ::** ****:****** *****.*************:****:*:****:* |
| P_brasilianum_hmfT3 | PMYAASLFAANDFFRSALACGSVLFAHPLFGNLGVARGTSLLGGLSVIGI |
| Pen_rube_XP_002560799 | PKYAASLFAANDFFRSALACGSVLFAHPLFGNLGVARGVSLLGGLSVIGI |
| Pen_oxal_EPS29964 | PQYAASLFAANDFFRSALACGSVLFAHPLFGNLGVARGTSLLGGLSVIGI |
| Asp_terr_XP_001212020 | PNYAASLFAANDFFRSALACGSVLFAHPLFGNLGVARGVSLLGGLSVIGI |
| Fus_oxys_ENH73763 | PMYAASLFAANDFFRSALACGSVLFAQPLFDNLGVAKGTSLLGGLSVIGI |
| Fus_oxys_EGU73369 | PMYAASLFAANDFFRSALACGSVLFAQPLFDNLGVAKGTSLLGGLSVIGI |
| Fus_oxys_EXL94287 | PMYAASLFAANDFFRSALACGSVLFAQPLFDNLGVAKGTSLLGGLSVIGI |
| Nec_haem_XP_003040064 | PMYAASLFAANDFFRSALACGSVLFAQPLFDNLGVAKGTSLLGGLSVIGI |
| Fus_pseu_XP_009258565 | PMYAASLFAANDFFRSALACGSVLFAQPLFDNLGVDKGTSLLGGLSVIGI |
| Fus_gram_XP_011323833 | PMYAASLFAANDFFRSALACGSVLFAQPLFDNLGVDKGTSLLGGLSVIGI |
| Fus_fuji_CCT64241 | PMYAASLFAANDFFRSALACGSVLFAQPLFDNLGVAKGTSLLGGLSVIGI |
| | * ************************:***.**** :*.*********** |
| P_brasilianum_hmfT3 | IGIWLLYYYGARLRSLSKFAISDD---- |
| Pen_rube_XP_002560799 | IGIWLLYFYGGRLRALSKFAISDPVE-- |
| Pen_oxal_EPS29964 | VGIWLLYVYGARLRSLSKFAISDD---- |
| Asp_terr_XP_001212020 | IGIWLLYFYGARLRALSKFALSPGASFE |
| Fus_oxys_ENH73763 | IGIWLLYFYGGRLRSLSKFAISDHVE-- |
| Fus_oxys_EGU73369 | IGIWLLYFYGGKLRSLSKFAISDHVE-- |
| Fus_oxys_EXL94287 | IGIWLLYFYGGKLRSLSKFAISDHVE-- |
| Nec_haem_XP_003040064 | IGIWLLYFYGARLRALSKFAVYEHVE-- |
| Fus_pseu_XP_009258565 | IGIWLLYFYGAKLRSLSKFAVSDHVE-- |
| Fus_gram_XP_011323833 | IGIWLLYFYGAKLRSLSKFAVSDHVE-- |
| Fus_fuji_CCT64241 | IGIWLLYFYGGKLRSLSKFAISDHVE-- |
| | :****** **.:**:*****: |

TABLE 7
Amino acid sequence alignment of Penicillium brasilianum hmfT4
and 10 closest orthologues.

| | |
|---|---|
| P_brasilianum_hmfT4 | -MSTTKEAFPHTDSDIMEDSEKNLPECEHIVSVEPTLKMRDGIVLMPQPS |
| Spo_sche_ERT02386 | ---MKSDEIPRPE--VIEANEK--VSDQDATSIGNNLKTRGGVVLMPQPS |
| Tog_mini_XP_007915981 | --MGTKQELDHVA--AMEHQEKS-GSDIEEPSLAPNLKKRDGVILMPQPS |
| Cap_coro_XP_007724585 | MASSEKAAIADTTKSASVSDQVDKGDVEQTTADVNLKRTKDGILLVPQPT |
| Spo_sche_ERS98342 | --MDTKHGVTVDAAGH-----HPS--SSDKTDGPPLKCNKHGIVLVPQPS |
| Asp_kawa_GAA83620 | --MVDVKESQAVEVLQ-----TKSVSSGDREADTRIKTTAQGIPLVPQPS |
| Cap_coro_XP_007725190 | MAVS----AADKTTSD------DQIAIEGGKDERVVKCRSDGIPLVPQPS |
| Asp_nige_XP_001389139 | --MADVKELQSVEVLQ-----EKSMSSGDPEANARIKTTAQGIPLVPQPS |
| Gro_clav_EFX04858 | -MNETKKIVAVDTERL-----DTSQEHSDKAEAPFVKHTKEGFLLVPQPS |
| Spo_sche_ERS94853 | MGQPGAIDIQEQPSSE-----DFRSEKHDKPEPVFLKATKDGIPLHPQPS |
| Asp_nige_EHA26600 | --MADVKELQSVEVLQ-----EKSMSSGDPEANARIKTTAQGIPLVPQPS |
| | *. * ***: |
| P_brasilianum_hmfT4 | DDPNDPLN------WSWFRKHAAMFTLSYLALVCYVAVTTLVTGTVPLAK |
| Spo_sche_ERT02386 | DDPADPLN------WSWFEKHAAMFTISYLALICYMSVTTLVAGTVNVAE |
| Tog_mini_XP_007915981 | DDPHDPLN------WSSFRKHMAMATISYLALTCYMTVTTLVPGTVELGK |
| Cap_coro_XP_007724585 | DDPEEPLN------WSFAKKHGALVVLALGSFFVKFTATILAPGAHSLAK |
| Spo_sche_ERS98342 | DDPEDPLN------WSFAKKHAAMFVLALESLLVKFSATLIAPGAHSLAA |
| Asp_kawa_GAA83620 | DDPEDPLRGNCLQNWSTFVKHAALVVLAFESFMTKMSNTLIAPDALELAK |
| Cap_coro_XP_007725190 | DDPEDPLN------WSSAKKHSAAVTLALMSFVLKFTTTLIAPGAHTLAA |
| Asp_nige_XP_001389139 | DDPEDPLN------WSQFTKIAALMVLAFESFLVKFSATLIAPDALELAE |
| Gro_clav_EFX04858 | DDPDDPLN------WSFSKKHVALFFLAMESLLVKFSATLISPGARTLAH |
| Spo_sche_ERS94853 | DDPEDPLN------WSPLRKHAALVVLAMESLIIKFSNTVIAPGAHTLAA |
| Asp_nige_EHA26600 | DDPEDPL----------------------------------APDALELAE |
| | *** :** ..: :. |
| P_brasilianum_hmfT4 | SMHVSKSTAVYLGNTPVALYAVAPWFWSPLSHFIGRRPVLLMCNIIAVVG |
| Spo_sche_ERT02386 | GLGVPKATAVYLGNTPVALYGVAPFLWSPLSHFIGRRPVLLLSNIMAMVG |
| Tog_mini_XP_007915981 | EFNVPKETAVYLGSTPVALYGVGPFLWSPLSHSIGRRPVLLLCNIIAIVG |
| Cap_coro_XP_007724585 | QFHVTAKRAVYIASASSIMPAVAPFFWIPMSHRYGRRPMLMAGSTMAIVF |
| Spo_sche_ERS98342 | QFHTAASKATYIGSAPSILYAIAPFFWIPLSHRVGRRPVLLASQVIALVA |
| Asp_kawa_GAA83620 | EFGVTKSTATYIGSAPPILNALTSFFWIPLSHRIGRRPVLLMGNLLALVS |
| Cap_coro_XP_007725190 | QFGTPASKATYIGSTPTIMFSVAPLLWIPLSSRYGRRPITLIGNFMAIWF |
| Asp_nige_XP_001389139 | EFNVPETTATYIGSVPSILNAITSFFWIPMSHRIGRRPVLLIGNLMTLVS |
| Gro_clav_EFX04858 | LFHVPLSKATYIGSAPTIMNAVGPFFWIPISHRIGRRPVLLMSQIIAMVA |
| Spo_sche_ERS94853 | QFGTAASTASYIGSAPSVLYAFAPFLWIPLSHRLGRRPVLLASHLVALLA |
| Asp_nige_EHA26600 | EFNVPETTATYIGSVPSILNAITSFFWIPMSHRIGRRPVLLIGNLMTLVS |
| | : .. * *:.... : .. . :* *:* ****: : ::: |
| P_brasilianum_hmfT4 | AVVVTTSKTYASCMVGRVILGAGGSAFWTLGPASIGDIFFRHEKGKKIGV |
| Spo_sche_ERT02386 | AGIVTSAENYGTAMAGRVILGAGGSAFWTLGPACIGDIFFRHEKGKKIGI |
| Tog_mini_XP_007915981 | TIIVATSHSYGACMAGRIILGLGGSAFWSLGPASIGDMFFRHEKGKKIGI |
| Cap_coro_XP_007724585 | ALIIARADTYAQALVCRLFMAFGASSAICIGPAAISDMFFLHEKGTRMGF |
| Spo_sche_ERS98342 | AIGVARSESYAQALGCRMVMGFGGSAGLCIGPAAISDMFFLHEKGSRMGV |
| Asp_kawa_GAA83620 | SIGVARSQTYAQALACRMVMTFGGSVGLSIGPAAISDMFFLHEKGSRMGV |
| Cap_coro_XP_007725190 | AIGVAESESYASALVCRIFMGFCGAAGLCLGPAGIADMFFLHEKGRHMGL |
| Asp_nige_XP_001389139 | AIGVARSQTYAQCLACRMLMNVGGSVGLSIGPAAISDMFFLHEKGSRMGV |
| Gro_clav_EFX04858 | AIGVGRSETYAQALGCRMVMGFGGSAGLCIGPAAISDMFFLHEKGTRMGI |
| Spo_sche_ERS94853 | AIGVGRAQSYSQALGCRMLMGFGGSAGLCISTAAISDMFFLHEKGTRLGL |
| Asp_nige_EHA26600 | AIGVARSQTYAQCLACRMLMNVGGSVGLSIGPAAISDMFFLHEKGSRMGV |
| | : : :..*. .: *:.: .: :..* *.*:** **** ::*. |
| P_brasilianum_hmfT4 | STLAIVIAPFLGTIIGGPIIENEKLGWPASQWIPLIFMAAGFIMQIFFLP |
| Spo_sche_ERT02386 | STLAIVVSPFLGTLVGGAIIENPHLGWPASQWIPLIFMGVGLVMQVFFLP |
| Tog_mini_XP_007915981 | STLAIVVSPFAGGIIGGAIIDSPKLGWRWSQWIPLILMAIGFAMQVVFLP |
| Cap_coro_XP_007724585 | NTILLITAPYLGGVVGGSIMYNPNLGWRWTMYIAAILLAGLLICQFLFVP |
| Spo_sche_ERS98342 | NSILLVVAPYVGGVAGGAIQQNPALGWRWSMYVSAITYAVQLTAQFCLVP |
| Asp_kawa_GAA83620 | NSILLVIGPYVGGVAGASIAYNPNLGWRWSMYIAAILYAAQFVFQFLFVP |
| Cap_coro_XP_007725190 | NTVLLVSAPYAGGVAGGAVQFNKSLGWRWSMYIAAIIYSGLFVAQLLLVP |
| Asp_nige_XP_001389139 | NSILLVISPYVGGVAGGSIAYNKSLGWRWSMYIAAILYATQFVAQIFFVP |
| Gro_clav_EFX04858 | QSILLVVAPYVGGVAGGSIQYNSKLGWRWSMYVSAICYSAQFVCQFFFVP |
| Spo_sche_ERS94853 | NGMLFVVAPYIGGVAGGAIQQNKHLGWRWAMYIAAICYAVQLVLQCLLVP |
| Asp_nige_EHA26600 | NSILLVISPYVGGVAGGSIAYNKSLGWRWSMYIAAILYATQFVAQIFFVP |
| | . : :: .*: * : *..: . *** : ::. * . : * ::* |
| P_brasilianum_hmfT4 | ETIYIRETRAHPAIMSTSTPGKPTFWDRYGIHIPKRSEEKQHSFLFIATR |
| Spo_sche_ERT02386 | ETIYVREVQGQRAGLASKT--KATLWDRYGVRIPQRTSDKKHSFFFIFSR |
| Tog_mini_XP_007915981 | ETVYVREIGSPGGVPQPVTPTKPTRWGRYGIHIPKRPADKRDGFWFIASR |
| Cap_coro_XP_007724585 | ETIFDR-ALA-KPVHEK---PPPTIAARLGFRRPTAT--RNENWGHTFTR |
| Spo_sche_ERS98342 | ETIYER-----GGHRR----QPQSVARRFGFRTPTNP--TGESWLQTFRR |
| Asp_kawa_GAA83620 | ETIYVRDENG-QGVSRSSEPKPTTFLSRLKFRPPPPP--KGESWGRTFIK |
| Cap_coro_XP_007725190 | ETLYPRPAAG-APAPKS---TTTGTLRKLGFRKPTYA--KDPTWLDLFSR |
| Asp_nige_XP_001389139 | ETIYTRNEKT-SAESKPSDRKKSTFLSRMKFRKPVVP--KEETWGQTFRK |
| Gro_clav_EFX04858 | ETIYEREVAA-AELPE----QKKTIWRRLGFRMPTNP--SGETWLQTFRR |
| Spo_sche_ERS94853 | ETIYNKDVAA-AEPPE----AKATLYRRLGFRTPKPA--PGETWAATFRM |
| Asp_nige_EHA26600 | ETIYTRNEKT-SAESKPSDRKKSTFLSRMKFRKPVVP--KEETWGQTFRK |
| | **:: : : .: * . : |
| P_brasilianum_hmfT4 | PFVLFKFPAVILSAFWFGIAYMMHVGITSEIPLIFEEH--YDFSVLEIGL |
| Spo_sche_ERT02386 | PFVLLRFPAITLGTFWFGVAYMMHVGITAEIPLIFEAK--FHFTVLDVGL |
| Tog_mini_XP_007915981 | PFVMFKFPVVVLTSFWFGLAYWCHVGITAELPLIFEPEP-FNFSVTDVGL |
| Cap_coro_XP_007724585 | PFAMFAYPAVVLPSFWFSVTAMTEVANTAGFPLNFGPGSRWHFNTQEIGF |
| Spo_sche_ERS98342 | PYAMFVYPAVVVPSFWVSTAVMTEVANTAGFTLNFGVTSRFHFTTAQVGY |
| Asp_kawa_GAA83620 | PYKMFAYPAVFLPSFWFGVACMTEVGNTAGFALNFGSDSRWGFNLAQVGF |
| Cap_coro_XP_007725190 | PVAMFAYPTVLLPSIWFSLAAMTEVANTAGFPLNFGEHTRWNFNTRSVGF |
| Asp_nige_XP_001389139 | PYKMFAYPAVVLPSFWFGVANMTEVGNTAGFALNFGSKSRFHFNLAQVGF |
| Gro_clav_EFX04858 | PFVMFAYPAVVLPSFWASVAVMTEVANTAGFAINFGASSRFHFNTAQVGF |
| Spo_sche_ERS94853 | PFSMFAYPAVVLPCFWASTCIMTEVANTAGLSLNFGSGTRFDFSVAQVGY |
| Asp_nige_EHA26600 | PYKMFAYPAVVLPSFWFGVANMTEVGNTAGFALNFGSKSRFHFNLAQVGF |
| | * :: :*.: : :* . .*. *: :.: * : *. .:* |
| P_brasilianum_hmfT4 | SGFSGLIGALLGEVYAGPSLDFIAKRTMKQGREWRPEYRLQAIWPALITV |
| Spo_sche_ERT02386 | SGLSGLIGALIGEAYAGPSIDYLARRSLKQGKEWRPEYRLKVIWPALVAI |
| Tog_mini_XP_007915981 | AAFSGLIGALIGEAYAGPAIDYIAKRCLKQGKEWRPEMRLKAIWPALVAT |
| Cap_coro_XP_007724585 | CSFSGFIGAIVGEFFAGPLCDLVAKRHLNKGTAWKPEYLLPLTISGLITV |
| Spo_sche_ERS98342 | CFLSGLIGAFSGELLAGPLCDLLVKRALKKEHGWRPETLLVLNVTGLVAI |
| Asp_kawa_GAA83620 | CYFSGVIGAALGEIFGGPLCDMLAKYSIRHGKEWKPERLLHLVWSGMVTI |
| Cap_coro_XP_007725190 | CSFSGFIGALLGEIFAGPLCDFIAGRALAKKRAWVPEKILPVTFISLVTI |
| Asp_nige_XP_001389139 | CYFSGIIGAGIGEIFGGPLCDMVAKYSLRRGQEWRPERLLHLAWSALITI |
| Gro_clav_EFX04858 | CFISGLIGAFTGEVCAGPLCDMAVRNSLRRNQVWRAEKLLKLAITGLVTI |
| Spo_sche_ERS94853 | CFFAGLIGSSLGEVCAGPLCDMTAKRSLRSGVAWVPEKLLKLFLSGLFTT |
| Asp_nige_EHA26600 | CYFSGIIGAGIGEIFGGPLCDMVAKYSLRRGQEWRPERLLHLAWSALITI |
| | . ::*.**: ** .** * . : * .* * .:.: |
| P_brasilianum_hmfT4 | PAGLIMFGTSIQFGN--VWITPLIGQAVYIFGIEIATTVIQTYILECYPR |
| Spo_sche_ERT02386 | PGGLVMFGTAIEFGN--SWVTPLVGQLIYIFGIEIATTIIQTYILESYPR |
| Tog_mini_XP_007915981 | PIGLIMFGVSIQFGN--AWITPLVGQGIYIFGIEIATTVWY--------- |
| Cap_coro_XP_007724585 | PAGLLLYGFELQWPT--GWAAALTGVAIFTAGQEILMTVLMTYMTDCYPG |
| Spo_sche_ERS98342 | VGGLLVYGIQLQGSAPGDWASPLAGMILFVFGQEIIVTVVMTYMTDCYPD |
| Asp_kawa_GAA83620 | SAGLLLYGLELEYGN--NWAAALTGIGLFTFGQEVLVTVLLTYMTDCYPE |
| Cap_coro_XP_007725190 | PAGLLLYGLELEYPT--GWAAALTGVAIFAFGQEVALTAIMTYLVDCYPQ |
| Asp_nige_XP_001389139 | SAGLLLYGLELEYGD--SWAAALTGIGLFTFGQEVLVTVLLTYMTECYPE |
| Gro_clav_EFX04858 | FAGLMLYGFELESSK--AWARPLAGMILFVFGQEVVVTIIMTYMTDCYPE |
| Spo_sche_ERS94853 | FAGLLVYGFTLEYVQTSQWAVPLVGLGLFVFGQEIVVTVLLAYMTECYRD |
| Asp_nige_EHA26600 | SAGLLLYGLELEYGD--SWAAALTGIGLFTFGQEVLVTVLLTYMTECYPE |
| | **:::* :: * .* * :: * *: * |
| P_brasilianum_hmfT4 | QGAEANLVFNLIRNLFSYISPFFVQPMIATLG-TTSPFGLSAALTAFFFP |
| Spo_sche_ERT02386 | QGAEANLIFNLVRNIFSYISPFFLTPFIAKVG-YAAPFGLFAALTVVFFP |
| Tog_mini_XP_007915981 | --------------IFEFLS--FV-------------------------- |
| Cap_coro_XP_007724585 | SASEVSIVFQCLLNAMAYHPPFYVPQWIAEPGGAKVPYIVFAVLPVVFFP |
| Spo_sche_ERS98342 | QAAEVAIVFQFFFNLMCFHPPFYTPGWIASAG-ARTPYIVYAVIPLALFP |
| Asp_kawa_GAA83620 | DAAEVTLVLQFFFAIQTFHVPFYLPQWIKQPGGAKVPYIVFAALPVVLYP |
| Cap_coro_XP_007725190 | RASECSVVFQFWRNLMAFHPPFYVPQWIESGGGAKVPYIVFACLAVGLFP |
| Asp_nige_XP_001389139 | DAAEVAIVFQFFFAVQTFHPPFYLPQWIKQPGGAKVPYIVFAALPIVLYP |
| Gro_clav_EFX04858 | HAAEVAVVFQFFFNLMCYHPPFYTPQWIASAG-SKVPYIVYAVLPVGLFP |
| Spo_sche_ERS94853 | RAVECTIVFQFFLNLMCFPPPFFTPLWIAKKGGAKVPYIVYALLPVAFFP |
| Asp_nige_EHA26600 | DAAEVAIVFQFFFAVQTFHPPFYLPQWIKQPGGAKVPYIVFAALPIVLYP |
| | : : |
| P_brasilianum_hmfT4 | FTVGVLMWRGKQIRDKGGDPGWSRD------------------------- |
| Spo_sche_ERT02386 | FTILVLMLRGKQLREKAGDPGWSRD------------------------- |
| Tog_mini_XP_007915981 | -------------------------------------------------- |
| Cap_coro_XP_007724585 | LTIGVLMWKGPQLRARGPWFTI---------------------------- |
| Spo_sche_ERS98342 | LLMGPFIWKGEQIRSKGPLFRLSK-------------------------- |
| Asp_kawa_GAA83620 | ICIWIFEWKGEKIRKRGPLFRI---------------------------- |
| Cap_coro_XP_007725190 | FGVGTLLWKGSNLRARGPMFSFSHKQ------------------------ |
| Asp_nige_XP_001389139 | FCISLFTWKGPQIRKRGPFFVL---------------------------- |
| Gro_clav_EFX04858 | ILIGPFMWKGSQIREKGPLFRFISFKRKATKTSFKASSKKFFKKLLGREK |
| Spo_sche_ERS94853 | LCILPFMLKGQAIRERGGVLAFWKRRQ----------------------- |
| Asp_nige_EHA26600 | FCISLFTWKGPQIRKRGPFFVL---------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | KDIASNFPSQGEVVFHPPAAKEESNIEAASEEPFASTLSNTPSVQANIVS |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | SSSQNAVPQTDDIPSTPEAATEALTVSPHPISNTSLIVADNAANPVSENV |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | VLSAPQTDDIASTPPPTTAEASPSDELWTRAFGLFREKEPELARDYMTHL |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | ATLHNSVDSVDLSASRSVKDLVDRLLEKREEKLWKVSILGKSVKIREQTE |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | KLVRLLVFFDPVVKEAVSSQPYAALAWSGVSLILPLLTSGTTQNEAMLKG |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | FDTIGNEQLYWNICEKTYLESAEHEIYKPLVEPLAQLYSDMIAFQALAIC |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | HYSKAQLSRAWENIAGSNDWDVRANKIEKQSTNIQRNILNLDKQEIRILW |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | NTQLQGIQESQFALNDVRQILSENNRLNQKRYDDEKERELLKELASAYES |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | YKNFNKQRVEGTCEWFFNDNRFRTWRDSKMSSLLWVSAGPGCGKSVLSRA |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | LVDEHRLSTNAATSTVCHFFFKDGDARRLRSTAALCAVLHQLFTQDHTGS |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | LIKHALPSYNEGMALRNNFPGLWKILINCANSPEAGQIICVLDALDECEI |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | QSRNELIGELKRFYCEQRELAKSSTLMFLITSRPYADLEFAFLKFNTTTY |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | LRFDGDEKSADIGKEISLVIDERVNTVAASFSEKHRLELADHLKSMENRT |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | YLWLHLVFSIIEGNFSYSRPLDIKKLLSQIPPEVSGAYEQILDKSSNKDL |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | TMKLLQLVLAAEHPLTLDEVNIALALADSPQDSAAELKSALWPKGNFQTT |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | VRNFCGLFVSVYDSKLFFIHQTAREFLLSSERDGNWKGHFALPECHSILS |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | RVCIDYLLFPDLVEHPLIVEDEENEKETRPSFFEYAARYWTSHYNSQEDA |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | NAYKSRKDACMLCHKINIEPMDTTKTSALQAASLQGQLKTIRLLIDRGAN |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | VNLQGGDYGSALQAASRNGYTEIVQILLNSGADVNLDGGAALKAASRNGH |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | TEIVQILLNSGADVNLQGGEYGSALQAASSFGYKEVVQILLNSGADVNLQ |
| Spo_sche_ERS94853 | -------------------------------------------------- |
| Asp_nige_EHA26600 | -------------------------------------------------- |
| P_brasilianum_hmfT4 | -------------------------------------------------- |
| Spo_sche_ERT02386 | -------------------------------------------------- |
| Tog_mini_XP_007915981 | -------------------------------------------------- |
| Cap_coro_XP_007724585 | -------------------------------------------------- |
| Spo_sche_ERS98342 | -------------------------------------------------- |
| Asp_kawa_GAA83620 | -------------------------------------------------- |
| Cap_coro_XP_007725190 | -------------------------------------------------- |
| Asp_nige_XP_001389139 | -------------------------------------------------- |
| Gro_clav_EFX04858 | GGEYGSALQAASIFRHKEVVQILLNSGADVNLDGGAALKAASRKGQTEIV |
| Spo_sche_ERS94853 | -------------------------------------------------- |
| Asp_nige_EHA26600 | -------------------------------------------------- |
| P_brasilianum_hmfT4 | -------------- |
| Spo_sche_ERT02386 | -------------- |
| Tog_mini_XP_007915981 | -------------- |
| Cap_coro_XP_007724585 | -------------- |
| Spo_sche_ERS98342 | -------------- |
| Asp_kawa_GAA83620 | -------------- |
| Cap_coro_XP_007725190 | -------------- |
| Asp_nige_XP_001389139 | -------------- |
| Gro_clav_EFX04858 | EMLHASANNKTEEL |
| Spo_sche_ERS94853 | -------------- |
| Asp_nige_EHA26600 | -------------- |

TABLE 8
Amino acid sequence alignment of Penicillium brasilianum hmfT5
and 10 closest orthologues.

| | |
|---|---|
| P_brasilianum_hmfT5 | MEDHEK--------EYDSTSPPGTATEE---------GNGGYFNTLTVPE |
| Pen_digi_EKV20717 | MEQHPGPDDASLHSEYGTEDEDNNQDLENSLVRKLNTHDFTSVETLRSPQ |
| Pen_digi_EKV19541 | MEQHPGPDDASLHSEYGTEDEDNNQDLENSLVRKLNTHDFTSVETLRSPQ |
| Pen_rube_XP_002565665 | MEQHPGLDDGSLHSEYQNEDENDNKSPDNQPIHKLNTHNFTSVETLHVPQ |
| Asp_oryz_KDE82314 | MEFH----------LHDEAPPASTAPTEYGDQSGEEFEAYSEKPTLGVPD |
| Asp_oryz_EIT77345 | MEFH----------LHDEAPPASTAPTEYGDQSGEEFEAYSEKPTLGVPD |
| Asp_flav_XP_002380612 | MEFH----------LHDEALPASTAPTEYGDQSGEEFEAYSGKPTLGVPD |
| Asp_terr_XP_001208847 | -MEK----------NFDTSDDFSSSP----------LPETKSYETLAVPN |
| Asp_kawa_GAA86951 | MNSH----------EFPEDEKSSDLP----------VPERKSLDTLNVPH |
| Asp_nige_XP_001400982 | MNPP----------EFPEDEKSSDLP----------IPERKSLDTLNVPH |
| Oph_pice_EPE02908 | MDQY-------------ENSDDSETPAD---------NDNYRPNRLSVPH |
| | . * *. |
| P_brasilianum_hmfT5 | INLREASSAETLTPH--ASVVQPPKKA-AEWHMTPQVIRNAERDEAAGFK |
| Pen_digi_EKV20717 | VNIHEAKSAETLNVA-NAETSLLPKKA-AEWSMTPQVIRNAERDEAAGFK |
| Pen_digi_EKV19541 | VNIHEAKSAETLNVA-NAETSLLPKKA-AEWSMTPQVIRNAERDEAAGFK |
| Pen_rube_XP_002565665 | ANIHEAKSSETLNVA-HADTSIPPKKT-AEWSMTPQVIRNAERDEAAGFK |
| Asp_oryz_KDE82314 | NNVREATSAETLAVHGSPHITPPPGRD-AEWSMTDQVIRNKERSEAAGYK |
| Asp_oryz_EIT77345 | NNVREATSAETLAVHGSPHITPPPGRD-AEWSMTDQVIRNKERSEAAGYK |
| Asp_flav_XP_002380612 | NNVREATSAETLAVHGSPHITPPPGRD-AEWSMTDQVIRNKERSEAAGYK |
| Asp_terr_XP_001208847 | LNIREASSAETLAAP-HANNTPTPGKDAAEWHMTPQVIQQQEREIAAGFK |
| Asp_kawa_GAA86951 | IDVREAPSSETLTVP-HANTTSPPGKD-AEWSMTPQVIRSQEREAAAGFK |
| Asp_nige_XP_001400982 | INVREAPSAETLIVP-HAVNASAPGKD-AEWSMTPQVIRSQEREAAAGFK |
| Oph_pice_EPE02908 | GNSPEASSSETLEALFPPTGSPPEKKKIAEWSMTPQVVRNAERDAAAGFK |
| | : ** *:*** . : *** ** **::. **. ***:* |
| P_brasilianum_hmfT5 | RRELGVTWQDLSVEVLAAEAAVKENMISQFNVPQLIKDFRRKPPLKSILS |
| Pen_digi_EKV20717 | RRELGVTWQNLTVDVLAAEAAVNENMISQFNVPQLIKDFRRKPPLKSILS |
| Pen_digi_EKV19541 | RRELGVTWQNLTVDVLAAEAAVNENMISQFNVPQLIKDFRRKPPLKSILS |
| Pen_rube_XP_002565665 | KRELGVTWQSLTVDVLAAEAAVNENMISQFNLPQLIKDFRRKPPLKSILS |
| Asp_oryz_KDE82314 | KRELGVTWQNLTVEVLAAEAAVKENQFTQYNIIQLIQDWRRKPPLKAILQ |
| Asp_oryz_EIT77345 | KRELGVTWQNLTVEVLAAEAAVKENQFTQYNIIQLIQDWRRKPPLKAILQ |
| Asp_flav_XP_002380612 | KRELGVTWQNLTVEVLAAEAAVKENQFTQYNIIQLIQDWRRKPPLKAILQ |
| Asp_terr_XP_001208847 | RRELGVTWENLSVDVLAAEAAVKENLFSQFNIPQLIKDWRRKPPMKSILS |
| Asp_kawa_GAA86951 | KRELGVTWKNLGVDVLAAEAAVNENLFSQFNVPQRIRDFTRKPPLKSILA |
| Asp_nige_XP_001400982 | KRELGVTWKNLGVDVLAAEAAVNENLFSQFNLPQRIRDFTRKPPLKSILT |
| Oph_pice_EPE02908 | KRELGVTWQNLSVDVIAAEAAVKENMVSQFNVPQLVKDYLHKPPLKSIVQ |
| | :*******:.* *:*:******:** .:*:*: * ::*: :***:*:*: |
| P_brasilianum_hmfT5 | NSHGCVKPGEMLLVLGRPGSGCTTLLKMLANRREG-YQNITGDVRFGNMT |
| Pen_digi_EKV20717 | DSHGCVKPGEMLLVLGRPGSGCTTLLKILSNRREG-YHTINGDVRFGNMT |
| Pen_digi_EKV19541 | DSHGCVKPGEMLLVLGRPGSGCTTLLKILSNRREG-YHTINGDVRFGNMT |
| Pen_rube_XP_002565665 | DSHGCVKPGEMLLVLGRPGSGCTTLLKMLSNRREG-YHTVNGDVRFGSMS |
| Asp_oryz_KDE82314 | DSHGCVKPGEMLLVLGRPGSGCTTLLKMLANRREG-YHSVHGDVSFGNMN |
| Asp_oryz_EIT77345 | DSHGCVKPGEMLLVLGRPGSGCTTLLKMLANRREG-YHSVHGDVSFGNMN |
| Asp_flav_XP_002380612 | DSHGCVKPGEMLLVLGRPGSGCTTLLKMLANRREG-YHSVHGDVSFGNMN |
| Asp_terr_XP_001208847 | DSHGCVKPGEMLLVLGRPGSGCTTLLKLLTNRRKG-YHTIRGDVRFGNMT |
| Asp_kawa_GAA86951 | ESHGCVKPGEMLLVLGRPGSGCTTLLNLLSNRRHG-YHTIKGDVSFGNMS |
| Asp_nige_XP_001400982 | ESHGCVKPGEMLLVLGRPGSGCTTLLNLLSNRRHG-YHTIKGDVSFGNMS |
| Oph_pice_EPE02908 | DSHGCVKPGEMLLVLGRPGSGCTTLLKMLSNHRDGGYKTINGDVRFGNMT |
| | :*************************::*:*:*.* *:.: *** **.*. |
| P_brasilianum_hmfT5 | PEEASRYQGQIVMNTEEELFYPRLTVGQTMDFATKLKVPYHLPGEGKSVA |
| Pen_digi_EKV20717 | PKEAEGYNGQIVMNTEEELFYPRLTVGQTMDFAARLKVPFHLPEGAQSVE |
| Pen_digi_EKV19541 | PKEAEGYNGQIVMNTEEELFYPRLTVGQTMDFAARLKVPFHLPEGAQSVE |
| Pen_rube_XP_002565665 | PKEAEDYNGQIVMNTEEELFYPRLTVGQTMDFAARLKVPFHLPEGVQSVD |
| Asp_oryz_KDE82314 | SEEAAHYRGQIVMNTEEELFYPRLTVGQTMDFATKLKVPAHLPAETKSVH |
| Asp_oryz_EIT77345 | SEEAAHYRGQIVMNTEEELFYPRLTVGQTMDFATKLKVPAHLPAETKSVH |
| Asp_flav_XP_002380612 | SEEAAHYRGQIVMNTEEELFYPRLTVGQTMDFATKLKVPAHLPAETKSVH |
| Asp_terr_XP_001208847 | HEEAVQYQSQIVMNTEEELFYPRLTVGQTMDFATRLKVPSHLPNDVKSVE |
| Asp_kawa_GAA86951 | HEEAAQYRSHIVMNTEEELFYPRLTVGQTMDFATRLKVPSHLPDGTASVS |
| Asp_nige_XP_001400982 | HEEAAQYRSHIVMNTEEELFYPRLTVGQTMDFATRLKVPSHLPDGAASVK |
| Oph_pice_EPE02908 | AEEALNYHGQIIMNTEEELFYPRLTVGQTIEFATKLKIPFHLPDGIKSVE |
| | :** *..:*:*****************::**::**:* *** ** |
| P_brasilianum_hmfT5 | EYTAETKQFLLESMGIAHTADTKVGNEFVRGVSGGERKRVSIIECLATRG |
| Pen_digi_EKV20717 | EYTAETKEFLLQSMGIAHTADTKVGNEFVRGVSGGERKRVSIIECLATRG |
| Pen_digi_EKV19541 | EYTAETKEFLLQSMGIAHTADTKVGNEFVRGVSGGERKRVSIIECLATRG |
| Pen_rube_XP_002565665 | EYTAETKQFLLESMGISHTADTKVGNEFVRGVSGGERKRVSIIECLATRG |
| Asp_oryz_KDE82314 | DYVAETKQFLLESMKIAHTADTKVGNEFVRGVSGGERKRVSIIECMATNG |
| Asp_oryz_EIT77345 | DYVAETKQFLLESMKIAHTADTKVGNEFVRGVSGGERKRVSIIECMATNG |
| Asp_flav_XP_002380612 | DYVAETKQFLLESMKIAHTADTKVGNEFVRGVSGGERKRVSIIECMATNG |
| Asp_terr_XP_001208847 | EYTAETKRFLLESMGIAHTADTKVGNEFVRGVSGGERKRVSIIEVLATKG |
| Asp_kawa_GAA86951 | EYTAETKQFLMESMGISHTADTKVGNEFVRGVSGGERKRVSIIECLATRG |
| Asp_nige_XP_001400982 | EYTAETKQFLMESMGISHTADTKVGNEFVRGVSGGERKRVSIIECLATRG |
| Oph_pice_EPE02908 | EYTDETRDFLLESMGITHTADTPVGNEYVRGVSGGERKRVSIIECLATRA |
| | :*. **: **::** *:***** ****:**************** :**.. |
| P_brasilianum_hmfT5 | SVFTWDNSTRGLDASTALEWAKALRAMTDVQGLSTIVTLYQAGNGIYNLF |
| Pen_digi_EKV20717 | SIYSWDNSTRGLDASTALEWAKALRAMTDILGLSTIVTLYQAGNGIYNLF |
| Pen_digi_EKV19541 | SIYSWDNSTRGLDASTALEWAKALRAMTDILGLSTIVTLYQAGNGIYNLF |
| Pen_rube_XP_002565665 | SVYSWDNSTRGLDASTALEWAKALRAMTDVLGLSTIVTLYQAGNGIYNLF |
| Asp_oryz_KDE82314 | SIFTWDNSTRGLDASTALEWAKALRAMTNVMGLTTIVTLYQAGNGIYNLF |
| Asp_oryz_EIT77345 | SIFTWDNSTRGLDASTALEWAKALRAMTNVMGLTTIVTLYQAGNGIYNLF |
| Asp_flav_XP_002380612 | SIFTWDNSTRGLDASTALEWAKALRAMTNVMGLTTIVTLYQAGNGIYNLF |
| Asp_terr_XP_001208847 | SVFCWDNSTRGLDASTALEWAKALRAMTDVQGLSTIVTLYQAGNGIYNLF |
| Asp_kawa_GAA86951 | SVFCWDNSTRGLDASTALEWAKALRAMTNVLGLSTIVTLYQAGNGIYNLF |
| Asp_nige_XP_001400982 | SVFCWDNSTRGLDASTALEWAKALRAMTNVLGLSTIVTLYQAGNGIYNLF |
| Oph_pice_EPE02908 | SVYCWDNSTRGLDASTALEWAKALRAMTDVLGLSTIVTLYQAGNGIYNLF |
| | *:: ************************:: **:**************** |
| P_brasilianum_hmfT5 | DKVLVLDEGKQIYYGPAAEAKPFMENLGFVYTDGANIGDFLTGLTVPTER |
| Pen_digi_EKV20717 | DKILVLDEGKQIYYGPAAAAKPFMEDLGFMYTDGANVGDFLTGLTVPTER |
| Pen_digi_EKV19541 | DKILVLDEGKQIYYGPAAAAKPFMEDLGFMYTDGANVGDFLTGLTVPTER |
| Pen_rube_XP_002565665 | DKVLVLDEGKQIYYGPAAAAKPFMEDLGFVYTDGANIGDFLTGVTVPTER |
| Asp_oryz_KDE82314 | DKVLVLDEGKQIYYGPAASAKPFMEDLGFVYSDGANVGDYLTGVTVPTER |
| Asp_oryz_EIT77345 | DKVLVLDEGKQIYYGPAASAKPFMEDLGFVYSDGANVGDYLTGVTVPTER |
| Asp_flav_XP_002380612 | DKVLVLDEGKQIYYGPAASAKPFMEDLGFVYSDGANVGDYLTGVTVPTER |
| Asp_terr_XP_001208847 | DKVLVLDEGKQIYYGPAQAAKPFMEELGFVYSDGANIGDYLTGVTVPTER |
| Asp_kawa_GAA86951 | DKALVLDEGKQIFYGPASAAKPFMENLGFVYTDGANVGDFLTGVTVPTER |
| Asp_nige_XP_001400982 | DKVLVLDEGKQIFYGPAAAAKPFMENLGFVYTDGANVGDFLTGVTVPTER |
| Oph_pice_EPE02908 | DKVLVLDEGKEIYYGPASEAKGFMESIGFVYSEGANIGDFLTGVTVPTER |
| | ** *******:*:**** ** ***.:**:*::***:**:***:****** |
| P_brasilianum_hmfT5 | KIRPGWENRFPRTADAILTEYQNSATYKNEVSLYGYPDTDLAAERTEAFK |
| Pen_digi_EKV20717 | KIRPGFENSFPRNADAILTEYIKSSTYRRMVSTYDYPDSELSRERTAAFK |
| Pen_digi_EKV19541 | KIRPGFENSFPRNADAILTEYIKSSTYRRMVSTYDYPDSELSRERTAAFK |
| Pen_rube_XP_002565665 | KIRPGYENTFPRNADAILAEYKKSSIYDRMVSTYDYPDSNLSRERTDAFK |
| Asp_oryz_KDE82314 | KIRPGYENRFPKNAEAILAEYQRSTLYQTMTREYDYPSSDAARQRTEEFK |
| Asp_oryz_EIT77345 | KIRPGYENRFPKNAEAILAEYQRSTLYQTMTREYDYPSSDAARQRTEEFK |
| Asp_flav_XP_002380612 | KIRPGFENRFPKNAEAILAEYQRSTLYQTMTREYDYPSSDAARQRTEEFK |
| Asp_terr_XP_001208847 | KIRPGREHRFPRNADAILAEYKNSPLYTHMISEYDYPNSEIAKARTEDFK |
| Asp_kawa_GAA86951 | RIRPGYENRFPRNADAIMAEYKASAIYSHMTAEYDYPTSAVARERTEAFK |
| Asp_nige_XP_001400982 | RIRPGYENRFPRNADSIMVEYKASAIYSHMTAEYDYPTSAIAQERTEAFK |
| Oph_pice_EPE02908 | KIKPGWENRFPRTAEAIFAEYQKSTICRDAMSEYDYPDTTLAATRTEDFK |
| | :*:** *: **:.*::*:.** *. *.** : : ** ** |
| P_brasilianum_hmfT5 | ESVAWEKSKHLPKGSDLTTSFWAQLMSCTARQYQILWGEKSTFLIKQILS |
| Pen_digi_EKV20717 | ESVAWEKSKHLPKSSSLTTSFWAQLVACTKRQYQILWGEKSTFITKQVLS |
| Pen_digi_EKV19541 | ESVAWEKSKHLPKSSSLTTSFWAQLVACTKRQYQILWGEKSTFITKQVLS |
| Pen_rube_XP_002565665 | ESVAWEKSSHLPKGSSLTTSFWVQLIACTKRQYQILWGEKSTFIIKQVLS |
| Asp_oryz_KDE82314 | ESVAWEKAKHLPNSSTLTVGFWDQLIACTIRQYQILWGEKSTFLIKQVLS |
| Asp_oryz_EIT77345 | ESVAWEKAKHLPNSSTLTVGFWDQLIACTIRQYQILWGEKSTFLIKQVLS |
| Asp_flav_XP_002380612 | ESVAWEKAKYLPNSSTLTVGFWDQLIACTIRQYQILWGEKSTFLIKQVLS |
| Asp_terr_XP_001208847 | ESVAFEKAKYLPKNTTLTTGFGTQLWACTIRQYQILWGEKSTFLIKQVLS |
| Asp_kawa_GAA86951 | ESVAFEKTTHQPQKSPFTTGFGTQVLACTRRQYQILWGEKSTFLIKQILS |
| Asp_nige_XP_001400982 | ESVAFEKTTHQPKKSPFTTGFGTQVLACTRRQYQILWGEKSTFLIKQILS |
| Oph_pice_EPE02908 | HSVAWEKSSHLPKGSRLTTSFWAQVMFCTHRQYQILWGERSTFLIRQVLS |
| | .***:**:.: *: : :*..* *: ** *********:***: :*:** |
| P_brasilianum_hmfT5 | CVMALIAGSCFYNSPDTSAGLFTKGGAVFFSLLYNCIVAMSEVTESFKGR |
| Pen_digi_EKV20717 | CAMALIAGSCFYDSPDTSEGLFTKGGAVFFSLLYNCIVAMSEVTESFKGR |
| Pen_digi_EKV19541 | CAMALIAGSCFYDSPDTSEGLFTKGGAVFFSLLYNCIFAMSEVTESFKGR |
| Pen_rube_XP_002565665 | CVMALIAGSCFYDSPDTSAGLFTKGGAVFFSLLYNCIVAMSEVTESFKGR |
| Asp_oryz_KDE82314 | VAMALIAGSCFYNSPDTTAGLFTKGGAVFFALLYNCIVAMSEVTESFKGR |
| Asp_oryz_EIT77345 | VAMALIAGSCFYNSPDTTAGLFTKGGAVFFALLYNCIVAMSEVTESFKGR |
| Asp_flav_XP_002380612 | VAMALIAGSCFYNSPDTTAGLFTKGGAVFFALLYNCIVAMSEVTESFKGR |
| Asp_terr_XP_001208847 | LSMALIAGSCFYNSPDTTAGLFTKGGAVFFSLLYNCIVAMSEVTESFKGR |
| Asp_kawa_GAA86951 | LVMALIAGSCFYNAPQTSAGLFTKGGAVFFSLLYNTIVAMSEVTESFKGR |
| Asp_nige_XP_001400982 | LVMALIAGSCFYNAPQTSAGLFTKGGAVFFSLLYNTIVAMSEVTESFKGR |
| Oph_pice_EPE02908 | LAMALIAGSCFYDAPDDSSGLFTKGGAVFFTLLYNSMAAMSEVTDSFKGR |
| | **********::*: : ***********:**** : ******:***** |
| P_brasilianum_hmfT5 | PILTKHKSFAMYHPAAFCLAQITADFPVLLFQCTIFSVVIYWMVGLKHTA |
| Pen_digi_EKV20717 | PVLIKHKDFAMYHPAAFCLAQIMADFPVLLFQCSIFSVVIYWMSGLKHTA |
| Pen_digi_EKV19541 | PVLIKHKDFAMYHPAAFCLAQIMADFPVLLFQCSIFSVVIYWMSGLKHTA |
| Pen_rube_XP_002565665 | PVLVKHKGFAMYHPAAFSLAQIMADFPVLLFQCTIFSVVIYWMSGLKHTA |
| Asp_oryz_KDE82314 | PVLIKHKSFAMYHPSAFCLAQITADLPVLLVQCTLFAVVIYWMTGLKHTA |
| Asp_oryz_EIT77345 | PVLIKHKSFAMYHPSAFCLAQITADLPVLLVQCTLFAVVIYWMTGLKHTA |
| Asp_flav_XP_002380612 | PVLIKHKSFAMYHPSAFCLAQITADLPVLLVQCTLFAVVIYWMTGLKHTA |
| Asp_terr_XP_001208847 | PVLVKHKGFGFYHPAAFCLAQITADFPVLLFQCTIFAIVMYFMVGLKVDA |
| Asp_kawa_GAA86951 | PVLIKHKGFAFYHPAAFCLAQITADFPVLLFQCTIFSVVLYWMVGLKATA |
| Asp_nige_XP_001400982 | PVLIKHKAFAFYHPAAFCLAQITADFPVLLFQCTIFSVVLYWMVGLKATA |
| Oph_pice_EPE02908 | PILTKHKRFAMHHPAAFCLAQITSDIPVILFQCTIFAVVLYWMTGLKSSA |
| | *:* *** *.::**:**.**** :*:**:*.**::*::*:*:* *** * |
| P_brasilianum_hmfT5 | AAFFTFWAILFTTTLCITALFRFIGAAFSSFEAASKISGTAVKAIVMYAG |
| Pen_digi_EKV20717 | AAFFTFWIILFTTILCITALFRFIGSAFSTFEAASKISGTAVKGIVMYAG |
| Pen_digi_EKV19541 | AAFFTFWIILFTTILCITALFRFIGSAFSTFEAASKISGTAVKGIVMYAG |
| Pen_rube_XP_002565665 | AAFFTFWIILFTTTLCITALFRFIGSAFSTFEAASKISGTAVKGIVMYAG |
| Asp_oryz_KDE82314 | AAFFTFWAILFTTTLCITALFRCIGAGFSTFEAASKISGTAVKGIVMYAG |
| Asp_oryz_EIT77345 | AAFFTFWAILFTTTLCITALFRCIGAGFSTFEAASKISGTAVKGIVMYAG |
| Asp_flav_XP_002380612 | AAFFTFWAILFTTTLCITALFRCIGAGFSTFEAASKISGTAVKGIVMYAG |
| Asp_terr_XP_001208847 | AAFFTFWAILFTTTLCITALFRFCGAAFSSFEAASKISGTAVKGIVMYAG |
| Asp_kawa_GAA86951 | AAFFTFWIILFTTTLCVTALFRCIGAAFSTFEAASKISGTAIKGIVMYAG |
| Asp_nige_XP_001400982 | AAFFTFWIILFTTTLCVTALFRCIGAGFSTFEAASKISGTAIKGIVMYAG |
| Oph_pice_EPE02908 | AAFFTFWAVLFTTTLCLTALFRFIGAAFSSFEAASKISGTVVKGLVMYAG |
| | ******* :**** **:***** *:.**:**********.:*.:***** |
| P_brasilianum_hmfT5 | YMIPKPEIKNWFLEFYYTNPFAYAFQAALTNEFHDQHIDCVGGNLIPSGP |
| Pen_digi_EKV20717 | YMIPKPEMKNWFLELYYTNPFAYAFQAALSNEFHDRHIPCVGKNLIPSGP |
| Pen_digi_EKV19541 | YMIPKPEMKNWFLELYYTNPFAYAFQAALSNEFHDRHIPCVGKNLIPSGP |
| Pen_rube_XP_002565665 | YMIPKPQMKNWFLELYYTNPFAYAFQAAMSNEFHGRHIPCVGNNLIPSGP |
| Asp_oryz_KDE82314 | YMIPKGRIKNWFLELYYTNPFAYAFQAALSNEFHGQTIPCVGNNLVPSGP |
| Asp_oryz_EIT77345 | YMIPKGRIKNWFLELYYTNPFAYAFQAALSNEFHGQTIPCVGNNLVPSGP |
| Asp_flav_XP_002380612 | YMIPKGRIKNWFLELYYTNPFAYAFQAALSNEFHGQTIPCVGNNLVPSGP |
| Asp_terr_XP_001208847 | YMIPKPHIKNWFLELYYTNPFAYAFQAALSNEFHDQVIPCVGNNLIPSGP |
| Asp_kawa_GAA86951 | YMIPKPKVKNWFLELYYTNPMAYAFQAALSNEFHGQVIPCVGKNIVPTGP |
| Asp_nige_XP_001400982 | YMIPKPKVKNWFLELYYTNPMAYAFQAALSNEFHGQHIPCVGKNIVPNGP |
| Oph_pice_EPE02908 | YMIPKPKVKNWFLELYYTNPFAYAFQAALSNEFHDQHVDCVGPNLIPNGP |
| | ***** .:******:*****:*******::****.: : *** *::*.** |
| P_brasilianum_hmfT5 | GYEDVGSGYKACAGVGGALPGADYVTGDQYLSSLHYKHSQLWRNFGVVWA |
| Pen_digi_EKV20717 | GYENVGAENQACAGVGGALPGANYVTGDQYLASLHYKHSQLWRNFGVVWG |
| Pen_digi_EKV19541 | GYENVGAENQACAGVGGALPGANYVTGDQYLASLHYKHSQLWRNFGVVWG |
| Pen_rube_XP_002565665 | GYEEVGAENQACAGVGGALPGANYVTGDQYLGSLHYKHSQMWRNFGVVWG |
| Asp_oryz_KDE82314 | GYENVSSANKACTGVGGALPGADYVTGDQYLLSLHYKHSQMWRNYGVLWG |
| Asp_oryz_EIT77345 | GYENVSSANKACTGVGGALPGADYVTGDQYLLSLHYKHSQMWRNYGVLWG |
| Asp_flav_XP_002380612 | GYENVSSANKACTGVGGALPGADYVTGDQYLLSLHYKHSQMWRNYGVLWG |
| Asp_terr_XP_001208847 | GYENVGTANKACAGVGGALPGADYVTGDQYLGSLHYKHSQLWRNYGVVWA |
| Asp_kawa_GAA86951 | GYEDVDSANKACTGVGGALPGADYVTGDQYLSSLHYKHSQLWRNFGVVWA |
| Asp_nige_XP_001400982 | GYEDVDSANKACTGVGGALPGADYVTGDQYLSSLHYKHSQLWRNFGVVWA |
| Oph_pice_EPE02908 | GYLDVDSAYKACAGVAGAMPGADFVTGDQYLSSLHYNHSQMWRNFGVIWV |
| | ** :*.: :**:**.**:***::******* ****:***:***:**:* |
| P_brasilianum_hmfT5 | WWGFFAVLTVVFTCFWKSGAASGSSLLIPRENLKKHQVGND--EEAQ-NN |
| Pen_digi_EKV20717 | WWGFFAILTIVFTSYWKSGAGSGASLLIPREKLKNSLAGIS--DEEAQRN |
| Pen_digi_EKV19541 | WWGFFAILTIVFTSYWKSGAGSGASLLIPREKLKNSLAGIS--DEEAQRN |
| Pen_rube_XP_002565665 | WWGFFAILTIVFTSYWKAGAGAGSSLLIPREKLKQHHAAVS--DEEAQNN |
| Asp_oryz_KDE82314 | WWGFFAVLTVICTCFWKGGAAAGASLLIPREKLKAHRAHLD--AEAQKEK |
| Asp_oryz_EIT77345 | WWGFFAVLTVICTCFWKGGAAAGASLLIPREKLKAHRAHLD--AEAQKEK |
| Asp_flav_XP_002380612 | WWGFFAVLTVICTCFWKGGAAAGASLLIPREKLKAHRAHLD--AEAQKEK |
| Asp_terr_XP_001208847 | WWGFFAVATIVCTCFWNAGAGSGAALLIPREKLKNHQRAAD--EESQ-VK |
| Asp_kawa_GAA86951 | WWGFFAVLTIICTTYWKAGAGGSASLLIPRENLKQHQKSID--EESQ-IK |
| Asp_nige_XP_001400982 | WWGFFAVLTIICTTYWKAGAGGSASLLIPRENLKQHQKSID--EESQ-VK |
| Oph_pice_EPE02908 | WWGLFAGLTVFFTSRWKDSGSSGSSLLIPRENLKAHEGKAKSGDEEAQNN |
| | ***:** *:. * *: .....::******:** . * : |
| P_brasilianum_hmfT5 | EKHAARTTTDEPVQVEDDNLVRNTSIFTWKNLTYTVKTPTGDRVLLDNIN |
| Pen_digi_EKV20717 | EKTTARETIDEPVQVDDENLTRNTSIFTWRNLTYTVQTPTGDRVLLDNIH |
| Pen_digi_EKV19541 | EKTTARETIDEPVQVDDENLTRNTSIFTWRNLTYTVQTPTGDRVLLDNIH |
| Pen_rube_XP_002565665 | EKSTTRETPDEPIQVDDENLNRNTSIFTWKNLTYTVQTPTGDRVLLDNIH |
| Asp_oryz_KDE82314 | DPAREKGSGDALTSADEGNLTHNTSIFTWKNLTYTVNTPTGERVLLDNIH |
| Asp_oryz_EIT77345 | DPAREKGSGDALTSADEGNLTHNTSIFTWKNLTYTVNTPTGERVLLDNIH |
| Asp_flav_XP_002380612 | DPAREKGSGDALTSADEGNLTHNTSIFTWKNLTYTVNTPTGERVLLDNIH |
| Asp_terr_XP_001208847 | EKEQTRGPAAGESTAQDDNLTRNTSIFTWKNLKYTVKTPTGDRLLLDNVH |
| Asp_kawa_GAA86951 | EKEQTKAATSDTTAEVDGNLSRNTAVFTWKNLKYTVKTPSGDRVLLDNIH |
| Asp_nige_XP_001400982 | EKEQAKAATSDTTAEVDGNLSRNTAVFTWKNLKYTVKTPSGDRVLLDNIH |
| Oph_pice_EPE02908 | EKNTPRPQADAPVEANDNSLVRNTSIFTWKDLTYTVNTPTGERVLLNQVN |
| | : : : .* :**::***::*.***:**:*:*:**:::: |
| P_brasilianum_hmfT5 | GWVKPGMLGALMGSSGAGKTTLLDVLAQRKTEGTIKGSILVDGRELPVSF |
| Pen_digi_EKV20717 | GWVKPGMLGALMGSSGAGKTTLLDVLAQRKTDGTIKGSIMVDGRELPVSF |
| Pen_digi_EKV19541 | GWVKPGMLGALMGSSGAGKTTLLDVLAQRKTDGTIKGSIMVDGRELPVSF |
| Pen_rube_XP_002565665 | GWVKPGMLGALMGSSGAGKTTLLDVLAQRKTDGTINGSIMVDGRELPVSF |
| Asp_oryz_KDE82314 | GWVKPGMLGALMGSSGAGKTTLLDVLAQRKTEGTIKGSVLVDGRELPVSF |
| Asp_oryz_EIT77345 | GWVKPGMLGALMGSSGAGKTTLLDVLAQRKTEGTIKGSVLVDGRELPVSF |
| Asp_flav_XP_002380612 | GWVKPGMLGALMGSSGAGKTTLLDVLAQRKTEGTIKGSVLVDGRELPVSF |
| Asp_terr_XP_001208847 | GWVKPGMLGALMGSSGAGKTTLLDVLAQRKTEGTINGSILVDGRPLPVSF |
| Asp_kawa_GAA86951 | GWVKPGMLGALMGSSGAGKTTLLDVLAQRKTEGTITGSIMVDGRPLPVSF |
| Asp_nige_XP_001400982 | GWVKPGMLGALMGSSGAGKTTLLDVLAQRKTEGTITGSIMVDGRPLPVSF |
| Oph_pice_EPE02908 | GWVKPGMLGALMGSSGAGKTTLLDVLAQRKTEGTIRGSILVDGRPLPLSF |
| | *******************************:*** **::**** **:** |
| P_brasilianum_hmfT5 | QRMAGYCEQLDVHESYATVREALEFSALLRQSRDTPKAEKLKYVDTIIDL |
| Pen_digi_EKV20717 | QRMAGYCEQLDVHEPFATVREALEFSALLRQSRNISKADKLKYVDTIIDL |
| Pen_digi_EKV19541 | QRMAGYCEQLDVHEPFATVREALEFSALLRQSRNISKADKLKYVDTIIDL |
| Pen_rube_XP_002565665 | QRMAGYCEQLDVHEPYATVREALEFSALLRQSRNTPKADKLKYVDTIIDL |
| Asp_oryz_KDE82314 | QRMAGYCEQLDVHEPYATVREALEFSALLRQSRDTPREEKLKYVDTIIDL |
| Asp_oryz_EIT77345 | QRMAGYCEQLDVHEPYATVREALEFSALLRQSRDTPREEKLKYVDTIIDL |
| Asp_flav_XP_002380612 | QRMAGYCEQLDVHEPYATVREALEFSALLRQSRDTPREEKLKYVDTIIDL |
| Asp_terr_XP_001208847 | QRMAGYCEQLDVHEPYATVREALEFSALLRQPRTTPKEEKLKYVDTIIDL |
| Asp_kawa_GAA86951 | QRMAGYCEQLDVHEPFATVREALEFSALLRQPRTTPREEKLKYVDTIIDL |
| Asp_nige_XP_001400982 | QRMAGYCEQLDVHEPFATVREALEFSALLRQPRTTPKEEKLKYVETIIDL |
| Oph_pice_EPE02908 | QRMAGYCEQLDVHEPYATVREALEFSALLRQSRDVPRAEKLKYVETIIDL |
| | **************.:***************.* .: :*****:***** |
| P_brasilianum_hmfT5 | LELHDLADTLIGSVGNGLSVEQRKRVTIGVELVSKPSILIFLDEPTSGLD |
| Pen_digi_EKV20717 | LELHDLADTLIGTVGNGLSVEQRKRVTIGVELVSKPSILIFLDEPTSGLD |
| Pen_digi_EKV19541 | LELHDLADTLIGTVGNGLSVEQRKRVTIGVELVSKPSILIFLDEPTSGLD |
| Pen_rube_XP_002565665 | LELDDLADTLIGTIGNGLSVEQRKRVTIGVELVSKPSILIFLDEPTSGLD |
| Asp_oryz_KDE82314 | LELHDLADTLIGTVGNGLSVEQRKRVTIGVELVSKPSILIFLDEPTSGLD |
| Asp_oryz_EIT77345 | LELHDLADTLIGTVGNGLSVEQRKRVTIGVELVSKPSILIFLDEPTSGLD |
| Asp_flav_XP_002380612 | LELHDLADTLIGTVGNGLSVEQRKRVTIGVELVSKPSILIFLDEPTSGLD |
| Asp_terr_XP_001208847 | LELHDLADTLIGTVGNGLSVEQRKRVTIGVELVSKPSILIFLDEPTSGLD |
| Asp_kawa_GAA86951 | LELHDLADTLIGTVGNGLSVEQRKRVTIGVELVSKPSILIFLDEPTSGLD |
| Asp_nige_XP_001400982 | LELHDLADTLIGTVGNGLSVEQRKRVTIGVELVSKPSILIFLDEPTSGLD |
| Oph_pice_EPE02908 | LELHDLADTLIGAVGNGLSVEQRKRVTIGVELVAKPSILIFLDEPTSGLD |
| | ***.********::*******************:**************** |
| P_brasilianum_hmfT5 | GQSAYNTVRFLRKLADVGQAVLVTIHQPSAQLFAQFDTLLLLARGGKTVY |
| Pen_digi_EKV20717 | GQSAYNTVRFLRKLADVGQAVTI--HQPSAQLFAQFDTLLLLAKGGKTVY |
| Pen_digi_EKV19541 | GQSAYNTVRFLRKLADVGQAVTI--HQPSAQLFAQFDTLLLLAKGGKTVY |
| Pen_rube_XP_002565665 | GQSAYNTVRFLRKLADVGQAV-----LPSAQLFAQFDTLLLLAKGGKTVY |
| Asp_oryz_KDE82314 | GQSAYNTVRFLRKLADVGQAVLVTIHQPSAQLFAQFDTLLLLARGGKTVY |
| Asp_oryz_EIT77345 | GQSAYNTVRFLRKLADVGQAVLVTIHQPSAQLFAQFDTLLLLARGGKTVY |
| Asp_flav_XP_002380612 | GQSAYNTVRFLRKLADVGQAVLVTIHQPSAQLFAQFDTLLLLARGGKTVY |
| Asp_terr_XP_001208847 | GQSAYNTVRFLRKLADVGQAVLVTIHQPSAQLFAQFDTLLLLARGGKTVY |
| Asp_kawa_GAA86951 | GQSAYNTVRFLRKLADVGQAVLVTIHQPSAQLFAQFDTLLLLARGGKTVY |
| Asp_nige_XP_001400982 | GQSAYNTVRFLRKLADVGQAVLVTIHQPSAQLFAQFDTLLLLARGGKTVY |
| Oph_pice_EPE02908 | GQSAFNTVRFLRKLADVGQAVLVTIHQPSAQLFAQFDTLLLLAKGGKTVY |
| | ****:**************** ****************:****** |
| P_brasilianum_hmfT5 | FGDIGDNGSTIKQYFGNYGAICPQEANPAEFMIDVVTGGIQEVKDKDWHQ |
| Pen_digi_EKV20717 | FGDIGENAATVKQYFGQYGAQCPTEANAAEFMIDVVTGGIEAVKDKDWHQ |
| Pen_digi_EKV19541 | FGDIGENAATVKQYFGQYGAQCPTEANAAEFMIDVVTGGIEAVKDKDWHQ |
| Pen_rube_XP_002565665 | FGDIGDNAACVKQYFGQYGAQCPTDANAAEFMIDVVTGGIESVKDKDWHQ |
| Asp_oryz_KDE82314 | FGDIGDNGAAIKQYFGKYGASCPIEANPAEFMIDVVTGGIEEVKDKDWHQ |
| Asp_oryz_EIT77345 | FGDIGDNGAAIKQYFGKYGASCPIEANPAEFMIDVVTGGIEEVKDKDWHQ |
| Asp_flav_XP_002380612 | FGDIGDNGAAIKQYFGKYGASCPIEANPAEFMIDVVTGGIEEVKDKDWHQ |
| Asp_terr_XP_001208847 | FGDIGENGQTIKEYFGKYGAQCPVEANPAEFMIDVVTGGIESVKHMDWHQ |
| Asp_kawa_GAA86951 | FGDIGDNGQTIKHYFGKYGAQCPVEANPAEFMIDVVTGGIESVKDKDWHQ |
| Asp_nige_XP_001400982 | FGDIGENGQTIKNYFGKYGAQCPIEANPAEFMIDVVTGGIESVKDKDWHH |
| Oph_pice_EPE02908 | FGDIGDNGATVKQYFGQYGAVCPEESNPAEFMIDVVTGGIEEVKDKDWHQ |
| | *****:*. :*.***:*** ** ::*.************: **. ***: |
| P_brasilianum_hmfT5 | IWLDSPEQHQMITELDRMIADAASKPPGTVND-GYEFSMPLWEQIKIVTQ |
| Pen_digi_EKV20717 | IWLDSPEQTRMIAELDGMIADAAAKPPGTVDD-GFEFSMPMWEQIKIVTQ |
| Pen_digi_EKV19541 | IWLDSPEQTRMIAELDGMIADAAAKPPGTVDD-GFEFSMPMWEQIKIVTQ |
| Pen_rube_XP_002565665 | IWLDSPEQTRMIAELDRMIADAASKPPGTVDD-GFEFSMPLWEQTKIVTH |
| Asp_oryz_KDE82314 | IWLESPEHEHMMVELDQLISDAAAKPPGTHDD-GYEFSMPLWDQVKIVTH |
| Asp_oryz_EIT77345 | IWLESPEHEHMMVELDQLISDAAAKPPGTHDD-GYEFSMPLWDQVKIVTH |
| Asp_flav_XP_002380612 | IWLESPEHEHMMVELDQLISDAAAKPPGTHDD-GYEFSMPLWDQVKIVTH |
| Asp_terr_XP_001208847 | VWLESPEHTRMLQELDHMVEDAASKPPGTVDD-GFEFSMSLWEQTKIVTR |
| Asp_kawa_GAA86951 | VWLESPEHQQMITELDHLISEAASKPSSVNDD-GCEFSMPLWEQTKIVTH |
| Asp_nige_XP_001400982 | VWLESPEHQQMITELDHLISEAASKPSGVNDD-GCEFSMPLWEQTKIVTH |
| Oph_pice_EPE02908 | VWMDSSEQREMATELNTMIEDAAGRPSQTSDDDGFEFAMPLWEQTKIVTY |
| | :*::*.*: .* **: :: :**.:*. . :* * **:*.:*:* **** |
| P_brasilianum_hmfT5 | RMNVSLFRNTAYVNNKFSLHIISALLNGFSFWRPGPSVSALQLKMFTIFN |
| Pen_digi_EKV20717 | RMNVALFRNTNYINNKFSLHIISAALNGFSFWRPGPSVTALNLKMFTIFN |
| Pen_digi_EKV19541 | RMNVALFRNTNYINNKFSLHIISAALNGFSFWRPGPSVTALNLKMFTIFN |
| Pen_rube_XP_002565665 | RMNVALFRNTNYVNNKFSLHIISAMLNGFSFWRPGPSVSALNLKMFTIFN |
| Asp_oryz_KDE82314 | RMNVALFRNTNYVNNKFSLHIISALLNGFSFWHTGPSVSALNLKMFTIFN |
| Asp_oryz_EIT77345 | RMNVALFRNTNYVNNKFSLHIISALLNGFSFWHTGPSVSALNLKMFTIFN |
| Asp_flav_XP_002380612 | RMNVALFRNTNYVNNKFSLHIISALLNGFSFWHTGPSVSALNLKMFTIFN |
| Asp_terr_XP_001208847 | RMNIALFRNTNYVNNKFMLHIISALLNGFSFWRVGPSVSALNLKMFTIFN |
| Asp_kawa_GAA86951 | RMNVALFRNTNYVNNKFSLHIISALLNGFSFWRVGPSVTALQLKMFTIFN |
| Asp_nige_XP_001400982 | RMNVALFRNTNYVNNKFSLHIISALLNGFSFWRVGPSVTALQLKMFTIFN |
| Oph_pice_EPE02908 | RMNVSLFRNTAYVNNKFSLHIISALLNGFSFWRLGKSANDLQLRLFTIFN |
| | ***::***** *:**** ****** *******: * *.. *:*::***** |
| P_brasilianum_hmfT5 | FVFVAPGVINQLQPLFIQRRDIYDAREKKSKMYSWVAFVTGLIVSEFPYL |
| Pen_digi_EKV20717 | FVFVAPGVINQLQPLFIQRRDIYDTREKKSKMYSWVAFVTGLVVSEFPYL |
| Pen_digi_EKV19541 | FVFVAPGVINQLQPLFIQRRDIYDTREKKSKMYSWVAFVTGLVVSEFPYL |
| Pen_rube_XP_002565665 | FVFVAPGVINQLQPLFIQRRDIYDTREKKSKMYSWVAFVTGLIVSEFPYL |
| Asp_oryz_KDE82314 | FVFVAPGVINQLQPLFIQRRDIYDAREKKSKMYSWVAFVTGLIVSEFPYL |
| Asp_oryz_EIT77345 | FVFVAPGVINQLQPLFIQRRDIYDAREKKSKMYSWVAFVTGLIVSEFPYL |
| Asp_flav_XP_002380612 | FVFVAPGVINQLQPLFIQRRDIYDAREKKSKMYSWVAFVTGLIVSEFPYL |
| Asp_terr_XP_001208847 | FVFVAPGVINQLQPLFIQRRDIYDAREKKSKMYSWVSFVIGLIVSEFPYL |
| Asp_kawa_GAA86951 | FVFVAPGVINQLQPLFIQRRDIYDAREKKSKMYSWISFVIGLIVSEFPYL |
| Asp_nige_XP_001400982 | FVFVAPGVINQLQPLFIQRRDIYDAREKKSKMYSWISFVIGLIVSEFPYL |
| Oph_pice_EPE02908 | FVFVAPGVINQLQPLFIQRRDIYDAREKKSKMYSWVAFVTALIVSEFPYL |
| | ************************:**********::** .*:******* |
| P_brasilianum_hmfT5 | CICAVLYFVCWYWPVWRLPHDSDRSGAIFFMMLIYEFIYTGIGQFIAAYA |
| Pen_digi_EKV20717 | CICAVLYFACWYYPVWRLPHASNRSGATFFMMLIYELIYTGIGQFVAAYS |
| Pen_digi_EKV19541 | CICAVLYFACWYYPVWRLPHASNRSGATFFMMLIYELIYTGIGQFVAAYS |
| Pen_rube_XP_002565665 | CICAVLYFVCWYYPVWRLPHESSRSGATFFMMLIYELIYTGIGQFVAAYS |
| Asp_oryz_KDE82314 | CVCAVLYFACWYYCVRKLPHDSKRSGATFFIMLIYEFIYTGIGQFVAAYA |
| Asp_oryz_EIT77345 | CVCAVLYFACWYYCVR-LPHDSKRSGATFFIMLIYEFIYTGIGQFVAAYA |
| Asp_flav_XP_002380612 | CVCAVLYFACWYYCVR-LPHDSNRSGATFFIMLIYEFIYTGIGQFVAAYA |
| Asp_terr_XP_001208847 | CVCAVLYFLCWYYCVK-LPHDSNKAGATFFIMLIYEFIYTGIGQFVAAYA |
| Asp_kawa_GAA86951 | CVCAVLYFLCWYYCVR-LPHDSNKAGATFFIMLIYEFIYTGIGQFIAAYA |
| Asp_nige_XP_001400982 | CVCAVLYFLCWYYCVR-LPHDSNKAGATFFIMLIYEFIYTGIGQFIAAYA |
| Oph_pice_EPE02908 | CICAVLYFVCWYWPVWTLPHDSNRSGAIFFMMWIYEFIYTGIGQFIAAYA |
| | *:****** ***: * *** *.::** **:* ***:********:***: |
| P_brasilianum_hmfT5 | PNPTFAALVNPLIISVLVLFCGVFVPYDQLNVFWKYWMYYLNPFNYVVNG |
| Pen_digi_EKV20717 | PNPTFAALVNPLIISTLILFCGVFVPYLQLNVFWRFWMYYLNPFNYVVSG |
| Pen_digi_EKV19541 | PNPTFAALVNPLIISTLILFCGVFVPYLQLNVFWRFWMYYLNPFNYVVSG |
| Pen_rube_XP_002565665 | PNPTFAALVNPLIISTLVLFCGIFVPYIQLNVFWRYWMYYLNPFNYVVSG |
| Asp_oryz_KDE82314 | PNPTFAALVNPLIISTLTLMCGIFVPYSQLTVFWRYWMYYLNPFNYVTSG |
| Asp_oryz_EIT77345 | PNPTFAALVNPLIISTLTLMCGIFVPYSQLTVFWRYWMYYLNPFNYVTSG |
| Asp_flav_XP_002380612 | PNPTFAALVNPLIISTLTLMCGIFVPYSQLTVFWRYWMYYLNPFNYVTSG |
| Asp_terr_XP_001208847 | PNPTFAALVNPLIISTLVLFCGIFVPYTQLNVFWKYWLYWLNPFNYVVSG |
| Asp_kawa_GAA86951 | PNPTFAALVNPMIISVLVLFCGIFVPYTQLNVFWKYWLYYLNPFNYVVSG |
| Asp_nige_XP_001400982 | PNPTFAALVNPMIISVLVLFCGIFVPYTQLNVFWKYWLYYLNPFNYVVSG |
| Oph_pice_EPE02908 | PNPTFAALINPLIISIMTLFCGVFVPYQQLNVFWKYWMYWINPFSYVVNG |
| | ********:**:*** : *:**:**** **.***::*:*::***.**..* |
| P_brasilianum_hmfT5 | MLTFGLWGQKVTCNESEYAVFDPLNG-TCGEYLATYMSGK--GSGVNLLN |
| Pen_digi_EKV20717 | MLTFGIWGAKVTCNEEEFAFFEPVNGTTCVEYLSDYMTGT--GSGINLIN |
| Pen_digi_EKV19541 | MLTFGIWGAKVTCNEEEFAFFEPVNGTTCVEYLSDYMTGT--GSGINLIN |
| Pen_rube_XP_002565665 | MLTFGLWGAKVTCNEDEFALFEPLNGTTCAQYLSDYMSGA--GSSINLVN |
| Asp_oryz_KDE82314 | MLVFGMWGAKVTCNEDEFAIFDPVNG-TCGDYLADYMAGS--GSRINLTN |
| Asp_oryz_EIT77345 | MLVFGMWGAKVTCNEDEFAIFDPVNG-TCGDYLADYMAGS--GSRINLTN |
| Asp_flav_XP_002380612 | MLVFGMWGAKVTCNEDEFAIFDPVNG-TCGDYLADYMAGS--GSRINLTN |
| Asp_terr_XP_001208847 | MLTFGIWDAKVTCNADEFAFFDPTNG-TCGEYLADYIRGD--GWRINLTN |
| Asp_kawa_GAA86951 | MLTFDMWDAKVTCNEDEFALFNPTNG-TCAEYLKDYIAGQ--GWRVNLTN |
| Asp_nige_XP_001400982 | MLTFDMWDAKVTCNEDEFALFNPTNG-TCAEYLKDYIAGQ--GWRVNLTN |
| Oph_pice_EPE02908 | MLTFGLWGQKVVCAEGEFAVFDPLNG-TCGEYLSTYMSANGMGSHVNLTN |
| | **.*.:*. **.* *:*.*:* ** ** :** *: . * :** * |
| P_brasilianum_hmfT5 | PDATSSCKVCEYTTGSDFLQTLNINHYYYGWRDAGITVIYAISGYALVFG |
| Pen_digi_EKV20717 | PDATSACKVCQYTDGSDFLRGLHIQNYTTGWRDIGISVIFAISGYALVFG |
| Pen_digi_EKV19541 | PDATSACKVCQYTDGSDFLRGLHIQNYTTGWRDIGISVIFAISGYALVFG |
| Pen_rube_XP_002565665 | PDATSACKVCQYTDGSDFLRNLNIMNYTTGWRDIGISVIFAISGYALVFG |
| Asp_oryz_KDE82314 | PDATSGCRVCEYRSGSDFLTTLNINHYYYGWRDAGICVIFAISGYALVFV |
| Asp_oryz_EIT77345 | PDATSGCRVCEYRSGSDFLTTLNINHYYYGWRDAGICVIFAISGYALVFV |
| Asp_flav_XP_002380612 | PDATSGCRVCEYRSGSDFLTTLNINHYYYGWRDAGICVIFAISGYALVFA |
| Asp_terr_XP_001208847 | PDATSACKVCQYREGSDFLTTLNINDYYYGWRDAGISVIFAISGYALVFG |
| Asp_kawa_GAA86951 | PDATSTCRVCEYRRGSDFLTTLNINHYFYGWRDAGISVIFAISGYALVFA |
| Asp_nige_XP_001400982 | PDATSTCRVCEYRRGSDFLTTLNINHYYYGWRNAGITVIFAISGYALVFA |
| Oph_pice_EPE02908 | PDATAGCRVCEYRDGSGFLSTLNVNHYYVGWRDAAISVLYAFSGYALVFG |
| | ****: *:**:* **.** *:: .* ***: .* *::*:******* |
| P_brasilianum_hmfT5 | LMKLRTKASKKAE----- |
| Pen_digi_EKV20717 | LMKLRTKASKKAE----- |
| Pen_digi_EKV19541 | LMKLRTKASKKAE----- |
| Pen_rube_XP_002565665 | LMKLRTKASKKAE----- |
| Asp_oryz_KDE82314 | LMKLRTKASKKAE----- |
| Asp_oryz_EIT77345 | LMKLRTKASKKAE----- |
| Asp_flav_XP_002380612 | LMKLRTKASKKAE----- |
| Asp_terr_XP_001208847 | LMKLRTKASKKAE----- |
| Asp_kawa_GAA86951 | LMKLRTKASKKAE----- |
| Asp_nige_XP_001400982 | LMKLRTKASKKAE----- |
| Oph_pice_EPE02908 | LMKLRTKASKKAEUSAGE |
| | ************* |

TABLE 9
Amino acid sequence alignment of Penicillium brasilianum hmfT6
and 10 closest orthologues.

| | |
|---|---|
| HmfT6 | ------------------------------------------------------------ |
| Pb_CEJ60583.1 | ------------------------------------------------------------ |
| Ps_OKO99970.1 | ------------------------------------------------------------ |
| As_OJJ53782.1 | ------------------------------------------------------------ |
| Av_OJJ07888.1 | ------------------------------------------------------------ |
| Pi_KGO73014.1 | ------------------------------------------------------------ |
| Ti_CRG83369.1 | MMGSVPKGLDEATSTLSSSGTELSSVRKSLNAYSTLEVVRPSDLEAYKNASETGFMPQWQ |
| Ar_EYE92060.1 | ------------------------------------------------------------ |
| Ag_OJJ86250.1 | ------------------------------------------------------------ |
| Ma_KID68223.1 | ------------------------------------------------------------ |
| Ma_KJK94474.1 | ------------------------------------------------------------ |
| HmfT6 | ------------------------------------------------------------ |
| Pb_CEJ60583.1 | ------------------------------------------------------------ |
| Ps_OKO99970.1 | ------------------------------------------------------------ |
| As_OJJ53782.1 | ------------------------------------------------------------ |
| Av_OJJ07888.1 | ------------------------------------------------------------ |
| Pi_KGO73014.1 | ------------------------------------------------------------ |
| Ti_CRG83369.1 | VKNDPYPTAGPATSEQGSTIPISSPPPPPPSQEMICGLPKRAFWVIFTVITSLCVIAIIV |
| Ar_EYE92060.1 | ------------------------------------------------------------ |
| Ag_OJJ86250.1 | ------------------------------------------------------------ |
| Ma_KID68223.1 | ------------------------------------------------------------ |
| Ma_KJK94474.1 | ------------------------------------------------------------ |
| HmfT6 | ------------------------------------------------------------ |
| Pb_CEJ60583.1 | ------------------------------------------------------------ |
| Ps_OKO99970.1 | ------------------------------------------------------------ |
| As_OJJ53782.1 | ------------------------------------------------------------ |
| Av_OJJ07888.1 | ------------------------------------------------------------ |
| Pi_KGO73014.1 | ------------------------------------------------------------ |
| Ti_CRG83369.1 | GGAVGGTRHHESQTSNDAVSKSSISSNPTAFVTVTVVPTSLSPSTTTTASVTTSSVSLPF |
| Ar_EYE92060.1 | ------------------------------------------------------------ |
| Ag_OJJ86250.1 | ------------------------------------------------------------ |
| Ma_KID68223.1 | ------------------------------------------------------------ |
| Ma_KJK94474.1 | ------------------------------------------------------------ |
| HmfT6 | ------------------MTPISRLLSRAVNKPYRTKDITDEIPPTLDEDGFVSFGPGDI |
| Pb_CEJ60583.1 | ------------------MTPISRLLSRAVNKPYRTKDISDEIPPTLDEDGFVSFGPGDI |
| Ps_OKO99970.1 | ------------------MTPISRILSRAVNKPYRTKDLTEQSPPTLDEDGYVGFAPGDI |
| As_OJJ53782.1 | ------------------MTAISRVLSYAVNKPYRTRDLRDEHSPYLNADGYIDFAPGDI |
| Av_OJJ07888.1 | ------------------MTAISRVLSYAVNKPYRTRDLRHDSSPYVNADGYIDFAPGDI |
| Pi_KGO73014.1 | ------------------MAAITRILSYAVNKPYRSQDIHYAPHPYINEDGYVDFAPGDI |
| Ti_CRG83369.1 | VVRRHAVSSFFVLPPAAVMSAISRVLSVAVNKPYRTRKLEDSFPPFINEDGFVDFAPDDI |
| Ar_EYE92060.1 | ------------------MAAITRILSYTVNKPYNSQDIRYEPHPYLNEDGYVDFAPGDI |
| Ag_OJJ86250.1 | ------------------MAAITRILSYAVNKPYRSQDIRYEPHPYLNGDGYVDFAPGDI |
| Ma_KID68223.1 | ------------------MTGLTRLLSRPVNKPYRTRDLDHTEPPFLDDAGFLAFKPDDI |
| Ma_KJK94474.1 | ------------------MTGLTRLLSRPVNKPYRTRDLDHTEPPFLDDAGFLAFKPDDI |
| | *: ::*:** *****.::.: * :: *:: * *.** |
| HmfT6 | ENPRNWSMARRAGVTMSAVLLVVNATFASSSPSGCFPSISKHFGVSTEVAGLTITLFLLG |
| Pb_CEJ60583.1 | ENPRNWSMARRAGVTMSAVLLVVNATFASSSPSGCFPSISKHFGVSTEVAGLTITLFLLG |
| Ps_OKO99970.1 | ENPRNWSMARRTGVTMAAVLLVVNATFASSSPSGCFPSISEHFGISSEVAGLTITLFLLG |
| As_OJJ53782.1 | ENPRNWSLPRRVAITITAILLVVNATFASSSPSGCFGSISEHFGVSTEVAGLTITLFLLG |
| Av_OJJ07888.1 | ENPRNWSLPRRVAITVTAVLLVVNATFASSSPSGCFGSISEHFEVSTEVAGLTITLFLLG |
| Pi_KGO73014.1 | ENPRNWSMARRITITMTAVLLVVNATFASSSPSGCFGSISKEFGISTEVAGLTITLFLLG |
| Ti_CRG83369.1 | ENPRNWSTGRRAIITITTVLLVLNATFASSSPSGCIASISKEFHVSTEAAGLTITLFLLG |
| Ar_EYE92060.1 | ENPRNWSMARRVTITITAILLVVNATFASSSPSGCFESISEQFGVSTEVAGLTITLFLLG |
| Ag_OJJ86250.1 | ENPRNWSMTRAVAITITAILLVVNATFASSSPSGCFGSISEQFGVSTEVAGLTITLFLLG |
| Ma_KID68223.1 | ENPQNWSTPRRIAVTISAVMLVMNATFASSAPSGCIPSIAKDFGISTEAAALTVTLFLLG |
| Ma_KJK94474.1 | ENPQNWSTPRRIAVTISAVMLVMNATFASSAPSGCIPSIAKDFGISTEAAALTVTLFLLG |
| | ***:*** ** :*:::::**:*******:****: **::.* :*:*.*.**:****** |
| HmfT6 | YCAGPLIFAPLSEFYGRAWIFYITFLLYLAFNFLCAFPPNFGSLLVGRFLTGTFVSAPLS |
| Ps_OKO99970.1 | YCAGPLIFAPLSEFYGRRYIFYITFLLYLAFTFLCAFPPNFGSLLVGRFLTGTFVSAPLS |
| Pb_CEJ60583.1 | YCAGPLIFAPLSEFYGRAWIFYITFLLYLAFNFLCAFPPNFGSLLVGRFLTGTFVSAPLS |
| As_OJJ53782.1 | YCAGPLIFAPLSEFYGRAWIFYITFSLYLAFNFLCAFAPNLGALLVGRFLTGTFVSAPLS |
| Av_OJJ07888.1 | YCAGPLIFAPLSEFYGRAWIFYITFALYLAFNFLCAFAPNLGALLVGRFLTGTFVSAPLS |
| Pi_KGO73014.1 | YCAGPLIFAPLSEFYGRRWIFYISFAFYLSFNFLCAFAPNLGALLVGRFLAGTFVSAPLS |
| Ti_CRG83369.1 | YCAGPLIFAPLSEFYGRAWIFYTTFLLYIVFNFLCAFAPNFGSLLVGRFLTGTFISAPLS |
| Ar_EYE92060.1 | YCAGPLIFAPLSEFYGRAWIFYISFALYLAVNFLCAFAPNLGALLVGRFLTGTFVSAPLS |
| Ag_OJJ86250.1 | YCAGPLIFAPLSEFYGRAWIFYISFALYLAFNFLCAFAPNLGALLVGRFLTGTFVSAPLS |
| Ma_KID68223.1 | YCAGPLLFAPLSELYGRAWIFYISFTIYIAFNFLCAFAPNFGSLLVGRFLIGILVSAPLS |
| Ma_KJK94474.1 | YCAGPLLFAPLSELYGRAWIFYISFTMYIAFNFLCAFAPNFGSLLVGRFLIGTLVSAPLS |
| | ******:******:****:*** :* :*: ..***** **:*:*******:**::***** |
| HmfT6 | NCPGVLADVWNPLERANAMAGFSAMVWIGPALGPVVAGFLQLKEDWRWSFYVLLWLGGAS |
| Pb_CEJ60583.1 | NCPGVLADVWNPLERANAMAGFSAMVWIGPALGPVVAGFLQLKEDWRWSFYVLLWLGGAS |
| Ps_OKO99970.1 | NCPGVLADVWSPLERANAMAGFSAMVWIGPALGPVVSGFLQLEKDWRWIFYVLLWLGGAS |
| As_OJJ53782.1 | NSPGVLADLWDPLQRANAMAGFSAMVYVGPALGPVIAGFLELEKDWRWSFYVLLWLGGAT |
| Av_OJJ07888.1 | NSPGVLADLWDPLQRSNAMAGFSAMVYVGPALGPVIAGFLELEKDWRWSFYVLLWLGGAT |
| Pi_KGO73014.1 | NAPGVLADLWDPLQRANAMAGFSAMVYVGPALGPVIAGFLELKKDWRWSFYVLLWLGGLT |
| Ti_CRG83369.1 | NGPGVLADLWNPLQRSNAMAGFSAMVWIGPALGPVVAGFLELTKDWRWSFYVLLWLGGAS |
| Ar_EYE92060.1 | NAPGVLADLWDPLQRANAMAGFSAMVYVGPALGPVIAGFLELKKDWRWSFYVLLWLGGVT |
| Ag_OJJ86250.1 | NAPGVLADLWDPLQRANAMAGFAAMVYVGPALGPVIAGFLELKEDWRWSFYVLLWLGGVT |
| Ma_KID68223.1 | NAPGVLADIWSPLERANAMALFSTMVWIGPALGPVVAGFLELKKDWHWAFYVLIWLGAGT |
| Ma_KJK94474.1 | NAPGVLADIWSPLERANAMALFSTMVWIGPALGPVVAGFLELKKDWHWAFYVLIWLGAGT |
| | * ******:*.**:*:**** *::**::*******::***:* :**:*:****:***. : |
| HmfT6 | AVIMLTIPETYAPIVLYNKARRIREAQIPGYENVKAPVEDGDRILVGIYKVALTRPWIIL |
| Pb_CEJ60583.1 | AVIMLTIPETYAPIVLYNKARRIREAQIPGYETVKAPVEDGDRILVGIYKVALTRPWIIL |
| Ps_OKO99970.1 | AVIMLTIPETYAPIVLYKKAQRIRDASIPGYENVKAPVEDSDRILVGIYKVALTRPWIIL |
| As_OJJ53782.1 | AILMLTIPETFAPIVLCNKAKRVRKAKIPGYENVKAQAEDSDRILVGIYKVALTRPWIIL |
| Av_OJJ07888.1 | AILMLTIPETYAPIVLCNKAKRIRKAKIPGYENVKAQAEDSDRILVGIYKVALTRPWIIL |
| Pi_KGO73014.1 | AILMLTIPETYAPTILYNKAKRIRKAKIPAYENVKAEVEDSDRILVGIYKVALTRPWIIL |
| Ti_CRG83369.1 | AILMLTIPETYAPTILCIKAKRIRKANIPGYENVRAAAEDNDISLVGIYKVALTRPWVIL |
| Ar_EYE92060.1 | AIVMLTIPETYAPTVLYNKAKRIRMAKIPGYESVKAQAEDSDRILVGIYKLALTRPWIIL |
| Ag_OJJ86250.1 | AIVMLTIPETYAPTVLYNKAKRIRMAKIPGYENVKAQAENSDRILAGIYKLALTRPWIIL |
| Ma_KID68223.1 | WLIMLTIPETHAPTILLHKAKRVRKAKIPGYENVKAPAEKQDRSITSVFGVALTRPWVIL |
| Ma_KJK94474.1 | WLIMLTIPETHAPTILLHKAKRVRKAKIPGYENVKAPAEKQDRSITSVFGVALTRPWVIL |
| | ::*******.** :* **:*:* *.**.**.*:* .*. * ::..:: :******:** |
| HmfT6 | FDPISLLCAIYMAFVYILLYMLFTIYPIVFQEKRGWNSGVGELPLLGTVVGALFGGVIVV |
| Pb_CEJ60583.1 | FDPISLLCAIYMAFVYILLYMLFTIYPIVFQEKRGWNSGVGELPLLGTVVGALFGGVIVV |
| Ps_OKO99970.1 | FDPISLLCAIYLAFVYILLYMLFTIYPIVFQEKRGWNSGVGELPLLGTVVGALFGGAIVL |
| As_OJJ53782.1 | FDPISLLCAIYMAVVYILLYMLFSIYPIVFQQARGWNSGVGELPLIGTVVGALIGGLIVL |
| Av_OJJ07888.1 | FDPISLLCAIYMAVVYILLYMLFSIYPIVFQERRGWNSGVGELPLIGTVVGALIGGLVVL |
| Pi_KGO73014.1 | FDPISLLCAIYMAVVYILLYMLFTIYPIVFQEKRGWNSGVGELPLVGIVVGALIGGLVVL |
| Ti_CRG83369.1 | FDPISLLCALYLAVVYILLYMLFSIYPIVFQERRGWNSGIGELPLIGTIVGALVGGAVVL |
| Ar_EYE92060.1 | FDPISLLCAIYMAVVYILLYMLFSIYPIVFQEKRGWNSGVGELPLIGTVVGALIGGLIVL |
| Ag_OJJ86250.1 | FDPISLLCAIYMAVVYALLYMLFSIYPIVFQEKRGWNSGVGELPLIGTVVGALIGGLIVL |
| Ma_KID68223.1 | FDPISLLCAIYLAVIYILLYMLFSIYPIVFQERRGWNSGVGELPLIGTVVGAILGGSIVL |
| Ma_KJK94474.1 | FDPISLLCAIYLAVIYILLYMLFSIYPIVFQERRGWNSGVGELPLIGTVVGAILGGSIVL |
| | *********:*:*.:*:******:*******::******:*****:**:***:.** :*: |
| HmfT6 | ADIRMRQKRIDNGTIKMEDAVPEDRLPLAMGGGIGFAVIMFWFAWSAEFNSVHWIVPILA |
| Pb_CEJ60583.1 | ADIRMRQKRIDNGTIKMEDAVPEDRLPLAMGGGIGFAVIMFWFAWSAEFNSVHWIVPILA |
| Ps_OKO99970.1 | ADIRMRQKKIDKGITKMEDAEPEDRLPLAIGGGIAFALTMFWFAWSAEFNSVHWIVPTIA |
| As_OJJ53782.1 | VDTRIRQRRIERGEKKMEDTVPEDRLTLAMIGGVGFPATMFWFAWSAEYNYVHWIVPILA |
| Av_OJJ07888.1 | ADTHIRQRRIERGEKKIEDTVPEDRLTLAMIGGVGFPATMFWFAWSAEYNYVHWIVPILA |
| Pi_KGO73014.1 | IDIRLRQRKIERGEKKMEDNVPEDRLILAMIGGIVFPATMFWFAWSAEYNSVHWIVPILA |
| Ti_CRG83369.1 | IDTRIRQKKIERGEMKMEDATPEDRLPLAIIGGFGFAIAMFWFAWSAEYNSVHWIVPTIA |
| Ar_EYE92060.1 | IDIRLRQRRIERGEKKMEDTVPEDRLTLAMIGGIGFPATMFWFAWSAEYNYVHWIVPILA |
| Ag_OJJ86250.1 | VDTRLRQRQIERGEKKMEDTVPEDRLTLAMIGGIGFPATMFWFAWSAEYNYVHWIVPILA |
| Ma_KID68223.1 | YDIRRRIKKIERGEIKADDMEPEDRLPLAMVGGIGFAAAMFWFSWTAEFNSVHWIVPTIA |
| Ma_KJK94474.1 | YDIRRRIKKIERGEIKADDMEPEDRLPLAMVGGIGFAAAMFWFSWTAEFNSVHWIVPTIA |
| | **: * ::*:.* * :* ***** **: **. * :****:*:**:* *******:* |
| HmfT6 | GVFLSSALLLIFVGFLNYLVDVYQMYAASAIAANTIARSACGAAAPLFTSQMFAALGVGG |
| Pb_CEJ60583.1 | GVFLSSALLLIFVGFLNYLVDVYQMYAASAIAANTIARSACGAAAPLFTSQMFAALGVGG |
| Ps_OKO99970.1 | GVFISSAMLLIFVGFLNYLVDVYQMYAASAIAANTIARSACGAAAPLFTSQMFAALGVGG |
| As_OJJ53782.1 | GCFLSSCLLLIFVSYLNYLVDCYLMYAASAIAANTIARSACGAAAPLFINQMFTAMGIGG |
| Av_OJJ07888.1 | GCFLSSCLLLIFVSYLNYLVDCYLMYAASAIAANTIARSACGAAAPLFINQMFTAMGIGG |
| Pi_KGO73014.1 | GCFLSSSLLLIFVAYLNYLVDCYLMYAASAIAANTIARSACGAAAPLFINQMFSALGVGG |
| Ti_CRG83369.1 | GGLLSASMLLIFVAYLNYLVDVYLMYAASAIAANTIARSACGAAAPLFINQMFTALGVGG |
| Ar_EYE92060.1 | GCFLSSCLLLIFVAYLNYLVDCYLMYAASAIAANTIARSACGAAAPLFINQMFSALGVGG |
| Ag_OJJ86250.1 | GCFLSSSLLLIFVAYLNYLVDCYLMYAASAIAANTIARSACGAAAPLFINQMFSALGVGG |
| Ma_KID68223.1 | GGLLSTFMLLIFVAYLNYLVDVYLMYAASAIAANTIARSASGAAAPLYISQMFHALGIGG |
| Ma_KJK94474.1 | GGLLSTFMLLIFVAYLNYLVDVYLMYAASAIAANTIARSASGAAAPLYISQMFHALGIGG |
| | * ::*: :*****.:****** * ****************.******:*.*** *:*:** |
| HmfT6 | GGSLIAGVAILLAAIPFLFYKYGKQIRMRSKFAPTIKEERPAEENKDEERGLGDGAVSSS |
| Pb_CEJ60583.1 | GGSLIAGVAILLAAIPFLFYKYGKQIRMRSKFAPTIKEERPAEENKDEERGLGDGAVSSS |
| Ps_OKO99970.1 | GGSLIAGVAILLAGIPFLFYKYGKQIRMRSKFAPIKKGEQPVEEDKDEERGLGNGPSSSV |
| As_OJJ53782.1 | GGSLIGGVATVLGIIPFLFYKYGKQIRVASKFAPALDVK--KNRTADEEAGAVSGGIQGE |
| Av_OJJ07888.1 | GGSLIGGVATVLGIIPFLFYKYGKQIRVASKFAPAFDVK--KNQTTDEEAGAVSGGVQDE |
| Pi_KGO73014.1 | GGSLIGGVASLLAVIPFLFYKYGKQIRFRSRFAPGQDAR--RQEKSDEEAVPPGAGIQSS |
| Ti_CRG83369.1 | GGSLIGGVATLLACIPFLFYKYGKQIRIRSKFAPTNEKR--VQQETDEESGTQEGQDGDK |
| Ar_EYE92060.1 | GGSLIGGVAALLACIPFLFNRYGKQIRIRSRFAPAPDTR--IQGGSDEEAGPRDEGIRSP |
| Ag_OJJ86250.1 | GGSLIGGVAALLVCIPFLFDKYGKQIRIRSRFAPAPDAR--VQGKSDEEAGPRDEGIRGP |
| Ma_KID68223.1 | GGSLVAGVATLLAVVPFVFYKYGKPIRIRSKFAPTNTKE--KNRVEDEEANPTDFAIQSS |
| Ma_KJK94474.1 | GGSLVAGVATLLAVVPFVFYKYGKPIRIRSKFAPTNTKE--KNRVEDEEANPTDFAIQSS |
| | ****:.***::* :**:* :*** **.**:*** . : *** . |
| HmfT6 | ILGAQ-------------------------------------------- |
| Pb_CEJ60583.1 | ILGSQ-------------------------------------------- |
| Ps_OKO99970.1 | LETQ--------------------------------------------- |
| As_OJJ53782.1 | GQRHRHEE------------------EGQNAVTEGTNMEGDGTTKEGNR |
| Av_OJJ07888.1 | TQRRSLSESTESTTSESTVGDQVHEAEGQNTVAEGAK-EGDGATREGTS |
| Pi_KGO73014.1 | ISPSDESETSIS-EASENAEVE--------------------SGEKVRD |
| Ar_EYE92060.1 | VPQSSQSAEVERGEKLRKPGIE--------------------EKSQHPQ |
| Ti_CRG83369.1 | ------------------------------------------------- |
| Ag_OJJ86250.1 | VSQFNESDEVERGEKN--------------------------------- |
| Ma_KID68223.1 | SEVDSSTDVDSSHTSP-----VANEKTEERDVSSGGQVLAPNERKGSRP |
| Ma_KJK94474.1 | SEVDSSTDVDSSHTSP-----VGNEKTEERDVSSGGQVLAPNERKGSRP |

TABLE 10
Amino acid sequence alignment of Penicillium brasilianum hmfT7
and 10 closest orthologues.

| | |
|---|---|
| HmfT7 | MAA--SNEHATPSIDSATTKTAPSVDSTPAMSDHVN--EDLEKGTATTDPVEAEKHDLNV |
| Pb_CEO59852.1 | MAV--SNEHATPSIDSATTKTAPSVDSTPAMSDHVN-EDLEKGTATTDPVEAEKHDLNV |
| Ps_OKP11238.1 | MGA--NHEHATPSIDSATTNIAPSDNSTPAMSDHVN--DDLEKGTRTADPIESEGRDVNG |
| Po_EPS33230.1 | MSEDNTHPAGGPSSDGSTTTRAPSERSTPSMINQEANDNDMEKGRSTADPIDSKATQVQD |
| Pa_OGE55472.1 | -----MTATNPPSIDSATTNIPPSDNSTPAMSVQGN--EDPEKGIQGPEKTEPAAD---- |
| Pr_XP_002564221.1 | --MANNETETTPSI--------DSATTTPAVSVHGN--NDPEKGIAPSEKHEAP------ |
| Pg_KXG53210.1 | ---MAHQTEIVSSTDS--DSIQLSNGSTPTMSMHEN--NDTEKGLGPLEKDIQS------ |
| Pn_KOS36679.1 | --MANNETEVAPSI--------DSATTIPVVNVSGN--NDPEKGLASSEKDEPQ------ |
| An_XP_015410789.1 | -MVAANREQDVPSIASTTINAISDDHSTPAVSVHLE--KGLEMGSQPDQQNTTELVTSKP |
| Pi_KGO73431.1 | --MANNETEVAPSI--------GSA-TTPAVSVYGN--SDPEKGLEPSEKDEP------- |
| Pe_KGO59243.1 | --MANNETAVAPSI--------DSATTMPVLGVHEN--NDPEKGLAPPEKDEQ------- |
| | * . : * : .. * * : |
| HmfT7 | NPLSEKIEG--TTPAPAGPPGPGPPPDGGAEAWLVVLGAFCGLFVSFGWINCIGVFQTYY |
| Pb_CEO59852.1 | NPLSEEIEG--TTPAPAGPPGPGPPPDGGAEAWLVVLGAFCGLFVSFGWINCIGVFQTYY |
| Ps_OKP11238.1 | EPLSEKSEA--VTPALAGPPSPGPPPDGGAEAWMSVLGAFCGLFVSFGWINCIGVFQTYY |
| Po_EPS33230.1 | ESLPEKTQIGTGPPALTATPSPGPPPDGGAEAWMSVLGAFCGLFVSFGWINCIGIFQTYY |
| Pa_OGE55472.1 | AQPSEKSVAAG--P-EPATPAIGPPPDGGAEAWLVVLGAFSGLFVSFGWINCIGVFQTYY |
| Pr_XP_002564221.1 | -PPTEKSIAAGPA---PATPVISPPPDGGAQAWLVVLGAFCGLFVSFGWINCIGVFQAYY |
| Pg_KXG53210.1 | --PSEKSVAAG--P-VPATPVIGPPPDGGTQAWLVVLGAFCGLFVSFGWINCIGVFQAYY |
| Pn_KOS36679.1 | -PPTEKSIATGPAP-APATPSIGPPPDGGAQAWLVVLGAFCGLFVSFGWINCIGVFQEYY |
| An_XP_015410789.1 | PRASPQPGEKREASGPPVTPAPGLPPDGGLQAWMTILGAFCGMFVSFGWTNCIGVFQAYY |
| Pi_KGO73431.1 | --PVEKSVAAGSV----PTPAIGPPPDGGAQAWLVVMGAFCGLFVSFGWINCIGVFQDYY |
| Pe_KGO59243.1 | --SSEKLIAAG--P-APGTSVIGPPPDGGAQAWLVVLGAFCGLFVSFGWINCIGVFQDYY |
| | : . ***** :**: ::***.*:****** ****:** ** |
| HmfT7 | ETHQLSNLSTSTVTWITSLETFVMFFAGPVFGTLFDSYGPRYILLGGTFLHVFGLMMTSL |
| Pb_CEO59852.1 | ETHQLSNLSTSTVTWITSLETFVMFFAGPVFGTLFDSYGPRYILLGGTFLHVFGLMMTSL |
| Ps_OKP11238.1 | ETHQLSDMSTSTVTWITSLETFVMFFAGPIFGTLFDNYGPRWILLGGTFFHVFGLMMASL |
| Po_EPS33230.1 | ETHQLRNLSTSTVTWITSLETFVMFFVGPIFGTLFDNYGPRAILLGGTFFHVFGLMMASL |
| Pa_OGE55472.1 | ESHQLSEYSTSTVTWITSLETFIMFFCGPIFGTMFDSYGPRWILLLGTFLHVFGLMMASL |
| Pr_XP_002564221.1 | QSHQLSEFSTSTVTWITSLETFMMFFCGPIFGTMFDSYGPRWILLIGTILHVFGLMMASL |
| Pg_KXG53210.1 | ESHQLKEFSTSTVTWITSLETFTMFFCGPMFGTLFDSYGPRWILLVGTILHVFGLMMASL |
| Pn_KOS36679.1 | QSHQLSEFSTSTVTWITSLETFMMFFCGPIFGTMFDSYGPRWILLLGTILHVFGLMMASL |
| An_XP_015410789.1 | ERHQLSHLSPSTIAWITSLETFVMFFAGPLFGTLFDNYGPRWILLAGTFFHVFGLMMASI |
| Pi_KGO73431.1 | QTHQLSEFSTSTVTWITSLETFMMFFCGPIFGTLFDSYGPRWILLLGTILHVFGLMMASL |
| Pe_KGO59243.1 | QAHQLSHFSTSTVTWITSLETFMMFFCGPVFGTIFDSYGPRWILLIGTVLHVFGLMMASL |
| | : *** . * **::******** *** **:***:**.**** *** **.:*******:*: |
| HmfT7 | STEYYQFILAQGICSPLGASAIFNASINSVSTWFAKRRAFALGVTASGSSLGGVIFPIMV |
| Pb_CEO59852.1 | STEYYQFILAQGICSPLGASAIFNASINSVSTWFAKRRAFALGVTASGSSLGGVIFPIMV |
| Ps_OKP11238.1 | STEYYQFILAQGICSPLGASAIFNASVNSVSTWFAKRRAFALGVTASGSSLGGVIFPIMV |
| Po_EPS33230.1 | STEYYQFILAQGICSPLGASAIFNASVNSVSTWFAKRRAFALGITASGSSLGGVIFPIMV |
| Pa_OGE55472.1 | STEYYQFILAQGICSPIGASAIFNASVNSVSTWFAKRRAFALGVTASGSSLGGVIFPIMV |
| Pr_XP_002564221.1 | STEYYQFILAQGICSPIGASAIFNASVNSVSTWFAKRRAFALGVTAAGSSLGGVIFPIMV |
| Pg_KXG53210.1 | STEYYQFILAQGICSPIGASAIFNASVNSVSTWFAKRRAFALGVTAAGSSLGGVIFPIMV |
| Pn_KOS36679.1 | STDYYQFILAQGICSPIGASAIFNASVNSVSTWFAKRAGFALGVTASGSSLGGVIFPIMV |
| An_XP_015410789.1 | STEYYQFILSQGICSPLGASAVFNASINSASGWFAKRRAFALGVAASGSSLGGVIFPIMV |
| Pi_KGO73431.1 | STEYYQFILAQGICSPIGASAIFNASINSVSTWFAKRAGFALGVTASGSSLGGVIFPIMV |
| Pe_KGO59243.1 | STEYYQFILAQGICSPIGASAIFNASVNSVSTWFAKRAGFALGVTASGSSLGGVIFPIMV |
| | **:******:******:****:****:**.* ******.****::*:************* |
| HmfT7 | TNLIPEVGFPWAMRICAFLILAMLGVSNLTLKSRLKHTRKPFNFMNFVRPLKDIKFVVTV |
| Pb_CEO59852.1 | TNLIPEVGFTWAMRICAFLILAMLGVSNLTLKSRLKHTRKPFNIMNFVRPLKDVKFVVTV |
| Ps_OKP11238.1 | SNLIPKVGFAWAMRICAFLILFMLGISNITLKSRLKHTKKPFDIMNFVRPLKDVKFVVTV |
| Po_EPS33230.1 | SNLIPKVGFPWAMRICAFVILAMLAIANATLKSRITHTKKPFDIMNFVRPLKDVKFVVTV |
| Pa_OGE55472.1 | TRLIPEVGFPWAMRICAFLILFMLGIANVTLKSRLPHRPKPFDILSFLRPLTELKFALTL |
| Pr_XP_002564221.1 | TQLIPKVGFPWAMRICAFLILFMLGIANLTLKSRLPHRPKPFDILSFLRPLAELKFALTL |
| Pg_KXG53210.1 | TQLIPKVGFPWAMRICAFLILFMLGITNLTLKSRLSHRPKPFDFLGFLRPLAELKFALTL |
| Pn_KOS36679.1 | TQLIPKVGFPWAMRICAFVILFMLGIANLTLKSRLPHRPKQFDILSFVRPLAELKFALTL |
| An_XP_015410789.1 | SKLIPQVDFGWAMRICAFLILFMLGITNLTLRSRLKPQNKAFDIMAFVRPLHDLKFVLTA |
| Pi_KGO73431.1 | TQLIPKVGFPWAMRICAFLILFMLGIANLTLKSRLPHRPKPFDILGFVRPLAELKFAITL |
| Pe_KGO59243.1 | TQLIPKVGFPWAMRICAFLILFMLGIANLTLKSRLPHRPKPFDILGFLRPLAELKFALTL |
| | :.***:*.* ********:** **.::* **:**: * *::: *:*** ::**.:* |
| HmfT7 | AAAFCFFWGMFLPFTFVITQAQRYGMSEHLSQYLIPILNAASVFGRTLPGYLADRVGRYN |
| Pb_CEO59852.1 | AAAFCFFWGMFLPFTFVITQAQRYGMSEHLSQYLIPILNAASVFGRILPGYLADRVGRYN |
| Ps_OKP11238.1 | AACFCFFWGMFLPFTFVITQAQRYGMSEHLSQYLIPILNAASVFGRIVPGYMADRVGRYN |
| Po_EPS33230.1 | AACFCFFWGMFLPFTFVITQAQRYGMSEHLSLYLIPILNAASIFGRILPGYLADRIGRYN |
| Pa_OGE55472.1 | AGAFCFFWGMFLPFTFVITQAERYGMSPGLAQYLIPILNAASIFGRILPGYFADKIGRYN |
| Pr_XP_002564221.1 | AAAFCFFWGMFLPFTFVITQAERYGMSSNLAGYLIPILNASSIFGRILPGYLADKVGRYN |
| Pg_KXG53210.1 | ASAFCFFWGMFLPFTFVITQAERYGMSANLAGYLIPILNASSIFGRILPGYLADKIGRYN |
| Pn_KOS36679.1 | AAAFCFFWGMFLPFTFVITQAERYGMSANLAGYLVPILNASSIFGRILPGYLADKIGRYN |
| An_XP_015410789.1 | AAAFCFFWGMFLPFTFVISSGERYGMSQNMSSYLLPILNAASIFGRILPGYIADRIGRYN |
| Pi_KGO73431.1 | AAAFCFFWGMFLPFTFVITQAERYGMSTNMAGYLIPILNASSIFGRILPGYLADKVGRYN |
| Pe_KGO59243.1 | AAAFCFFWGMFVPFTFVITQAQRYGMSANLAGYLIPILNASSIFGRILPGYLADKVGRYN |
| | *..********:..:***** :: **:*****:*:****:***:**::**** |
| HmfT7 | VMIFFSYLSGILVLALWLPSRSNAPAIVFSALYGFGSGAFVSLAPALIAQISDVREVGVR |
| Pb_CEO59852.1 | VMIFFSYLSGILVLALWLPSRSNAPAIVFSALYGFGSGAFVSLAPALIAQISDVREVGVR |
| Ps_OKP11238.1 | VMIFFSYLSAILVLALWLPSRSNAPAIVFSAMYGFGSGAFVSLAPALIAQISDLREVGVR |
| Po_EPS33230.1 | IMIFFSYLSAILVLALWLPSRSNVPAIIFSALYGFSSGAFVSIVPALIAQISDLREVGVR |
| Pa_OGE55472.1 | MMVVTIFFSAILVLALWLPSRGNAPAIVFSALYGFGSGAFVSLAPACIAQISDLRQVGVR |
| Pr_XP_002564221.1 | MMVITIFFSAILVLALWLPSKGNAPAIVFSALYGFGSGAFVSLAPALVAQISDLRQVGVR |
| Pg_KXG53210.1 | MMVLTIFFSSILVLALWLPSRGNAPAIVFSALYGFGSGAFVSLAPALVAQISDLRQVGVR |
| Pn_KOS36679.1 | MMVITIFFSSVLVLALWLPSRGNVPAILFSALYGFGSGAFVSLAPALIAQISDLRQVGVR |
| An_XP_015410789.1 | VMIIFSYFSAILVLALWLPSRGNIPIIIFSALYGFGSGAFVSLIPALIAQISDLHEVGAR |
| Pi_KGO73431.1 | MMVMTIFFSSILVLALWLPSRGNVPVIVFSALYGFGSGAFVSLAPALVAQISDLRQVGVR |
| Pe_KGO59243.1 | MMVLTIFFSAILVLALWLPSRGNIPAILFSALYGFGSGAFVSLAPALIAQISDLRQVGVR |
| | :*:. :::*.:*********:.* * *:***:***.******: ** :*****:::**.* |
| HmfT7 | NGTCFSIIAFAALIGTPIGGALVPDVLIGSYTRLQVFSGVVMLAGATLFVVARLVVGG-- |
| Pb_CEO59852.1 | NGTCFSIIAFAALIGTPIGGALVPDVLIGSYTRLQVFSGVVMLAGATLFVVARLVVGG- |
| Ps_OKP11238.1 | NGTCFSIISFAALIGTPIGGALVPDVLIGSYTRLQVFSGVVMLAGASLFVVARIVVGG-- |
| Po_EPS33230.1 | NGTCFSIISFAALIGTPIGGALVPDVLIGSYTKLQVFCGVVMMAGSALFVVARIVVGG-- |
| Pa_OGE55472.1 | NGIFFAIISFAALIGTPIGGALVPDVLHGSYTKLQIFCGVVMIAGSTLFVLARVVVGGGF |
| Pr_XP_002564221.1 | NGIFFAVISFAALIGTPIGGALVPDVLHGSYTRLQIFCGVVMIVGSTLFAFARGAVGG-- |
| Pg_KXG53210.1 | NGIFFAVISFAALIGTPIGGALVPDVLHDDYTRLQIFAGVVMIAGSVMFVFARGAVGGFK |
| Pn_KOS36679.1 | NGIFFAVISFAALIGTPIGGALVPDVLHGDYTRLQIFCGVVMIVGSVLFVFARGAIGG- |
| An_XP_015410789.1 | NGTCFSIISIAALIGSPIGGALVPDMLHGSYTRLQVFCGVVMMAGATLFVAARIVVGGMN |
| Pi_KGO73431.1 | NGIFFAIISFAALIGTPIGGALVPDVLHDDYTRQQIFCGVVMIVGSTLFVFARGVVGG- |
| Pe_KGO59243.1 | NGIFFAVISFAALIGTPIGGALVPDVLHGDYTRLQIFCGVVMTVGSTLFVFARGAVGAPM |
| | *** *::*::*****:*********:* ..**: *:*.**** .*: :*. ** .:*. |
| HmfT7 | ------------------------VKFGKSLGSGVLMGCISNGLIEHDRRMHWHYHVVIC |
| Pb_CEO59852.1 | ------------------------VKFGKV------------------------------ |
| Ps_OKP11238.1 | ------------------------VKMGKV------------------------------ |
| Po_EPS33230.1 | ------------------------WKLGKV------------------------------ |
| Pa_OGE55472.1 | KLNKV------------------------------------------------------- |
| Pr_XP_002564221.1 | ------------------------FKLTKV------------------------------ |
| Pg_KXG53210.1 | LTKV-------------------------------------------------------- |
| Pn_KOS36679.1 | ------------------------FKLNKV------------------------------ |
| An_XP_015410789.1 | LKKV-------------------------------------------------------- |
| Pi_KGO73431.1 | ------------------------FKMTKALRGVNPIFLTDQHVPGINLIDKWIIISGSN |
| Pe_KGO59243.1 | SHVVHIPQCKGNIYPNMASIFAWLYDYLQAARGINTTFLTDKHVPGANLIGKWIIISGSN |
| HmfT7 | CAATDS------------------------------------------------------ |
| Pb_CEO59852.1 | ------------------------------------------------------------ |
| Ps_OKP11238.1 | ------------------------------------------------------------ |
| Po_EPS33230.1 | ------------------------------------------------------------ |
| Pa_OGE55472.1 | ------------------------------------------------------------ |
| Pr_XP_002564221.1 | ------------------------------------------------------------ |
| Pg_KXG53210.1 | ------------------------------------------------------------ |
| Pn_KOS36679.1 | ------------------------------------------------------------ |
| An_XP_015410789.1 | ------------------------------------------------------------ |
| Pi_KGO73431.1 | NGVGFEAAKSFASWGANIILACREPPAWELHPTAAVNECRDLAAANGHSSAIEWWQIDMA |
| Pe_KGO59243.1 | NGVGFEAAKSFASWGANLILACREPPAWELHPTAAVDECKALAAASGHSSTIEWWQIDMA |
| HmfT7 | ------------------------------------------------------------ |
| Pb_CEO59852.1 | ------------------------------------------------------------ |
| Ps_OKP11238.1 | ------------------------------------------------------------ |
| Po_EPS33230.1 | ------------------------------------------------------------ |
| Pa_OGE55472.1 | ------------------------------------------------------------ |
| Pr_XP_002564221.1 | ------------------------------------------------------------ |
| Pg_KXG53210.1 | ------------------------------------------------------------ |
| Pn_KOS36679.1 | ------------------------------------------------------------ |
| An_XP_015410789.1 | ------------------------------------------------------------ |
| Pi_KGO73431.1 | DLSSVEAFCERWLQSDRVLDILCNNAGIPETTKRTYITKDGFQLVHQTLINIPPQVNFLS |
| Pe_KGO59243.1 | DLSSIEAFCQRWLECDRTLDILCNNAGIPESTKQTYMTKDGFQLVHQ--------VNLLS |
| HmfT7 | ------------------------------------------------------------ |
| Pb_CEO59852.1 | ------------------------------------------------------------ |
| Ps_OKP11238.1 | ------------------------------------------------------------ |
| Po_EPS33230.1 | ------------------------------------------------------------ |
| Pa_OGE55472.1 | ------------------------------------------------------------ |
| Pr_XP_002564221.1 | ------------------------------------------------------------ |
| Pg_KXG53210.1 | ------------------------------------------------------------ |
| Pn_KOS36679.1 | ------------------------------------------------------------ |
| An_XP_015410789.1 | ------------------------------------------------------------ |
| Pi_KGO73431.1 | HVLLTLALLPSLARSVEPRIICTTSCLHHLGVFDLDHENGGPGQKGEDYPNNKLYFQMWV |
| Pe_KGO59243.1 | HVLLTLALLPSLARSAEPRVICTTSCYHHLGVFDLDHENGGPGQKGRDYQNNKLYFQMWI |
| HmfT7 | ------------------------------------------------------------ |
| Pb_CEO59852.1 | ------------------------------------------------------------ |
| Ps_OKP11238.1 | ------------------------------------------------------------ |
| Po_EPS33230.1 | ------------------------------------------------------------ |
| Pa_OGE55472.1 | ------------------------------------------------------------ |
| Pr_XP_002564221.1 | ------------------------------------------------------------ |
| Pg_KXG53210.1 | ------------------------------------------------------------ |
| Pn_KOS36679.1 | ------------------------------------------------------------ |
| An_XP_015410789.1 | ------------------------------------------------------------ |
| Pi_KGO73431.1 | AELQSALLKNPEYLHITINGVHPGEVASGIWNGLQNTGKPPSGLSELLCYVAITPQQGGL |
| Pe_KGO59243.1 | AELQSALLKNPEYLHITINGVHPGEVASGIWHGLQNTGKAPGGLNELLRYVAITSQQGGL |
| HmfT7 | ------------------------------------------------------------ |
| Pb_CEO59852.1 | ------------------------------------------------------------ |
| Ps_OKP11238.1 | ------------------------------------------------------------ |
| Po_EPS33230.1 | ------------------------------------------------------------ |
| Pa_OGE55472.1 | ------------------------------------------------------------ |
| Pr_XP_002564221.1 | ------------------------------------------------------------ |
| Pg_KXG53210.1 | ------------------------------------------------------------ |
| Pn_KOS36679.1 | ------------------------------------------------------------ |
| An_XP_015410789.1 | ------------------------------------------------------------ |
| Pi_KGO73431.1 | AISHAATGVEFGPDPKRQGVGAENGRGGGRYINRIWEGPAKSYCSDTEARSRLWIKLDEE |
| Pe_KGO59243.1 | AISHAATASEFGPDPRKQGVGAENGRGGGR---------------DTEARSRLWIKLDEE |
| HmfT7 | |
| Pb_CEO59852.1 | -------------- |
| Ps_OKP11238.1 | -------------- |
| Po_EPS33230.1 | -------------- |
| Pa_OGE55472.1 | -------------- |
| Pr_XP_002564221.1 | -------------- |
| Pg_KXG53210.1 | -------------- |
| Pn_KOS36679.1 | -------------- |
| An_XP_015410789.1 | -------------- |
| Pi_KGO73431.1 | LGLQEKGLLTSLGL |
| Pe_KGO59243.1 | LGLQEKGLLTGLGI |

TABLE 11
Amino acid sequence alignment of Penicillium brasilianum hmfR
and 10 closest orthologues.

| | |
|---|---|
| P_brasilianum_hmfR | -------------------------------------------------- |
| Spo_sche_ERT02388 | MSHPAGHAAPATASVTSTRRLRRVADTSRKRSVQSCDFCRKRRCKCVPQP |
| Sce_apio_KEZ45621 | -------MADSPPDAAARRRLRRVPEQLRKRSAHSCDLCRKRRCKCVPGP |
| Sta_chlo_KFA62280 | ---------MPESSAAAKRRMRRIPAQLRKRNLQSCDWCRKRRCKCVPST |
| Ver_alfa_XP_003000413 | ---------MSESVSAAKRRQRRIPDEFR--------------------- |
| Fus_oxys_EXL68817 | ---------MSES-ANAKRRLRRIPDESRKRNAQSCDRCRKRRCKCVPDP |
| Fus_oxys_EXK46473 | ---------MSES-ANAKRRLRRIPDESRKRNAQSCDRCRKRRCKCVPDP |
| Fus_oxys_EGU75021 | ---------MSES-ANAKRRLRRIPDESRKRNAQSCDRCRKRRCKCVPDP |
| Fus_oxys_EXM14771 | ---------MSES-ANAKRRLRRIPDESRKRNAQSCDRCRKRRCKCVPDP |
| Fus_oxys_EXM09676 | -------------------------------------------------- |
| Fus_oxys_EXK77862 | ---------MSES-ANAKRRLRRIPDESRKRNAQSCDRCRKRRCKCVPDP |
| P_brasilianum_hmfR | ------MCQDHDLECSYTLPRKTRFYGSVDDLSDRYKCLEAIVRAAFPND |
| Spo_sche_ERT02388 | AGDGCLMCHTQGVACSYTLPRKARFYGSVEDLSDRFKCLEAIVRGAFPSD |
| Sce_apio_KEZ45621 | AGRGCATCEKHNVECSYALPRKSRFYGSVDDLGDRHKCLEAIVRGAFPGE |
| Sta_chlo_KFA62280 | TGQGCVSCEQHDVQCSYTAPRKTRFYGSLDELSLRYRCLEAVVKGAFHND |
| Ver_alfa_XP_003000413 | ---------------------------------------------AFPND |
| Fus_oxys_EXL68817 | SGAGCVNCLEHNVTCSYTAPRKTRFYGSVDDLSDRYRCLEAIVRGAFPNE |
| Fus_oxys_EXK46473 | SGVGCVNCLEHNVTCSYTAPRKTRFYGSVDDLSDRYRCLEAIVRGAFPNE |
| Fus_oxys_EGU75021 | SGAGCVNCLEHNVTCSYTAPRKTRFYGSVDDLSDRYRCLEAIVRGAFPNE |
| Fus_oxys_EXM14771 | SGAGCVNCLEHNVTCSYTAPRKTRFYGSVDDLSDRYRCLEAIVRGAFPNE |
| Fus_oxys_EXM09676 | -----MNCLEHNVTCSYTAPRKTRFYGSVDDLSDRYRCLEAIVRGAFPNE |
| Fus_oxys_EXK77862 | SGAGCVNCLEHNVTCSYTAPRKTRFYGSVDDLSDRYRCLEAIVRGAFPNE |
| | ** .: |
| P_brasilianum_hmfR | GISTVPELIRLGERMGYAMPDLSQ-KSGESPRIEELVR--------DFPT |
| Spo_sche_ERT02388 | PIATVPELLRLGHRLGVTMPDLAD-DARAKLSLDDLVNTPSKSVTSDQTT |
| Sce_apio_KEZ45621 | PTATVADLRKLGERMGYSMPEPTI-PSTRPLESSEPTIS--------YPS |
| Sta_chlo_KFA62280 | DIATAAELVQLGRRLGYAMPDINHKATYSEVKLDEIIRAP--------AV |
| Ver_alfa_XP_003000413 | LTATAEDLVELGRRMGYAMPDFSQ-PRRKGVKIEDLVRAP--------DP |
| Fus_oxys_EXL68817 | TLDHVSDLAQLGQKMGYKMPDVSD-PNRAHIRVEDLVQNP--------SS |
| Fus_oxys_EXK46473 | TLDHVSDLAQLGQKMGYKMPDVSD-PNRTHIRVEDLVQNP--------SS |
| Fus_oxys_EGU75021 | TLDHVSDLAQLGQKMGYKMPDVSD-PNRAHIRVEDLVQNP--------SS |
| Fus_oxys_EXM14771 | TLDHVSDLAQLGQKMGYKMPDVSD-PNRTHIRVEDLVQNP--------SS |
| Fus_oxys_EXM09676 | TLDHVSDLAQLGQKMGYKMPDVSD-PNRTHIRVEDLVQNP--------SS |
| Fus_oxys_EXK77862 | TLDHVSDLAQLGQKMGYKMPDVSD-PNRTHIRVEDLVQNP--------SS |
| | . :* .**.::* **: .: |
| P_brasilianum_hmfR | EAGDQGLAGSTQCTSSPPRTGAVNVPTESER------------------- |
| Spo_sche_ERT02388 | AVEGAVDGGGSGGGGGDRRPSMTNAPTQSDAGHVNARPLATEPESADTVN |
| Sce_apio_KEZ45621 | SEAPIRRPLVPSHEAVSRRNSCPDVFG----------------------- |
| Sta_chlo_KFA62280 | TPLPIPRTPES-------DSSGQSDCVE---------------------- |
| Ver_alfa_XP_003000413 | VGSSRHNSVAESKISGSELGTGSKAPSE---------------------- |
| Fus_oxys_EXL68817 | K----ERTP----------STGPDIITA---------------------- |
| Fus_oxys_EXK46473 | K----ERTP----------STGPDIITA---------------------- |
| Fus_oxys_EGU75021 | K----ERTP----------STGPDIITA---------------------- |
| Fus_oxys_EXM14771 | K----ERTP----------STGPDIITA---------------------- |
| Fus_oxys_EXM09676 | K----ERTP----------STGPDIITA---------------------- |
| Fus_oxys_EXK77862 | K----ERTP----------STGPDIITA---------------------- |
| P_brasilianum_hmfR | ----------------RHSSSQVQENNSCPD----EPVGLIRDTTGREHF |
| Spo_sche_ERT02388 | TDNTHNTGNSGNTDNTRHTTTTDGTASSNPQDESSEAIGLVRDTTGQEHF |
| Sce_apio_KEZ45621 | -----------------ARVPEGVDGDSSPDD--AESLGLIRDPTGRQHY |
| Sta_chlo_KFA62280 | -----------------RGGGETWRPRTRVN-SEEPHVSLIRDTSGNEHY |
| Ver_alfa_XP_003000413 | -----------------VGTDDAVSAAAAASGAEDAQLSLIRDTSGNEHY |
| Fus_oxys_EXL68817 | -----------------DSRADTSPRSSKSH-SEEPQSSLVKDNSGHEHY |
| Fus_oxys_EXK46473 | -----------------HSRADTSPRSSKSH-SEEPQSSLVKDNSGHEHY |
| Fus_oxys_EGU75021 | -----------------DSRADTSPRSSKSH-SEEPQSSLVKDNSGHEHY |
| Fus_oxys_EXM14771 | -----------------DSRADTSPRSSKSH-SEEPQSSLVKDNSGHEHY |
| Fus_oxys_EXM09676 | -----------------DSRADTSPRSSKSH-SEEPQSSLVKDNSGHEHY |
| Fus_oxys_EXK77862 | -----------------DSRADTSPRSSKSH-SEEPQSSLVKDNSGHEHY |
| | : .*::* :*.:*: |
| P_brasilianum_hmfR | IGPSGSLQFLGQLRRLLLISR-----------------SGDAVESRAPAR |
| Spo_sche_ERT02388 | IGSSGSLQFLGQLRRLLLLSQHDNMSRNSSYHGIGYPCSGYSAPGRASQR |
| Sce_apio_KEZ45621 | IGPSGSLQFLSQLRRLLISRN------------------QRLPVNNDNSP |
| Sta_chlo_KFA62280 | IGPSGTLNFLSQLRKLFDTDT-----------------TANPALAAAACP |
| Ver_alfa_XP_003000413 | IGPSGTLNFLSQLRRLMVSSE-----------------GTPEAQPEV--- |
| Fus_oxys_EXL68817 | IGPSGTLNFWNQLRNLVDSNN-----------------SPHPSPGRE--- |
| Fus_oxys_EXK46473 | IGPSGTLNFWNQLRNLVDSNN-----------------SPYPSPGRE--- |
| Fus_oxys_EGU75021 | IGPSGTLNFWNQLRNLVDSNN-----------------SPHPSPGRE--- |
| Fus_oxys_EXM14771 | IGPSGTLNFWNQLRNLVDSNN-----------------SPHPSPGRE--- |
| Fus_oxys_EXM09676 | IGPSGTLNFWNQLRNLVDSNN-----------------SPYPSPGRE--- |
| Fus_oxys_EXK77862 | IGPSGTLNFWNQLRNLVDSNN-----------------SPYPSPGRE--- |
| | **.**:*:* .***.*. |
| P_brasilianum_hmfR | -LTATFTDEDAAQALEAD---GDQSELAALPSGGTGN-----GGDEGQEI |
| Spo_sche_ERT02388 | -LSTTFTEEDAAQALEAD---NSHDGSDAPPTLHHHT-----PLMD---- |
| Sce_apio_KEZ45621 | -TASKFTEDDTARALEADSITVDTTDPVVAAADHGGV-----AGDVVAAQ |
| Sta_chlo_KFA62280 | AGATKFAQDDAAQALEAEGEPRDEERHDEAEAGDAMNCSRDSVPRVPQPQ |
| Ver_alfa_XP_003000413 | --VTKFTQDDTAQALEADDSPGAPGALHPATQTDG-------------PL |
| Fus_oxys_EXL68817 | -GATKFTQDNTSRLLEADGQDEDDQPPRTAAT----------------PP |
| Fus_oxys_EXK46473 | -GATKFTQDNTSRLLEADGQDEDDQPPRTAAT----------------PQ |
| Fus_oxys_EGU75021 | -GATKFTQDNTSRLLEADGQDEDDQPPRTAAT----------------PP |
| Fus_oxys_EXM14771 | -GATKFTQDNTSRLLEADGQDEDDQPPRTAAT----------------PQ |
| Fus_oxys_EXM09676 | -GATKFTQDNTSRLLEADGQDEDDQPPRTAAT----------------PQ |
| Fus_oxys_EXK77862 | -GATKFTQDNTSRLLEADGQDEDDQPPRTAAT----------------PQ |
| | :.*::::::: ***: |
| P_brasilianum_hmfR | DERSPASLG--SALVRDFSSIPVNDIDEMRRQLPPRHVLDSLMRVYFKNV |
| Spo_sche_ERT02388 | DRPSPMSSS--SALARECATIQPEDVNGIMAQLPPRHVLDGLIRVYFKSV |
| Sce_apio_KEZ45621 | DELSPGSIS--SSIARDFTMQPWDAAGDLFRKLPSRLVTDSLLQSYFKNA |
| Sta_chlo_KFA62280 | DGPSPGTVT--STIARDFTQLPAADMDDMLAQFPPNHVLETLTHSYFKNV |
| Ver_alfa_XP_003000413 | DGPSPASVTSVTSIAKDFTRMPTVDLDETLRGLPADETLELLVQSYFKNV |
| Fus_oxys_EXL68817 | DGPSPGSIT--SAIARDFTRLPTADMDEILSQFPSNEILDLLIHSYFKNV |
| Fus_oxys_EXK46473 | DGPSPGSIT--SAIARDFTRLPTADMDEILGQFPSNEILDLLIQSYFKNV |
| Fus_oxys_EGU75021 | DGPSPGSIT--SAIARDFTRLPTADMDEILSQFPSNEILDLLIHSYFKNV |
| Fus_oxys_EXM14771 | DGPSPGSIT--SAIARDFTRLPTADMDEILGQFPSNEILDLLIQSYFKNV |
| Fus_oxys_EXM09676 | DGPSPGSIT--SAIARDFTRLPTADMDEILGQFPSNEILDLLIQSYFKNV |
| Fus_oxys_EXK77862 | DGPSPGSIT--SAIARDFTRLPTADMDEILGQFPSNEILDLLIHSYFKNV |
| | * ** : :::.:: : . :*. : * : ***.. |
| P_brasilianum_hmfR | HPDFALFHRGTFEEEYETFMSKGRYYHQHARAGVH---LSSPTLPEPGWL |
| Spo_sche_ERT02388 | HPDFPLFHRGTFEEEYERYIPDFESFYHPRRR-------TDTPTADPGWL |
| Sce_apio_KEZ45621 | HEDFPLFHRGTFEEEYESYWALLKQRITAPEP------CLQASQMEWGWV |
| Sta_chlo_KFA62280 | HSDFPLFHRATFEDEYELFVVQARR--RPPGRRQRP-------APDWGWI |
| Ver_alfa_XP_003000413 | HDDYPLFHRATFEDEYELYIVQARRRLQFLPQSQAQPQNRSNAVPDWGWM |
| Fus_oxys_EXL68817 | HDDFPLFHRATFEEEYESFIVEARRSSRLPSRPLR--------LPDWGWI |
| Fus_oxys_EXK46473 | HDDFPLFHRATFEEEYESFIVEARRSSRLPSRPLR--------LPDWGWI |
| Fus_oxys_EGU75021 | HDDFPLFHRATFEEEYESFIVEARRSSRLPSRPLR--------LPDWGWI |
| Fus_oxys_EXM14771 | HDDFPLFHRATFEEEYESFIVEARRSSCLPSRPLR--------LPDWGWI |
| Fus_oxys_EXM09676 | HDDFPLFHRATFEEEYESFIVEARRSSRLPSRPLR--------LPDWGWI |
| Fus_oxys_EXK77862 | HDDFPLFHRATFEEEYESFIVEARRSSRLPSRPLR--------LPDWGWI |
| | * *:.****.***:*** : . : **: |
| P_brasilianum_hmfR | GCLHMMIAFASLN------------------------GSVDVAPDLDLTS |
| Spo_sche_ERT02388 | GCLHMILAFASLVTPAVSSSASHHRPPPSTATPSTAASSRQTHDDVDLAA |
| Sce_apio_KEZ45621 | ATLQMLIVFGSMCDP--------------------------SIPGIDHTT |
| Sta_chlo_KFA62280 | GCLHMMCVFGSISDP--------------------------GATGLDHSE |
| Ver_alfa_XP_003000413 | GCLHMILVFGSIARP--------------------------DIPGVDHSH |
| Fus_oxys_EXL68817 | GCLHMIVVFGSIADR--------------------------SIPNVDHSA |
| Fus_oxys_EXK46473 | GCLHMIVVFGSIADR--------------------------SIPNVDHSA |
| Fus_oxys_EGU75021 | GCLHMIVVFGSIADR--------------------------SIPNVDHSA |
| Fus_oxys_EXM14771 | GCLHMIVVFGSIADR--------------------------SIPNVDHSA |
| Fus_oxys_EXM09676 | GCLHMIVVFGSIADR--------------------------SIPNVDHSA |
| Fus_oxys_EXK77862 | GCLHMIVVFGSIADR--------------------------SIPNVDHSA |
| | . *:*: .*.*: .:* : |
| P_brasilianum_hmfR | LCRHCASLTR-QLLPQFISKCTLSNVRALLLLSLFLHNHNERNAAWNLVG |
| Spo_sche_ERT02388 | LRKHCVSLTRFRLLPRFISRCTLANIRALLLLALYLHNHNERNAAWNLVG |
| Sce_apio_KEZ45621 | LRRQCVSVTR-SLLPQLVSKCTLSNVRALLLLSLFLHNNNERNAAWNLVG |
| Sta_chlo_KFA62280 | LRRRCVMATR-MLLPQFVSKCTLSNVRVLLLLSLFLHNNNERNAAWNLVG |
| Ver_alfa_XP_003000413 | LRRRSVAAAR-TLLPQFISKCTLSNVRVLMLLSLFLHNNNERNAAWNLVG |
| Fus_oxys_EXL68817 | LRRRSIAVAR-GLLPQFISKCSLTNVRVLLLLSLFLHNNNERNAAWNIAG |
| Fus_oxys_EXK46473 | LRRRSIAVAR-GLLPQFISKCSLTNVRVLLLLSLFLHNNNERNAAWNIAG |
| Fus_oxys_EGU75021 | LRRRSIAVAR-GLLPQFISKCSLTNVRVLLLLSLFLHNNNERNAAWNIAG |
| Fus_oxys_EXM14771 | LRRRSIAVAR-GLLPQFISKCSLTNVRVLLLLSLFLHNNNERNAAWNIAG |
| Fus_oxys_EXM09676 | LRRRSIAVAR-GLLPQFISKCSLTNVRVLLLLSLFLHNNNERNAAWNIAG |
| Fus_oxys_EXK77862 | LRRRSIAVAR-GLLPQFISKCSLTNVRVLLLLSLFLHNNNERNAAWNIAG |
| | * ::. :* ***:::*:*:*:*:*.*:**:*:***:********:.* |
| P_brasilianum_hmfR | TAMRLSFAMGLHRASDNGSHFRPIEREVRKRVFCTLYGFEQFLASSLGRP |
| Spo_sche_ERT02388 | TATRAAFAMGLHRCTVGAEHLRPVEREVRRRVFCTLFGLEQFLASSLGRP |
| Sce_apio_KEZ45621 | TATRISFALGLHRR-DVAAYFRPIEREVRKRVFCTLYSFEQFLASSLGRP |
| Sta_chlo_KFA62280 | TATRISFALGLHRA-TMLASLRPQEREVRKWVFCTLYAFEQFLASSLGRP |
| Ver_alfa_XP_003000413 | TATRIAFALGLHRS-DMRSSLRPLDREVRKWVFCTLYSFEQFLASSLGRP |
| Fus_oxys_EXL68817 | TATRISFALGLHRS-DMSVSFRPLEREVRKWVFCTLYSFEQFLASSLGRP |
| Fus_oxys_EXK46473 | TATRISFALGLHRS-DMSVSFRPLEREVRKWVFCTLYSFEQFLASSLGRP |
| Fus_oxys_EGU75021 | TATRISFALGLHRS-DMSVSFRPLEREVRKWVFCTLYSFEQFLASSLGRP |
| Fus_oxys_EXM14771 | TATRISFALGLHRS-DMSVSFRPLEREVRKWVFCTLYSFEQFLASSLGRP |
| Fus_oxys_EXM09676 | TATRISFALGLHRS-DMSASFRPLEREVRKWVFCTLYSFEQFLASSLGRP |
| Fus_oxys_EXK77862 | TATRISFALGLHRS-DMSVSFRPLEREVRKWVFCTLYSFEQFLASSLGRP |
| | ** * :**:**** :** :****: *****:.:*********** |
| P_brasilianum_hmfR | SGFY---------DFEDVEIVPPREGVLDSG-----QDEDDEVMKLSLRL |
| Spo_sche_ERT02388 | SGLSGLSALSSANDANEVEVVPPRAEILDGGGSADADDDDGAMATLLLRL |
| Sce_apio_KEZ45621 | SGLN----------DFDVEIALPREGLLGTG--------TDRVVALSLKL |
| Sta_chlo_KFA62280 | SGLQ----------DVDVEVVPPRDGFLDVG--------DAQLARLSLRL |
| Ver_alfa_XP_003000413 | SGLQ----------EMDVEIVPPREGFLDAGT-----GTDAKLVFLSLRL |
| Fus_oxys_EXL68817 | SGLQ----------ELDVEVVPPREGFVEGGI-----GTDARLVSWSVKL |
| Fus_oxys_EXK46473 | SGLQ----------ELDVEVVPPREGFVEGGV-----GTDARLVSWSVKL |
| Fus_oxys_EGU75021 | SGLQ----------ELDVEVVPPREGFVEGGI-----GTDARLVSWSVKL |
| Fus_oxys_EXM14771 | SGLQ----------ELDVEVVPPREGFVEGGI-----GTDARLVSWSVKL |
| Fus_oxys_EXM09676 | SGLQ----------ELDVEVVPPREGFVEGGI-----GTDARLVSWSVKL |
| Fus_oxys_EXK77862 | SGLQ----------ELDVEVVPPREGFVEGGI-----GTDARLVSWSVKL |
| | **: :**:. ** .: * : ::* |
| P_brasilianum_hmfR | QVILAKARVSLAVKTLAVANERGNIDGLARQQQSSRETLEILKAWREDLA |
| Spo_sche_ERT02388 | QTILAGARVSAAVKTVGLGSRR------LRQEQSAREILQRLDEWRTAVA |
| Sce_apio_KEZ45621 | QNILGRARISQAVRSLASGNTDT-----QRHEESAKETISALKAWRDEVA |
| Sta_chlo_KFA62280 | DGILAKARLLHAGRARGTAADG------AGSPPDLEGVLGALEEWKKEAA |
| Ver_alfa_XP_003000413 | QAILARTRFAYARPQRRPDAEGQD----VVPRPSVDDIMRSLAAWKRDVA |
| Fus_oxys_EXL68817 | QAILARTRLLHVDINR-------------SSGPTLDEILTALNGWKRDIG |
| Fus_oxys_EXK46473 | QAILARTRLLHVDINR-------------SSGPTLDEILTALNGWKRDIG |
| Fus_oxys_EGU75021 | QAILARTRLLHVDINR-------------SSGPTLDEILTALNGWKRDIG |
| Fus_oxys_EXM14771 | QAILARTRLLHVGINQ-------------SLGPTLDEILTALDGWKRDIG |
| Fus_oxys_EXM09676 | QAILARTRLLHVDINR-------------SSGPTLDEILTALNGWKRDIG |
| Fus_oxys_EXK77862 | QAILARTRLLHVGINQ-------------SSGPTLDEILTALNGWKRDIG |
| | : **. :*. . : * *: . |
| P_brasilianum_hmfR | SHHILNIPLISETDDP-------LCQYAEEIPRMSLQDLKAMMGWQSRPR |
| Spo_sche_ERT02388 | GCRCLDIPQITETTDSGRDAFVADAPPSTSTPSMDLDSLKNMLAWQSRPR |
| Sce_apio_KEZ45621 | ASQSLNIPSISEPDDA--------FKEDDAPVTMSFTEIKLLLSWQDRTR |
| Sta_chlo_KFA62280 | RQAGCDVPWVRTG---------KAFP--AKTAAVDMDELKAMLSWKTRAQ |
| Ver_alfa_XP_003000413 | ENPSFHMPDIQTRVSL-RGRGSSASLHDEDGDAMEFDELKVVLSWKTRAQ |
| Fus_oxys_EXL68817 | KAPGLDVSWIKMEG--------PALESIDHEGAVDMEELKVSLAWKTRAQ |
| Fus_oxys_EXK46473 | KAPGLDVSWIKMEG--------PALESIDHEGAVAMEELKVSLAWKTRAQ |
| Fus_oxys_EGU75021 | KAPGLDVSWIKMEG--------PALESIDHEGAVDMEELKVSLAWKTRAQ |
| Fus_oxys_EXM14771 | KAPGLDVSWIKMEG--------PALESIDHEGAVDMEELKVSLAWKTRAQ |
| Fus_oxys_EXM09676 | KAPGLDVSWIKMEG--------PALESIDHEGAVDMEELKVSLARKTRAQ |
| Fus_oxys_EXK77862 | KAPGLDVSWIKMEG--------PALESIDHEGAVDMEGLKVSLTWKTRAQ |
| | .:. : : : :* : : *.: |
| P_brasilianum_hmfR | LRAALVLHLQYRYIAVLVTRSSLLRYVASAQRGEPEHEALLSRNEARTDP |
| Spo_sche_ERT02388 | LRAALVLHMQYRYVAVLSTRSALL-YSMAARAARTAPVAHDGGPAPSPSP |
| Sce_apio_KEZ45621 | LRAALVLNMQYRYIAIMVARPFLLRDTAMAR-----VVARTDNKNTTNDT |
| Sta_chlo_KFA62280 | LRAVLLLHIQYYYINIVATRPLLLRDIAKLG---------ATTADPAPPG |
| Ver_alfa_XP_003000413 | LRAVLMLHIQYHYIAIVATRPILLREIAAAR---------KALRDESAG- |
| Fus_oxys_EXL68817 | LRAVLLLHIHFHYIAIVATRPLLLRDVAAAR---------KEDAPKTP-- |
| Fus_oxys_EXK46473 | LRAVLLLHIHFHYIAIVATRPLLLREVAAAR---------KEDAPKTP-- |
| Fus_oxys_EGU75021 | LRAVLLLHIHFHYIAIVATRPLLLRDVAAAR---------KEDAPKTP-- |
| Fus_oxys_EXM14771 | LRAVLLLHIHFHYIAIVATRPLLLRDVAAAR---------KEDAPKTP-- |
| Fus_oxys_EXM09676 | LRAVLLLHIHFHYIAIVATRPLLLRDVAAAR---------KEDAPKTP-- |
| Fus_oxys_EXK77862 | LRAVLLLHIHFHYIAIVATRPLLLRDVAAAR---------KEDAPKTP-- |
| | ***.*:*:::: *: :: :*. ** |
| P_brasilianum_hmfR | YNSEAGERLSDICVTHATQLCRLILLADSFGLVNGISAMDVFYVYCGVMV |
| Spo_sche_ERT02388 | ATSAAPPTLADLCVQNAVQLCRLVLLADSFGLINGVSAMDVFYAYCAAMV |
| Sce_apio_KEZ45621 | PRSDAHSHLASVCVQNACQLAKIVLLLAEFELLNGVCGMDVFYAYSASM- |
| Sta_chlo_KFA62280 | AGVPALSPHAESCVRHACQLAHLVVLLDGFGVINGLSGLDVFYAYCAAMV |
| Ver_alfa_XP_003000413 | APPPAMSAVADACVRHAVQLTYMVLFLDGFELVNGLSGLDVFYAYCAAMV |
| Fus_oxys_EXL68817 | -----VPTHAALCVKHACQLSYLMILLDHFDVINGLSGLDIFYAYCSAMI |
| Fus_oxys_EXK46473 | -----VPTHAALCVKHACQLSYLMILLDHFDVINGLSGLDIFYAYCSAMI |
| Fus_oxys_EGU75021 | -----VPTHAALCVKHACQLSYLMILLDHFDVINGLSGLDIFYAYCSAMI |
| Fus_oxys_EXM14771 | -----VPTHAALCVKHACQLSYLMILLDHFDVINGLSGLDIFYAYCSAMI |
| Fus_oxys_EXM09676 | -----VPTHAALCVKHACQLSYLMILLDHFDVINGLSGLDIFYAYCSAMI |
| Fus_oxys_EXK77862 | -----VPTHAALCVKHACQLSYLMILLDHFDVINGLSGLDIFYAYCSAMI |
| | : ** :* ** :::: * ::**:..:*:**.*.. * |
| P_brasilianum_hmfR | LILRSLRISS---SASHYHDQREAHLQLELRKLIAQTREVLIRVNKCSTM |
| Spo_sche_ERT02388 | LILRSLNGGSEQDQGAVSVSAADAAYCAELRRLIARTRQVLMRVDKCSTM |
| Sce_apio_KEZ45621 | --------------------------------LIQSIRLVVSKVPKSGTM |
| Sta_chlo_KFA62280 | LILRLAR--AGRQDDGGEEEEEEKMLG-AVRELVSDLRRVMNRTQKGGSM |
| Ver_alfa_XP_003000413 | LILRLLRR-PPAAEGAEASDQQEEQIQVVIRELVRKSQSVLNRTNKSGSM |
| Fus_oxys_EXL68817 | LILRLLR--LRPGESAESIGPDEVMLQSKVRRLVATLRNVINHTDKCGSM |
| Fus_oxys_EXK46473 | LILRLLR--LRPGEGAESIGPDEVILQSKVRRLVATLRNVINHTDKCGSM |
| Fus_oxys_EGU75021 | LILRLLR--LRPGESAESIGPDEVMLQSKVRRLVATLRNVINHTDKCGSM |
| Fus_oxys_EXM14771 | LILRLLR--LRPGEGAESIGPDEVMLQSKVRRLVATLRNVINHTDKCGSM |
| Fus_oxys_EXM09676 | LILRLLR--LRPG---EGIGPDEVMLQSKVRRLVATLRNVINHTDKCGSM |
| Fus_oxys_EXK77862 | LILRLLR--LRPGEGAESIGPDEVMLQSKVRRLVATLRNVINHTDKCGSM |
| | *: : *: :. * .:* |
| P_brasilianum_hmfR | KRFARVVATFEDGSR---QDNIRPADGSTNRS--------TANCEMRTAR |
| Spo_sche_ERT02388 | KRFSRVVATFEEGSRRVGRDDVHQNSNTANTANTAGDGTVPAHPSSTTAH |
| Sce_apio_KEZ45621 | KRFARVMATFEDSVFN---HDALPHAATPRKD------------------ |
| Sta_chlo_KFA62280 | RRFARVVDTFFEAVDKP--SPRLKMSGHG-----------HNGPSMQGVP |
| Ver_alfa_XP_003000413 | KRFASVVDAFAECTSQTPGTQEDKVRALPGSA-------WSRGFSGGGVS |
| Fus_oxys_EXL68817 | KRLAQVVDTFSECANNP--TDPPGIANLP-----------PQGINMNNPP |
| Fus_oxys_EXK46473 | KRLAQVVDTFSECANNP--TDPPGIANLP-----------PQGINMNNPP |
| Fus_oxys_EGU75021 | KRLAQVVDTFSECANNP--TDPPGIANLP-----------PQGINMNNPP |
| Fus_oxys_EXM14771 | KRLAQVVDTFSECANNP--TDPPGIANLP-----------PQGINMNNPP |
| Fus_oxys_EXM09676 | KRLAQVVDTFSECANNP--TDPPGIANLP-----------PQGINMNNPP |
| Fus_oxys_EXK77862 | KRLAQVVDTFSECANNP--TDPPGIANLP-----------PQGINMNNPP |
| | :*:: *: :* : |
| P_brasilianum_hmfR | --------QASRDPRGRFN---------------HSIHAALDGGRASNLA |
| Spo_sche_ERT02388 | PRHPPPSPYAPPAPRQRQTPAHGPAAVHTPSQAPPSVTRRLASMSSQSSA |
| Sce_apio_KEZ45621 | -----------------------------------SGTQLHATGQDIPAI |
| Sta_chlo_KFA62280 | APHLQ--------------------------QQQNTSFFYPYGQRQQQMT |
| Ver_alfa_XP_003000413 | ALPR-----------------------------------QPAALDAGQFP |
| Fus_oxys_EXL68817 | ---------------------------------------YPAGWSADQVQ |
| Fus_oxys_EXK46473 | ---------------------------------------YPDGWSAEKIQ |
| Fus_oxys_EGU75021 | ---------------------------------------YPAGWSADQVQ |
| Fus_oxys_EXM14771 | ---------------------------------------YPAGWSAEQVQ |
| Fus_oxys_EXM09676 | ---------------------------------------YPAGWSAEQVQ |
| Fus_oxys_EXK77862 | ---------------------------------------YPAGWSAEQVQ |
| P_brasilianum_hmfR | IFPGAGGSLDTSSS--LPVSQQE----PLNFQHGYGNGIGPRLG------ |
| Spo_sche_ERT02388 | LHVDESQRLHMSPS--QTSQTTQTTLPPQNQAHFASAGVGALCSNGYDQY |
| Sce_apio_KEZ45621 | HHLGSTDPLLLAP---QPATASAFLDPSFPMMAGWPQGDWSTFG------ |
| Sta_chlo_KFA62280 | LN-DQGLVLGPDLLGEHAGAAPRLGDAGTFGDAWLELLPLSTFGGS---- |
| Ver_alfa_XP_003000413 | YGMMGTGVIG------------VPPGQAFSMTAPMGFGQATTYGVLN--- |
| Fus_oxys_EXL68817 | AQQGQGMALG-------------------SMEGLLDFLPFPGFG------ |
| Fus_oxys_EXK46473 | AQQDQGMALG-------------------SMEGLLDFLPFPGFG------ |
| Fus_oxys_EGU75021 | AQQGQGMALG-------------------SMEGLLDFLPFPGFG------ |
| Fus_oxys_EXM14771 | AQHGQGMALG-------------------SMEGLLDFLPFPGFG------ |
| Fus_oxys_EXM09676 | AQQGQGMALG-------------------SMEGLLDFLPFPGFG------ |
| Fus_oxys_EXK77862 | AQHGQGMALG-------------------SMEGLLDFLPFPGFG------ |
| | : . . |
| P_brasilianum_hmfR | ----------ISDPFW-------QPNLLTSFDGEPEANGWMMDPFL-AMD |
| Spo_sche_ERT02388 | GHAQSHLHPHSSFPPWPGQPMGPQPGLTSLFDGEPEENQWVMDTFL-GMG |
| Sce_apio_KEZ45621 | ------------------------------ADDGREFGGWIASLLQPAMD |
| Sta_chlo_KFA62280 | ----------------------------------RIVEGMFPNLEG-ASE |
| Ver_alfa_XP_003000413 | --------------------------------VQLDDGGFYFHPFN-GSE |
| Fus_oxys_EXL68817 | -----------------------------------MAEGSMAQYVP-GSE |
| Fus_oxys_EXK46473 | -----------------------------------MAEGSMAQYVP-GSE |
| Fus_oxys_EGU75021 | -----------------------------------MAEGSMAQYVP-GSE |
| Fus_oxys_EXM14771 | -----------------------------------MAEGSMAQYVP-GSE |
| Fus_oxys_EXM09676 | -----------------------------------MAEGSMAQYVP-GSE |
| Fus_oxys_EXK77862 | -----------------------------------MAEGSMAQYVP-GSE |
| | . |
| P_brasilianum_hmfR | G---------------------TGVVDWGDIESLLSRNPGQ--------- |
| Spo_sche_ERT02388 | MGMGMHPGSGGSVEGDIDGVFSAGMLDWPDMDAIMRNG------------ |
| Sce_apio_KEZ45621 | T---------------------PMVTEFGDMDSILRNAPM---------- |
| Sta_chlo_KFA62280 | G---------------------VGGHDWVDMQILLGAYGGQGP------- |
| Ver_alfa_XP_003000413 | T---------------------TAPPEWGDMEMVMAGYGMPRS------- |
| Fus_oxys_EXL68817 | ----------------------MEMTGWHDMEFLMEGYGDQSR------- |
| Fus_oxys_EXK46473 | ----------------------MEMTGWHDMEFLMEGYGDQSR------- |
| Fus_oxys_EGU75021 | ----------------------MEMTGWHDMEFLMEGYGDQIIGEGVEPV |
| Fus_oxys_EXM14771 | ----------------------MEMTGWHDMEFLMEGYGDQSK------- |
| Fus_oxys_EXM09676 | ----------------------MEMTGWHDMEFLMEGYGDQSR------- |
| Fus_oxys_EXK77862 | ----------------------MEMTGWHDMEFLMEGYGDQSK------- |
| | : *:: :: |
| P_brasilianum_hmfR | ---------------------------------- |
| Spo_sche_ERT02388 | ---------------------------------- |
| Sce_apio_KEZ45621 | ---------------------------------- |
| Sta_chlo_KFA62280 | ---------------------------VM----- |
| Ver_alfa_XP_003000413 | ---------------------------------- |
| Fus_oxys_EXL68817 | ---------------------------TNY---- |
| Fus_oxys_EXK46473 | ---------------------------TNY---- |
| Fus_oxys_EGU75021 | DVWRSQLQATVALEADDEPSSIQEGLTPNYTMDI |
| Fus_oxys_EXM14771 | ---------------------------TNY---- |
| Fus_oxys_EXM09676 | ---------------------------INY---- |
| Fus_oxys_EXK77862 | ---------------------------TNY---- |

TABLE 1A
Percentage amino acid sequence identity among Penicillium brasilianum hmfL1 orthologogues and accession numbers thereof.

| Species | Accession | | | | | | | | | | | |
|---|---|---|---|---|---|---|---|---|---|---|---|---|
| Penicillium | SEQ ID NO: 1 | 100.00 | 73.80 | 49.70 | 48.30 | 48.40 | 49.80 | 51.30 | 41.50 | 41.50 | 43.30 | 43.40 |
| brasilianum | | | | | | | | | | | | |
| hmfL1 | | | | | | | | | | | | |
| Sporothrix | ERT02385 | 73.80 | 100.00 | 48.60 | 49.10 | 49.00 | 50.80 | 51.00 | 40.20 | 40.20 | 44.60 | 43.80 |
| schenckii | | | | | | | | | | | | |
| ATCC 58251 | | | | | | | | | | | | |
| Aspergillus | GAA84694 | 49.70 | 48.60 | 100.00 | 61.40 | 84.90 | 61.50 | 84.70 | 38.10 | 38.10 | 35.50 | 39.90 |
| kawachii | | | | | | | | | | | | |
| IFO 4308 | | | | | | | | | | | | |
| Byssochlamys | GAD98038 | 48.30 | 49.10 | 61.40 | 100.00 | 60.90 | 66.60 | 62.30 | 41.80 | 41.80 | 39.00 | 40.50 |
| spectabilis | | | | | | | | | | | | |
| No. 5 | | | | | | | | | | | | |
| Aspergillus | XP_001397354 | 48.40 | 49.00 | 84.90 | 60.90 | 100.00 | 60.00 | 99.70 | 36.60 | 36.60 | 34.50 | 38.00 |
| niger | | | | | | | | | | | | |
| CBS 513.88 | | | | | | | | | | | | |
| Eutypa lata | XP_007796771 | 49.80 | 50.80 | 61.50 | 66.60 | 60.00 | 100.00 | 62.10 | 38.90 | 38.90 | 35.60 | 38.30 |
| UCREL1 | | | | | | | | | | | | |
| Aspergillus | EHA21652 | 51.30 | 51.00 | 84.70 | 62.30 | 99.70 | 62.10 | 100.00 | 39.00 | 39.00 | 35.80 | 39.00 |
| niger | | | | | | | | | | | | |
| ATCC 1015 | | | | | | | | | | | | |
| Fusarium | EYB30957 | 41.50 | 40.20 | 38.10 | 41.80 | 36.60 | 38.90 | 39.00 | 100.00 | 99.70 | 41.20 | 41.30 |
| graminearum | | | | | | | | | | | | |
| Fusarium | XP_011318199 | 41.50 | 40.20 | 38.10 | 41.80 | 36.60 | 38.90 | 39.00 | 99.70 | 100.00 | 41.50 | 41.30 |
| graminearum | | | | | | | | | | | | |
| PH-1 | | | | | | | | | | | | |
| Rhizobium | WP_016737077 | 43.30 | 44.60 | 35.50 | 39.00 | 34.50 | 35.60 | 35.80 | 41.20 | 41.50 | 100.00 | 67.70 |
| phaseoli | | | | | | | | | | | | |
| Dyella | WP_038619920 | 43.40 | 43.80 | 39.90 | 40.50 | 38.00 | 38.30 | 39.00 | 41.30 | 41.30 | 67.70 | 100.00 |
| jiangningensis | | | | | | | | | | | | |

TABLE 2A
Percentage amino acid sequence identity among Penicillium brasilianum hmfL2 orthologogues and accession numbers thereof.

| Species | Accession | | | | | | | | | | | |
|---|---|---|---|---|---|---|---|---|---|---|---|---|
| Penicillium | SEQ ID NO: 2 | 100.00 | 69.30 | 68.10 | 67.70 | 64.00 | 64.30 | 63.70 | 64.00 | 67.10 | 67.00 | 63.90 |
| brasilianum | | | | | | | | | | | | |
| hmfL2 | | | | | | | | | | | | |
| Coccidioides | XP_001244132 | 69.30 | 100.00 | 97.30 | 96.70 | 67.60 | 68.10 | 68.40 | 67.80 | 70.10 | 66.10 | 69.20 |
| immitis RS | | | | | | | | | | | | |
| Coccidioides | XP_003068662 | 68.10 | 97.30 | 100.00 | 98.20 | 65.00 | 65.70 | 67.80 | 65.30 | 67.60 | 66.60 | 69.50 |
| posadasii C735 | | | | | | | | | | | | |
| delta SOWgp | | | | | | | | | | | | |
| Coccidioides | EFW20539 | 67.70 | 96.70 | 98.20 | 100.00 | 64.70 | 65.30 | 67.40 | 65.00 | 67.20 | 66.20 | 69.10 |
| posadasii | | | | | | | | | | | | |
| str. Silveira | | | | | | | | | | | | |
| Trichophyton | XP_003235253 | 64.00 | 67.60 | 65.00 | 64.70 | 100.00 | 97.60 | 65.20 | 97.30 | 89.10 | 65.20 | 82.00 |
| rubrum | | | | | | | | | | | | |
| CBS 118892 | | | | | | | | | | | | |
| Trichophyton | EGE05431 | 64.30 | 68.10 | 65.70 | 65.30 | 97.60 | 100.00 | 64.60 | 99.70 | 88.80 | 65.20 | 82.50 |
| equinum | | | | | | | | | | | | |
| CBS 127.97 | | | | | | | | | | | | |
| Chaetomium | XP_001220755 | 63.70 | 68.40 | 67.80 | 67.40 | 65.20 | 64.60 | 100.00 | 64.30 | 66.80 | 64.90 | 63.30 |
| globosum | | | | | | | | | | | | |
| CBS 148.51 | | | | | | | | | | | | |
| Trichophyton | EGD92820 | 64.00 | 67.80 | 65.30 | 65.00 | 97.30 | 99.70 | 64.30 | 100.00 | 88.50 | 64.90 | 82.20 |
| tonsurans | | | | | | | | | | | | |
| CBS 112818 | | | | | | | | | | | | |
| Microsporum | XP_003173798 | 67.10 | 70.10 | 67.60 | 67.20 | 89.10 | 88.80 | 66.80 | 88.50 | 100.00 | 65.60 | 85.20 |
| gypseum | | | | | | | | | | | | |
| CBS 118893 | | | | | | | | | | | | |
| Endocarpon | XP_007800835 | 67.00 | 66.10 | 66.60 | 66.20 | 65.20 | 65.20 | 64.90 | 64.90 | 65.60 | 100.00 | 67.60 |
| pusillum | | | | | | | | | | | | |
| Z07020 | | | | | | | | | | | | |
| Arthroderma | XP_002844685 | 63.90 | 69.20 | 69.50 | 69.10 | 82.00 | 82.50 | 63.30 | 82.20 | 85.20 | 67.60 | 100.00 |
| otae CBS | | | | | | | | | | | | |
| 113480 | | | | | | | | | | | | |

TABLE 3A
Percentage amino acid sequence identity among Penicillium brasilianum hmfN1 orthologogues and accession numbers thereof.

| Species | Accession | | | | | | | | | | | |
|---|---|---|---|---|---|---|---|---|---|---|---|---|
| Penicillium | SEQ ID NO: 3 | 100.00 | 70.80 | 64.90 | 62.80 | 64.20 | 62.70 | 61.90 | 62.50 | 62.50 | 57.90 | 60.80 |
| brasilianum | | | | | | | | | | | | |
| hmfN1 | | | | | | | | | | | | |
| Sporothrix | ERT02387 | 70.80 | 100.00 | 59.90 | 58.10 | 62.10 | 60.10 | 59.30 | 59.90 | 60.10 | 52.90 | 57.50 |
| schenckii | | | | | | | | | | | | |
| ATCC 58251 | | | | | | | | | | | | |
| Scedosporium | KEZ45623 | 64.90 | 59.90 | 100.00 | 64.30 | 67.20 | 64.70 | 65.30 | 64.30 | 64.50 | 59.90 | 61.00 |
| apiospermum | | | | | | | | | | | | |
| Podospora | XP_001908521 | 62.80 | 58.10 | 64.30 | 100.00 | 64.20 | 63.50 | 67.70 | 63.50 | 63.50 | 58.10 | 60.20 |
| anserina | | | | | | | | | | | | |
| S mat+ | | | | | | | | | | | | |
| Eutypa lata | XP_007794079 | 64.20 | 62.10 | 67.20 | 64.20 | 100.00 | 69.00 | 64.70 | 68.40 | 68.60 | 63.20 | 66.70 |
| UCREL1 | | | | | | | | | | | | |
| Stachybotrys | KEY72856 | 62.70 | 60.10 | 64.70 | 63.50 | 69.00 | 100.00 | 63.30 | 99.20 | 99.60 | 62.20 | 92.80 |
| chartarum | | | | | | | | | | | | |
| IBT 7711 | | | | | | | | | | | | |
| Gaeumannomyces | XP_009217152 | 61.90 | 59.30 | 65.30 | 67.70 | 64.70 | 63.30 | 100.00 | 63.30 | 63.10 | 59.10 | 61.20 |
| graminis var. | | | | | | | | | | | | |
| tritici R3111a-1 | | | | | | | | | | | | |
| Stachybotrys | KFA73399 | 62.50 | 59.90 | 64.30 | 63.50 | 68.40 | 99.20 | 63.30 | 100.00 | 98.80 | 62.20 | 92.00 |
| chartarum | | | | | | | | | | | | |
| IBT 40288 | | | | | | | | | | | | |
| Stachybotrys | KFA53356 | 62.50 | 60.10 | 64.50 | 63.50 | 68.60 | 99.60 | 63.10 | 98.80 | 100.00 | 61.80 | 92.60 |
| chartarum | | | | | | | | | | | | |
| IBT 40293 | | | | | | | | | | | | |
| Cyphellophora | XP_008712551 | 57.90 | 52.90 | 59.90 | 58.10 | 63.20 | 62.20 | 59.10 | 62.20 | 61.80 | 100.00 | 59.50 |
| europaea | | | | | | | | | | | | |
| CBS 101466 | | | | | | | | | | | | |
| Stachybotrys | KFA62282 | 60.80 | 57.50 | 61.00 | 60.20 | 66.70 | 92.80 | 61.20 | 92.00 | 92.60 | 59.50 | 100.00 |
| chlorohalonata | | | | | | | | | | | | |
| IBT 40285 | | | | | | | | | | | | |

TABLE 4A
Percentage amino acid sequence identity among Penicillium brasilianum hmfK1 orthologogues and accession numbers thereof.

| Species | Accession | | | | | | | | | | | |
|---|---|---|---|---|---|---|---|---|---|---|---|---|
| Penicillium | SEQ ID NO: 4 | 100.00 | 82.20 | 80.60 | 73.90 | 73.90 | 80.00 | 78.50 | 74.20 | 74.50 | 72.80 | 69.80 |
| brasilianum | | | | | | | | | | | | |
| hmfK1 | | | | | | | | | | | | |
| Scedosporium | KEZ45619 | 82.20 | 100.00 | 79.60 | 76.10 | 76.10 | 77.50 | 78.50 | 76.30 | 71.70 | 73.10 | 68.40 |
| apiospermum | | | | | | | | | | | | |
| Togninia | XP_007916105 | 80.60 | 79.60 | 100.00 | 75.60 | 75.60 | 76.10 | 82.60 | 76.30 | 70.30 | 75.60 | 70.50 |
| minima | | | | | | | | | | | | |
| UCRPA7 | | | | | | | | | | | | |
| Stachybotrys | KEY72859 | 73.90 | 76.10 | 75.60 | 100.00 | 99.80 | 73.40 | 73.50 | 95.40 | 68.40 | 73.80 | 67.40 |
| chartarum | | | | | | | | | | | | |
| IBT 7711 | | | | | | | | | | | | |
| Stachybotrys | KFA53358 | 73.90 | 76.10 | 75.60 | 99.80 | 100.00 | 73.40 | 73.50 | 95.20 | 68.40 | 73.80 | 67.40 |
| chartarum | | | | | | | | | | | | |
| IBT 40293 | | | | | | | | | | | | |
| Sporothrix | ERT02390 | 80.00 | 77.50 | 76.10 | 73.40 | 73.40 | 100.00 | 72.50 | 72.90 | 75.50 | 69.10 | 66.00 |
| schenckii | | | | | | | | | | | | |
| ATCC 58251 | | | | | | | | | | | | |
| Eutypa lata | XP_007794919 | 78.50 | 78.50 | 82.60 | 73.50 | 73.50 | 72.50 | 100.00 | 74.30 | 67.70 | 72.10 | 69.70 |
| UCREL1 | | | | | | | | | | | | |
| Stachybotrys | KFA62283 | 74.20 | 76.30 | 76.30 | 95.40 | 95.20 | 72.90 | 74.30 | 100.00 | 67.50 | 73.00 | 67.10 |
| chlorohalonata | | | | | | | | | | | | |
| IBT 40285 | | | | | | | | | | | | |
| Grosmannia | EFX06428 | 74.50 | 71.70 | 70.30 | 68.40 | 68.40 | 75.50 | 67.70 | 67.50 | 100.00 | 65.30 | 64.20 |
| clavigera | | | | | | | | | | | | |
| kw1407 | | | | | | | | | | | | |
| Cyphellophora | XP_008712555 | 72.80 | 73.10 | 75.60 | 73.80 | 73.80 | 69.10 | 72.10 | 73.00 | 65.30 | 100.00 | 70.60 |
| europaea | | | | | | | | | | | | |
| CBS 101466 | | | | | | | | | | | | |
| Byssochlamys | GAD98036 | 69.80 | 68.40 | 70.50 | 67.40 | 67.40 | 66.00 | 69.70 | 67.10 | 64.20 | 70.60 | 100.00 |
| spectabilis | | | | | | | | | | | | |
| No. 5 | | | | | | | | | | | | |

TABLE 5A
Percentage amino acid sequence identity among Penicillium brasilianum hmfM orthologogues and accession numbers thereof.

| Species | Accession | | | | | | | | | | | |
|---|---|---|---|---|---|---|---|---|---|---|---|---|
| Penicillium | SEQ ID NO: 5 | 100.00 | 73.50 | 64.90 | 64.50 | 60.40 | 64.50 | 59.60 | 60.80 | 60.80 | 60.40 | 60.40 |
| brasilianum | | | | | | | | | | | | |
| hmfM | | | | | | | | | | | | |
| Aspergillus | XP_664054 | 73.50 | 100.00 | 60.80 | 62.90 | 60.40 | 69.00 | 58.80 | 58.40 | 58.40 | 58.00 | 58.00 |
| nidulans | | | | | | | | | | | | |
| FGSC A4 | | | | | | | | | | | | |
| Eutypa lata | XP_007797627 | 64.90 | 60.80 | 100.00 | 65.00 | 58.50 | 56.90 | 61.80 | 65.40 | 65.00 | 65.40 | 65.40 |
| UCREL1 | | | | | | | | | | | | |
| Thielavia | XP_003656972 | 64.50 | 62.90 | 65.00 | 100.00 | 58.50 | 61.80 | 55.70 | 65.40 | 64.60 | 65.00 | 65.00 |
| terrestris | | | | | | | | | | | | |
| NRRL 8126 | | | | | | | | | | | | |
| Trichoderma | EHK50353 | 60.40 | 60.40 | 58.50 | 58.50 | 100.00 | 59.80 | 85.00 | 57.30 | 58.10 | 57.70 | 57.70 |
| atroviride | | | | | | | | | | | | |
| IMI 206040 | | | | | | | | | | | | |
| Aspergillus | XP_001212987 | 64.50 | 69.00 | 56.90 | 61.80 | 59.80 | 100.00 | 59.30 | 58.90 | 58.90 | 58.50 | 57.40 |
| terreus | | | | | | | | | | | | |
| NIH2624 | | | | | | | | | | | | |
| Trichoderma | XP_006962638 | 59.60 | 58.80 | 61.80 | 55.70 | 85.00 | 59.30 | 100.00 | 55.70 | 56.50 | 56.10 | 56.10 |
| reesei QM6a | | | | | | | | | | | | |
| Fusarium | EMT67544 | 60.80 | 58.40 | 65.40 | 65.40 | 57.30 | 58.90 | 55.70 | 100.00 | 99.20 | 99.20 | 99.60 |
| oxysporum | | | | | | | | | | | | |
| f. sp. cubense | | | | | | | | | | | | |
| race 4 | | | | | | | | | | | | |
| Fusarium | EGU79882 | 60.80 | 58.40 | 65.00 | 64.60 | 58.10 | 58.90 | 56.50 | 99.20 | 100.00 | 99.20 | 99.60 |
| oxysporum | | | | | | | | | | | | |
| Fo5176 | | | | | | | | | | | | |
| Fusarium | EXL52390 | 60.40 | 58.00 | 65.40 | 65.00 | 57.70 | 58.50 | 56.10 | 99.20 | 99.20 | 100.00 | 99.60 |
| oxysporum | | | | | | | | | | | | |
| f. sp. radicis- | | | | | | | | | | | | |
| lycopersici | | | | | | | | | | | | |
| 26381 | | | | | | | | | | | | |
| Fusarium | ENH63602 | 60.40 | 58.00 | 65.40 | 65.00 | 57.70 | 57.40 | 56.10 | 99.60 | 99.60 | 99.60 | 100.00 |
| oxysporum | | | | | | | | | | | | |
| f. sp. cubense | | | | | | | | | | | | |
| race 1 | | | | | | | | | | | | |

TABLE 6A
Percentage amino acid sequence identity among Penicillium brasilianum hmfT3 orthologogues and accession numbers thereof.

| Species | Accession | | | | | | | | | | | |
|---|---|---|---|---|---|---|---|---|---|---|---|---|
| Penicillium | SEQ ID NO: 6 | 100.00 | 85.10 | 81.90 | 81.70 | 80.50 | 75.90 | 75.90 | 80.20 | 75.20 | 75.20 | 75.20 |
| brasilianum | | | | | | | | | | | | |
| hmfT3 | | | | | | | | | | | | |
| Penicillium | XP_002560799 | 85.10 | 100.00 | 78.50 | 80.80 | 79.60 | 75.20 | 75.20 | 79.60 | 75.90 | 76.00 | 74.30 |
| rubens | | | | | | | | | | | | |
| Wisconsin 54- | | | | | | | | | | | | |
| 1255 | | | | | | | | | | | | |
| Penicillium | EPS29964 | 81.90 | 78.50 | 100.00 | 77.40 | 77.10 | 72.60 | 72.70 | 76.60 | 71.90 | 72.00 | 71.60 |
| oxalicum | | | | | | | | | | | | |
| 114-2 | | | | | | | | | | | | |
| Aspergillus | XP_001212020 | 81.70 | 80.80 | 77.40 | 100.00 | 78.30 | 73.90 | 73.90 | 79.00 | 74.60 | 74.60 | 73.00 |
| terreus | | | | | | | | | | | | |
| NIH2624 | | | | | | | | | | | | |
| Fusarium | ENH73763 | 80.50 | 79.60 | 77.10 | 78.30 | 100.00 | 99.80 | 99.60 | 88.00 | 93.10 | 92.90 | 98.60 |
| oxysporum | | | | | | | | | | | | |
| f. sp. cubense | | | | | | | | | | | | |
| race 1 | | | | | | | | | | | | |
| Fusarium | EGU73369 | 75.90 | 75.20 | 72.60 | 73.90 | 99.80 | 100.00 | 99.70 | 87.70 | 91.90 | 91.50 | 98.20 |
| oxysporum | | | | | | | | | | | | |
| Fo5176 | | | | | | | | | | | | |
| Fusarium | EXL94287 | 75.90 | 75.20 | 72.70 | 73.90 | 99.60 | 99.70 | 100.00 | 87.70 | 91.70 | 91.40 | 97.90 |
| oxysporum | | | | | | | | | | | | |
| f. sp. cubense | | | | | | | | | | | | |
| tropical race | | | | | | | | | | | | |
| 4 54006 | | | | | | | | | | | | |
| Nectria | XP_003040064 | 80.20 | 79.60 | 76.60 | 79.00 | 88.00 | 87.70 | 87.70 | 100.00 | 87.60 | 88.30 | 87.30 |
| haematococca | | | | | | | | | | | | |
| mpVI 77-13-4 | | | | | | | | | | | | |
| Fusarium | XP_009258565 | 75.20 | 75.90 | 71.90 | 74.60 | 93.10 | 91.90 | 91.70 | 87.60 | 100.00 | 99.00 | 91.30 |
| pseudograminearum | | | | | | | | | | | | |
| CS3096 | | | | | | | | | | | | |
| Fusarium | XP_011323833 | 75.20 | 76.00 | 72.00 | 74.60 | 92.90 | 91.50 | 91.40 | 88.30 | 99.00 | 100.00 | 91.00 |
| graminearum | | | | | | | | | | | | |
| PH-1 | | | | | | | | | | | | |
| Fusarium | CCT64241 | 75.20 | 74.30 | 71.60 | 73.00 | 98.60 | 98.20 | 97.90 | 87.30 | 91.30 | 91.00 | 100.00 |
| fujikuroi | | | | | | | | | | | | |
| IMI 58289 | | | | | | | | | | | | |

TABLE 7A
Percentage amino acid sequence identity among Penicillium brasilianum hmfT4 orthologogues and accession numbers thereof.

| Species | Accession | | | | | | | | | | | |
|---|---|---|---|---|---|---|---|---|---|---|---|---|
| Penicillium | SEQ ID NO: 7 | 100.00 | 68.90 | 66.40 | 38.60 | 38.50 | 36.90 | 37.40 | 37.50 | 39.10 | 36.50 | 37.80 |
| brasilianum | | | | | | | | | | | | |
| hmfT4 | | | | | | | | | | | | |
| Sporothrix | ERT02386 | 68.90 | 100.00 | 67.00 | 35.90 | 38.40 | 37.90 | 35.90 | 38.50 | 39.50 | 38.50 | 38.80 |
| schenckii | | | | | | | | | | | | |
| ATCC 58251 | | | | | | | | | | | | |
| Togninia | XP_007915981 | 66.40 | 67.00 | 100.00 | 36.20 | 40.30 | 40.50 | 39.10 | 41.00 | 38.80 | 38.50 | 42.10 |
| minima | | | | | | | | | | | | |
| UCRPA7 | | | | | | | | | | | | |
| Capronia | XP_007724585 | 38.60 | 35.90 | 36.20 | 100.00 | 57.40 | 56.80 | 60.00 | 56.40 | 57.30 | 54.10 | 56.40 |
| coronata | | | | | | | | | | | | |
| CBS 617.96 | | | | | | | | | | | | |
| Sporothrix | ERS98342 | 38.50 | 38.40 | 40.30 | 57.40 | 100.00 | 59.20 | 55.30 | 59.60 | 71.10 | 61.80 | 58.90 |
| schenckii | | | | | | | | | | | | |
| ATCC 58251 | | | | | | | | | | | | |
| Aspergillus | GAA83620 | 36.90 | 37.90 | 40.50 | 56.80 | 59.20 | 100.00 | 53.40 | 80.60 | 60.30 | 56.20 | 81.30 |
| kawachii | | | | | | | | | | | | |
| IFO 4308 | | | | | | | | | | | | |
| Capronia | XP_007725190 | 37.40 | 35.90 | 39.10 | 60.00 | 55.30 | 53.40 | 100.00 | 53.40 | 55.70 | 52.90 | 53.60 |
| coronata | | | | | | | | | | | | |
| CBS 617.96 | | | | | | | | | | | | |
| Aspergillus | XP_001389139 | 37.50 | 38.50 | 41.00 | 56.40 | 59.60 | 80.60 | 53.40 | 100.00 | 61.70 | 56.00 | 100.00 |
| niger CBS | | | | | | | | | | | | |
| 513.88 | | | | | | | | | | | | |
| Grosmannia | EFX04858 | 39.10 | 39.50 | 38.80 | 57.30 | 71.10 | 60.30 | 55.70 | 61.70 | 100.00 | 61.40 | 61.40 |
| clavigera | | | | | | | | | | | | |
| kw1407 | | | | | | | | | | | | |
| Sporothrix | ERS94853 | 36.50 | 38.50 | 38.50 | 54.10 | 61.80 | 56.20 | 52.90 | 56.00 | 61.40 | 100.00 | 55.70 |
| schenckii | | | | | | | | | | | | |
| ATCC 58251 | | | | | | | | | | | | |
| Aspergillus | EHA26600 | 37.80 | 38.80 | 42.10 | 56.40 | 58.90 | 81.30 | 53.60 | 100.00 | 61.40 | 55.70 | 100.00 |
| niger | | | | | | | | | | | | |
| ATCC 1015 | | | | | | | | | | | | |

TABLE 8A
Percentage amino acid sequence identity among Penicillium brasilianum hmfT5 orthologogues and accession numbers thereof.

| Species | Accession | | | | | | | | | | | |
|---|---|---|---|---|---|---|---|---|---|---|---|---|
| Penicillium | SEQ ID NO: 8 | 100.00 | 84.00 | 83.90 | 84.30 | 82.20 | 82.30 | 82.20 | 82.40 | 81.00 | 81.10 | 81.00 |
| brasilianum | | | | | | | | | | | | |
| hmfT5 | | | | | | | | | | | | |
| Penicillium | EKV20717 | 84.00 | 100.00 | 99.90 | 91.40 | 79.10 | 79.20 | 79.30 | 80.80 | 79.10 | 79.10 | 76.80 |
| digitatum Pd1 | | | | | | | | | | | | |
| Penicillium | EKV19541 | 83.90 | 99.90 | 100.00 | 91.30 | 79.10 | 79.10 | 79.20 | 80.70 | 79.00 | 79.00 | 76.80 |
| digitatum | | | | | | | | | | | | |
| PHI26 | | | | | | | | | | | | |
| Penicillium | XP_002565665 | 84.30 | 91.40 | 91.30 | 100.00 | 80.10 | 80.20 | 80.00 | 81.60 | 80.90 | 80.40 | 77.90 |
| rubens | | | | | | | | | | | | |
| Wisconsin | | | | | | | | | | | | |
| 54-1255 | | | | | | | | | | | | |
| Aspergillus | KDE82314 | 82.20 | 79.10 | 79.10 | 80.10 | 100.00 | 100.00 | 99.60 | 82.40 | 81.60 | 81.30 | 75.60 |
| oryzae | | | | | | | | | | | | |
| 100-8 | | | | | | | | | | | | |
| Aspergillus | EIT77345 | 82.30 | 79.20 | 79.10 | 80.20 | 100.00 | 100.00 | 99.60 | 82.40 | 81.60 | 81.30 | 75.70 |
| oryzae | | | | | | | | | | | | |
| 3.042 | | | | | | | | | | | | |
| Aspergillus | XP_002380612 | 82.20 | 79.30 | 79.20 | 80.00 | 99.60 | 99.60 | 100.00 | 82.50 | 81.60 | 81.30 | 75.70 |
| flavus | | | | | | | | | | | | |
| NRRL3357 | | | | | | | | | | | | |
| Aspergillus | XP_001208847 | 82.40 | 80.80 | 80.70 | 81.60 | 82.40 | 82.40 | 82.50 | 100.00 | 84.90 | 84.80 | 76.10 |
| terreus | | | | | | | | | | | | |
| NIH2624 | | | | | | | | | | | | |
| Aspergillus | GAA86951 | 81.00 | 79.10 | 79.00 | 80.90 | 81.60 | 81.60 | 81.60 | 84.90 | 100.00 | 97.40 | 75.70 |
| kawachii | | | | | | | | | | | | |
| IFO 4308 | | | | | | | | | | | | |
| Aspergillus | XP_001400982 | 81.10 | 79.10 | 79.00 | 80.40 | 81.30 | 81.30 | 81.30 | 84.80 | 97.40 | 100.00 | 75.40 |
| niger | | | | | | | | | | | | |
| CBS 513.88 | | | | | | | | | | | | |
| Ophiostoma | EPE02908 | 81.00 | 76.80 | 76.80 | 77.90 | 75.60 | 75.70 | 75.70 | 76.10 | 75.70 | 75.40 | 100.00 |
| piceae | | | | | | | | | | | | |
| UAMH 11346 | | | | | | | | | | | | |

TABLE 9A
Percentage amino acid sequence identity among Penicillium brasilianum hmfT6 orthologues and accession numbers thereof.

| Species | Accession | | | | | | | | | | | |
|---|---|---|---|---|---|---|---|---|---|---|---|---|
| Penicillium brasilianum hmfT6 | SEQ ID NO: 9 | 100 | 99.4 | 90.7 | 78.8 | 78.1 | 79.2 | 78.1 | 78.6 | 78.2 | 72.8 | 72.8 |
| Penicillium brasilianum | CEJ60583.1 | 99.4 | 100 | 90.3 | 78.6 | 77.9 | 79 | 77.9 | 78.6 | 78 | 72.6 | 72..6 |
| Penicillium subrubescens PENSUB_8187 | OKO99970.1 | 90.7 | 90.3 | 100 | 77.2 | 76.9 | 77.8 | 77.8 | 76.6 | 76.2 | 72.1 | 72.1 |
| Aspergillus sydowii CBS 593.65 | OJJ53782.1 | 78.8 | 78.6 | 77.2 | 100 | 94 | 80.8 | 79.4 | 83.4 | 85.8 | 69.1 | 69.1 |
| Aspergillus versicolor CBS 583.65 | OJJ07888.1 | 78.1 | 77.9 | 76.9 | 94 | 100 | 81 | 80.4 | 82.7 | 84.3 | 67.1 | 67.1 |
| Penicillium italicum | KGO73014.1 | 79.2 | 79 | 77.8 | 80.8 | 81 | 100 | 78.8 | 86.5 | 88.2 | 69 | 69 |
| Talaromyces islandicus | CRG83369.1 | 78.1 | 77.9 | 77.8 | 79.4 | 80.4 | 78.8 | 100 | 77.7 | 77.7 | 74.6 | 74.6 |
| Aspergillus ruber CBS 135680 | EYE92060.1 | 78.6 | 78.6 | 76.6 | 83.4 | 82.7 | 86.5 | 77.7 | 100 | 94.6 | 67.8 | 67.8 |
| Aspergillus glaucus CBS 516.65 | OJJ86250.1 | 78.2 | 78 | 76.2 | 85.8 | 84.3 | 88.2 | 77.7 | 94.6 | 100 | 69.2 | 69.2 |
| Metarhizium anisopliae | KID68223.1 | 72.8 | 72.6 | 72.1 | 69.1 | 67.1 | 69 | 74.6 | 67.8 | 69.2 | 100 | 99.6 |
| Metarhizium anisopliae BRIP 53293 | KJK94474.1 | 72.8 | 72.6 | 72.1 | 69.1 | 67.1 | 69 | 74.6 | 67.8 | 69.2 | 99.6 | 100 |

TABLE 10A
Percentage amino acid sequence identity among Penicillium brasilianum hmfT7 orthologues and accession numbers thereof.

| Species | Accession | | | | | | | | | | | |
|---|---|---|---|---|---|---|---|---|---|---|---|---|
| Penicillium brasilianum hmfT7 | SEQ ID NO: 10 | 100 | 98.7 | 89.1 | 80.5 | 76.1 | 76.4 | 74.9 | 73.9 | 71.5 | 70.6 | 70 |
| Penicillium brasilianum PMG11_04505 | CEO59852.1 | 98.7 | 100 | 89.3 | 80.8 | 76.1 | 76.4 | 74.5 | 73.9 | 71.7 | 75.5 | 75 |
| Penicillium subrubescens | OKP11238.1 | 89.1 | 89.3 | 100 | 84.5 | 77 | 76.6 | 74.7 | 74.3 | 73.2 | 75.5 | 74.8 |
| Penicillium oxalicum 114-2 | EPS33230.1 | 80.5 | 80.8 | 84.5 | 100 | 74.2 | 73.4 | 72.5 | 73.4 | 69.6 | 72.7 | 72.8 |
| Penicillium arizonense PENARI_c004G06722 | OGE55472.1 | 76.1 | 76.1 | 77 | 74.2 | 100 | 86.6 | 84.9 | 84.6 | 69.7 | 84.9 | 82.4 |
| Penicillium rubens Wisconsin 54-1255 | XP_002564221.1 | 76.4 | 76.4 | 76.6 | 73.4 | 86.6 | 100 | 89.3 | 92.2 | 70.8 | 91 | 90.2 |
| Penicillium griseofulvum | KXG53210.1 | 74.9 | 74.5 | 74.7 | 72.5 | 84.9 | 89.3 | 100 | 87.2 | 69.1 | 87.5 | 86.5 |
| Penicillium nordicum | KOS36679.1 | 73.9 | 73.9 | 74.3 | 73.4 | 84.6 | 92.2 | 87.2 | 100 | 69.1 | 91 | 90 |
| Aspergillus nomius NRRL 13137 | XP_015410789.1 | 71.5 | 71.7 | 73.2 | 69.6 | 69.7 | 70.8 | 69.1 | 69.1 | 100 | 71.2 | 69.1 |
| Penicillium italicum | KGO73431.1 | 70.6 | 75.5 | 75.5 | 72.7 | 84.9 | 91 | 87.5 | 91 | 71.2 | 100 | 88.4 |
| Penicillium expansum | KGO59243.1 | 70 | 75 | 74.8 | 72.8 | 82.4 | 90.2 | 86.5 | 90 | 69.1 | 88.4 | 100 |

TABLE 11A
Percentage amino acid sequence identity among Penicillium brasilianum hmfR orthologogues and accession numbers thereof.

| Species | Accession | | | | | | | | | | | |
|---|---|---|---|---|---|---|---|---|---|---|---|---|
| Penicillium | SEQ ID NO: 11 | 100.00 | 52.30 | 46.40 | 41.70 | 40.60 | 41.70 | 41.80 | 41.70 | 41.70 | 41.70 | 41.70 |
| brasilianum | | | | | | | | | | | | |
| hmfR | | | | | | | | | | | | |
| Sporothrix | ERT02388 | 52.30 | 100.00 | 43.10 | 39.60 | 36.70 | 41.80 | 41.90 | 41.80 | 41.90 | 40.30 | 41.80 |
| schenckii | | | | | | | | | | | | |
| ATCC 58251 | | | | | | | | | | | | |
| Scedosporium | KEZ45621 | 46.40 | 43.10 | 100.00 | 43.50 | 42.30 | 45.90 | 45.70 | 45.90 | 46.10 | 44.80 | 45.90 |
| apiospermum | | | | | | | | | | | | |
| Stachybotrys | KFA62280 | 41.70 | 39.60 | 43.50 | 100.00 | 51.50 | 56.30 | 55.90 | 56.30 | 56.00 | 55.30 | 56.30 |
| chlorohalonata | | | | | | | | | | | | |
| IBT 40285 | | | | | | | | | | | | |
| Verticillium | XP_003000413 | 40.60 | 36.70 | 42.30 | 51.50 | 100.00 | 53.90 | 54.00 | 53.90 | 54.00 | 53.10 | 53.70 |
| alfalfae | | | | | | | | | | | | |
| VaMs.102 | | | | | | | | | | | | |
| Fusarium | EXL68817 | 41.70 | 41.80 | 45.90 | 56.30 | 53.90 | 100.00 | 97.90 | 99.60 | 98.40 | 98.60 | 98.50 |
| oxysporum | | | | | | | | | | | | |
| f. sp. | | | | | | | | | | | | |
| conglutinans | | | | | | | | | | | | |
| race 2 54008 | | | | | | | | | | | | |
| Fusarium | EXK46473 | 41.80 | 41.90 | 45.70 | 55.90 | 54.00 | 97.90 | 100.00 | 97.50 | 97.80 | 98.20 | 97.90 |
| oxysporum | | | | | | | | | | | | |
| f. sp. melonis | | | | | | | | | | | | |
| 26406 | | | | | | | | | | | | |
| Fusarium | EGU75021 | 41.70 | 41.80 | 45.90 | 56.30 | 53.90 | 99.60 | 97.50 | 100.00 | 98.10 | 98.30 | 98.30 |
| oxysporum | | | | | | | | | | | | |
| Fo5176 | | | | | | | | | | | | |
| Fusarium | EXM14771 | 41.70 | 41.90 | 46.10 | 56.00 | 54.00 | 98.40 | 97.80 | 98.10 | 100.00 | 98.30 | 99.10 |
| oxysporum | | | | | | | | | | | | |
| f. sp. | | | | | | | | | | | | |
| vasinfectum | | | | | | | | | | | | |
| 25433 | | | | | | | | | | | | |
| Fusarium | EXM09676 | 41.70 | 40.30 | 44.80 | 55.30 | 53.10 | 98.60 | 98.20 | 98.30 | 98.30 | 100.00 | 98.40 |
| oxysporum | | | | | | | | | | | | |
| f. sp. cubense | | | | | | | | | | | | |
| tropical race | | | | | | | | | | | | |
| 4 54006 | | | | | | | | | | | | |
| Fusarium | EXK77862 | 41.70 | 41.80 | 45.90 | 56.30 | 53.70 | 98.50 | 97.90 | 98.30 | 99.10 | 98.40 | 100.00 |
| oxysporum | | | | | | | | | | | | |
| f. sp. raphani | | | | | | | | | | | | |
| 54005 | | | | | | | | | | | | |

### DESCRIPTION OF THE FIGURES

FIG. 1. Split marker approach to create hmfK1 (top) and hmfK3 (bottom) deletions, using hygB and phleo antibiotic markers, respectively.

FIG. 2. Concentrations of HMF, FDCA and intermediates measured in supernatants of cultures of wild type P. brasilianum C1 (A), and the P. brasilianum C1 disruption mutant strains ΔhmfK1 #26 (B), ΔhmfK1 #30 (C) and ΔhmfK3 #43 (D), grown in minimal medium supplemented with 5 mM glucose and 6 mM HMF at 30° C. in shake flasks at 250 rpm.

FIG. 3. Concentrations of HMF, FDCA and intermediates measured in supernatants of cultures of wild type P. brasilianum C1 (A), and the P. brasilianum C1 disruption mutant strains ΔhmfK1 #26 (B), ΔhmfK1 #30 (C) and ΔhmfK3 #43 (D), grown in minimal medium supplemented with 5 mM citrate and 6 mM HMF at 30° C. in shake flasks at 250 rpm. After 4 days ˜1% of the glucose containing culture of #26 and #30 was used to re-inoculate the citrate containing flasks.

### EXAMPLES

### Introduction

In co-pending International application PCT/EP2016/072406 the inventors have described that the isolation from Dutch soil of a Penicillium brasilianum strain. This P. brasilianum, referred to P. brasilianum C1 in application PCT/EP2016/072406, was isolated by growth selection on HMF, a precursor for FDCA. Herein, the P. brasilianum C1 strain will be referred to as the wild type (WT) WT P. brasilianum strain or the WT strain. International application PCT/EP2016/072406 describes the sequencing and annotation of the genome of P. brasilianum C1, as well as the sequencing of the transcriptome of the strain grown on HMF and on citric acid. Based on the genome annotation and blasting genes against public databases as well as on differential expression RNA-sequencing results, a list has been compiled of candidate genes that are involved in encoding enzymes that are involved in the catabolism of HMF by P. brasilianum C1 via a proposed HMF to furoic acid (via FDCA) pathway, including a salicylate hydroxylase hmfK1, two alcohol dehydrogenases hmfL1 and hmfL2 and a salicylaldehyde dehydrogenase hmfN1. By heterologous expression in a Pseudomonas strain, the Examples of PCT/EP2016/072406 show that the P. brasilianum hmfK1 hydroxylase indeed acts as a decarboxylating monooxygenase on FDCA and thus is involved in the degradation of FDCA in P. brasilianum. Furthermore, by heterologous expression in yeast of the P. brasilianum hmfL1 and hmfL2 alcohol dehydrogenases and the P. brasilianum hmfN1 salicylaldehyde dehydrogenase, the Examples of PCT/EP2016/072406 show that these enzymes indeed have ability to efficiently oxidise HMF to FDCA.

In addition the P. brasilianum hmfK1, hmfL1, hmfL2 and hmfN1 genes, the Examples of PCT/EP2016/072406 described a number of further genes encoding protein or enzymes involved in involved in the catabolism of HMF in P. brasilianum, which are listed in Table 12.

TABLE 12
Genes in the P. brasilianum C1 genome identified as being involved in HMF catabolism

| Gene | | Function | role inHMF | aa | aa | nt |
|---|---|---|---|---|---|---|
| name | Contig | (annotated) | catabolism | SEQ ID NO: | length | SEQ ID NO: |
| hmfL1 | 82 | alcohol | HMFCA oxidation | 1 | 351 | 12 |
| | | dehydrogenase | to FFCA | | | |
| | | Zn-binding | | | | |
| hmfL2 | 153 | alcohol | HMFCA oxidation | 2 | 339 | 13 |
| | | dehydrogenase | to FFCA | | | |
| | | Zn-binding | | | | |
| hmfN1 | 730 | salicylaldehyde | HMF/FFCA oxidation | 3 | 505 | 14 |
| | | dehydrogenase | to HMFCA/FDCA | | | |
| hmfK1 | 730 | salicylate | FDCA | 4 | 427 | 15 |
| | | hydroxylase | decarboxylation | | | |
| | | FAD binding | | | | |
| | | monooxygenase | | | | |
| hmfM | 273 | short chain | reduction of | 5 | 245 | 16 |
| | | dehydrogenase | HMF/FFCA to | | | |
| | | | the corresponding | | | |
| | | | alcohol | | | |
| hmfT3 | 254 | major superfamily | furan transport | 6 | 581 | 17 |
| | | facilitator protein | | | | |
| hmfT4 | 730 | major superfamily | furan transport | 7 | 513 | 18 |
| | | facilitator protein | | | | |
| hmfT5 | 1 | ABC transporter | furan transport | 8 | 1435 | 19 |
| hmfT6 | 226 | major superfamily | furan transport | 9 | 527 | 20 |
| | | facilitator protein | | | | |
| hmfT7 | 570 | major superfamily | furan transport | 10 | 514 | 21 |
| | | facilitator protein | | | | |
| hmfR | 730 | transcriptional | induction furan | 11 | 872 | 22 |
| | | activator | catabolism genes | | | |

### Example 1: Attempt to Delete hmfK1 and Identification of hmfK3

Initial attempts were made to delete the first salicylate hydroxylase gene hmfK1 using a nia1 selection marker. Although diagnostic PCR confirmed the deletion of hmfK1, RNAseq analysis revealed that hmfK1 is still expressed, indicating that the deletion was not successful. Interestingly, in the analysed presumed mutant strain yet another salicylate hydroxylase, hmfK3 (SEQ ID NO.: 23, encoded by SEQ ID NO.: 24), was upregulated that was not identified as being upregulated in the Examples of PCT/EP2016/072406. The presumed hmfK1 deletion transformants were next analysed in more detail. From this analysis we concluded that that both hmfK1 deletion transformants were not correct. Most likely the deletion construct had integrated at other site(s), possibly up/downstream of hmfK1 and/or at the nia1 gene itself (different in both mutants).

### Example 2: Evaluation of RNAseq Data

We obtained RNAseq data from the WT and the presumed hmfK1 deletion transformant ΔSH #2E4 (which turned out not to have correct hmfK1 deletion) from chemostat cultures grown on citrate and then replacing the citrate by HMF. A set of genes possibly involved in the degradation of HMF via FDCA was identified. The hmfK1 gene was the gene which showed the strongest induction in the HMF-fed culture in the WT strain. A number of HMF-induced genes are located on the same contig (no. 730) as hmfK1 is indicating the presence of a gene cluster encoding a HMF degradation pathway. As already discussed above, hmfK1 was still strongly induced in transformant ΔSH #2E4. In addition, a second hydroxylase gene with homology to hmfK1, now termed hmfK3, was discovered to be induced in this transformant, but not in the WT strain. Since no FDCA accumulation was observed in both the WT and mutant cultures, we reanalysed the available RNAseq RPKM data for identification of potential alternative targets for improved FCDA strain design (e.g. degradation specific regulator gene).

Data re-analysis was focused on (log₂) ratios of:
1. WT_HMF vs WT_Citrate
2. M_HMF vs M_Citrate
3. M_HMF vs WT_HMF
4. M_Citr vs WT_Citr

The data was first floored (+2) to be able to look at genes with ratio “0” and “div0”. Selections of genes were made by using arbitrary cut off values to get comparable amount (˜100) of induced genes (see Table 2 and the Supplementary Excel File).

In the mutant (compared to the WT) culture large sets of genes were induced and repressed similarly in both HMF and citrate conditions. This suggests the induction/repression of these genes are HMF independent and mutant and/or culture specific. An exception is the induction of the salicylate hydroxylase hmfK3.

The previously obtained results (induction of hmfK1, hmfK3, alcohol dehydrogenases, transcriptional activator, lactonase etc.) were confirmed in the HMF-treated cultures. Salicyl related genes and decarboxylases are only induced by HMF and oxidoreductases mainly regulated by HMF. HMF also induced various transporters, like g10375 (contig 570) and g5964 (contig_226), encoding Major Facilitator Superfamily proteins, which could have a role in the transport of HMF, or its derivative into and out of the cell.

The BLAST results of the hmf genes previously identified in the Examples of PCT/EP2016/072406, and possibly involved in HMF degradation via FDCA were re-evaluated. Interesting to see is that the HMF-induced gene cluster around hmfK1 (contig730) shows a clear resemblance with a fungal second metabolite cluster. The highest identity (70%) was discovered with an orthologue gene cluster in Sporothrix schenckii ATCC 58251. The hmfL1 gene (encoding an alcohol dehydrogenase lies on a different contig (82) than hmfK1 (contig 730). However, the hmfL1 orthologue in S. schenckii is in the same gene cluster. In the publicly available P. brasilianum MG11 genome (Horn et al., 2015. Genome Announc 3(5):e00724-15. doi:10.1128/genomeA.00724-15; GenBank acc. no. CDHK01000001.1) the hmf gene cluster (containing hmfK1) also includes hmfL1. Therefore we conclude contigs 82 and 730 are adjacent to each other.

The co-factor dependence of dehydrogenases in the hmf gene cluster (contigs 730 and 82) was also investigated. For an economically viable production of FDCA in fungi NAD as the co-factor is preferred, since fungi have a higher capacity to re-generate NAD than NADP.

| Gene | NCBI | Annotation | Co-factor |
|---|---|---|---|
| hmfL1 | CEJ57635 | alcohol dehydrogenase | NAD |
| (contig_82) | | | |
| hmfN1 | CEJ57637 | aldehyde dehydrogenase | NAD |
| (contig_730) | | | (NADP?) |

BLAST results with hmfL1 show a preference for NAD as the co-factor. The BLAST results with hmfN1 does not discriminate for NAD and NADP dependence. Best match for hmfN1 however is with a salicylaldehyde dehydrogenase (DoxF, SaliADH, EC=1.2.1.65) involved in the upper naphthalene catabolic pathway of Pseudomonas strain C18. For DoxF NAD seems the preferred co-factor.

Based on re-evaluation results of the RNAseq data we also conclude that salicylate hydroxylases hmfK1 and secondly hmfK3 are the best initial candidates to delete in order construct a host cell that oxidises HMF to FDCA but which does not degrade the FDCA produced.

### Example 3: Development of hmfK1 & hmfK3 Single and Double Mutant Strains

### Gene Deletion Design and Synthesis

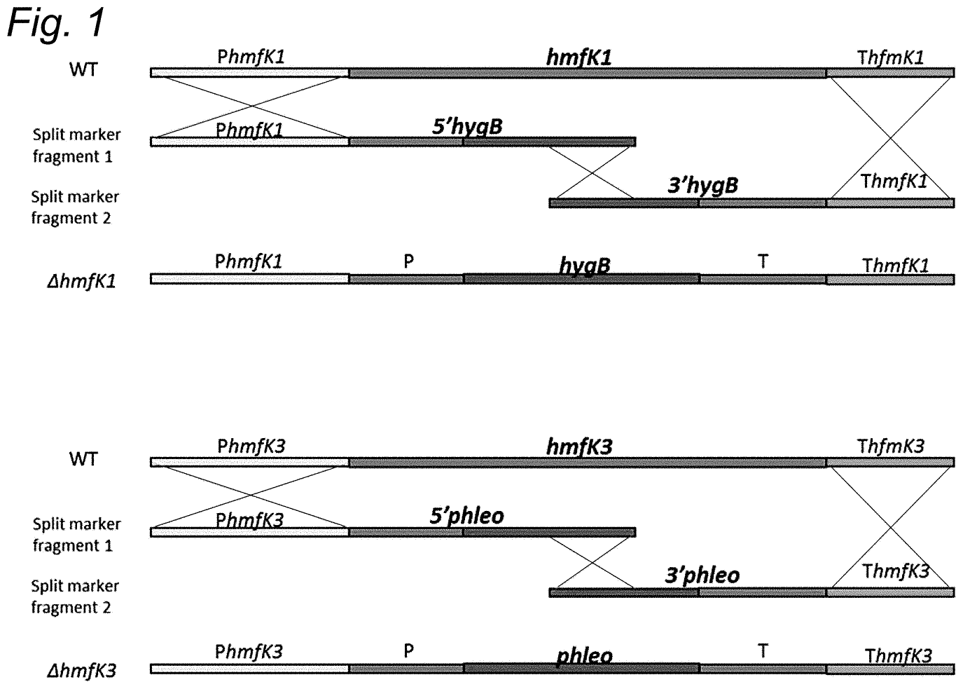

The WT P. brasilianum strain has been tested for phleomycin and hygromycin sensitivity to confirm the suitability of phleo or hygB antibiotic selection markers for gene disruption (see above). Gene deletion design has been performed by DDNA and is based on a split marker approach (Arentshorst et al., 2015, In: “Genetic Transformation Systems in Fungi”, Volume 1. van den Berg M. A., Maruthachalam K., (eds). Switzerland: Springer International Publishing, pp. 263-272) using the hygB selection marker for the hmfK1 deletion mutant and the phleo selection marker for the hmfK3 deletion mutant (FIG. 1). The split marker approach is applied to reduce the frequency of integration at incorrect (not hmfK1 or hmfK3) sites, since only DNA fragments that have undergone homologous recombination will result in an intact antibiotic selection marker gene. An attempt to create a hmfK1 hmfK3 double mutant was made using both selection markers in one transformation experiment. Synthesis of the gene disruption-fragments as outlined in FIG. 1 was outsourced to GeneArt, ThermoFisher Scientific. Sequences of the fragments are provided in the sequence listing (SEQ ID NO.'s: 30-33).

### Transformation & Selection

Before the transformations were performed an FDCA degradation screen was set up for the WT P. brasilianum strain. The WT strain is able to use FDCA as a carbon source. Creating mutants that are unable to do this will not grow on media containing FDCA as the sole C-source. This assay will facilitate screening for correct hmfK1 (and hmfK3) deletion transformants (assuming deletion of these genes will impair FDCA degradation).

Corbion minimal medium was used, which contains the following per liter of demineralized water: 2.0 g of (NH₄)2SO₄, 0.1 g of MgCl₂ 6H₂0, 10 mg of EDTA, 2 mg of ZnSO₄ 0.7H₂0, 1 mg of CaCl₂). 2H₂0, 5 mg of FeSO₄ 7H₂0, 0.2 mg of Na₂MoO₄. 2H₂0, 0.2 mg of CuSO₄ 5H₂0, 0.4 mg of CoCl₂ 6H₂0, and 1 mg of MnCl₂ 2H₂0. 25 mM KH₂PO₄ and 25 mM NaH₂PO₄. One or more of the following C-sources were added depending on the experiment: 0.2 g/l of yeast extract, 15 mM of FDCA, 5 mM glucose, 5 mM citric acid, 6 mM HMF. pH was set at 3.0, except when FDCA was used as sole C-source, to avoid precipitation of FDCA.

WT P. brasilianum was able to grow on Corbion minimal medium with 15 mM FDCA as sole C-source, both in liquid medium as well as on agar plates. The negative control (minimal medium without any C-source) showed a minimal amount of background growth, which must be due to the ability of this strain to grow on agar components. This is a beneficial side-affect, because in a screen with transformants this background growth is a positive control for actual transfer of spores. We decided to initially screen the transformants (see below) on agar plates with FDCA and without YE (more clear results and faster), i.e. with 15 mM FDCA as sole C-source.

Gene fragments were transformed to the WT P. brasilianum strain using a standard DDNA fungal transformation protocol (Punt and van den Hondel, 1992, Methods in Enzymol. 261:447-457). Protoplasts of the WT strain were transformed with the fragments to create ΔhmfK1 and plated on selection plates containing HygB. In addition, protoplasts of the WT strain were transformed with fragments to create both ΔhmfK1 and ΔhmfK3 and plated on selection plates containing HygB alone, Phleo alone, and both HygB and Phleo. In total, 48 primary transformants were generated on the various selection plates. These primary transformants were screened based on no or reduced growth on plates containing FDCA as the sole C-source. Seventeen out of the 35 (˜50%) HygB+ (ΔhmfK1) transformants showed a strong phenotype on plates (no growth on FDCA), suggesting FDCA degradation route involves an intact hmfK1 gene. Potential ΔhmfK3 mutants showed no phenotype on plates. All transformants were additionally tested for the presence of antibiotic markers.

Next, the 17 HygB+(ΔhmfK1) clones that showed a phenotype on plates and all hmfK1+hmfK3 transformants were purified on plates and tested in liquid medium ±15 mM FDCA (−YE). For all ΔhmfK1 clones, that showed a strong phenotype on agar plates, growth was completely abolished in liquid medium with FDCA as the only C-source.

Diagnostic PCR analysis was performed with the different hmfK1 deletion transformants to discriminate between an intact and correctly deleted hmfK1 gene. Different primer combinations were tested (see Tables 13, 14 and 15) and the results showed that all hmfK1 clones showing a phenotype (no growth on FDCA) are correct (data not shown). Vice versa, hmfK1+hmfK3 transformants #31 and #33, showing no phenotype still produced WT hmfK1 PCR bands. Diagnostic PCR analysis was also performed on all hmfK1+hmfK3 transformants to test for correct hmfK3 deletion (see Tables 14 and 16). Only transformant #43 (ΔhmfK1+ΔhmfK3; HygB+Phleo selected) turned out to have a correct hmfK3 deletion based on PCR (data not shown). Although this transformant is HygB resistant, it does not contain a hmfK1 deletion (based on PCR results). All purified hmfK1+hmfK3 transformants were also tested in liquid cultures similarly as the hmfK1 transformants. Transformant #43 (which is a correct hmfK3 deletion) did grow on FDCA.

From the results we conclude that correct hmfK1 deletions were generated and that deletion of the hmfK1 gene results in a 100% inability to grow on FDCA as sole C-source under the conditions tested, confirming that hmfk1 is a key gene in FDCA degradation. Additionally, we can conclude that the hmfK3 deletion is correct in 1 clone (#43) and shows no phenotype, suggesting hmfk3 is not involved in FDCA degradation, at least not under the conditions tested.

TABLE 13
Primers for diagnostic PCR for verification of hmfK1 deletion.

| Primer No. | Name | Sequence | SEQ ID NO: |
|---|---|---|---|
| 180 | salHupA-430 F | GGTAGAAAAGGGGTTGCGAT | 34 |
| 181 | salHupB-1,954 F | GAAGCAATCGTCGGAAGT | 35 |
| 182 | salHdownB-3,693 R | CGCGAGGATGTATGGTATGAT | 36 |
| 183 | salHdownA-5,280 R | GAAAGTGAGATTGTGGATGGA | 37 |
| 184 | salH-53 R | TGTCGTTCATGGCTCCC | 38 |
| 185 | salH-1,583 F | AAGGTGCGAACTGGAGAG | 39 |
| 186 | niaD-2,876 F | GGAACAATCGGCAAAGAAAGTG | 40 |
| 187 | niaD-94 R | CAACTTCTTGTGGCGTTGG | 41 |

TABLE 14
Primers for diagnostic PCR for verification of hmfK1::hygB and
hmfK3::phleo deletion-and split marker amplification.

| Primer No. | Name | Sequence | SEQ ID NO: |
|---|---|---|---|
| 212 | 5flanksalH1-489 F | CTCGGTCGCTCTTTTGGGTA | 42 |
| 213 | 5flanksalH1-1,499 R | CAGGACATTGTTGGAGCCGA | 43 |
| 214 | 3flanksalH1-57 F | TCCGGAAGTGCTTGACATTG | 44 |
| 215 | 3flanksalH1-1,455 R | CCATCCCGAGTATTCTTTCGAG | 45 |
| 216 | 5flanksalH2-1,350 F | CCGTGCGCTATAATAACCTTCG | 46 |
| 217 | 5flanksalH2-1,156 R | GCGTCCCGGAAGTTCG | 47 |
| 218 | 3flanksalH2-15 F | GAGCGGTCGAGTTCTGGA | 48 |
| 219 | 3flanksalH2-1,508 R | GGTGAAAGAGTGATATATGAGGC | 49 |
| 220 | con.salH2 5flank-956 F | TCTCAGGACTTGCAGATGTTG | 50 |
| 221 | con.salH2 3flank-2,838 R | GCCAAGTCATCATCCTCGC | 51 |
| 222 | phleo-249 R | CTTACTGCCGGTGATTCGAT | 52 |
| 223 | phleo-712 F | CTACAGGACACACATTCATCGT | 53 |
| 224 | salH2-106 R | TGCCAGCCCTCCTATTCC | 54 |

TABLE 15
Expected sizes of PCR products of the designed primer combinations
for verification of the hmfK1::hygB deletion.

| Primers | ΔhmfK3 (bp) | wild type (bp) |
|---|---|---|
| 212 + 215 | 5,504 | 4,580 |
| 180 + 213 | 3,164 | — |
| 183 + 214 | 3.142 | — |
| 182 + 181 | 2,641 | 1,740 |

TABLE 16
Expected sizes of PCR products of the designed primer combinations
for verification of the hmfK3::phleo deletion.

| Primers | ΔhmfK3 (bp) | wild type (bp) |
|---|---|---|
| 216 + 219 | 4,900 | 4,566 |
| 216 + 224 | — | 1,606 |
| 220 + 222 | 1,752 | — |
| 223 + 221 | 2,126 | — |

### Example 4: Conversion of HMF and Production of FDCA

We next set up experiments to monitor conversion of HMF and production of FDCA in time. From the results above the WT strain, confirmed hmfK1 deletions #26 and #30, and confirmed hmfK3 deletion #43 were chosen to be analysed for fermentation in shake flask cultures. Strains were grown on the Corbion minimal medium w/o yeast extract, containing 5 mM glucose or 5 mM citrate, supplemented with 6 mM HMF. Cultures were grown for 7 days and samples were collected after 1, 2, 3, 4 and 7 days. Supernatant from the samples was analysed for HMF, FDCA and intermediates by HPLC as described in the Examples of PCT/EP2016/072406.

All strains grew well on glucose containing medium. The hmfK1 deletion strains #26 and #30 did not significantly grow on citrate containing medium. Therefore, on day 4, ˜1% of the glucose containing culture (of #26 and #30) was used to re-inoculate the citrate containing flasks (=citr*). However, even than no (clear) further growth was observed in these cultures. The hmfK3 deletion strain #43 grew on citrate containing medium comparable as the WT strain. The pH was measured after 1 week (data not shown). Interestingly, the pH of the cultures of the hmfK1 deletion strains after 1 week incubation was lower than of the WT and hmfK3 deletion strain cultures in both media, indicating acid (FDCA) production.

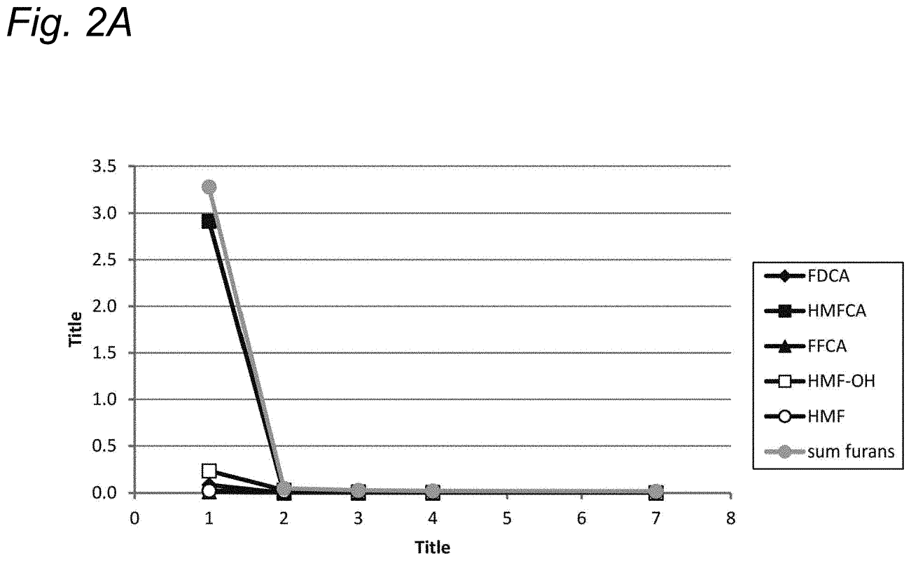

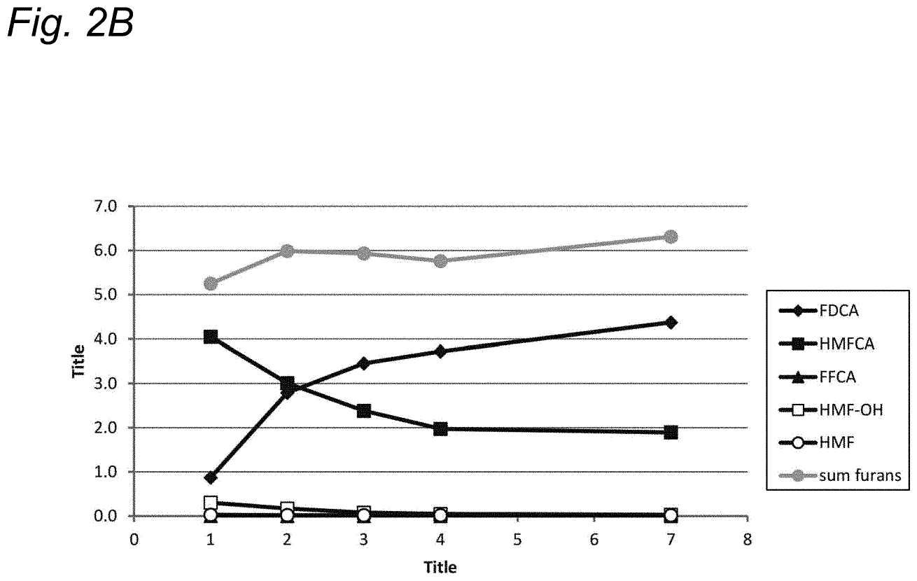

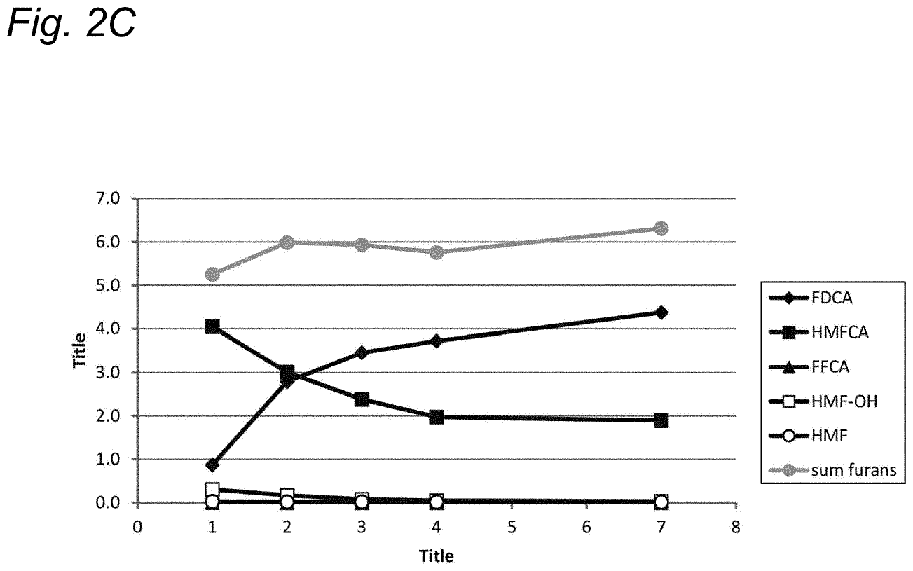

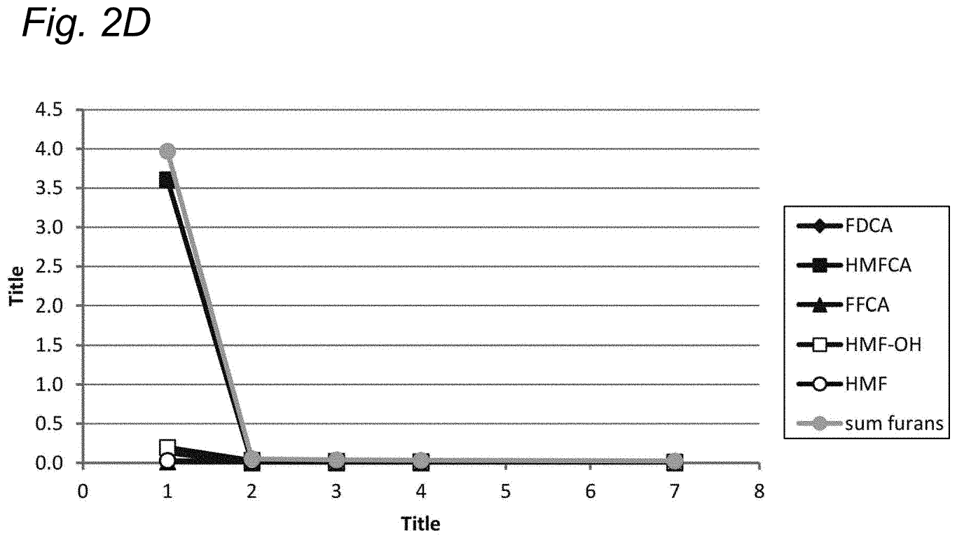

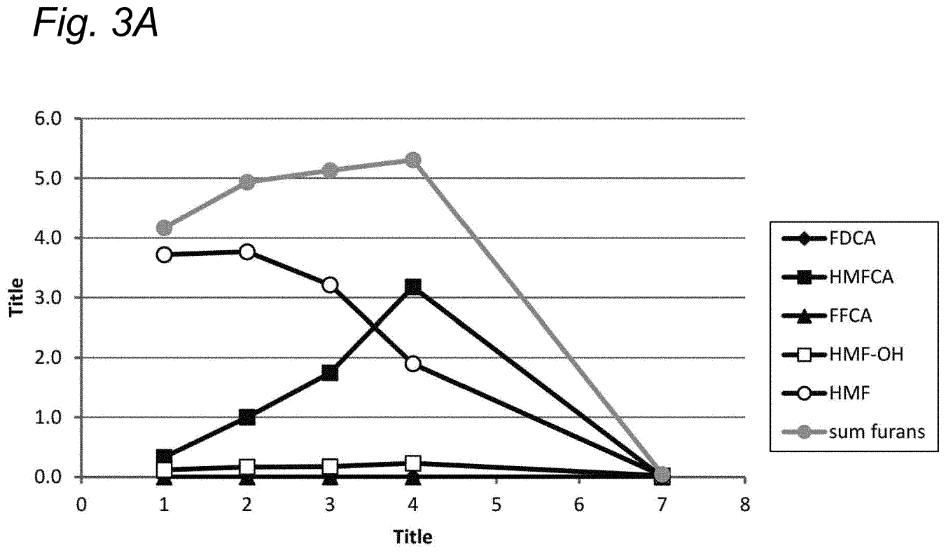

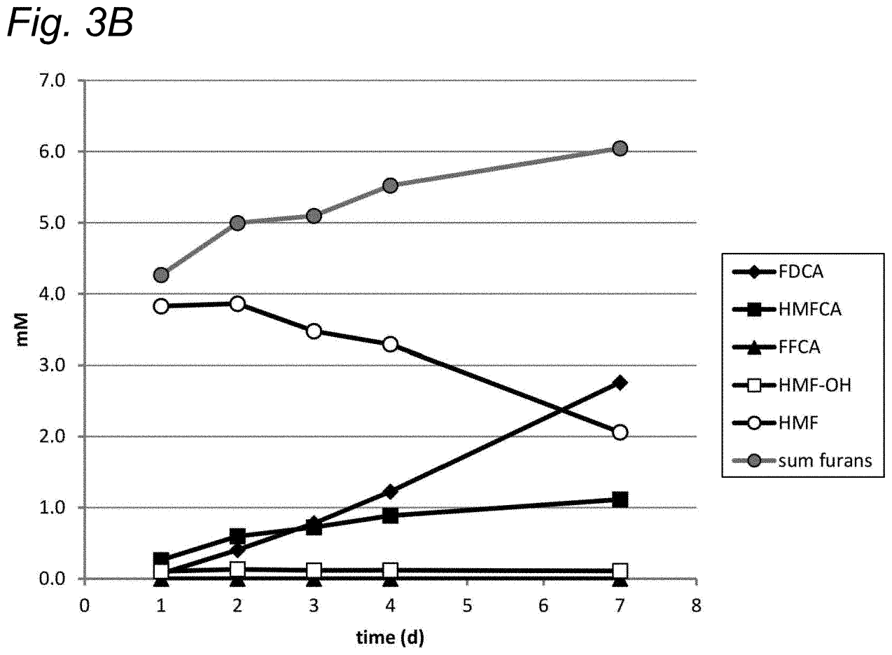

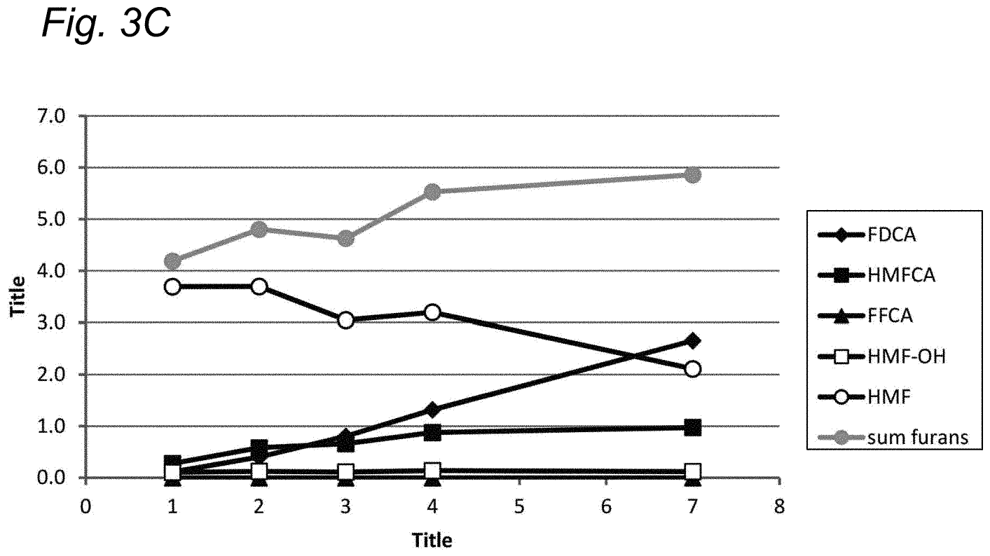

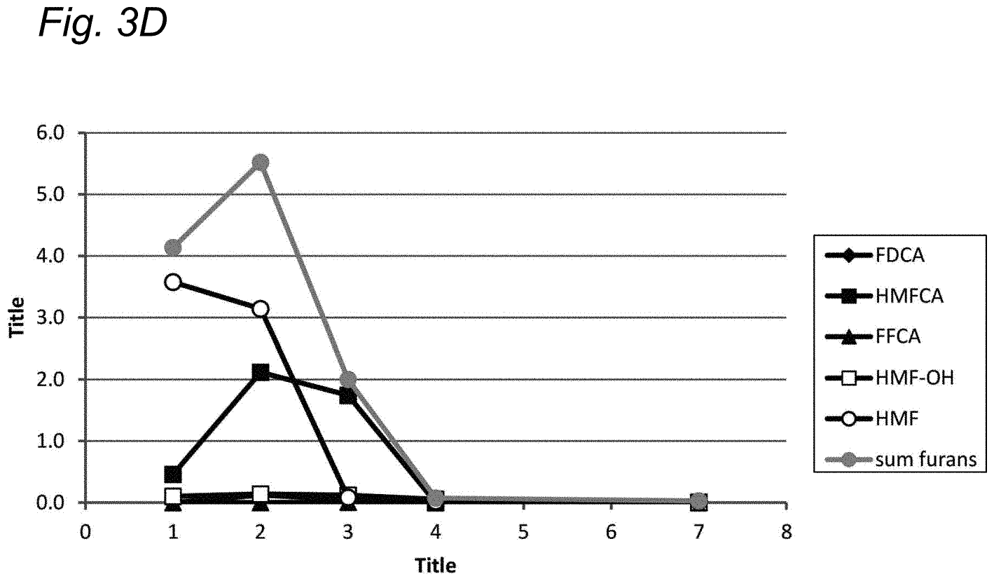

FIGS. 2 and 3 show the results of the HPLC analysis of the cultures grown on glucose or citrate, respectively. In the cultures containing glucose immediate HMF conversion was observed. Only low traces of HMF were measured even in the first time samples analysed. In both the WT and ΔhmK3 cultures all furan levels dropped to very low levels in a similar fashion and as expected, no FDCA accumulation was observed (FIGS. 2A and 2D). On the other hand, very reproducible FDCA accumulation was measured in both hmfK1 deletion strains (FIGS. 2B and 2C). HMF to FDCA conversion of up to 60-70% was reached after 7 days. The HMF-acid was a major transient intermediate compound observed in the hmfK1 deletion cultures grown on glucose.

In the citrate containing cultures also no FDCA accumulation was measured in both the WT and ΔhmK3 cultures (FIGS. 3A and 3D). All furans were degraded over time albeit at a slower rate than in the glucose containing cultures. Although no significant growth for the hmfK1 deletions #26 and #30 in citrate containing medium was observed after 4 days, about 50% HMF→HMFCA→FDCA conversion was measured (FIGS. 3B and 3D). This indicates the possible bio-conversion of HMF to FDCA performed by (germinated) spores.

From these results we conclude that hmfK1 is the key gene in FDCA degradation. Deletion of hmfK1 leads to FDCA accumulation when HMF is provided to the cultures. (Germinated) spore-based bioconversion seems possible and suggests no growth of cells is needed. However, in cultures growing in glucose containing medium FDCA accumulation is faster. Under the conditions tested, the hmfK3 gene is not involved in FDCA degradation.

### Example 5: Conversion of HMF into Crystalline FDCA at Low pH

To further demonstrate the potential of the hmfK1 deletion mutant to produce FDCA under more controlled conditions, biotransformation experiments were performed in 7-L glass fermenters. Cells were cultivated at 30° C. in a mineral salts medium (per L: KCl, 1.3 g; KH₂PO₄, 3.8 g; MgSO₄.7H₂0, 1.23 g; Na₂EDTA.2H₂O, 125 mg; ZnSO₄.7H₂O, 55 mg; H₃BO₃, 27.5 mg; MnCl₂.4H₂0, 12.5 mg; FeSO₄.7H₂O, 12.5 mg; CoCl₂.6H₂O, 4.25 mg; CuSO₄.5H₂O, 4 mg; Na₂MoO₄.2H₂O, 3.75 mg, glucose, 10 g; (NH₄)₂SO₄, 7.6 g or NaNO₃, 9.8 g). Antifoam was added as needed and the dissolved oxygen tension was maintained at 20% of air saturation level. During growth, HMF was fed to a final concentration of 1 mM to induce the FDCA producing enzyme system. After the initial glucose was depleted, glucose was fed continuously until the biomass density reached 20 g/L of cell dry weight. The biotransformation phase was initiated by switching on an HMF feed at a starting rate of 2.5 mmol/L broth/h which was manually adjusted to prevent accumulation of HMFCA or HMF. Along with HMF, a feed of glucose was provided to cover the energy demands of the cells during the biotransformation. Glucose was monitored throughout, and the feed rate was manually adjusted such that no glucose accumulated. The pH was allowed to drop from the initial value (5.5) to a setpoint of 2.7 after which it was controlled by automated addition of NaOH. Alternatively the pH was allowed to drop without adjustment, in which case the value stabilized at around 2.3. During the biotransformation, HMF was transformed into FDCA which crystallized as a solid compound below pH 3.5. Transient accumulation of HMFCA was observed when HMF was fed at a too high rate; upon reducing the feed rate the accumulated HMFCA would typically be oxidized to FDCA. The biotransformations yielded FDCA up to an overall titer of 90 g/L.

1. A fungal cell comprising a genetic modification that reduces specific 2,5-furandicarboxylic acid (FDCA) decarboxylating monooxygenase activity in the cell, as compared to a corresponding parent cell lacking the genetic modification, wherein the genetic modification eliminates the expression of an endogenous gene encoding an FDCA decarboxylating monooxygenase by deletion of at least a part of at least one of the promoter and the coding sequence of the gene.

2. The fungal cell according to claim 1, wherein the endogenous gene in the corresponding parent cell encodes a FDCA decarboxylating monooxygenase comprising an amino acid sequence with at least 45% sequence identity to at least one of SEQ ID NO: 4.

3. The fungal cell according to claim 1, wherein at least the complete coding sequence of an endogenous gene encoding an FDCA decarboxylating monooxygenase is deleted.

4. The fungal cell according to claim 1, wherein the expression of all copies of the endogenous gene encoding an FDCA decarboxylating monooxygenase is eliminated.

5. The fungal cell according to claim 1, wherein the cell has the natural ability to oxidize HMF to FDCA.

6. The fungal cell according to claim 1, wherein the cell comprises a further genetic modification that is at least one of:
a) a genetic modification that confers to the cell the ability to oxidize 5-hydroxymethyl-2-furancarboxylic acid (HMFCA) to 5-formyl-2-furoic acid (FFCA) or that increases in the cell the specific activity of an enzyme that oxidizes HMFCA to FFCA as compared to a corresponding wild type cell lacking the genetic modification; and,
b) a genetic modification that confers to the cell the ability to oxidize furanic aldehydes to the corresponding furanic carboxylic acids or a genetic modification that increases in the cell the specific activity of an enzyme that oxidizes furanic aldehydes to the corresponding furanic carboxylic acids, as compared to a corresponding wild type cell lacking the genetic modification.

7. The fungal cell according to claim 6, wherein the genetic modification in a) is a modification that increases expression of a nucleotide sequence encoding a polypeptide with HMFCA dehydrogenase activity, which polypeptide comprises an amino acid sequence that has at least 45% sequence identity with the amino acid sequence of at least one of SEQ ID NO.'s: 1 and 2; and/or,
wherein the genetic modification in b) is a modification that increases expression of a nucleotide sequence encoding a polypeptide having furanic aldehyde dehydrogenase activity, which aldehyde dehydrogenase has at least one of the abilities of i) oxidizing HMF to HMFCA, ii) oxidizing DFF to FFCA, and, iii) oxidizing FFCA into FDCA, which polypeptide comprises an amino acid sequence that has at least 45% sequence identity with the amino acid sequence of SEQ ID NO.: 3.

8. The fungal cell according to claim 1, wherein the cell further comprises a genetic modification selected from:
a) a genetic modification that reduces or eliminates the expression of a gene encoding a short chain dehydrogenase that reduces HMF and/or FFCA to the corresponding alcohol, wherein preferably the gene is at least one of a gene encoding polypeptide comprising an amino acid sequence with at least 45% sequence identity to at least one of SEQ ID NO: 5 and 25;
b) a genetic modification that increases expression of a nucleotide sequence encoding a polypeptide that transports at least one furanic compound, which polypeptide preferably comprises an amino acid sequence that has at least 45% sequence identity with the amino acid sequence of at least one of SEQ ID NO.'s: 6-10; and,
c) a genetic modification that alters the expression of a gene encoding a transcriptional activator of genes involved in furan catabolism, wherein preferably the gene is a gene encoding a polypeptide comprising an amino acid sequence with at least 45% sequence identity to SEQ ID NO: 11.

9. A fungal cell having the ability to oxidize HMF to FDCA and comprising a genetic modification that increases expression of a nucleotide sequence encoding a polypeptide that transports at least one furanic compound, which polypeptide preferably comprises an amino acid sequence that has at least 45% sequence identity with the amino acid sequence of at least one of SEQ ID NO.'s: 9 and 10.

10. The fungal cell according to claim 9, wherein the cell further comprises a genetic modification selected from:
a) a genetic modification that eliminates or reduces specific FDCA decarboxylating monooxygenase activity in the cell, as compared to a corresponding parent cell lacking the genetic modification;
b) a genetic modification that confers to the cell the ability to oxidize HMFCA to FFCA or that increases in the cell the specific activity of an enzyme that oxidizes HMFCA to FFCA as compared to a corresponding wild type cell lacking the genetic modification;
c) a genetic modification that confers to the cell the ability to oxidize furanic aldehydes to the corresponding furanic carboxylic acids or a genetic modification that increases in the cell the specific activity of an enzyme that oxidizes furanic aldehydes to the corresponding furanic carboxylic acids, as compared to a corresponding wild type cell lacking the genetic modification;
d) a genetic modification that reduces or eliminates the expression of a gene encoding a short chain dehydrogenase that reduces HMF and/or FFCA to the corresponding alcohol, wherein preferably the gene is at least one of a gene encoding polypeptide comprising an amino acid sequence with at least 45% sequence identity to at least one of SEQ ID NO: 5 and 25;
e) a genetic modification that increases expression of a nucleotide sequence encoding a polypeptide that transports at least one furanic compound, which polypeptide preferably comprises an amino acid sequence that has at least 45% sequence identity with the amino acid sequence of at least one of SEQ ID NO.'s: 6-8; and,
f) a genetic modification that alters the expression of a gene encoding a transcriptional activator of genes involved in furan catabolism, wherein preferably the gene is a gene encoding a polypeptide comprising an amino acid sequence with at least 45% sequence identity to SEQ ID NO: 11.

10.-16. (canceled)

17. The cell according to claim 1, wherein the cell is a filamentous fungal cell selected from a genus from the group consisting of:
Acremonium, Aspergillus, Aureobasidium, Cryptococcus, Fihbasidium, Fusarium, Humicola, Magnaporthe, Mucor, Mycehophthora, Neocalhmastix, Neurospora, Paecilomyces, Penicillium, Piromyces, Schizophyllum, Talaromyces, Thermoascus, Thielavia, Tolypocladium, Trichoderma, and Ustilago, preferably the cell is a filamentous fungal cell selected from a species from the group consisting of: Aspergillus niger, Aspergillus awamori, Aspergillus foetidus, Aspergillus sojae, Aspergillus fumigatus, Talaromyces emersonii, Aspergillus oryzae, Mycehophthora thermophila, Trichoderma reesei, Penicillium chrysogenum, Penicillium simplicissimum and Penicillium brasihanum;
or, wherein the cell is a yeast cell selected from a genus from the group consisting of:
Saccharomyces, Kluyveromyces, Candida, Pichia, Schizosaccharomyces, Hansenula, Kloeckera, Schwanniomyces, Yarrowia, Cryptococcus, Debaromyces, Saccharomycecopsis, Saccharomycodes, Wickerhamia, Debayomyces, Hanseniaspora, Ogataea, Kuraishia, Komagataella, Metschnikowia, Williopsis, Nakazawaea, Torulaspora, Bullera, Rhodotorula, and Sporobolomyces, preferably the cell is a yeast cell selected from a species from the group consisting of Kluyveromyces lactis, S. cerevisiae, Hansenula polymorpha, Yarrowia hpolytica, Candida tropicalis and Pichia pastoris.

18. A process for oxidizing HMFCA to FFCA, the process comprising the step of incubating a fungal cell according to claim 5, in the presence of HMFCA, under conditions conducive to the oxidation of HMFCA by the cell, wherein the cell expresses enzymes that have the ability to oxidize HMFCA to FFCA.

19. A process for producing FDCA, the process comprising the step of incubating a fungal cell according to claim 5, in a medium comprising one or more furanic precursors of FDCA, preferably under conditions conducive to the oxidation of furanic precursors of FDCA by the cell to FDCA, and, optionally recovery of the FDCA, wherein preferably, at least one furanic precursor of FDCA is selected from the group consisting of HMF, 2,5-dihydroxymethyl furan (DHF), HMFCA, FFCA and 2,5-diformyl furan (DFF), of which HMF is most preferred,
wherein the furanic precursors of FDCA are obtained from one or more hexose sugars, preferably one or more hexose sugars obtained from lignocellulosic biomass, preferably by acid-catalyzed dehydration, and,
wherein preferably the FDCA is recovered from the medium by a process comprising acid precipitation followed by cooling crystallization and/or solvent extraction.

20. The process according to claim 19, wherein the medium has a pH in the range of 2.0-3.0, wherein preferably the FDCA precipitates from the acidic medium in which it is produced and is recovered from the medium by a process comprising acid precipitation followed by cooling crystallization.

21. A process for producing a polymer from at least two FDCA monomers, the process comprising the steps of:
a) preparing an FDCA monomer in a process according to claim 19; and,
b) producing a polymer from the FDCA monomer obtained in a).

22. The process according to claim 21, wherein the polymer is produced by mixing the FDCA monomer and a diol monomer and bringing the mixture in a condition under which the FDCA and diol monomers polymerise.

23. Use of a fungal cell according to claim 5, for the biotransformation of one or more of furanic precursors to FDCA or a fungal cell expressing one or more bacterial enzymes with the ability to convert a furanic precursors of FDCA into FDCA, wherein preferably, at least one furanic precursor of FDCA is selected from the group consisting of HMF, DHF, HMFCA, FFCA and DFF, of which HMF is most preferred.


SEQUENCE LISTING (55 sequences)

SEQ ID NO: 1 - protein, 351 aa, Penicillium brasilianum
MGSLSLPETSLAAIQDKETKAISVAKRPTPVPVGTQVLVKLHYSGVCATDLHLARGSVPYLQPKVSVGGHEGTGVIASLGPDVDAAEWHVGDRVAVRWVHIVCGKCEVCTTGFENLCQSRKLAGKDVEGTFAEYAIADSSYMVRLPAGVSDADAAPILCAGVTVYKALKIASLRAGSWVAVAGAGGGLGHLAIQYARAMGLKVVALDARKRDLCLSLGAESYIDVLETDDCVAQVIKVTDGGAHGALICASSGQAYDDAVKFLRWTGTLVCIGLPPKPTLLSLGPADFVARGIKVMGTSTGDRQDTVEALAFVAKGQVKPQLTERRLEDVEEILKEIENGTMQGKAVIRIA

SEQ ID NO: 2 - protein, 339 aa, Penicillium brasilianum
MSLPSHYKRAAFKEAGGPLTIEEVDLTMPDAGEVLVKVEACGVCFSDTVPQAHGLGGKFPIVPGHEIIGHVVATGDGVSDWEVGDRIGEGWHGGHDGTCPSCRQGHFQMCDNQSINGVTKNGGYAQYCILRSEAAVRIPTHVSAAEYAPILCAGVTVFNSMRQIGVKPGSTVAIQGLGGLGHLAIQYANRFGFRVVAISRDDQKERFVRDLGAHEYINTSEEDVGSALQKLGGASLIVATAPNARAISPLLKGLRPLGKLLILAVPGEIPLDTRLMVARGLSVHGWPSGHALDSEETIRFTELEDIKCMIQTYSLDRANEAFDAMISGSVRFRAVITME

SEQ ID NO: 3 - protein, 505 aa, Penicillium brasilianum
MTQTNVHVNKSDTSLAAPQQLFISGKYQNSQRNGTFPVKNPMTGETIYECVSASLDDYAAAIEEADAAQPSWARLGPSARRLILLKAADIMETYIETDAPAILSAEVSATRGWVRANILSTAGVFRETAALATHIKGEIVPADRPGTTILVSREPVGVVLAISPWNMPATLTARAICCPLICGNSVVLRPSEFSPKSQHLVVRALTEAGLPAGCLQFLPTSTADTPRAIEFAIRHPKVSRANFTGSDRVGRIIAGLSASCLKPCVLELGGKAPVVVLEDADVEAAVEAVVYGAMSNSGQICMSTERAIVHRSLAADFKALLVKRAESLRVGNHLEDPDVQLSGLFTAASAERVLGLIKGAVNAGATLLAGDLALHGPCQTIMAPHILTGVTRDMDLFHRETFGPVLFVSEFDTDDEAIAQANDTEFSLCASVFSRDVLRAMDTAKRIRTGSCHVNGPTVYIEAPLPNGGVGGGSGYGRFGGVAGIEEFTERQIVSLAKPGIKYAF

SEQ ID NO: 4 - protein, 427 aa, Penicillium brasilianum
MPHASRSLNVLIVGAGLGGLAAGLALQTDGHKVTIIDAAPEFAEAGAGIRIPPNSSRLLMRWGVDLERMKKSTSQRYHFIRWKDGSTIFDLPFNNIVETHGAPYWLVHRADLHAALLDATLKAGVKVLNNKLVTSYDFEAPSATTQDGETFKADLIVGADGIKSICRPLLTGQPDVPRDTGDVAYRILIPGEKLLADPDLAHLIRDPCTTSWCGPDAHLVGYPIRNGEMYNIVMCATSYNETTDEVWVVKGDNSELCKRFASWEPQVRKLCALTGDFMKWRLCDLPNLARWTHPSGKAVLLGDSCHPMLPYLAQGAAQAVEDAAVLRQVLAQDMDMAAALKQYEQIRMPRASLVQAKTREHQYILHVDDGHEQQDRDKKLALDAAENPVFWGYDDRRKWLFSHDAEVIQKEGANWRDGPNMNGVHVA

SEQ ID NO: 5 - protein, 245 aa, Penicillium brasilianum
MSLSGKVVLITGSSKGIGKAAALRVASEGANVVINYLRDPVAANNLVDQIGADRALAVQADASKLADLDRLVNAAVAQFGKIDVLIPNAGILPLRDLEHTSEEDFDRTYNLMVKGPYFLAQKAVKHMPPGGRIIFVSTSTARFASVAPAYLLYTSSKGAIEQMTRIMAKDLARKGILVNAVAPGPTSTELFLEGKPEQMIKAISGFSPFNRIGEPEEIAAVMAFLSGKDSSWISGQVVAVNGAMA

SEQ ID NO: 6 - protein, 581 aa, Penicillium brasilianum
MASLIREAPFGQIVRYLTNNKYFQYPEEKPDFKLPDTWLQLLNESDAATIADPEKTEPEPEGQGYDATSEAISRASTQNSLPFTEARLEADEQHEIEKIKSIPIQPKKTKDGAILVDWYYTDDAENPHNWSNRKRALLTTLICLYTFVVYTTSAIYTSSVPGIMKEFGVSDLVATLGLSLYVLGYGTGPLIFSPLSEIPVIGRNPVYIVTMFLFVILSIPTAFVGNFAGLMVLRFLQGFFGSPCLASGGASIGDMYSLMSLPYAMMSWVSAAYCGPALGPLISGFAVPAETWRWSLFESIWMSAPVLILMFFFLPETSSATILLRRAARLRKIHNNARFMAQSEIDQRNMKVSAVAVDALIKPLEITIKDPAVLFVQVYTAIIYGIYYSFFEVFPLVYPVDYGMNLGQVGLVFLCILVSCIIGIAIYWSYLYFWMNPRIERFGFPAQESRLIPALPASIGPTIGLFLFAWTARASIHWIAPTIGITIYGATVFIVMQCLFVYIPLSYPMYAASLFAANDFFRSALACGSVLFAHPLFGNLGVARGTSLLGGLSVIGIIGIWLLYYYGARLRSLSKFAISDD

SEQ ID NO: 7 - protein, 513 aa, Penicillium brasilianum
MSTTKEAFPHTDSDIMEDSEKNLPECEHIVSVEPTLKMRDGIVLMPQPSDDPNDPLNWSWFRKHAAMFTLSYLALVCYVAVTTLVTGTVPLAKSMHVSKSTAVYLGNTPVALYAVAPWFWSPLSHFIGRRPVLLMCNIIAVVGAVVVTTSKTYASCMVGRVILGAGGSAFWTLGPASIGDIFFRHEKGKKIGVSTLAIVIAPFLGTIIGGPIIENEKLGWPASQWIPLIFMAAGFIMQIFFLPETIYIRETRAHPAIMSTSTPGKPTFWDRYGIHIPKRSEEKQHSFLFIATRPFVLFKFPAVILSAFWFGIAYMMHVGITSEIPLIFEEHYDFSVLEIGLSGFSGLIGALLGEVYAGPSLDFIAKRTMKQGREWRPEYRLQAIWPALITVPAGLIMFGTSIQFGNVWITPLIGQAVYIFGIEIATTVIQTYILECYPRQGAEANLVFNLIRNLFSYISPFFVQPMIATLGTTSPFGLSAALTAFFFPFTVGVLMWRGKQIRDKGGDPGWSRD

SEQ ID NO: 8 - protein, 1435 aa, Penicillium brasilianum
MEDHEKEYDSTSPPGTATEEGNGGYFNTLTVPEINLREASSAETLTPHASVVQPPKKAAEWHMTPQVIRNAERDEAAGFKRRELGVTWQDLSVEVLAAEAAVKENMISQFNVPQLIKDFRRKPPLKSILSNSHGCVKPGEMLLVLGRPGSGCTTLLKMLANRREGYQNITGDVRFGNMTPEEASRYQGQIVMNTEEELFYPRLTVGQTMDFATKLKVPYHLPGEGKSVAEYTAETKQFLLESMGIAHTADTKVGNEFVRGVSGGERKRVSIIECLATRGSVFTWDNSTRGLDASTALEWAKALRAMTDVQGLSTIVTLYQAGNGIYNLFDKVLVLDEGKQIYYGPAAEAKPFMENLGFVYTDGANIGDFLTGLTVPTERKIRPGWENRFPRTADAILTEYQNSATYKNEVSLYGYPDTDLAAERTEAFKESVAWEKSKHLPKGSDLTTSFWAQLMSCTARQYQILWGEKSTFLIKQILSCVMALIAGSCFYNSPDTSAGLFTKGGAVFFSLLYNCIVAMSEVTESFKGRPILTKHKSFAMYHPAAFCLAQITADFPVLLFQCTIFSVVIYWMVGLKHTAAAFFTFWAILFTTTLCITALFRFIGAAFSSFEAASKISGTAVKAIVMYAGYMIPKPEIKNWFLEFYYTNPFAYAFQAALTNEFHDQHIDCVGGNLIPSGPGYEDVGSGYKACAGVGGALPGADYVTGDQYLSSLHYKHSQLWRNFGVVWAWWGFFAVLTVVFTCFWKSGAASGSSLLIPRENLKKHQVGNDEEAQNNEKHAARTTTDEPVQVEDDNLVRNTSIFTWKNLTYTVKTPTGDRVLLDNINGWVKPGMLGALMGSSGAGKTTLLDVLAQRKTEGTIKGSILVDGRELPVSFQRMAGYCEQLDVHESYATVREALEFSALLRQSRDTPKAEKLKYVDTIIDLLELHDLADTLIGSVGNGLSVEQRKRVTIGVELVSKPSILIFLDEPTSGLDGQSAYNTVRFLRKLADVGQAVLVTIHQPSAQLFAQFDTLLLLARGGKTVYFGDIGDNGSTIKQYFGNYGAICPQEANPAEFMIDVVTGGIQEVKDKDWHQIWLDSPEQHQMITELDRMIADAASKPPGTVNDGYEFSMPLWEQIKIVTQRMNVSLFRNTAYVNNKFSLHIISALLNGFSFWRPGPSVSALQLKMFTIFNFVFVAPGVINQLQPLFIQRRDIYDAREKKSKMYSWVAFVTGLIVSEFPYLCICAVLYFVCWYWPVWRLPHDSDRSGAIFFMMLIYEFIYTGIGQFIAAYAPNPTFAALVNPLIISVLVLFCGVFVPYDQLNVFWKYWMYYLNPFNYVVNGMLTFGLWGQKVTCNESEYAVFDPLNGTCGEYLATYMSGKGSGVNLLNPDATSSCKVCEYTTGSDFLQTLNINHYYYGWRDAGITVIYAISGYALVFGLMKLRTKASKKAE

SEQ ID NO: 9 - protein, 527 aa, Penicillium brasilianum
MTPISRLLSRAVNKPYRTKDITDEIPPTLDEDGFVSFGPGDIENPRNWSMRRRAGVTMSAVLLVVNATFASSSPSGCFPSISKHFGVSTEVAGLTITLFLLGYCAGPLIFAPLSEFYGRRWIFYITFLLYLAFNFLCAFPPNFGSLLVGRFLTGTFVSAPLSNCPGVLADVWNPLERANAMAGFSAMVWIGPALGPVVAGFLQLKEDWRWSFYVLLWLGGASAVIMLTIPETYAPIVLYNKARRIREAQIPGYENVKAPVEDGDRTLVGIYKVALTRPWIILFDPISLLCAIYMAFVYTLLYMLFTIYPIVFQEKRGWNSGVGELPLLGTVVGALFGGVIVVADTRMRQKRIDNGTTKMEDAVPEDRLPLAMGGGIGFAVTMFWFAWSAEFNSVHWIVPTLAGVFLSSALLLIFVGFLNYLVDVYQMYAASAIAANTIARSACGAAAPLFTSQMFAALGVGGGGSLIAGVATLLAAIPFLFYKYGKQIRMRSKFAPTTKEERPAEENKDEERGLGDGAVSSSILGAQ

SEQ ID NO: 10 - protein, 514 aa, Penicillium brasilianum
MAASNEHATPSIDSATTKTAPSVDSTPAMSDHVNEDLEKGTRTTDPVEAEKHDLNVNPLSEKIEGTTPAPAGPPGPGPPPDGGAEAWLVVLGAFCGLFVSFGWINCIGVFQTYYETHQLSNLSTSTVTWITSLETFVMFFAGPVFGTLFDSYGPRYILLGGTFLHVFGLMMTSLSTEYYQFILAQGICSPLGASAIFNASINSVSTWFAKRRAFALGVTASGSSLGGVIFPIMVTNLIPEVGFPWAMRICAFLILAMLGVSNLTLKSRLKHTRKPFNFMNFVRPLKDIKFVVTVAAAFCFFWGMFLPFTFVITQAQRYGMSEHLSQYLIPILNAASVFGRTLPGYLADRVGRYNVMIFFSYLSGILVLALWLPSRSNAPAIVFSALYGFGSGAFVSLAPALIAQISDVREVGVRNGTCFSIIAFAALTGTPIGGALVPDVLTGSYTRLQVFSGVVMLAGATLFVVARLVVGGVKFGKSLGSGVLMGCISNGLIEHDRRMHWHYHVVICCAATDS

SEQ ID NO: 11 - protein, 872 aa, Penicillium brasilianum
MCQDHDLECSYTLPRKTRFYGSVDDLSDRYKCLEAIVRAAFPNDGISTVPELIRLGERMGYAMPDLSQKSGESPRIEELVRDFPTEAGDQGLAGSTQCTSSPPRTGAVNVPTESERRHSSSQVQENNSCPDEPVGLIRDTTGREHFIGPSGSLQFLGQLRRLLLISRSGDAVESRAPARLTATFTDEDAAQALEADGDQSELAALPSGGTGNGGDEGQEIDERSPASLGSALVRDFSSIPVNDIDEMRRQLPPRHVLDSLMRVYFKNVHPDFALFHRGTFEEEYETFMSKGRYYHQHARAGVHLSSPTLPEPGWLGCLHMMIAFASLNGSVDVAPDLDLTSLCRHCASLTRQLLPQFISKCTLSNVRALLLLSLFLHNHNERNAAWNLVGTAMRLSFAMGLHRASDNGSHFRPIEREVRKRVFCTLYGFEQFLASSLGRPSGFYDFEDVEIVPPREGVLDSGQDEDDEVMKLSLRLQVILAKARVSLAVKTLAVANERGNIDGLARQQQSSRETLEILKAWREDLASHHILNIPLISETDDPLCQYAEEIPRMSLQDLKAMMGWQSRPRLRAALVLHLQYRYIAVLVTRSSLLRYVASAQRGEPEHEALLSRNEARTDPYNSEAGERLSDICVTHATQLCRLILLADSFGLVNGISAMDVFYVYCGVMVLILRSLRISSSASHYHDQREAHLQLELRKLIAQTREVLIRVNKCSTMKRFARVVATFEDGSRQDNIRPADGSTNRSTANCEMRTARQASRDPRGRFNHSIHAALDGGRASNLAIFPGAGGSLDTSSSLPVSQQEPLNFQHGYGNGIGPRLGISDPFWQPNLLTSFDGEPEANGWMMDPFLAMDGTGVVDWGDIESLLSRNPGQ

SEQ ID NO: 12 - DNA, 1056 bp, Penicillium brasilianum
atgggctcgctatctctaccggagacgtctcttgcggcaatccaagataaggagaccaaggccatctccgtcgccaaacgcccaactccagtccccgtcggcactcaagtcctcgtcaagctacactactccggtgtctgcgccacagatctccatctggcccgcggcagcgtcccctatctgcagcccaaggtctcggtaggcggccatgagggcacgggggtaatcgccagcctcggccccgacgtggacgccgcggagtggcacgtcggcgaccgggtcgcggtgcggtgggtgcacatcgtctgcgggaagtgcgaggtgtgcactaccggctttgagaacctgtgccaaagccggaagctcgctggcaaggacgttgagggcacctttgccgagtacgctattgcggacagctcgtacatggtccgcctgccggctggggtgagcgacgccgatgccgctccgatactgtgcgccggtgtcaccgtctacaaggcgctcaagatcgccagcctccgggcgggctcgtgggtcgccgtggccggggctggtggcggcttgggtcatctggctattcaatatgcacgtgccatggggctcaaggtcgtggccctggatgctaggaaacgggatctctgcctgagcctgggcgcggaatcgtacatcgacgtgctcgaaacggacgactgcgtagcccaggtgatcaaggtcacggatgggggcgctcatggcgcactcatctgcgcctcgtccggccaggcctatgacgatgccgtcaaattcctaagatggaccggcaccctggtgtgtatcggcctccccccaaagccgacactgctctccctaggccccgcggacttcgtcgcccgtggcatcaaggtcatgggcacatccacaggtgaccgccaggacacggtggaggccctcgcctttgtcgccaagggccaggtaaaaccgcagttaactgagagaaggctggaggatgttgaggagatcttaaaggagatcgagaatgggactatgcagggtaaagctgtgatcaggatagcctga

SEQ ID NO: 13 - DNA, 1020 bp, Penicillium brasilianum
atgagtctgccgtctcactacaaacgggccgcctttaaagaggctggcggcccgctcaccattgaagaagtcgacttgaccatgccggatgctggcgaagtgctggtgaaggtggaagcttgcggagtttgtttctccgacacggttccacaggctcatggcctgggggggaaatttccaatcgtacccggtcatgaaatcatcggacatgtggttgcgacaggtgacggtgtctcggactgggaggttggcgatcgtatcggcgaaggatggcacggcggccatgacggaacttgtccctcctgtcgccagggccatttccaaatgtgcgacaatcagagcatcaatggggtgaccaaaaacggcggatatgcccaatactgcattctccgtagtgaagctgctgtcagaattcccactcatgtctcagccgccgagtatgccccaattctctgcgcgggcgtgaccgtcttcaactcaatgcgtcaaataggggtcaaacctggctctaccgttgccattcagggtctgggaggtctcggccatcttgccatccaatatgcgaatcgcttcggcttccgagtggtggccatctctcgcgatgatcaaaaggagcgatttgtgcgagacctcggtgcccacgagtatatcaatacaagcgaagaagatgtcggaagcgcgctccagaaactgggaggtgcttctctgattgtagccaccgcgcccaatgcccgggccatttcccctctattgaagggccttagacctctgggtaagcttcttattctcgccgttccgggtgaaatccccctcgatacacggcttatggtcgcccgaggtctttctgtccacggatggccgagtgggcatgcacttgattctgaggaaaccattcgctttacagaattagaggatatcaagtgtatgatccagacatattctcttgatcgtgccaatgaagcattcgatgccatgatttcggggtcggttcgattccgtgcggtgattaccatggagtaa

SEQ ID NO: 14 - DNA, 1518 bp, Penicillium brasilianum
atgactcaaaccaacgtccatgtcaacaagtctgatacttcactggcagctccgcaacagttattcatctcgggaaaataccagaacagccagaggaatgggaccttccccgtgaagaaccccatgacgggcgagaccatatacgagtgcgtctcagcctctctagacgactacgccgccgccatcgaggaagcagatgcagcccaaccttcgtgggctcgcctcggcccctcagcccggcggttgatccttctcaaggccgccgacatcatggagacgtacatcgagacagacgcgccggccatcctttcagccgaggtttcggccactaggggatgggtcagggccaacattctgtccacagccggcgtcttccgcgagaccgccgccctggcaacgcatatcaagggagagatcgttcccgcggaccggccgggcaccacgatcctggtgagccgcgagcccgtcggcgtggtcctggccatcagcccctggaatatgcccgccacactgacggccagggcgatctgctgcccgctgatctgcgggaatagcgtggtcctgaggccgtctgagttcagccccaagtcgcagcacctcgtcgtgcgcgctctgaccgaggctggactgccggcggggtgcctgcagttcctgcccaccagcaccgccgacacgccgcgagcgatcgagtttgcaatccgacacccgaaggtgagccgcgcaaacttcacgggcagcgaccgcgttggccgcatcatcgccgggctatcggcatcctgtctgaagccgtgcgtgctggagctaggcggcaaggcgcccgtcgtggtcctggaggatgccgatgtggaggccgccgtggaggcagtggtgtatggcgcgatgtccaacagcgggcagatatgtatgtccacagagcgggcgatcgtgcatcgctccctggcagcggacttcaaggccctgttggtgaaacgggcggagagcctgcgggtagggaaccacctcgaggacccggacgtgcagctctcgggccttttcactgccgcctccgccgagcgtgtactcggcctgatcaagggcgccgtcaacgcaggtgccacgctcctggcgggcgatctggctcttcacggaccgtgccagacaatcatggctccccacatcctcacgggcgtcacgcgggatatggacctcttccatcgggagacgttcggccccgtgctcttcgtgtccgagttcgacacggacgatgaggccatcgcgcaggccaacgacaccgagttctctctgtgtgccagcgtcttctcgcgtgacgtcctgcgcgccatggacacggccaagcggatccggacagggagctgccacgtcaatgggccgaccgtgtacatcgaggcgccgctgcccaacgggggtgtcggcggcgggagcggctacggccggttcggtggtgtggcgggcatcgaggagtttacggagaggcagatcgtgagcttagcgaagccggggattaagtatgcgttctag

SEQ ID NO: 15 - DNA, 1284 bp, Penicillium brasilianum
atgcctcacgcatccagatcattgaacgttctcatcgtcggtgctggcttgggcggcctcgcggcgggattagctttgcagacggacggccacaaggtcacaatcattgatgccgctcctgagttcgcagaggccggagcggggatcaggatcccacccaactcgagccggctgctcatgcgatggggcgttgatctggagaggatgaagaagtcgacttcacagagatatcacttcatccgctggaaggatggcagcaccatcttcgacttgcccttcaacaatatcgtcgagacacacggggcgccttactggcttgtccatagagccgacttgcacgccgctctgctcgatgcgaccctgaaggccggtgtcaaggttctaaacaacaagcttgtcacgtcctacgacttcgaggcaccgagcgccaccacacaggatggcgagaccttcaaggccgatcttatcgtcggtgcggacggcataaagtccatctgccgaccccttcttaccggtcagccggacgtcccgcgggacacgggcgacgtcgcctatcggattctcatccctggtgagaaactgctggccgacccggacctggcccatctgatccgcgacccttgcacgacatcgtggtgtggcccggacgcgcacctggtcggctacccgatccgcaacggcgagatgtacaacatcgtcatgtgtgccacctcctacaacgagaccacggacgaggtctgggtcgtcaagggcgacaacagcgagctgtgcaagcgcttcgccagctgggaaccccaggtgcggaagctctgcgccctcacgggcgacttcatgaagtggcgcctgtgcgacctgcccaacctcgcccgctggacgcacccctcgggcaaggccgtgctgctgggcgacagctgccaccccatgctgccttacctggcccagggcgctgcccaggccgtcgaggatgccgccgtcctgcgccaggtgctcgcccaggacatggacatggccgcggccctaaagcagtatgagcagatccgcatgccgcgggcgagcctggtgcaggccaagactcgcgagcaccagtacatcctccacgtcgacgacggtcacgagcagcaagaccgggacaagaagttggctctcgatgcggcggagaacccggtcttctggggctacgacgaccggagaaaatggctctttagccatgatgcagaggtgatccaaaaagaaggtgcgaactggagagacgggcccaacatgaatggcgtgcatgttgcttag

SEQ ID NO: 16 - DNA, 738 bp, Penicillium brasilianum
atgtctctgtctggcaaagtcgtcctcatcaccggctcctccaagggaatcggcaaagctgctgcccttcgtgtcgccagcgaaggtgccaacgtcgtgatcaactatcttcgtgacccggttgcagccaacaatctcgtcgaccaaatcggtgccgaccgcgcccttgctgttcaagctgatgcttcaaagctggccgacctcgatcgcctcgtcaatgccgctgtcgcccagttcggcaagatagatgttcttattccaaatgccggaatcctcccgcttagagacttggagcatactagcgaagaggactttgacagaacctacaatctaatggtaaagggaccatacttcctggcccagaaagccgtgaagcatatgccccctggaggacgaatcatctttgtctctacctcaaccgcccgattcgcaagcgtggctcccgcatacctactctacacctcctccaaaggcgcgatcgagcagatgacccgaatcatggccaaggatttggcgcgaaagggaatcttggtgaatgcggtcgctcccggtccaacttcaaccgaactcttcctcgagggaaagccggagcaaatgatcaaggctatctctgggtttagtccattcaatcggatcggagagcccgaggaaattgccgcagtcatggcgttcttgtctgggaaggacagcagttggatctcaggacaggttgtggctgtgaacggagctatggcatga

SEQ ID NO: 17 - DNA, 1746 bp, Penicillium brasilianum
atggcatctctcattcgagaggccccctttgggcaaatagttcgatatctcaccaacaacaaatatttccaatatcccgaagaaaagccggacttcaaacttcccgatacatggcttcaattattgaacgaatcggatgccgcgacgatagccgatcccgagaaaacggagccggagccagagggtcagggctacgatgcaaccagcgaggcgattagtcgcgcatcaacccaaaactccttgcccttcacagaagctcgactggaagcggatgagcagcatgagatcgaaaagatcaagtccattcctattcagcctaaaaagaccaaggatggtgcgattttggtcgactggtactataccgatgatgcagagaacccacacaattggtcgaatcgaaagcgagcacttctgacgacgctcatttgtctttatacctttgtggtatacacgacctctgcaatttacacatcctccgtgcccgggatcatgaaggagttcggcgtcagtgacttggttgctacactgggactgtccttgtatgtccttggctatggaacgggtcccctgatcttctcgcctctgagtgagatccccgtgattggtcggaacccggtctatatcgtgaccatgttcctctttgtgattctctccattcccactgcttttgtgggtaactttgcaggactcatggtgcttcgtttcctgcaaggattcttcggctcgccctgtctcgcttctggaggtgcttcaattggcgacatgtacagtctcatgtctctcccttatgccatgatgagctgggtgtctgccgcttactgtggtcccgccctgggtcctctcatcagtggcttcgcagttcccgccgagacctggcgctggtccctgtttgaatccatttggatgtcagctccagtcctcattctgatgttcttcttcctccccgagactagcagtgcaactatcctgctccgtcgtgccgctcgtctccgcaagatccataacaacgcacgcttcatggctcagtccgagattgaccagcgtaacatgaaggtctcggccgttgctgtcgacgccctgattaagcctttggagattaccatcaaggatcccgcggtgctcttcgtccaggtctacaccgccattatctacggcatctactactccttcttcgaggtcttccccctggtctaccccgtcgattatggcatgaatcttggccaagttggcctggtcttcctgtgtatcctggtgtcctgcatcatcggtatcgccatctactggtcttacctttacttctggatgaaccctcgcattgaacgcttcggattcccagctcaagagtcccgtcttatccccgccctgccagcttctattggacccaccattggcttgttcctctttgcctggacagcccgtgcctcaatccactggattgccccgacaatcggaatcaccatctacggtgcgacggtcttcatcgtgatgcagtgcttgtttgtctacatccccttgagctatccaatgtacgccgctagtctgttcgctgcgaacgacttcttccgcagtgctctggcttgcggtagtgtcctgtttgctcacccgttgtttggcaacctcggtgtcgctcggggtaccagtctactcggtggtctgagtgtgattggtattatcggaatatggctgctttactactatggtgcccggcttcgctctttgagcaagtttgccatctccgatgattga

SEQ ID NO: 18 - DNA, 1542 bp, Penicillium brasilianum
atgagcacgaccaaggaagctttcccacatacggatagcgacataatggaggactcggagaagaatctcccagagtgcgagcatatcgtctccgtggagcccaccctcaagatgcgcgacggcatcgtcctgatgccgcaaccgtccgacgaccccaacgacccgctcaactggtcctggttccgcaagcacgccgccatgttcaccctctcgtatttggccctcgtctgttacgtggctgtgaccacgctggttacgggaacagtgcccctagccaagtccatgcatgtctccaagtcgacggccgtctatctgggcaacacgcccgtcgctctctacgccgtggcgccctggttctggagcccgctgagccactttataggccgtcgcccggtgctgctgatgtgtaatatcatcgccgtcgtcggggcggtcgttgttacgacgtccaagacatatgcgtcttgcatggttggccgcgtcatcctcggtgccggtggttcggccttctggacactggggccagccagtattggggacattttcttccgccacgagaagggcaagaagattggtgtgtcgaccttggccattgtgatcgccccattcttggggacaatcatcggcggacccatcatagaaaacgagaagctgggctggccggcctcccagtggatccccctgattttcatggccgccggcttcatcatgcagatcttcttcctcccggagaccatctacatccgagagacacgcgcgcatcctgcaatcatgtccacatctacgccgggcaagcccacgttctgggaccgctatgggatccacatccccaagcgctcggaggagaagcagcacagctttctcttcatcgcgacgcgccctttcgtcctcttcaagttccccgcggtgatactgtcggccttctggttcggcatcgcctacatgatgcacgtgggcatcacgtccgagatcccgctcatcttcgaggagcactacgatttctccgtgctcgagatcgggctgtcgggcttctcgggactcatcggcgccctgctcggcgaggtatacgcgggaccctcgctggatttcatcgctaagcggaccatgaagcagggtcgcgagtggcgccccgagtaccgcctgcaggcgatctggccggcgcttattaccgtgccggctggtctgatcatgttcggcacatcgatccagttcggaaatgtttggatcactcctctgatcgggcaggccgtttatatcttcggcatcgagattgccaccacggttattcaaacctacattttagaatgttaccctcgccagggcgccgaggcgaacttggtcttcaacctcatccgcaacctgttctcctatataagtccgttctttgtgcagccaatgatcgccaccctcggtaccacctctccgtttggtctctctgctgccttgactgccttcttcttccctttcactgtgggtgtcttgatgtggcggggcaaacagatccgggataaaggaggcgacccgggctggagcagggattaa

SEQ ID NO: 19 - DNA, 4308 bp, Penicillium brasilianum
atggaggaccacgaaaaagaatacgatagtacctcacctcccggaacggccacggaagaagggaatggaggttacttcaataccctcaccgttcctgaaatcaacctccgagaagccagcagtgccgaaaccttaactcctcacgcctccgtcgtccaacctcccaaaaaggccgcagaatggcatatgacaccacaagtcattcgcaatgccgaacgtgatgaagcagctggttttaaaaggcgtgagctgggtgtcacttggcaagacctgtcggtcgaggttctcgctgctgaagcggctgtcaaagagaacatgatctcccaattcaacgttcctcaactcatcaaggatttccgccgcaaaccaccactcaagtcgatcttgtccaacagccatggatgtgttaagcctggagaaatgcttctcgttcttggaagacccggatccggatgcactacccttctcaaaatgcttgccaaccgtcgtgagggatatcagaacatcaccggagatgtaagattcggaaatatgactccggaagaagcatcaagatatcaaggccagattgtgatgaataccgaggaggagcttttctaccctcgtttgacggtgggtcagacaatggattttgctaccaagcttaaggtcccatatcaccttcctggagaagggaagagcgttgctgagtataccgccgaaacgaagcaattcctcctcgagtctatgggaatcgcccatactgccgatacaaaagttggcaatgaattcgtccgaggtgtcagtggtggagaaagaaagcgagtgtctattattgagtgccttgccacaagaggctctgtttttacttgggataactcaacgagaggactcgatgcttccacggccttagagtgggccaaagcccttcgcgccatgaccgacgtccaaggtctttcgactattgtaacgctctatcaagctggaaatggaatttataatcttttcgacaaagttctcgtcctcgacgaaggaaagcagatctattacggccctgccgcggaagcgaaacctttcatggagaaccttggctttgtttacactgatggtgccaacattggtgatttcctcacggggttgactgttccgaccgagcgaaagatcagacctggttgggaaaatcggttccccaggacggccgacgccattttgaccgagtaccagaactcggcgacatataagaatgaagtctcactatacggatatcccgacactgaccttgctgccgaacgcactgaggccttcaaggaatctgtggcctgggaaaagtctaaacacttacccaagggtagtgacctgactactagtttctgggcccagctcatgtcatgcacggctagacagtaccagatcctctggggcgagaagagcacgttcctgatcaaacagattctgtcttgtgtcatggccttgattgccgggtcttgcttctacaactctccagatacctctgcaggtctcttcaccaagggtggtgccgttttcttctcgttgctttacaactgcattgtggccatgtccgaggtcaccgaatctttcaaaggtcgtcctattttgacgaaacacaaatcttttgccatgtatcacccggctgctttctgtctggcccaaattactgcggatttcccggtgttactgttccaatgcacgatcttctcggtcgttatctattggatggttggattgaagcataccgcggctgcatttttcactttctgggcaatccttttcactacgaccttgtgcatcacagcgttattcagatttatcggcgctgctttcagtagcttcgaagctgcatccaagatcagtggcaccgctgttaaggcaatcgtcatgtatgcaggttacatgattccaaagccagagatcaagaactggttcctcgagttctactacaccaatccgtttgcttatgcattccaagctgctttgaccaacgaattccacgaccagcatatcgactgcgttggcggtaatcttattcccagtggtcctggatacgaggatgttggatccggctataaagcatgtgctggagttggtggtgctcttcctggtgcagattatgtgactggagatcagtatctttcttctctacactacaagcattctcaattgtggcgaaacttcggcgttgtctgggcctggtggggcttcttcgctgttctcacggtcgtcttcacttgcttctggaaatctggtgctgcatctggatcttcgcttctcattccccgcgagaaccttaagaaacaccaagttggcaatgatgaggaagcccaaaacaatgagaaacatgccgctcggacgaccaccgatgagccagttcaagtcgaggatgacaatcttgtgcgcaacacatctatcttcacatggaagaatctcacctatacagtcaaaacaccaactggcgaccgagtcctcctggacaacatcaatggatgggtgaaacctggtatgcttggtgcactcatgggatcttccggagccggcaaaacaactctacttgatgttcttgcacagcgcaagacggaaggtaccatcaaaggctccattttggttgacggtcgcgaattacctgtctctttccaaagaatggccggctactgtgagcaattggatgttcatgagtcttacgccactgtgagagaagccctggagttttctgctctcctgcgacagtctcgagatactcctaaagctgagaaacttaaatacgtcgacacaattattgacctcttggagctgcatgatctcgccgacactctgatcggttccgtgggtaatggcttgtctgttgaacagcgcaaacgtgtgacaatcggtgtggagctcgtgtcgaagcctagtattctcatcttcttggatgagcccacttcaggtcttgatggtcaatctgcgtataacactgtcagatttcttcggaaactagccgatgtgggccaagcagttcttgtcacgattcaccagccttcagctcagctattcgctcaattcgatacccttctactccttgcgagaggtggcaaaacggtctactttggtgacatcggggacaatggatccacgattaagcagtactttggcaattacggggctatctgccctcaagaggcaaatccagcagagttcatgattgacgtcgttaccggtggtatccaagaagtgaaggacaaggattggcatcagatctggcttgactctcccgagcagcatcagatgatcaccgaattggacagaatgattgcagatgccgctagtaagccaccgggaactgtcaatgatggctatgagttttcaatgcctctctgggaacagatcaagattgtcactcagcgcatgaacgtctcgcttttccgtaatacggcctatgtcaacaacaagttctcgcttcacatcatctcagcattgttgaacggattctctttctggcgacctggtcctagtgtgagtgcgttgcagctgaagatgttcaccatcttcaattttgttttcgtcgctccaggtgtcatcaatcaactccaacccctcttcatccagcgccgcgatatctacgatgctcgcgaaaagaagtccaagatgtattcctgggtagctttcgtcactgggcttatcgtttcggagttcccatatctctgcatctgcgcagttctatactttgtttgctggtactggcccgtctggagattgcctcatgactctgaccgttctggagccatcttcttcatgatgttgatctacgagttcatctacactggtattggccagttcattgctgcgtatgcaccgaacccaacctttgcggcacttgtcaacccacttatcatcagtgttcttgttctcttctgcggtgtgtttgtgccatatgaccagctgaacgtattctggaagtactggatgtattacctcaacccattcaactatgtcgtcaacggcatgttgactttcggtctctggggccagaaagtcacctgcaatgagagcgagtatgcagtctttgatccgctcaatggtacttgcggcgagtatctggcgacttacatgagtggcaagggcagcggagtcaatctgctcaacccggacgctacctcgagctgcaaggtctgtgagtacacaactggaagtgacttcctacagacgctcaacatcaaccattactattatggatggagagatgcgggcatcactgttatctatgctatctcgggctatgcacttgtgtttggtctgatgaagctccggaccaaggcgtccaagaaggcagagtaa

SEQ ID NO: 20 - DNA, 1584 bp, Penicillium brasilianum
atgacacctatctcgcgcctattgtcccgtgcggtcaataaaccctaccgaacgaaagatatcacggacgagattccgcctacactcgatgaagatggatttgtgagctttggcccgggggacattgagaaccctcggaattggtcgatgcgccgccgagctggtgtgacaatgtctgcggtgcttttagttgtgaatgccacttttgcctcgagctcgccgagcggatgtttcccatcaatctcaaagcatttcggtgtttccactgaagttgccggattgacgataactctgttcctgctagggtactgtgctgggcctttgattttcgctccgctgagcgagttctacggtcgccgatggatcttctacattacctttctgctttatctggccttcaactttctttgtgcattcccgccgaattttggaagcttgttggttggaagatttctgacggggacctttgtgtctgctccattgagtaactgccccggtgttttggccgatgtatggaatccgcttgagcgcgccaatgctatggctgggttctcggcaatggtctggattgggccagcactcggacctgtggttgcaggtttcctgcagttgaaagaagattggagatggagcttctatgtgcttctctggctcggtggagcatcagccgtcatcatgttgacaatacccgagacttacgcgccgattgttctgtataacaaagcacgacggatccgcgaggcacagattcccggatatgaaaacgtgaaagcccctgtcgaggacggtgaccggactctcgtgggcatctataaggttgctctgactcggccgtggatcattctgttcgacccgatctccttgctgtgtgcaatttacatggcgttcgtctatacattgctctacatgttgtttaccatataccccattgtgtttcaggaaaaacggggctggaactctggtgttggggagctgccgttgctgggaaccgtcgtaggtgccctatttggtggtgttatcgttgtagcagacacccggatgcgccagaaaaggattgacaacggtacgacaaagatggaagatgctgtgccggaagaccgtctcccgctagcgatgggtggaggcatcggctttgccgtgacgatgttctggttcgcctggtcggctgagttcaactctgtccattggattgtcccaactctggcaggagtgtttctctccagtgccctgctactcatcttcgttgggttcctcaactatctcgtggatgtttatcaaatgtacgctgcatccgccattgctgcgaacacgatcgctcgttctgcctgtggagcggcagcaccactattcacatcccaaatgttcgcggccttgggcgtgggaggaggaggcagcttgatcgcgggtgtggccaccctccttgccgctattcctttcttgttctataagtatgggaagcagattagaatgcgcagcaaatttgccccgactaccaaagaagaacggcccgccgaagagaataaagatgaagagcgtggactgggagacggtgctgtcagcagtagtatcttgggggcgcaataa

SEQ ID NO: 21 - DNA, 1545 bp, Penicillium brasilianum
atggctgccagtaatgagcatgcgactccctccatcgacagtgctaccacaaaaaccgctccgtcagtggattcaacgccggccatgagcgaccatgtgaacgaggaccttgagaaggggactcgtacgaccgatcccgtcgaggcagagaagcacgatctcaatgtaaacccgctatcagagaaaattgagggcaccactccagctcccgctggtcctccggggcctggccctccaccggatggaggagctgaagcatggttggtcgtgctaggagccttttgcggactgttcgtcagttttggctggatcaactgtatcggtgtcttccaaacgtactacgaaacccaccagctcagtaatttgtccaccagtacggtgacatggatcacatcattggagactttcgtcatgttcttcgctggtcccgtctttggtactctctttgatagctatggcccccgatacatcctcctgggtggcacattcctccacgtcttcggcctcatgatgacctccctctcaacggagtactaccaattcatcctcgcgcaaggaatctgcagtccactcggtgcgagtgccatattcaacgccagcattaactctgtcagtacctggttcgccaaacgccgcgccttcgcactgggcgtcacagcttccggatcgagtctgggaggagtgatcttccccatcatggtgacaaatctgattccagaggttgggttcccttgggcaatgcgcatttgcgcgttcttgatcctagctatgctgggcgtttccaatctcaccctcaagtcgaggctgaagcatacaaggaagccgtttaattttatgaatttcgttcggccgctgaaggatatcaaattcgtcgtcacggttgctgcggccttttgtttcttctggggtatgttcttgccttttacgtttgttatcactcaggcgcagcgatatggcatgtcggagcatttgtcgcagtatctcattccgatcttgaatgctgctagtgtctttggccgtacactcccaggttacctggcagaccgcgttggtcgatataacgtgatgatcttcttctcttatctctccggcatcctcgtcctagccctctggcttccatcacgcagcaacgcccccgccatcgtctttagcgcattgtacggctttggctccggtgccttcgtgtccctcgccccagcccttatcgcccaaatctctgatgttcgtgaggttggtgtgcgcaacggcacctgcttctcgatcattgctttcgccgctcttaccggtacccctattggcggtgccttggttcctgatgttctgactggctcgtacacgaggctgcaggtcttcagtggcgtggtcatgcttgcaggtgcgacactgtttgtggtcgctcggcttgttgttggtggcgtgaagtttggcaagtccctgggctctggtgttttaatggggtgcatttcaaacggtttaattgagcatgatagacggatgcattggcattatcacgtagtgatttgttgtgctgccactgacagctaa

SEQ ID NO: 22 - DNA, 2619 bp, Penicillium brasilianum
atgtgccaggaccacgacctggaatgttcttatacacttccgcgaaagaccaggttctacggcagcgtggatgatctgagtgatcgatataaatgcttagaggccatagtacgtgctgcatttcccaatgacggcatctccaccgtcccggagctcatccggctgggagagcgcatgggatatgccatgccggacctatctcagaaatcaggggagtcacctaggatagaggagctggtgagagacttccccaccgaagctggagaccagggccttgctgggtccacacagtgtacctcttcgccaccaagaacgggtgccgtgaatgtccctactgagagtgaaagaaggcactcttccagccaagtccaggaaaacaactcctgtccagatgaacctgtcggactaattagagacaccactgggcgagaacatttcatcggcccgtctggaagcctgcagtttctgggccagctccggaggctacttcttatatcccgcagcggggatgccgtggagtcccgggcacccgctcgccttacggccacgttcaccgatgaagatgcagcgcaagccctggaagcagacggtgaccagagtgagctcgccgctttgccttctggaggcactggcaacggtggcgatgaaggccaggagattgacgagcgctcccctgcctccttgggttctgctcttgtcagggatttttccagcattcccgtcaatgacatcgacgagatgagaaggcagcttccgcctcgccacgttctcgattctctgatgcgagtatatttcaagaatgtccatccggactttgccctattccaccgcggtaccttcgaggaagaatacgagaccttcatgtccaaagggcggtactatcatcagcacgctcgagctggtgtacatttatcctctcccacgttacctgaaccaggctggttaggctgtttgcacatgatgattgcctttgcctcgctaaatggctctgtcgatgtcgctcccgacctggatcttacttccctctgtcgccattgtgccagtcttacccgccagctcctgcctcaatttatctcaaagtgcaccctctccaacgtccgggccctcctacttctatcccttttccttcataaccacaatgaacgtaacgcggcttggaacctggttgggactgccatgcgcctttccttcgccatggggctgcaccgggccagcgacaacgggtcgcacttccggcctatagaaagggaggtgcgcaagcgcgtcttctgcacgctctacggctttgagcaattcctcgcgtccagcctggggagaccaagcgggttctacgacttcgaggatgtggagatagttcctccgcgtgagggagtgctggacagtgggcaggacgaggacgacgaggtcatgaaactttcgctgaggctgcaagttatcctggccaaggccagggtctcccttgccgtcaagacactggccgtggccaatgagaggggcaacatcgacggtctggctcggcaacagcagtcttcaagggagacgctggagatcttgaaggcgtggagggaggatcttgcctcccaccacatcttaaacatcccgttgatcagtgagacagatgatccgctttgccagtacgccgaggagataccgcgaatgtcactccaagatctcaaggccatgatgggttggcaaagccgacctcgacttcgggctgccctggtcctgcacctccaatacaggtacattgctgtcttggtgacgcggtcttctttgctgcgatacgtcgcgtccgcgcaacgtggtgaaccagagcacgaagccctgctcagccggaacgaggctagaaccgacccgtataacagcgaggcaggagaacggctatcggacatctgcgtcacgcacgccacgcagctatgccggctcatcttgcttgcagactcctttggcttggtgaatggcatatcggccatggacgtcttctacgtctactgcggcgtcatggtgctcatattgcggtcgctacggatttctagctctgcaagccactaccatgaccagcgcgaggcgcatctgcagctggagttgcgcaagctgatcgcacaaacgagagaagtgctcatacgtgtgaacaaatgcagcacaatgaagcgattcgcgcgcgtggtggctaccttcgaggatggatcaaggcaggataacatcaggcctgccgatggttctaccaatcggtcaacggccaactgtgagatgcgaacggctcggcaggcatctcgtgaccctcggggccggttcaaccattctatccacgccgccttagatggcgggcgagcgagcaacctggcaatctttccgggtgcaggaggatcgctggacacgtcctcctccctgccagtctcgcagcaagagccgcttaacttccagcacggatatggtaatggaattgggccgaggctcggtatatcagatcctttctggcaaccaaacttattgacgtcctttgacggggagccagaggcaaacggttggatgatggacccattcctcgcgatggacgggaccggcgttgtggactggggcgatattgagtccctcttgtcacggaatcctggtcaatag

SEQ ID NO: 23 - protein, 458 aa, Penicillium brasilianum
MANQRVHVGIVGAGIGGLAAAIAFRRAGVDVTVLEAADELLEMTPNVSVLLQKWGVADVIGDNLVQIEELNMRRKDGTLVGHTEIPIVEKALGRPWWLVHRAHLHEGLATVAKRLGAVIHINSRVISLDYQEGGEVTVETQKGDKYHFDLCVGADGVNSIVRSTLFPGIKPDAKTTNCAYRAIIPYERIRQDPIAKELIQKLTMEVWMGHNAYIITYPISGGETFNLVLSHHRPEKLYSTQPNVPVEEMRDAYKDFDPRIKRIVDMITETSRWPLLVTGPLQSWSSPQKNVVLMGDAAHSMVNHMAQGAATSMEDGAFLAKCVGAVTRGKLSLREAINIYELERMPKAFMKQQVSFLNGAIWHLPDGPKQQARDAAMAPSLDGKYLVRSSNLYDDPQTVLDVYGYDVEAHAEHALAKFAHGREALYPGTGLAPFMEDKYMGWFSKCQQNDRYAAVGKL

SEQ ID NO: 24 - DNA, 1377 bp, Penicillium brasilianum
atggccaatcagcgtgttcatgttggaatcgtcggcgcaggaataggagggctggcagccgccattgcctttcgacgtgcaggagtggatgtcactgtcctggaagcagcggatgaattgctcgaaatgacgccgaatgtatcggttctgctgcagaaatggggcgttgcagatgtgattggagacaacttggttcaaattgaagaactgaatatgcgccgcaaagatggcactcttgtcggacataccgaaataccgattgttgaaaaggcattgggcagaccgtggtggcttgttcatcgtgctcatctccacgaaggcctcgcgactgtggccaagcgtctgggagccgtgattcatatcaacagtcgcgtcatttcacttgactatcaggaggggggtgaagtcacggtcgaaacacaaaaaggggataagtatcatttcgacctctgtgtgggtgctgatggcgtcaacagcattgtccgaagcacactgtttccgggcataaagccggacgcgaaaaccaccaactgtgcttaccgggcgattataccgtatgagcgtatccgacaggacccaattgcaaaggagctcattcaaaagttgacgatggaggtctggatgggacataatgcctatattatcacctatcccatcagtggaggcgaaacattcaacctggtgctctcgcatcatcgtccagagaagctctatagtacccaaccgaatgttcctgtcgaggaaatgcgggatgcgtacaaggactttgaccctcggatcaaacgcattgttgatatgatcaccgagacttcacgctggccgctattggtgaccgggcccttgcaatcatggtcctccccacagaagaatgtggttttgatgggggatgcagcgcactcaatggtaaaccatatggcgcaaggggctgctacgtccatggaagatggcgcttttctcgcaaagtgcgtaggggcagttactcgtggaaagctgagtcttcgcgaagccatcaatatctacgagctcgagcggatgccaaaagccttcatgaaacaacaggtctcttttctgaatggtgccatctggcacttaccggatggacctaagcaacaggctcgtgatgcggcaatggcaccgtcacttgatgggaaatatttggttcgcagcagcaatctctacgatgaccctcagacggtgctggatgtatatggatatgacgttgaagcgcatgcagaacatgcgctggcaaagtttgcccacgggcgtgaggcgttgtatccaggcactggtttggcgcctttcatggaggacaaatacatggggtggttctcaaagtgtcagcaaaatgaccgctacgccgcagttgggaagctttag

SEQ ID NO: 25 - protein, 360 aa, Saccharomyces cerevisiae
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFTLVGYDKEFSD

SEQ ID NO: 26 - protein, 422 aa, Aspergillus niger
MCLKFEPLNILIVGAGLGGLFASLALRQDGHRITLLDASAEFKEAGAGIRIPPNSTRLLRRWGVDFDKMRKCVCQGYHFRRWQDGSTITEINPEDMIACHGAPYYLVHRADLHATLLAVAIEAGVVIHKNRHVTYYDFSVPIAVTADSEKWTADLIICADGIKSTARPLLTSQPDHPRDTGDIAYRILINGQDLLADPDLAGLITHPATTSWCGPDAHLVGYPIRDGELYNIVVCATSKGETTDEIWVVEGDNEELCARFSGWEPRVQKLCRLTKSFMKWRLCDLPALSTWIHPSGKACLLGDSCHPMLPYLAQGAAQAAEDAATICRALAEDTDITSALKRYERIRSPRASLIQSKTREHQYILHVDDGEEQCQRDELMRHDSPSSPIFWGYELRREWLFSHDAEKMEEATAEPRLRTAAL

SEQ ID NO: 27 - DNA, 1056 bp, Artificial
  hmfL1 coding sequence optimised for expression in yeast
atgggttccttatccttaccagaaacatcattagccgcaatccaagacaaagaaacaaaagctatctcagtcgccaaaagacctacaccagtacctgttggtacccaagtcttagtaaaattgcattattccggtgtttgtgccactgatttgcacttagctagaggttctgttccatacttacaacctaaggtttcagtcggtggtcatgaaggtaccggtgttattgcttctttgggtccagatgtcgacgcagcagaatggcatgtaggtgacagagtagcagttagatgggtacacatagtttgtggtaaatgcgaagtttgtactacaggtttcgaaaatttgtgccaatctagaaagttggctggtaaagatgttgaaggtactttcgccgaatatgcaattgccgactcttcatacatggttagattaccagctggtgtctcagatgcagacgccgctcctatcttgtgtgctggtgtcacagtatacaaagccttgaagatcgcttctttgagagcaggttcatgggttgctgtcgcaggtgctggtggtggtttaggtcatttggcaatccaatatgctagagcaatgggtttaaaagttgtcgcattggatgccagaaagagagacttgtgcttatccttgggtgctgaaagttacatcgacgttttagaaactgatgactgtgtcgcacaagtaattaaagttacagatggtggtgctcacggtgcattaatatgcgcttccagtggtcaagcctacgatgacgctgttaaatttttgagatggaccggtactttagtctgtataggtttgccacctaagccaacattgttatccttaggtcctgctgattttgtagccagaggtatcaaggttatgggtacaagtaccggtgacagacaagacacagttgaagccttggctttcgtcgctaaaggtcaagtaaagcctcaattaaccgaaagaagattggaagatgttgaagaaatcttaaaggaaatagaaaatggtacaatgcaaggtaaagccgtaatcagaatcgcatag

SEQ ID NO: 28 - DNA, 1518 bp, Artificial
  hmfN1 coding sequence optimised for expression in yeast
atgacccaaaccaatgtccacgtaaacaaatccgacacttccttagctgctccacaacaattattcatctccggtaaatatcaaaactctcaaagaaatggtacatttccagtcaaaaaccctatgactggtgaaacaatctatgaatgtgtttctgcatcattagatgactacgctgctgctatagaagaagctgatgccgcacaaccatcatgggctagattaggtccttccgcaagaagattgattttgttaaaggccgctgatataatggaaacatacatcgaaaccgacgctccagcaatcttgagtgctgaagtttctgcaacaagaggttgggtcagagccaatatattatctaccgctggtgttttcagagaaactgctgctttggcaacacatatcaaaggtgaaattgttccagctgatagacctggtactacaatcttagtttcaagagaaccagtcggtgttgtcttggctatttccccttggaatatgcctgcaaccttaactgccagagctatctgttgccctttaatttgtggtaactctgtagttttaagaccatccgaattttctcctaaatctcaacatttggtcgtaagagccttaacagaagctggtttgccagcaggttgcttgcaattcttaccaacatcaaccgcagatacccctagagccatagaatttgctatcagacaccctaaggttagtagagctaatttcactggttctgatagagtcggtagaattatagcaggtttatccgccagttgtttgaaaccatgcgttttggaattgggtggtaaagcccctgttgtcgtattagaagatgctgatgtcgaagctgctgttgaagcagttgtctacggtgccatgtctaactcaggtcaaatttgtatgagtacagaaagagctatagttcatagatcattggccgctgattttaaagcattgttagtaaagagagccgaatcattaagagttggtaatcacttggaagatccagacgttcaattgtcaggtttgtttactgctgcttccgcagaaagagtcttgggtttgattaaaggtgctgtaaacgcaggtgccaccttgttagctggtgacttggcattacatggtccatgccaaactataatggctcctcacatcttaaccggtgttactagagatatggacttgtttcatagagaaacattcggtccagtattgttcgttagtgaatttgatactgatgacgaagctatagcacaagccaatgacacagaattttctttatgtgcttcagtattctccagagatgttttgagagctatggataccgctaagagaataagaactggttcatgccacgtcaatggtcctactgtatatatcgaagcaccattgcctaacggtggtgttggtggtggttctggttacggtagatttggtggtgttgctggtattgaagagtttacagaaagacaaatagttagtttagccaagccaggtatcaagtatgccttttag

SEQ ID NO: 29 - DNA, 2139 bp, Artificial
  synthetic fragment comprising hmfN1 for expression in yeast
ctcccccgggttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaaatgacccaaaccaatgtccacgtaaacaaatccgacacttccttagctgctccacaacaattattcatctccggtaaatatcaaaactctcaaagaaatggtacatttccagtcaaaaaccctatgactggtgaaacaatctatgaatgtgtttctgcatcattagatgactacgctgctgctatagaagaagctgatgccgcacaaccatcatgggctagattaggtccttccgcaagaagattgattttgttaaaggccgctgatataatggaaacatacatcgaaaccgacgctccagcaatcttgagtgctgaagtttctgcaacaagaggttgggtcagagccaatatattatctaccgctggtgttttcagagaaactgctgctttggcaacacatatcaaaggtgaaattgttccagctgatagacctggtactacaatcttagtttcaagagaaccagtcggtgttgtcttggctatttccccttggaatatgcctgcaaccttaactgccagagctatctgttgccctttaatttgtggtaactctgtagttttaagaccatccgaattttctcctaaatctcaacatttggtcgtaagagccttaacagaagctggtttgccagcaggttgcttgcaattcttaccaacatcaaccgcagatacccctagagccatagaatttgctatcagacaccctaaggttagtagagctaatttcactggttctgatagagtcggtagaattatagcaggtttatccgccagttgtttgaaaccatgcgttttggaattgggtggtaaagcccctgttgtcgtattagaagatgctgatgtcgaagctgctgttgaagcagttgtctacggtgccatgtctaactcaggtcaaatttgtatgagtacagaaagagctatagttcatagatcattggccgctgattttaaagcattgttagtaaagagagccgaatcattaagagttggtaatcacttggaagatccagacgttcaattgtcaggtttgtttactgctgcttccgcagaaagagtcttgggtttgattaaaggtgctgtaaacgcaggtgccaccttgttagctggtgacttggcattacatggtccatgccaaactataatggctcctcacatcttaaccggtgttactagagatatggacttgtttcatagagaaacattcggtccagtattgttcgttagtgaatttgatactgatgacgaagctatagcacaagccaatgacacagaattttctttatgtgcttcagtattctccagagatgttttgagagctatggataccgctaagagaataagaactggttcatgccacgtcaatggtcctactgtatatatcgaagcaccattgcctaacggtggtgttggtggtggttctggttacggtagatttggtggtgttgctggtattgaagagtttacagaaagacaaatagttagtttagccaagccaggtatcaagtatgccttttagactagtgtga

SEQ ID NO: 30 - DNA, 3011 bp, Artificial
  Synthetic split marker fragment 5-flank hmfK1 with Hygromicin B marker
ctcggtcgctcttttgggtagatggtaggataactgacaggaagtctatgccagtactgcggaatcaaagaagcgtcggagcaatgaaccgtatgcacgtcggaatgggggaatgacacggagttatactcccggttcaagccccgcttatcaaccctgggcaggaattgcggggaagcaatgcggggaagctacaacgcttccccgcatggtctacgtctactcttggcacaacacatggaaatatccagcgaagtggttgactggtatcgagacttggtccgtttccagtcaaaatcacaccttcgtagaatccctggcgttaccgcctctcacgtcttttggctctgcttgtcagctggacgcagcacgcatcaattggcaagatgtcgtctacaagcgagtccttcactctcccaaatgggcgacagatggcttataccctctcgcccggtggttcctccgatcgcgtggtccttctctccaactccttggccgaagatctgacctcctgggagcgcgtcgtacctgtggtggaaaaccaaggcttccgcgtcctccgctacgaccagccaggccatggacgctcgggtgcacctactgaagccgaattgacctccatgacattcgagaccttggtcgatgacgtctaccgccttctcggacacctcaaaatcaacaatttgcatgcctgggtcggcgtctcgatgggcggcatcaaggccgtgtacttcaccgcgcgccacccaggcatcgtgaacaagatcgtcgtggccgacgccatcgccgcttccccgtccgtggtctgcatccccgataacttcgccgcgcgggtaagcgctgtgaagcagtcgggttccatctcggacgacctgtcaaatactcggaagcggtggttcggtgaagactggatggcgaagcatccggaggagacggcgaggatggagaagtccatggcgacaactacaattcagggtctcgaggcgtgctgtgctgcccttagcagcccgtcttttgacctgaggccgctatatacgaaggtgggacatggctgcgaagaagcactcattgtcgccggggagaaggatgcggaccttccagtcaagatgcaggagatgcggcaggcaatcgaggagagcctccggagctgtggaaagaaggtgcccgtgaggatggagattatcaagggtgccgggcatgttccctacattgatggtttcgaggacttctgcgaaatcatcaccaaattccttgcctagatagcactcgatctgcttcacgattgtgaaatgtcttaaattttcttgtcaatctagactttgcattaaaccaatcttgagacaaatcattcttcagtcgtattcccggagtgtgtgatgtcctggtcccatatttctcatttctcatcttcgctatggctagtccgggtagcaagtgcgcgcaagtgcgcgctagtaggcaaatagaagcaatcgtcggaagttgggcccgactaggataacctggactaggggcctgctgtacagtgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctg

SEQ ID NO: 31 - DNA, 2886 bp, Artificial
  Synthetic split marker fragment 3′-flank hmfK1 with Hygromicin B marker
tccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagaagtcctcgtgtactgtgtaagcgcccactccacatctccactcgagcctatcactgtggcaatgccttagactcttttattcttcaatcataccatacatcctcgcgttttgcattagtcctcggcgattcacctcgcagttttcctcctcctttggttacatcttctcagcttaggcggctggtacgtcttctgtatttatttgaggcagctgtaggaaactttcacgttgaaagttgacaagacgacttccacagcgtctgtgacgagggccacaagcctgttttttggtccatagaagttttctatctttgtttcgaacagcgtcttcctgtaacgggccagacccagcagtgaaaccatgtgccatggcctttcggacaccgcctcaacaggcaggattctaggggattctatcgcgaagattccatcagggacgagaccgattctctccatctccttcgacactaaggattctaaactattccaaaggattatatcagagacgagactgactcactttcctttcttcaatattgttgtaaaaagcatataggcctaacataattgaattaatgctacaacaccccttcgtactttcttcttgtaggctccagggatatagtcgttcctctatcgtaagagacgacgcctttctttagccttctcatcacgcttttagaaaaaaggccttttgacccataaccggatattctaaaagtaagctcaaagggtaggaggcagactcagaggagtagattttgaaacaagcaatgaaatggacgttgcttttaatgaggccgtagatgggataaagtctcctttaatattttgtcacaggaagcatgactagtgcaaaggcctctttgaactgcattatcttgaacaggacgggccacctcacttgtgtcgttttggccagttttgtttagatggagacctctagaatgggatattcacgcgctagttatcacagaatagatgaccctgtggggcatgtcctttttgggtatagtctgacttggtctaagtgatcactgagcgatcacatgactgcactcatgagcgcgtgctgacatttactcactgagcctgagcctgagtgccctcattaaagcggcggtagcccagacaggaaagccctaccggacaaaatccggtgtatataggcgcatattgcatcattgggtgtgagtctgaggtagcacaacttattatcttggccatgtggagaggtctataatgggctttgtccggatgcggcgatcagtcgacattgtactttgtagacgtgtatgggtggatggatggagatcggattgcgaagtatggagatcgcgattcttcagatgacgcacggagtcgatctcccgagctgctctcttatttacgtgtcaagagtacaataacaattgagctggacgatagatagtcttggaaatcaactatgacattgttaactagacatctattctaatgtacctcgaaagaatactcgggatgg

SEQ ID NO: 32 - DNA, 2658 bp, Artificial
  Synthetic split marker fragment 5-flank hmfK3 with Phleomycin marker
ccgtgcgctataataaccttcgaatcctggtgcatcgccgctgcttggaaagccttcttgacccagttcagatagaaggtgacacttcggtgacgtccgaaatgcggttgcttcagcaaatgggactgagcagcgtcatgatctgtgttgagtctgctatgtctatcatctcgattgtacacaatatgtctgccggtagtggctggcgacgtgagttcttgggtgcatggaattattcactctattacagtaggttgggttctgtaagatttgacctccagggagttgaaactaacgtaacacagctttcaatgcagcactagttattttcggctccctcattgtggcatcgaaggagcgtgaccgcaacccgtcagcctggatcatgatcagccattcccggtcgtatattgacacggcagctgaagcgctgcgtcagcttgacccgggaaaccctgttgttgaacggtgtcaagagtatctttcgcagctatctgcaacattggatcctctcagttagttatcccgcacagatgcaaacctccctttcgtcccatccgtgccatttcaagactggccctgcccgcgcttcaacaattgttccaaaaacctgctgacagcccttgcctacacaatgcacagtttcagaagagccatattgtgataataggcttgctttcgactttcctaccgaatattccaccgggctgttttctcttcacagtgccgacttagtaccgcctatggactttggcgaattcatgattggttagggttgtagcttttcggtacggccctgggaatagcgaggcattgactgctagtcaactgtagtggagcaaacagagaagggaccacgtaggtacaagctgggcgatgcagctgtccgaccctctcgagatgggttcgccgcggaaggagcaaatagtctcagtatccagctttgccgtaggaatgagaaaactgaactcaagacagaaacctcagcgtaccttttcaaagatgatgtaaaagatgtctctctcagagaaggatagtatagtaatgatcagatccgctggaaaatagtgcggcaccgaacccctgacggcccggcggcccggcgggtatggtaatgacggtaaattatctagggcatcgctatgatcatttgacaagacccgtgtgtcggcattatcattcggacatgattcaacgcctcaattttaccccatatgtgccaaccattccatctcgcgtgggcttagacatcccacgggcaaacgtcccgtccagccccagtcagcgaatgcggggataatccggggtttcctacccccatgggctgggccggaatgcctaactccccgaattggctgcccggggagtgcaaataaaatagttgcttgtatacatatatatatatccttcggtatatccaagacttcctgttgcatcaagcgcaatcctaagcttttttctccctaccacaagtagctctacaaacccgaaacctagaacaggcctgctgtacagtgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcc

SEQ ID NO: 33 - DNA, 2472 bp, Artificial
  Synthetic split marker fragment 3-flank hmfK3 with Phleomycin marker
gcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagaagtcctcgtgtactgtgtaagcgcccactccacatctccactcgagcctatcattcagcaatcagactctcctatctcttccttgcatgcaataactgtgtaaatatacgttttgctctatgagattgtacctttggacgaacttggagccccgccgtggcctgtgtttgtttctattttgatctgtggagaggctagggtatttaggtgtgcagattcatcattgtaaactggtttgtatggtcggggaactgttggatcactcttgcgccaccgttgccctgttgccccgttgccccgttgccctgttgcctggcaggtgacatgattagatccactgcatttcctcgccgtttttaaattcaactatctctttctctctgataatttgcgtgggtgacaatcatggcagtccatagacatcgtcagcttcagctggagctgattctgatggtcgcagactatttgcgacctttagatcttttaaatttggttcagggcatacctcaaattgcttcccttctctcctctcgacatatgcgagcccaagatgacaatggattcactatatttcatctgattgtggaacaggggcttaaaagcctaatagaacgcctcgataaatggatcccacggggctttattcccgacaacgaagggtggacacctcttcatcaagaagttaggaaggctaattaccggatggtaaaagcactgattgatgctggctcagatttatcagcccggggttataatggaaaaacttcccttcaccttgcctgcggtggaaatgcagttgaaattgtcaggttgctcttagaccatggtgcaaacccgtccgcaaaggattacgacgaaaggacgcctttgcacgacgcctattctcatgacatatctgtccgtcagatgcttattacagccggcgcggaccttgacccctgccaagtgccacgggggttgacccccctttattatgaacctatgatagcccgagaaagcgttattaggattcttcttgaggctggggcagacctttccattcaagatatgagtggggatactatattacaccgcgctacgatccacaatttcgctaatgtcgtccggctgttacttgaattcggtgttgatatcactgtgcgaaatgtccacggttataccccggttttggttgctgcaattggcgggtcagatgaatgtctacgactgctcctcaaggccggcgcagacctttcagtcatggataatcatggacgtagcgctttgcatatagcagctcgggcgggaagggaatctacagtccgactgcttgtgaaacaaggaatggacatcgccgcacgggacagccgccgtggctggactccgatgtgctgggcggtaaactacgaacagaaaggggtaatccaagttttagaagatacgcaaacaaaccgatttgcgaagtttgcacggcgagtacgcatcaaattttaaatgccatttttgctcagagcgctaggcatgtcgtggttccggatttcctgtagccagataagcctcatatatcactctttcacca

SEQ ID NO: 34 - DNA, 20 bp, Artificial
  Primer 180
ggtagaaaaggggttgcgat

SEQ ID NO: 35 - DNA, 18 bp, Artificial
  Primer 181
gaagcaatcgtcggaagt

SEQ ID NO: 36 - DNA, 21 bp, Artificial
  Primer 182
cgcgaggatgtatggtatgat

SEQ ID NO: 37 - DNA, 21 bp, Artificial
  Primer 183
gaaagtgagattgtggatgga

SEQ ID NO: 38 - DNA, 17 bp, Artificial
  Primer 184
tgtcgttcatggctccc

SEQ ID NO: 39 - DNA, 18 bp, Artificial
  Primer 185
aaggtgcgaactggagag

SEQ ID NO: 40 - DNA, 22 bp, Artificial
  Primer 186
ggaacaatcggcaaagaaagtg

SEQ ID NO: 41 - DNA, 19 bp, Artificial
  Primer 187
caacttcttgtggcgttgg

SEQ ID NO: 42 - DNA, 20 bp, Artificial
  Primer 212
ctcggtcgctcttttgggta

SEQ ID NO: 43 - DNA, 20 bp, Artificial
  Primer 213
caggacattgttggagccga

SEQ ID NO: 44 - DNA, 20 bp, Artificial
  Primer 214
tccggaagtgcttgacattg

SEQ ID NO: 45 - DNA, 22 bp, Artificial
  Primer 215
ccatcccgagtattctttcgag

SEQ ID NO: 46 - DNA, 22 bp, Artificial
  Primer 216
ccgtgcgctataataaccttcg

SEQ ID NO: 47 - DNA, 16 bp, Artificial
  Primer 217
gcgtcccggaagttcg

SEQ ID NO: 48 - DNA, 18 bp, Artificial
  Primer 218
gagcggtcgagttctgga

SEQ ID NO: 49 - DNA, 23 bp, Artificial
  Primer 219
ggtgaaagagtgatatatgaggc

SEQ ID NO: 50 - DNA, 21 bp, Artificial
  Primer 220
tctcaggacttgcagatgttg

SEQ ID NO: 51 - DNA, 19 bp, Artificial
  Primer 221
gccaagtcatcatcctcgc

SEQ ID NO: 52 - DNA, 20 bp, Artificial
  Primer 222
cttactgccggtgattcgat

SEQ ID NO: 53 - DNA, 22 bp, Artificial
  Primer 223
ctacaggacacacattcatcgt

SEQ ID NO: 54 - DNA, 18 bp, Artificial
  Primer 224
tgccagccctcctattcc

SEQ ID NO: 55 - protein, 447 aa, Cupriavidus basilensis
MEAVAKKRTETISEALPAATNRQVFGAVTASCMGWALDLFDLFILLFVAPVIGRLFFPSEHAMLSLAAVYASFAVTLLMRPLGSAIFGTYADRHGRKGAMVVAVTGVGLSTAAFGLLPTVGQVGLLAPALFILLRLVQGIFVGGVVASTHTIGTESVPPSWRGAVSGLVGGGGAGIGALLASITYMAMTALFPGEAFDAWGWRCMFFSGIISSVLGLFIFNSLEESPLWKQLQAAKGHAAPVENPLRVIFSRQYRGVLFVNILLTVGGGSAYYLTSGYLPTFLKVVVKAPAGASAAILMASSVGVIVASIIAGHLSTLIGRKRAFLLIGALNVVLLPLIYQRMPAAPDVTTLGIYAVALAMLGSTGFAPILIFLNERFPTSIRATGTGLSWNIGFAIGGMMPTFASLCASTPADLPKVLGIFVAVVTAIYLAGAAIVPETAGRLGEK